(19) United States
(12) Patent Application Publication
(10) Pub. No.: US 2025/0331419 A1
(43) Pub. Date: Oct. 23, 2025

(54) COMPOUND, LIGHT EMITTING MATERIAL, AND ORGANIC LIGHT EMITTING DEVICE

(71) Applicant: KYULUX, INC., Fukuoka-shi, Fukuoka (JP)

(72) Inventors: Umamahesh BALIJAPALLI, Fukuoka-shi, Fukuoka (JP); Saidalimu IBRAYIM, Fukuoka-shi, Fukuoka (JP); Yoshitake SUZUKI, Fukuoka-shi, Fukuoka (JP); Takahiro KASHIWAZAKI, Fukuoka-shi, Fukuoka (JP); Takuya HIGA, Fukuoka-shi, Fukuoka (JP); Yuta WATABIKI, Fukuoka-shi, Fukuoka (JP); Aiko GOTO, Fukuoka-shi, Fukuoka (JP); Tomoki YUKAWA, Fukuoka-shi, Fukuoka (JP); Masataka YAMASHITA, Fukuoka-shi, Fukuoka (JP); Kousei KANAHARA; Yuba Raj GAIHRE, Fukuoka-shi, Fukuoka (JP); Hiroaki OZAWA, Fukuoka-shi, Fukuoka (JP)

(21) Appl. No.: 18/859,286

(22) PCT Filed: Mar. 14, 2023
(86) PCT No.: PCT/JP2023/009782

§ 371 Date: Oct. 23, 2024

(30) Foreign Application Priority Data
Apr. 28, 2022 (JP) 2022-073952

(51) Int. Cl. H10K 85/60; C09K 11/02; H10K 50/11; H10K 101/20

(52) U.S. Cl. CPC H10K 85/6572; C09K 11/02; H10K 85/657; H10K 50/11; H10K 2101/20

(57) ABSTRACT

The compound represented by the following general formula is useful for a light emitting device. Ar¹ represents a benzene ring, a naphthalene ring, a phenanthrene ring, etc.; D represents 5H-benzofuro[3,2-c]carbazol-5-yl group, etc.; A represents a cyano group, a phenyl group, a pyrimidyl group, a triazyl group, etc.; m is 1 to 3; n is 0 to 2; R¹ to R⁴ each represent H, an aryl group, a cyano group, etc.

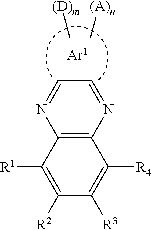

### TECHNICAL FIELD

The present invention relates to a compound having good light emission characteristics. Also, the present invention relates to a light emitting material and an organic light emitting device using the compound.

### BACKGROUND ART

An organic light emitting device is a light emitting device using an organic material, which can be produced by coating and which does not use a rare element, and therefore, attention has recently been paid to the organic light emitting device. Above all, an organic electroluminescent device (organic EL device) emits self-luminous light and does not require a backlight, and is therefore advantageous in that it can be a lightweight and flexible device. In addition, it has features of high responsiveness and high visibility, and is expected as a next generation light source. Consequently, studies relating to development of materials useful for organic light emitting devices such as organic electroluminescent devices have been promoted actively. In particular, studies relating to light emitting materials have been carried out actively (for example, NPL 1).

### CITATION LIST

### Non-Patent Literature

NPL 1: Chem. Soc. Rev., 2017,46,915

### SUMMARY OF INVENTION

### Technical Problem

On the other hand, there is still room for improvement in the light emission characteristics of organic light emitting devices, and further enhancement of emission properties is desired.

Accordingly, the present inventor have promoted assiduous studies for the purpose of developing a novel compound capable of contributing toward improvement of light emission characteristics of organic light emitting devices.

### Solution to Problem

As a result of assiduous studies, the present inventors have found that a compound having a specific skeleton with groups each having a characteristic structure bonding to the skeleton is a compound useful for light emitting devices. The present invention has been proposed on the basis of such findings, and has the following constitution.

[1] A compound represented by the following general formula (1).

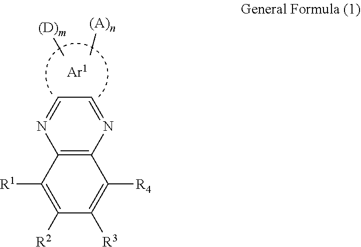

In the general formula (1), Ar¹ represents a cyclic structure, and represents a benzene ring, a naphthalene ring, an anthracene ring, or a phenanthrene ring. D represents a donor group, and at least one D is a group represented by the following general formula (2). A represents one or a combination of two or more groups selected from the group consisting of a cyano group, a phenyl group, a pyrimidyl group, a triazyl group and an alkyl group (except for a substituted alkyl group). m represents 1, 2 or 3, n represents 0, 1 or 2. When m is 2 or 3, plural D's can be the same or different. When n is 2, two A's can be the same or different. R¹ to R⁴ each independently represent a hydrogen atom, a deuterium atom, or one or a combination of two or more groups selected from the group consisting of an alkyl group, an aryl group, a heteroaryl group and a cyano group. R¹ and R², and R³ and R⁴ each can bond to each other to form a cyclic structure selected from the group consisting of a benzene ring, a naphthalene ring and a pyridine ring, and the formed cyclic structure can be substituted with one or a combination of two or more groups selected from the group consisting of an alkyl group, an aryl group, a heteroaryl group and a cyano group.

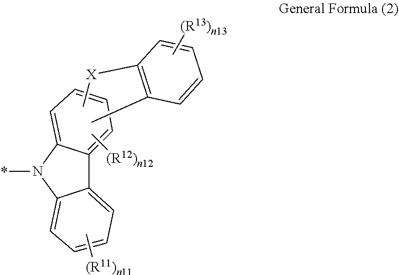

In the general formula (2), X represents O, S or N—R¹⁴. R¹¹ to R¹³ each independently represent a deuterium atom, or a substituent. R¹⁴ represents an aryl group optionally substituted with one or more selected from the group consisting of a deuterium atom, an alkyl group and an aryl group, or an alkyl group optionally substituted with one or more selected from the group consisting of a deuterium atom and an aryl group. R¹¹ to R¹³ do not bond to any of R¹¹ to R¹⁴ to form a cyclic structure. n11 and n13 each independently represent an integer of 0 to 4, and n12 represents an integer of 0 to 2.

[2] The compound according to [1], represented by the following general formula (3).

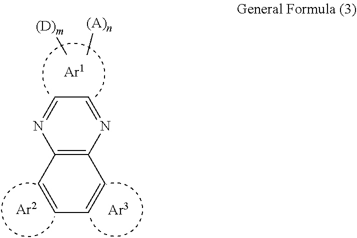

In the general formula (3), Ar¹ represents a cyclic structure, and represents a benzene ring, a naphthalene ring, an anthracene ring, or a phenanthrene ring. D represents a donor group, and at least one D is a group represented by the above general formula (2). A represents one or a combination of two or more groups selected from the group consisting of a cyano group, a phenyl group, a pyrimidyl group, a triazyl group and an alkyl group (except for a substituted alkyl group). m represents 1, 2 or 3, n represents 0, 1 or 2. When m is 2 or 3, plural D's can be the same or different. When n is 2, two A's can be the same or different. Ar² and Ar³ each can independently form a cyclic structure selected from the group consisting of a benzene ring, a naphthalene ring and a pyridine ring, and the formed cyclic structure can be substituted with one or a combination of two or more groups selected from the group consisting of an alkyl group, an aryl group, a heteroaryl group and a cyano group.

[3] The compound according to [1], having a skeleton of any of the following:

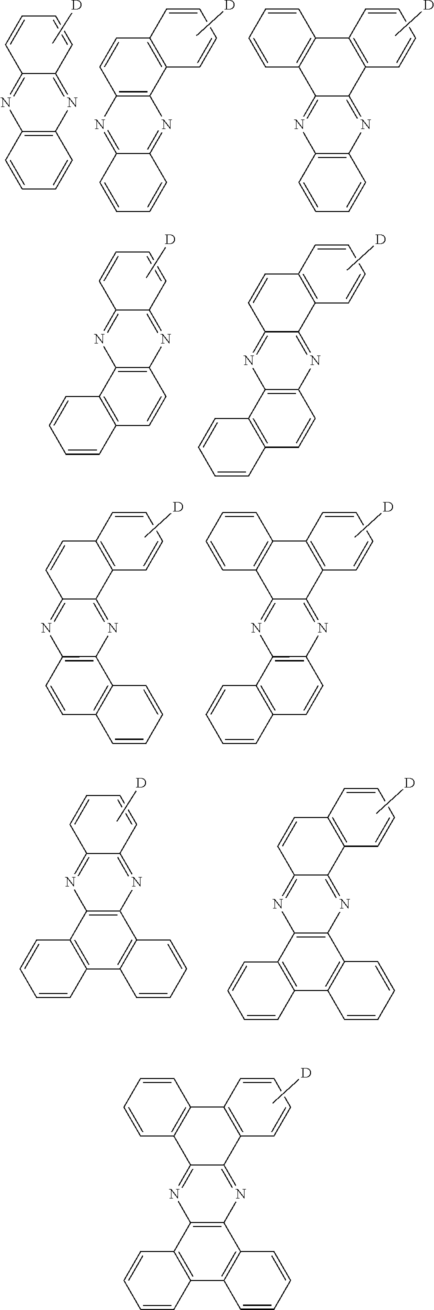

The above skeletons each can have a substituent within the range of the general formula (1), but any further ring is not fused with the skeletons.

[4] The compound according to [1], represented by any of the following general formulae (4a) to (4g).

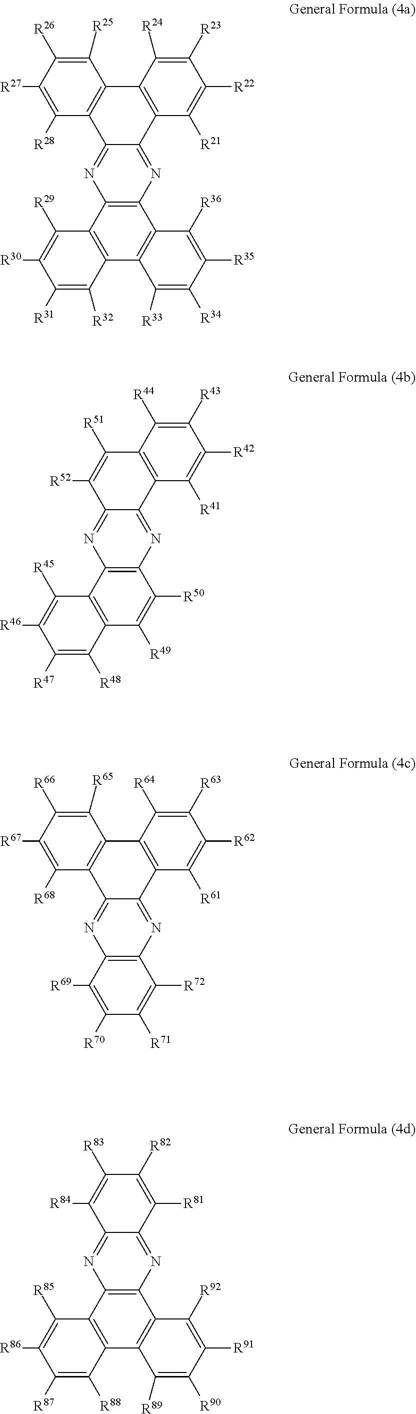

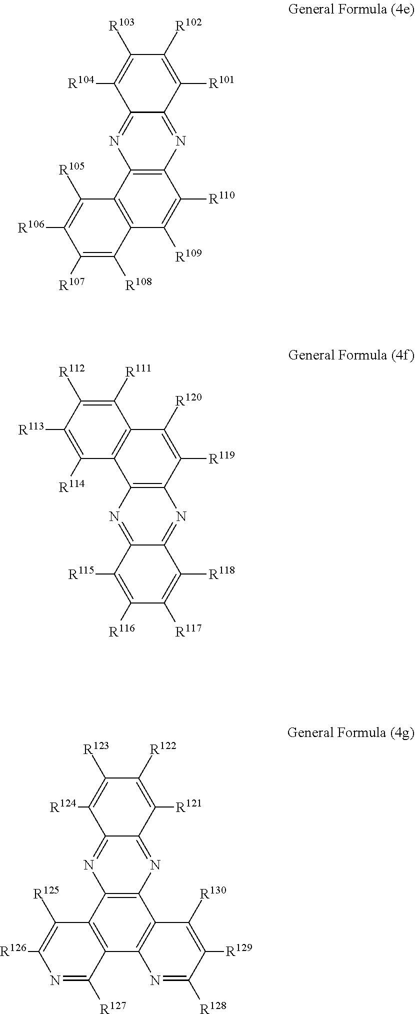

In the general formulae (4a) to (4g), R²¹ to R²⁸, R⁴¹ to R⁴⁴, R⁵¹, R³², R⁶¹ to R⁶⁸, R⁸¹ to R⁸⁴, R¹⁰¹ to R¹⁰⁴, R¹¹¹ to R¹¹⁴, R¹¹⁹, R¹²⁰ and R¹²¹ to R¹²⁴ each independently represent a hydrogen atom, a deuterium atom, D or A. Provided that 1 to 3 of R²¹ to R²⁸ are D, and 0 to 2 are A; 1 to 3 of R⁴¹ to R⁴⁴, R⁵¹ and R⁵² are D, and 0 to 2 are A; 1 to 3 of R⁶¹ to R⁶⁸ are D, and 0 to 2 are A; 1 to 3 of R⁸¹ to R⁸⁴ are D, and 0 to 2 are A; 1 to 3 of R¹⁰¹ to R¹⁰⁴ are D and 0 to 2 are A; 1 to 3 of R¹¹¹ to R¹¹⁴, R¹¹⁹ and R¹²⁰ are D, and 0 to 2 are A; 1 to 3 of R¹²¹ to R¹²⁴ are D, and 0 to 2 are A. R²⁹ to R³⁶, R⁴⁵ to R⁵⁰, R⁶⁹ to R⁷², R⁸⁵ to R⁹², R¹⁰⁵ to R¹¹⁰, R¹¹⁵ to R¹¹⁸, and R¹²⁵ to R¹³⁰ each independently represent a hydrogen atom, a deuterium atom, or one or a combination of two or more groups selected from the group consisting of an alkyl group, an aryl group and a cyano group.

[5] The compound according to any one of [1] to [4], wherein n is 0.

[6] A light emitting material containing the compound of any one of [1] to [5].

[7] A host material containing the compound of any one of [1] to [5].

[8] A film containing the compound of any one of [1] to [5].

[9] An organic semiconductor device containing the compound of any one of [1] to [5].

[10] An organic light emitting device containing the compound of any one of [1] to [5].

[11] The organic light emitting device according to [10], wherein the device has a layer containing the compound and the layer also contains a host material.

[12] The organic light emitting device according to [11], wherein the layer containing the compound also contains a delayed fluorescent material in addition to the host material, and the lowest excited singlet energy of the delayed fluorescent material is lower than that of the host material and higher than that of the compound.

[13] The organic light emitting device according to [10], wherein the device has a layer containing the compound, and the layer also contains a light emitting material having a structure different from that of the compound.

[14] The organic light emitting device according to any one of [10] to [13], wherein, among the materials contained in the device, the amount of light emission from the compound is the maximum.

[15] The organic light emitting device according to [13], wherein the amount of light emission from the light emitting material is larger than the amount of light emission from the compound.

[16] The organic light emitting device according to any one of [10] to [15], which emits delayed fluorescence.

### Advantageous Effects of Invention

The compound of the present invention is a compound useful for light emitting devices. The compound of the present invention includes a compound having excellent light emission characteristics (for example, a compound having a high light emission efficiency), and a compound excellent as a host material in a light emitting layer. The compound of the present invention can be used as a light emitting material and a host material in a light emitting device, and using the compound of the present invention, an organic light emitting device can be produced. An organic light emitting device using the compound of the present invention has excellent light emission characteristics.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the present description, a numerical range expressed using “to” means a range that includes the numerical values described before and after “to” as the lower limit and the upper limit.

A part or all of hydrogen atoms existing in the molecule of the compound for use in the present invention can be substituted with deuterium atoms (²H, deuterium D). In the chemical structural formulae in the present description, the hydrogen atom is expressed as H, or the expression thereof is omitted. For example, when expression of the atoms bonding to the ring skeleton-constituting carbon atoms of a benzene ring is omitted, H is considered to bond to the ring skeleton-constituting carbon atom at the site having the omitted expression. In the present description, the term “substituent” means an atom or an atomic group except a hydrogen atom and a deuterium atom. On the other hand, the term “substituted or unsubstituted” means that a hydrogen atom can be substituted with a deuterium atom or a substituent.

### [Compound Represented by General Formula (1)]

The compound of the present invention is a compound represented by the following general formula (1).

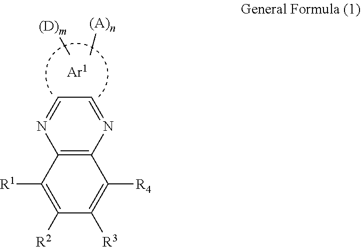

In the general formula (1), Ar¹ represents a cyclic structure, and represents a benzene ring, a naphthalene ring, an anthracene ring, or a phenanthrene ring. For example, when Ar¹ represents a benzene ring, the benzene ring is fused with the pyrazine ring to be a quinoxaline structure. When Ar¹ represents a naphthalene ring, any of a 1,2-naphtho ring or a 2,3-naphtho ring can be fused with the pyrazine ring. When a 1,2-naphtho ring is fused with the pyrazine ring, the 1-positioned and 2-positioned carbon atoms of the naphthalene ring covalently bond to the 2-positioned and 3-positioned carbon atoms, respectively, constituting the pyrazine ring. When Ar¹ represents an anthracene ring, a 2,3-anthracene ring is fused with the pyrazine ring. When Ar¹ represents a phenanthrene ring, any of a 1,2-phenanthrene ring, a 2,3-phenanthrene ring, a 3,4-phenanthrene ring or a 9,10-phenanthrene ring can be fused with the pyrazine ring. In a preferred embodiment of the present invention, any of a benzene ring, a 2,3-naphtho ring, or a 9,10-phenanthrene ring is fused with the pyrazine ring. In a more preferred embodiment of the present invention, any of a 2,3-naphtho ring or a 9,10-phenanthrene ring is fused with the pyrazine ring. For example, a 2,3-naphtho ring can be fused, or a 9,10-phenanthrene ring can be fused.

In the cyclic structure that Ar¹ represents, m D's and n A's bond to the ring skeleton as substituents. When Ar¹ represents a naphthalene ring, an anthracene ring or a phenanthrene ring, D and A can bond to any benzene ring constituting these rings. m D's and n A's can bond to any one benzene ring alone, and neither D nor A cannot bond to the other benzene rings. Or a part of m D's and n A's can bond to one benzene ring, and the rest thereof can bond to the other one benzene ring. In one preferred embodiment of the present invention, n is 0 and m D's bond to one benzene ring alone. In another preferred embodiment of the present invention, n is 0, and a part of m D's bond to one benzene ring and the rest thereof bond to the other one benzene ring. When Ar¹ represents a naphthalene ring, an anthracene ring or a phenanthrene ring, in one preferred embodiment of the present invention, neither D nor A bonds to the benzene ring directly fused with the pyrazine ring, and m D's and n A's bond to only the remaining benzene ring (that is, the benzene ring not directly fused with the pyrazine ring). When Ar¹ represents a naphthalene ring, an anthracene ring or a phenanthrene ring, in one preferred embodiment of the present invention, n is 0, and D does not bond to the benzene ring directly fused with the pyrazine ring, and m D's bond to only the remaining benzene ring (that is, the benzene ring not directly fused with the pyrazine ring).

In the general formula (1), m is 1, 2 or 3, and n is 0, 1 or 2. When m is 2 or 3, plural D's can be the same or different. Two D's can bond to the same benzene ring, or to different benzene rings. When n is 2, two A's can be the same or different. Two A's can bond to the same benzene ring, or to different benzene rings. In one preferred embodiment of the present invention, n is 0. For example, m is 1 and n is 0. For example, m is 2 and n is 0. For example, m is 3 and nis 0. In one aspect of the present invention, m is 1 or 2. In one aspect of the present invention, m is 3. When Ar¹ represents a naphthalene ring, an anthracene ring or a phenanthrene ring, and n is 1 or 2, in one embodiment of the present invention, A does not bond to the benzene ring to which D bonds, and D does not bond to the benzene ring to which A bonds.

In the general formula (1), D represents a donor group. The donor group can be selected from groups having a negative Hammett's σp value. The Hammett's σp value is proposed by L. P. Hammett and quantifies the influence of a substituent on the reaction rate or equilibrium of a para-substituted benzene derivative. Specifically, the value is a constant (σp) peculiar to the substituent in the following equation that is established between a substituent and a reaction rate constant or an equilibrium constant in a para-substituted benzene derivative:

$$log ( k / {k}_{0} ) = p σ p$$

$$or$$

$$log ( K / {K}_{0} ) = p σ p$$

In the above equations, ko represents a rate constant of a benzene derivative not having a substituent; k represents a rate constant of a benzene derivative substituted with a substituent; K₀ represents an equilibrium constant of a benzene derivative not having a substituent; K represents an equilibrium constant of a benzene derivative substituted with a substituent; and p represents a reaction constant to be determined by the kind and the condition of reaction. Regarding the description relating to the “Hammett's σp value” and the numerical value of each substituent in the present invention, reference can be made to the description relating to σp value in Hansch, C. et. al., Chem. Rev., 91, 165-195 (1991).

The donor group which D can represent preferably has σp of −0.3 or less, more preferably −0.5 or less, and even more preferably −0.7 or less. For example, the value can be selected from a range of −0.9 or less, or from a range of −1.1 or less.

The donor group in the present invention is preferably a group containing a substituted amino group. The donor group can be a substituted amino group, or can be a substituted amino group-bonded aryl group, especially a substituted amino group-bonded phenyl group. In one preferred aspect of the present invention, the donor group is a substituted amino group.

The substituent bonding to the nitrogen atom of a substituted amino group is preferably a substituted or unsubstituted alkyl group, a substituted or unsubstituted alkenyl group, a substituted or unsubstituted aryl group, or a substituted or unsubstituted heteroaryl group, more preferably a substituted or unsubstituted aryl group or a substituted or unsubstituted heteroaryl group. Especially, the substituted amino group is preferably a substituted or unsubstituted diarylamino group, or a substituted or unsubstituted diheteroarylamino group. As referred to herein, the two aryl groups constituting the diarylamino group can bond to each other, and the two heteroaryl groups constituting the diheteroarylamino group can bond to each other.

The “aryl group” can be a monocyclic ring or a fused ring in which two or more rings are fused. In the case of a fused ring, the number of fused rings is preferably 2 to 6, and can be selected from, for example, 2 to 4. Specific examples of the ring include a benzene ring, a naphthalene ring, an anthracene ring, a phenanthrene ring, and a triphenylene ring. In one aspect of the present invention, the aryl group is a substituted or unsubstituted phenyl group, a substituted or unsubstituted naphthalen-1-yl group, or a substituted or unsubstituted naphthalen-2-yl group, and is preferably a substituted or unsubstituted phenyl group. For example, the substituent for the aryl group can be selected from Substituent Group A, can be selected from Substituent Group B, can be selected from Substituent Group C, can be selected from Substituent Group D, or can be selected from Substituent Group E. In one aspect of the present invention, the substituent for the aryl group is at least one selected from the group consisting of an alkyl group, an aryl group and a deuterium atom. In one preferred aspect of the present invention, the aryl group is unsubstituted.

The “heteroaryl group” can be a monocyclic ring or a fused ring in which two or more rings are fused. In the case of a fused ring, the number of fused rings is preferably 2 to 6, and can be selected from, for example, 2 to 4. Specific examples of the ring include a pyridine ring and a pyrimidine ring, and these rings can be fused with any other ring. Specific examples of the heteroaryl group include a 2-pyridyl group, a 3-pyridyl group and a 4-pyridyl group. The number of the ring skeleton-constituting atoms of the heteroaryl group is preferably 4 to 40, more preferably 5 to 20, and can be selected from a range of 5 to 14, or can be selected from a range of 5 to 10.

The “alkyl group” can be any of linear, branched or cyclic ones. Two or more of a linear moiety, a cyclic moiety and a branched moiety can exist therein as combined. The carbon number of the alkyl group can be, for example 1 or more, 2 or more, or 4 or more. The carbon number can also be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, an isohexyl group, a 2-ethylhexyl group, an n-heptyl group, an isoheptyl group, an n-octyl group, an isooctyl group, an n-nonyl group, an isononyl group, an n-decanyl group, an isodecanyl group, a cyclopentyl group, a cyclohexyl group, and a cycloheptyl group. The alkyl group which is the substituent can be further substituted with, for example, a deuterium atom, an aryl group, an alkoxy group, an aryloxy group, and a halogen atom. In one aspect of the present invention, the substituent for the alkyl group is at least one selected from the group consisting of an aryl group and a deuterium atom. In one preferred aspect of the present invention, the alkyl group is unsubstituted.

The “alkenyl group” can be linear, branched or cyclic. Two or more of a linear moiety, a cyclic moiety and a branched moiety can exist therein as combined. The carbon number of the alkenyl group can be, for example 2 or more, or 4 or more. The carbon number can also be 30 or less, 20 or less, 10 or less, 6 or less, or 4 or less. Specific examples of the alkenyl group include an ethenyl group, an n-propenyl group, an isopropenyl group, an n-butenyl group, an isobutenyl group, an n-pentenyl group, an isopentenyl group, an n-hexenyl group, an isohexenyl group, and a 2-ethylhexenyl group. The alkenyl group which is the substituent can be further substituted with a substituent.

The donor group which D can represent is preferably a group represented by the following general formula (a).

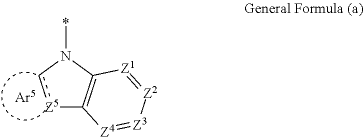

In the general formula (a), Z¹ represents C—R^{1A} or N, Z² represents C—R^{2A} or N, Z³ represents C—R^{3A} or N, and Z⁴ represents C—R^{4A} or N. Z⁵ represents C or N, Ar⁵ represents a substituted or unsubstituted aromatic ring or a substituted or unsubstituted heteroaromatic ring. R^{1A} and R^{2A}, R^{2A} and R^{3A}, and R^{3A} and R^{4A} each can bond to each other to form a cyclic structure.

Among Z¹ to Z⁴, the number of groups represented by N is preferably 0 to 3, and preferably 0 to 2. In one aspect of the present invention, among Z¹ to Z⁴, the number of groups represented by N is 1. In one aspect of the present invention, among Z¹ to Z⁴, the number of groups represented by Nis 0.

R^{1A} to R^{4A} each independently represent a hydrogen atom, a deuterium atom, or a substituent.

The substituent can be selected from, for example, Substituent Group A, or can be selected from Substituent Group B, or can be selected from Substituent Group C, or can be selected from Substituent Group D, or can be selected from Substituent Group E. When two or more of R^{1A} to R^{4A} represent substituents, these two or more substituents can be the same or different. 0 to 2 of R^{1A} to R^{4A} are preferably a substituent, and for example, one can be a substituent, or zero can be a substituent (R^{1A} to R^{4A} are a hydrogen atom or a deuterium atom).

R^{1A} and R^{2A}, R^{2A} and R^{3A}, and R^{3A} and R^{4A} each can bond to each other to form a cyclic structure. The cyclic structure can be any of an aromatic ring, a heteroaromatic ring, an aliphatic hydrocarbon ring, or an aliphatic heteroring, and can also be a fused ring thereof. The structure is preferably an aromatic ring or a heteroaromatic ring. Examples of the aromatic ring include a substituted or unsubstituted benzene ring. Another benzene ring can be further fused to the benzene ring, and a heterocyclic ring such as a pyridine ring can be fused to the benzene ring. The heteroaromatic ring means a ring exhibiting aromaticity including a heteroatom as a ring skeleton-constituting atom, and is preferably a 5- to 7-membered ring, and for example, a 5-membered ring or a 6-membered ring can be employed. In one embodiment of the present invention, the heteroaromatic ring includes a furan ring, a thiophene ring and a pyrrole ring. In one preferred embodiment of the present invention, the cyclic structure is a furan ring of a substituted or unsubstituted benzofuran, a thiophene ring of a substituted or unsubstituted benzothiophene, or a pyrrole ring of a substituted or unsubstituted indole. Benzofuran, benzothiophene and indole as referred to herein can be unsubstituted or can be substituted with a substituent selected from Substituent Group A, or can be substituted with a substituent selected from Substituent Group B, or can be substituted with a substituent selected from Substituent Group C, or can be substituted with a substituent selected from Substituent Group D, or can be substituted with a substituent selected from Substituent Group E. It is preferable that a substituted or unsubstituted aryl group bonds to the nitrogen atom constituting the pyrrole ring of indole, and examples of the substituent include a substituent selected from any of Substituent Group A to Substituent Group E. The cyclic structure can be a substituted or unsubstituted cyclopentadiene ring. In one aspect of the present invention, a pair of R^{1A} and R^{2A}, R^{2A} and R^{3A}, and R^{3A} and R^{4A} each bond to each other to form a cyclic structure. In one aspect of the present invention, R^{1A} and R^{2A}, R^{2A} and R^{3A}, and R^{3A} and R^{4A} each do not bond to each other to form a cyclic structure.

In the general formula (a), Z⁵ represents C or N, Ar⁵ represents a substituted or unsubstituted aromatic ring or a substituted or unsubstituted heteroaromatic ring. In one aspect of the present invention, Z⁵ is C, and Ar⁵ is a substituted or unsubstituted aromatic ring or a substituted or unsubstituted heteroaromatic ring. In one aspect of the present invention, Z⁵ is N, and Ar⁵ is a substituted or unsubstituted heteroaromatic ring.

Examples of the aromatic ring which Ar⁵ can represent include a benzene ring. Another benzene ring can be further fused to the benzene ring, and a heterocyclic ring such as a pyridine ring can be fused to the benzene ring. The heteroaromatic ring which Ar⁵ can represent is preferably a 5- to 7-membered ring, and for example, a 5-membered ring or a 6-membered ring can be employed. In one aspect of the present invention, as the heteroaromatic ring, a furan ring, a thiophene ring, a pyrrole ring, an imidazole ring, a pyridine ring, a pyridazine ring, a pyrimidine ring, or a pyrazine ring can be employed. In one aspect of the present invention, Z⁵ is C, and the heteroaromatic ring is a furan ring of a substituted or unsubstituted benzofuran, a thiophene ring of a substituted or unsubstituted benzothiophene, a pyridine ring of a substituted or unsubstituted quinoline, or a pyridine ring of a substituted or unsubstituted isoquinoline. In one aspect of the present invention, Z⁵ is N, and the heteroaromatic ring is a pyrrole ring of a substituted or unsubstituted indole, or an imidazole ring of a substituted or unsubstituted benzimidazole. The benzofuran, benzothiophene, quinoline, isoquinoline, indole and benzimidazole referred to herein can be unsubstituted, or can be substituted with a substituent selected from Substituent Group A, can be substituted with a substituent selected from Substituent Group B, can be substituted with a substituent selected from Substituent Group C, can be substituted with a substituent selected from Substituent Group D, and can be substituted with a substituent selected from Substituent Group E.

In one preferred aspect of the present invention, all D's in the general formula (1) are substituted amino groups, and more preferably substituted or unsubstituted carbazol-9-yl groups. In one aspect of the present invention, at least one D is a substituted amino group not containing a carbazole structure.

In the general formula (1), at least one D represents a group represented by the following general formula (2).

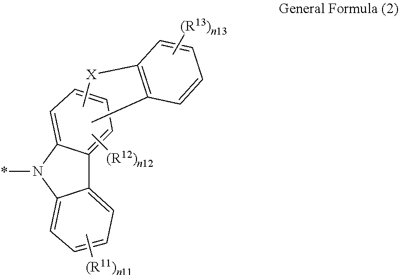

In the general formula (2), X represents O, S or N—R¹⁴. R¹⁴ represents an aryl group optionally substituted with one or more selected from the group consisting of a deuterium atom, an alkyl group and an aryl group, or an alkyl group optionally substituted with one or more selected from the group consisting of a deuterium atom and an aryl group. Here, the aryl group as a substituent for the aryl group and the alkyl group can be selected from, for example, an aryl group having 6 to 22 carbon atoms, and the alkyl group as a substituent for the alkyl group and the aryl group can be selected from, for example, an alkyl group having 1 to 20 carbon atoms. In one preferred aspect of the present invention, X is O. In one preferred embodiment of the present invention, X is N—R¹⁴. For example R¹⁴ of N—R¹⁴ is an aryl group (for example, having 6 to 22 carbon atoms). The aryl group can be not substituted with or can be substituted with at least one atom or group selected from the group consisting of a deuterium atom, an alkyl group (for example, having 1 to 20 carbon atoms), and an aryl group (for example, having 6 to 22 carbon atoms). X can be an oxygen atom or a sulfur atom.

In the general formula (2), the two bonds bonding to one benzene ring of the carbazole ring bond to the neighboring positions of the benzene ring to form a fused ring structure of the carbazole ring and the hetero-fused ring containing X. For example, in the case where X is O, a benzofurocarbazole ring is formed as a fused ring structure, in the case where X is S, a benzothienocarbazole ring is formed as a fused ring structure, and in the case where X is N—R¹⁴, an indolocarbazole ring is formed as a fused ring structure. The positions to which the two bonds bond can be the 1-position and the 2-position of the carbazole ring, or can be 2-position and the 3-position of the carbazole ring, or can be 3-position and the 4-position of the carbazole ring. In the case where the bonding positions of the two bonds are the 1-position and the 2-position, the position at which the bond of X bonds can be the 1-position or the 2-position, in the case where the bonding positions of the two bonds are the 2-position and the 3-position, the position at which the bond of X bonds can be the 2-position or the 3-position, and in the case where the bonding positions of the two bonds are the 3-position and the 4-position, the position at which the bond of X bonds can be the 3-position or the 4-position.

In the general formula (2), * indicates a bonding position.

In the general formula (2), R¹¹ to R¹³ each independently represent a deuterium atom or a substituent. R¹¹ to R¹³ do not bond to any of R¹¹ to R¹⁴ to form a cyclic structure. The substituent can be selected from, for example, Substituent Group A, or can be selected from Substituent Group B, or can be selected from Substituent Group C, or can be selected from Substituent Group D, or can be selected from Substituent Group E. In one preferred embodiment of the present invention, the substituent means one or a combination of two or more groups selected from the group consisting of an alkyl group (for example, having 1 to 20 carbon atoms), an aryl group (for example, having 6 to 22 carbon atoms), and a cyano group. For example, the substituent can be a cyano group, or an aryl group optionally substituted with one or a combination of two or more groups selected from the group consisting of a cyano group and an alkyl group.

n11 and n13 each independently represent an integer of 0 to 4, and n12 represents an integer of 0 to 2. When n11 is 2 or more, two or more R¹¹'s can be the same or different. When n13 is 2 or more, two or more R¹³'s can be the same or different. When n12 is 2, two R¹²'s can be the same or different. n11 and n13 can be any number of 0, 1, 2, 3 or 4, and n12 can be any number of 0, 1 or 2. When n11 is 1, R¹¹ can be a deuterium atom, or can be a substituent. When n11 is 2 or more, all two or more R¹¹'s can be deuterium atoms, or all can be substituents, or a part thereof can be deuterium atoms and the remaining ones can be substituents. When n13 is 1, R¹³ can be a deuterium atom, or can be a substituent. When n13 is 2 or more, all two or more R¹³'s can be deuterium atoms, or all can be substituents, or a part thereof can be deuterium atoms and the remaining ones can be substituents. When n12 is 1, R¹² can be a deuterium atom, or can be a substituent. When n12 is 2, both two R¹²'s can be deuterium atoms, or both can be substituents, or one of the two can be a deuterium atom and the other can be a substituent.

Hereinafter, specific examples of D represented by the general formula (2) will be given. However, D that can be employed in the present invention should not be limitatively interpreted by the following specific examples. In the following specific examples, * indicates a bonding position. A methyl group is not shown. Consequently, D75 to D92, D167 to D184 and D244 to D258 represent structures substituted with a methyl group.

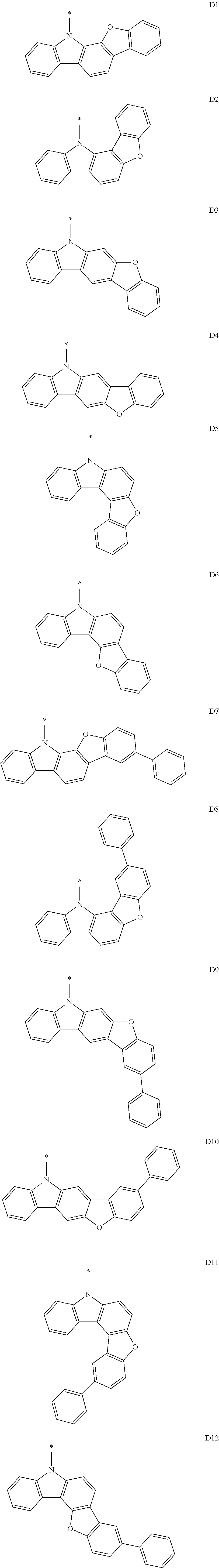

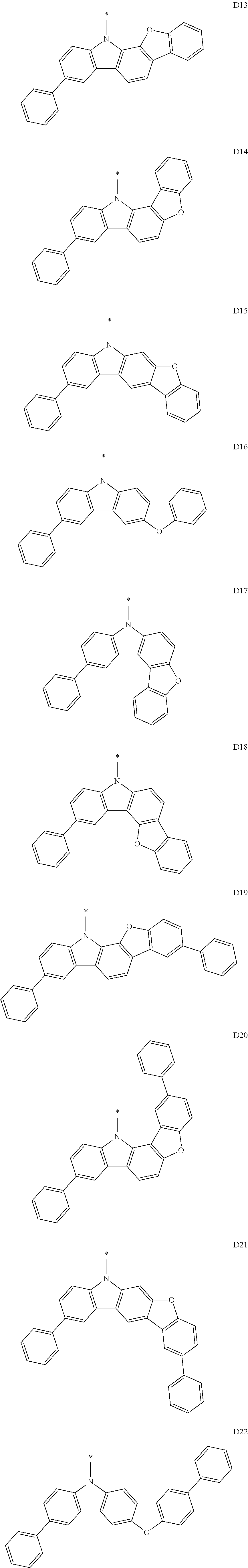

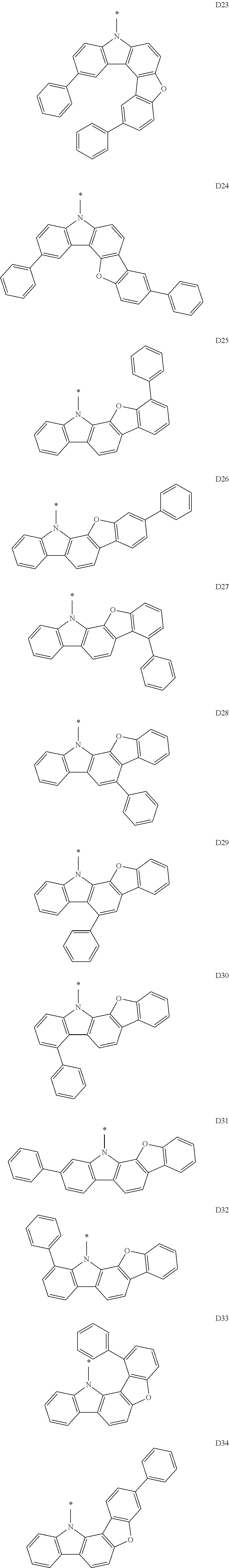

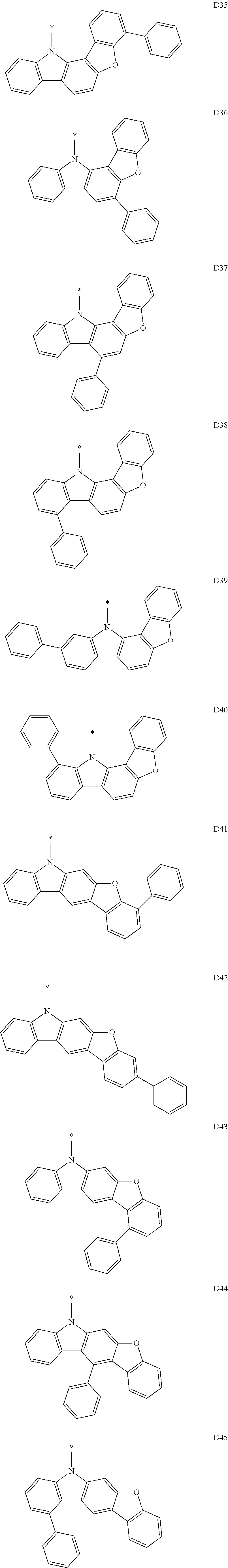

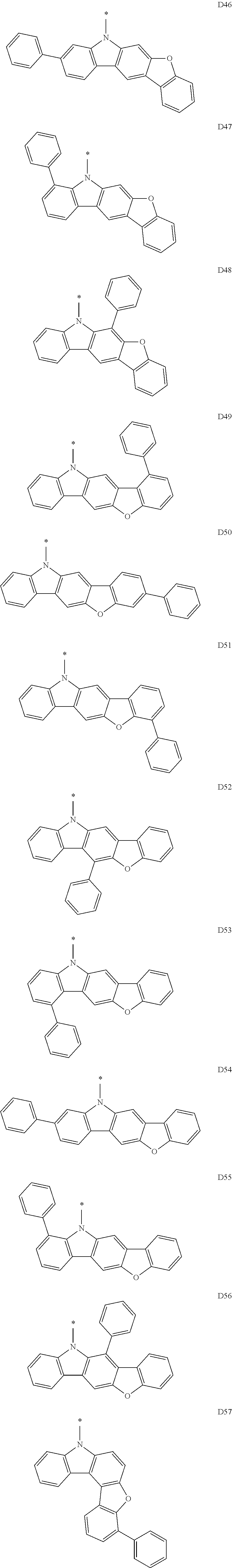

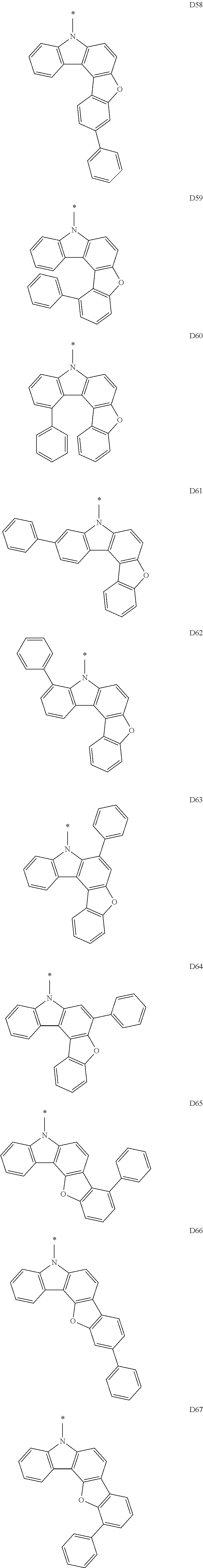

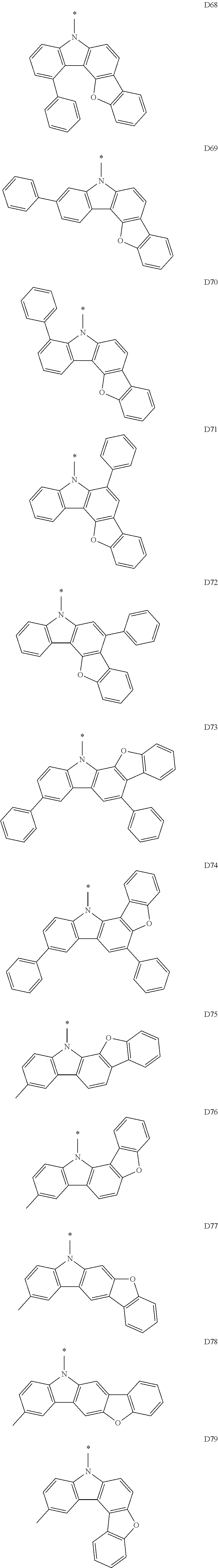

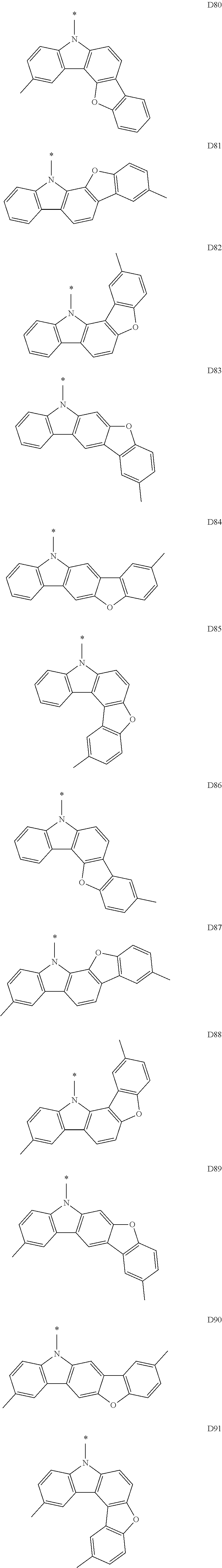

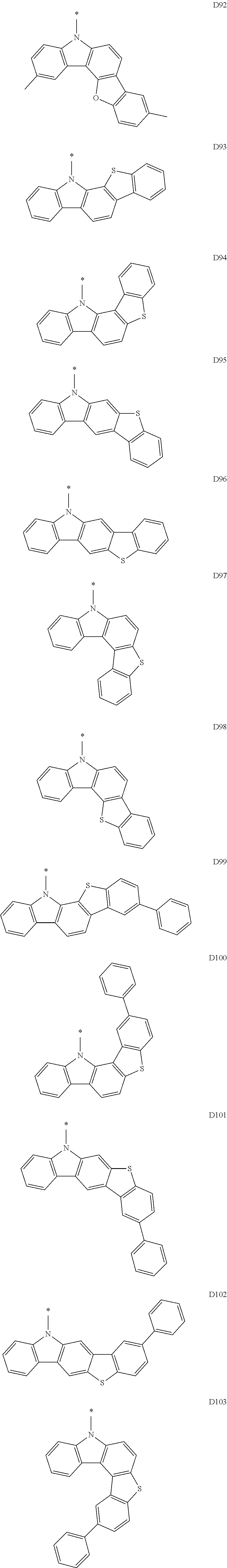

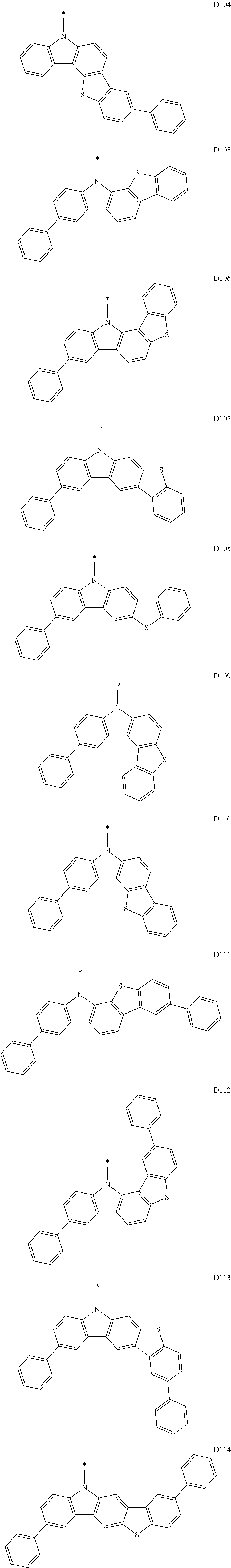

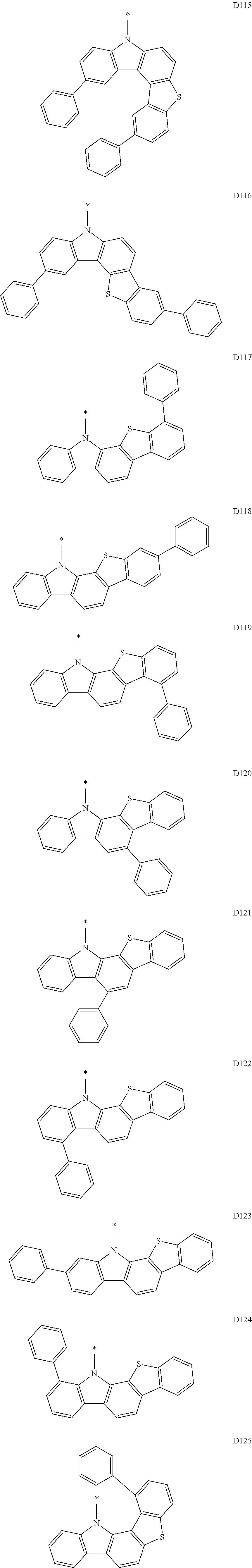

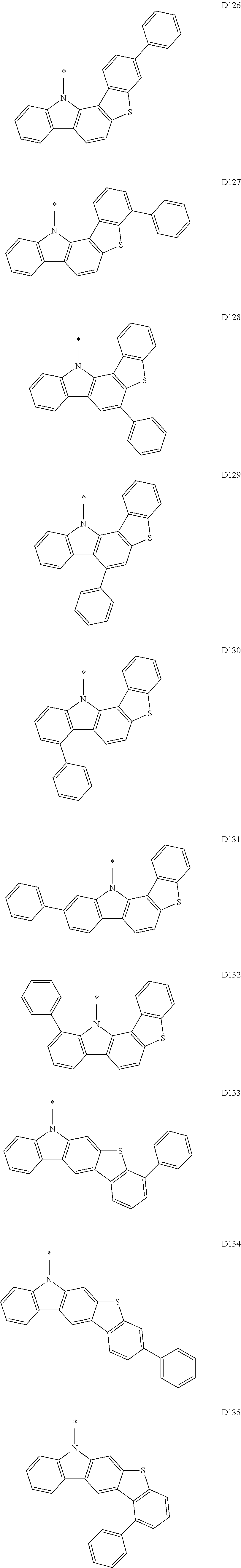

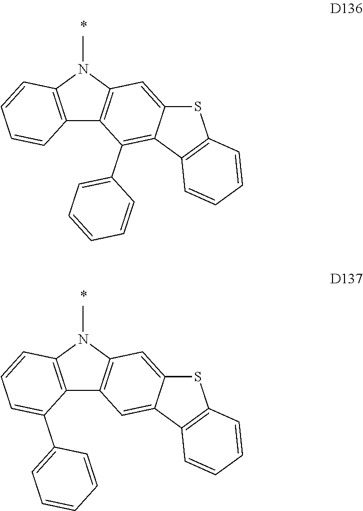 [SMILES: Cn1c2ccccc2c2c(-c3ccccc3)c3c(cc21)sc1ccccc13 ; Cn1c2cc3sc4ccccc4c3cc2c2c(-c3ccccc3)cccc21]

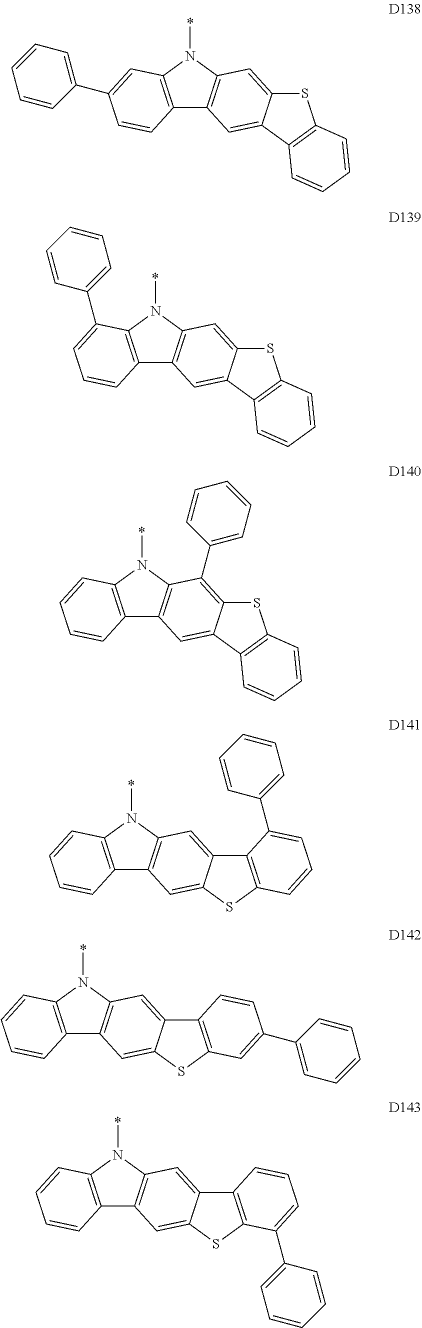

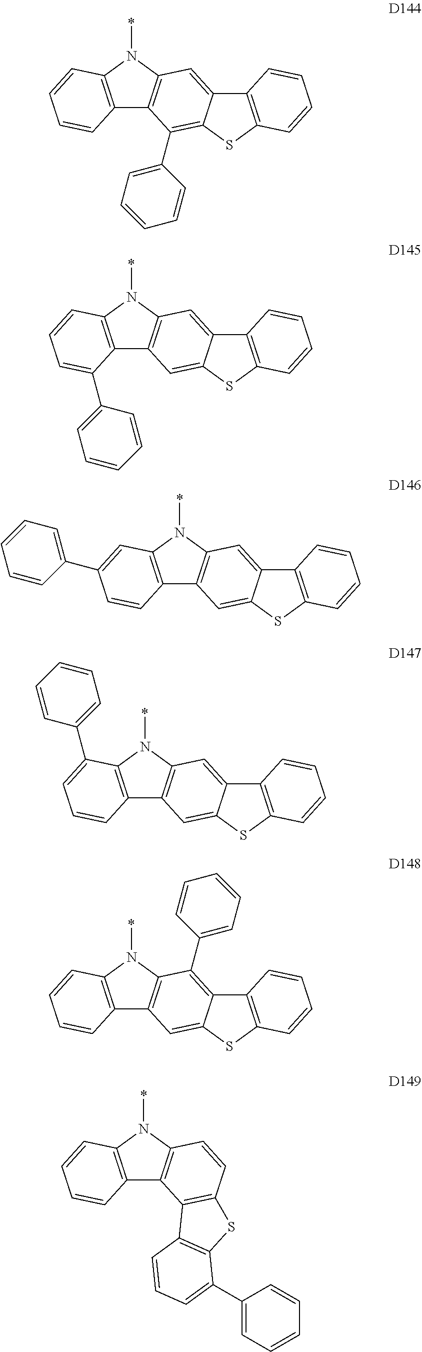

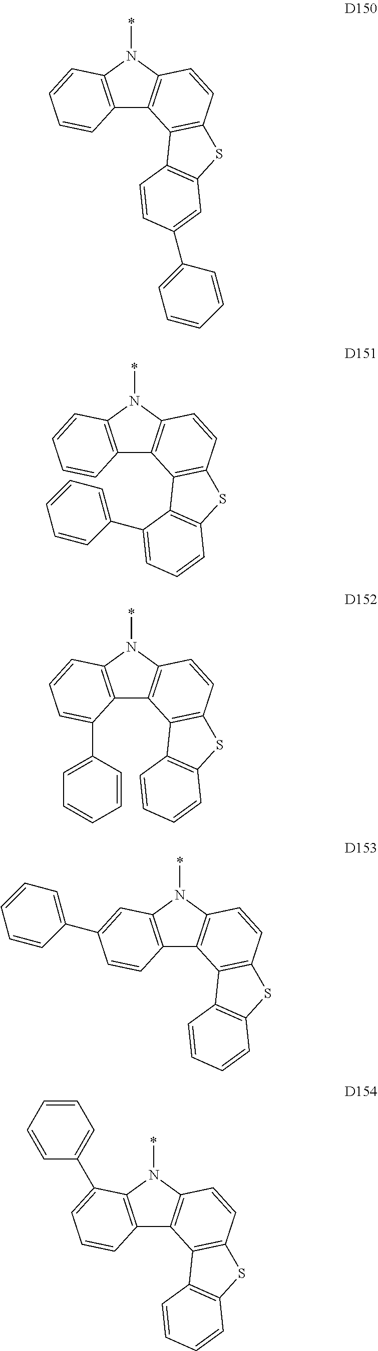

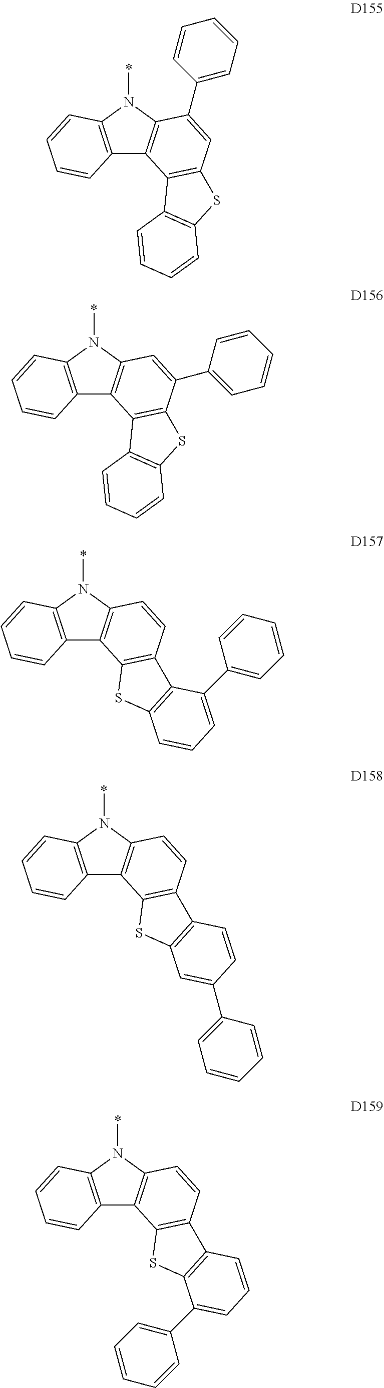 [SMILES: *n1c2ccccc2c2c3c(cc(-c4ccccc4)c21)sc1ccccc13 ; *n1c2ccccc2c2c3c(sc4ccccc43)c(-c3ccccc3)cc21 ; *n1c2ccccc2c2c3sc4cccc(-c5ccccc5)c4c3ccc21 ; *n1c2ccccc2c2c3sc4cc(-c5ccccc5)ccc4c3ccc21 ; *n1c2ccccc2c2c3sc4c(-c5ccccc5)cccc4c3ccc21]

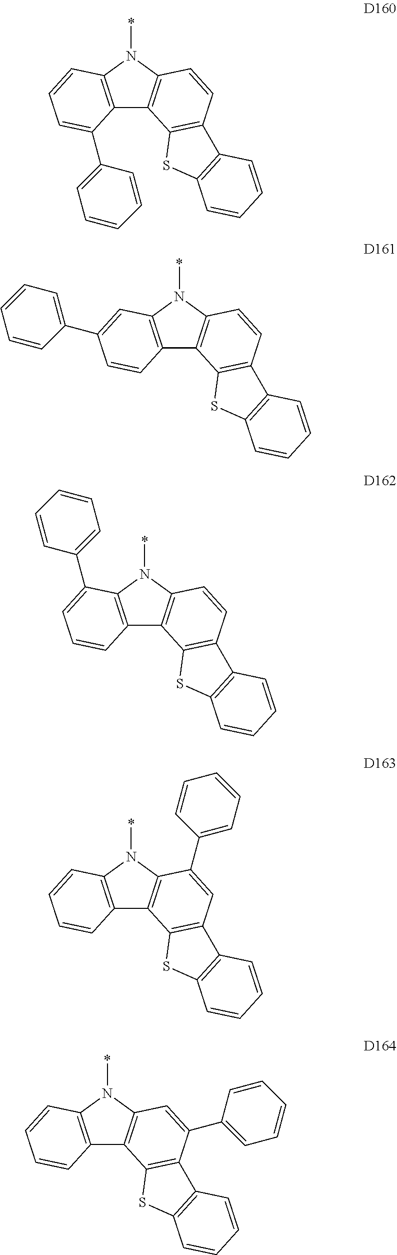

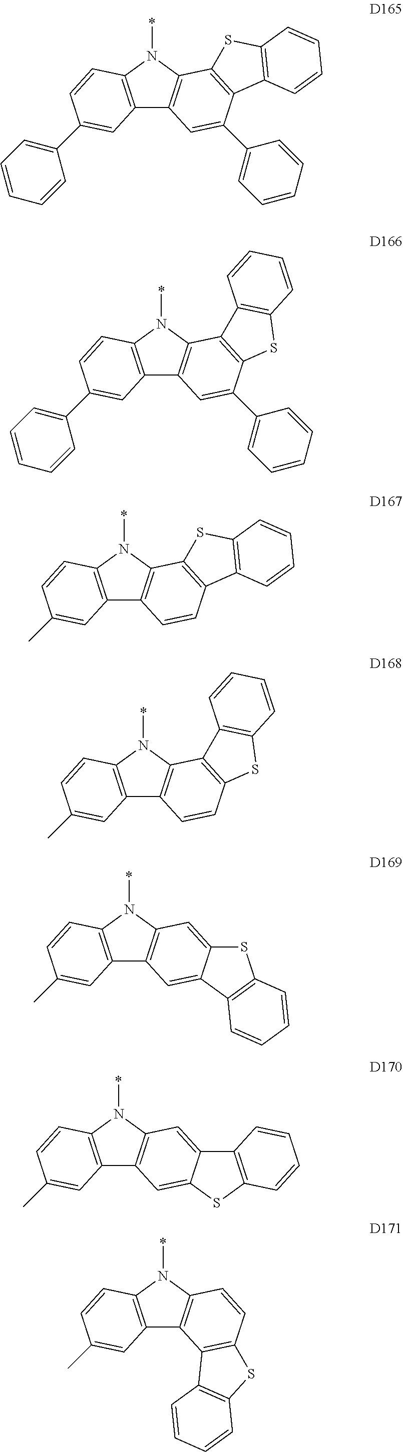

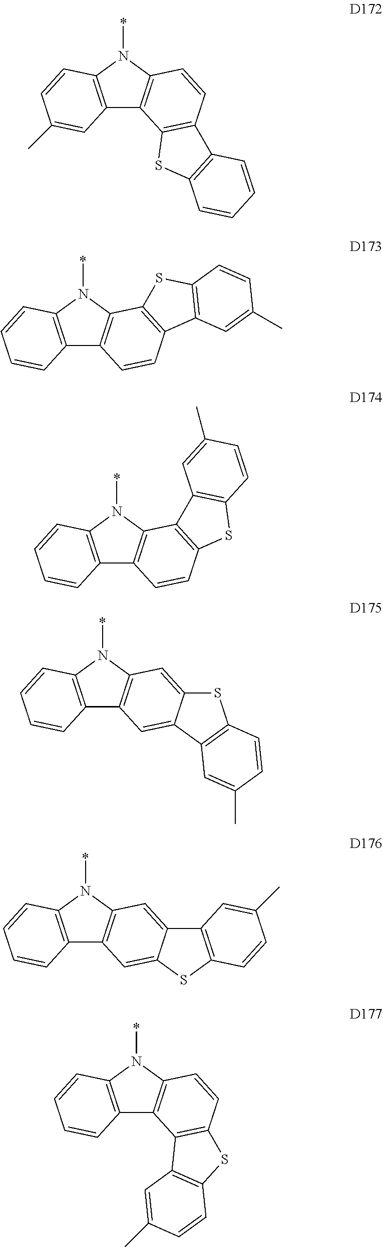

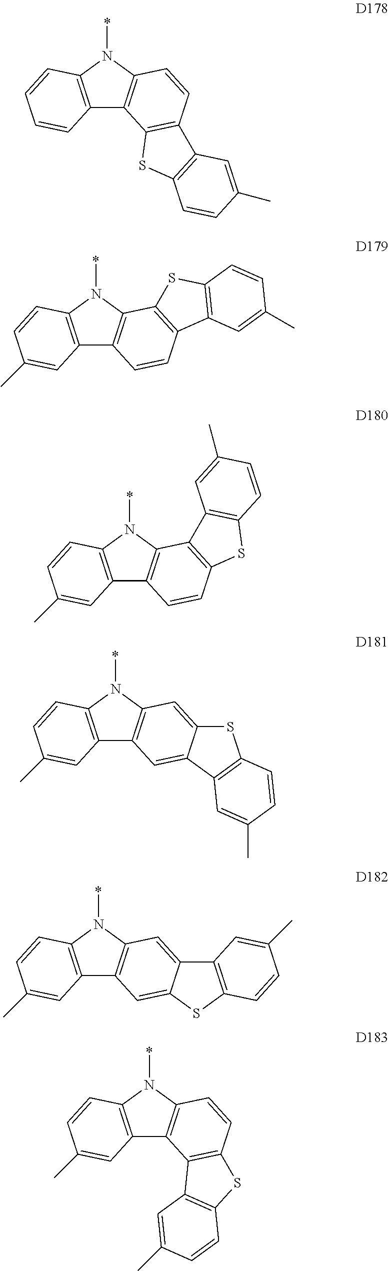

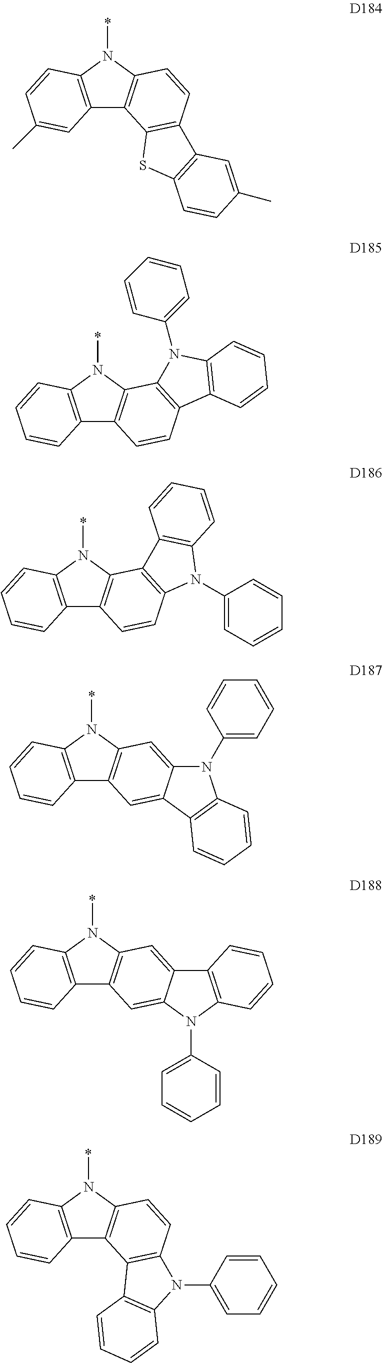

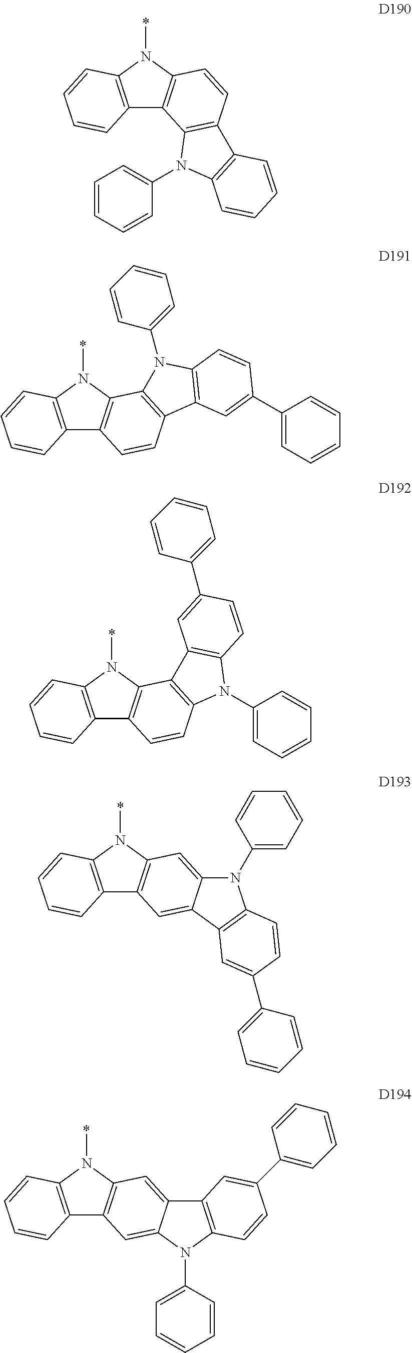

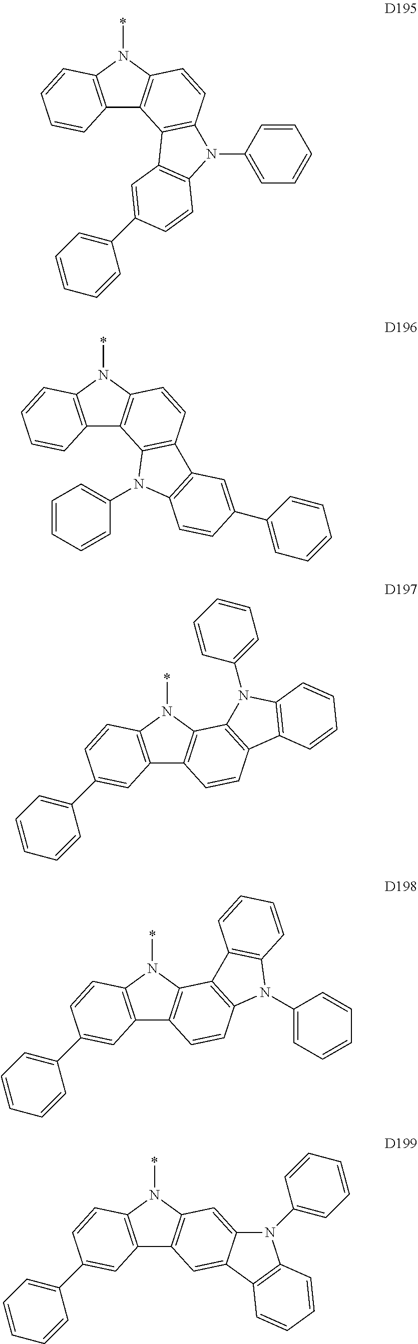

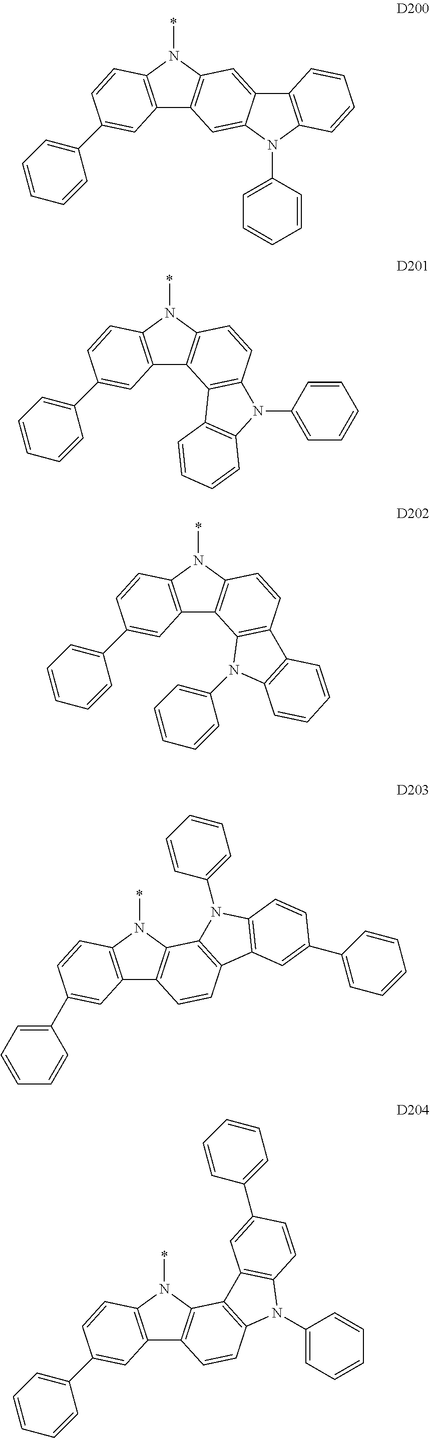

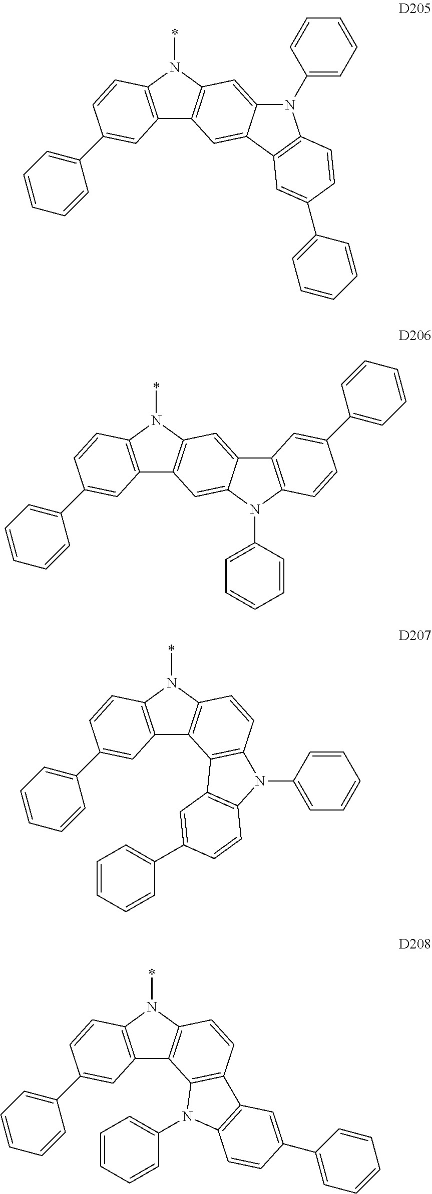 [SMILES: *n1c2ccc(-c3ccccc3)cc2c2cc3c4cc(-c5ccccc5)ccc4n(-c4ccccc4)c3cc21 ; *n1c2ccc(-c3ccccc3)cc2c2cc3c(cc21)c1cc(-c2ccccc2)ccc1n3-c1ccccc1 ; *n1c2ccc(-c3ccccc3)cc2c2c3c4cc(-c5ccccc5)ccc4n(-c4ccccc4)c3ccc21 ; *n1c2ccc(-c3ccccc3)cc2c2c1ccc1c3cc(-c4ccccc4)ccc3n(-c3ccccc3)c12]

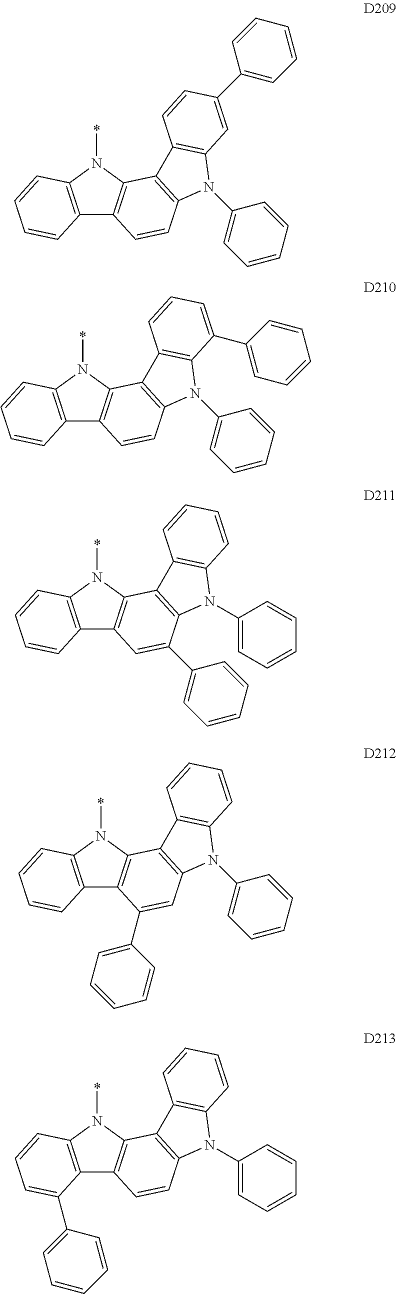

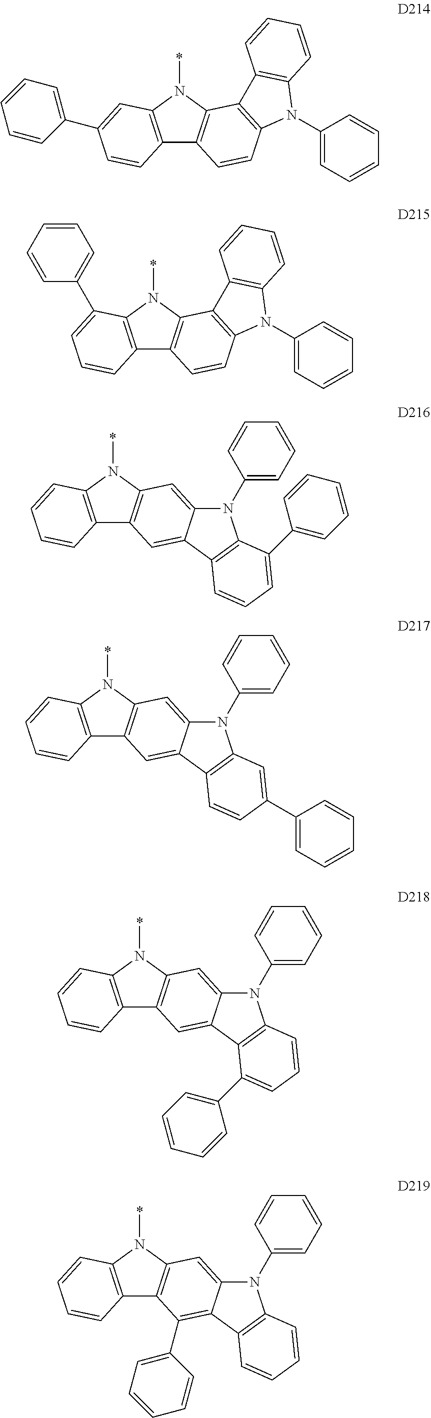

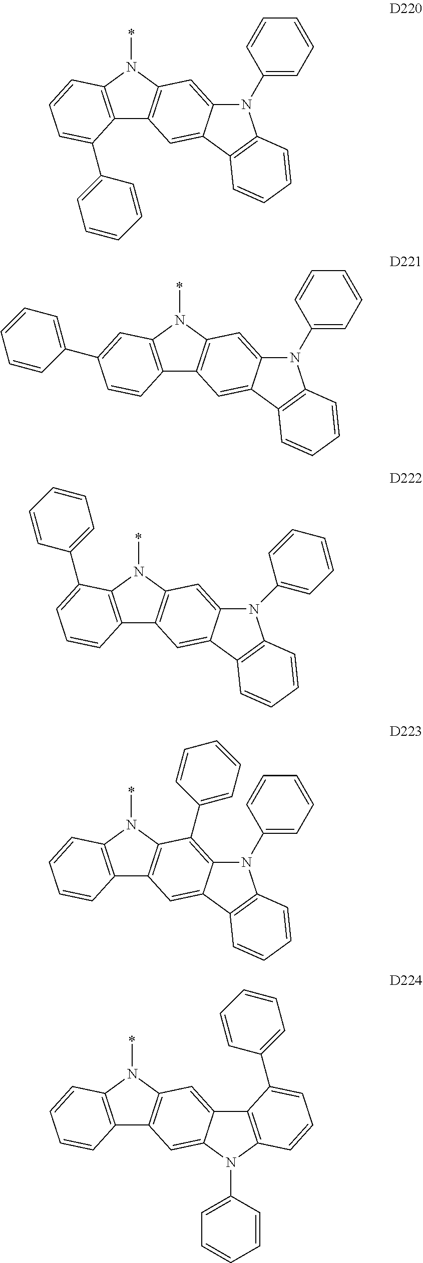

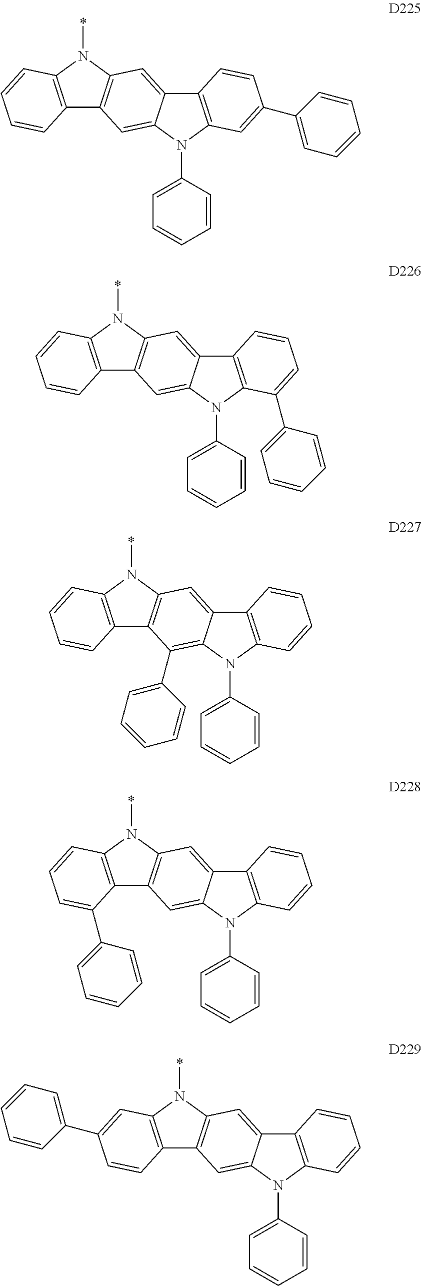 [SMILES: *n1c2ccccc2c2cc3c(cc21)c1ccc(-c2ccccc2)cc1n3-c1ccccc1 ; *n1c2ccccc2c2cc3c(cc21)c1cccc(-c2ccccc2)c1n3-c1ccccc1 ; *n1c2ccccc2c2c(-c3ccccc3)c3c(cc21)c1ccccc1n3-c1ccccc1 ; *n1c2cc3c4ccccc4n(-c4ccccc4)c3cc2c2c(-c3ccccc3)cccc21 ; *n1c2cc(-c3ccccc3)ccc2c2cc3c(cc21)c1ccccc1n3-c1ccccc1]

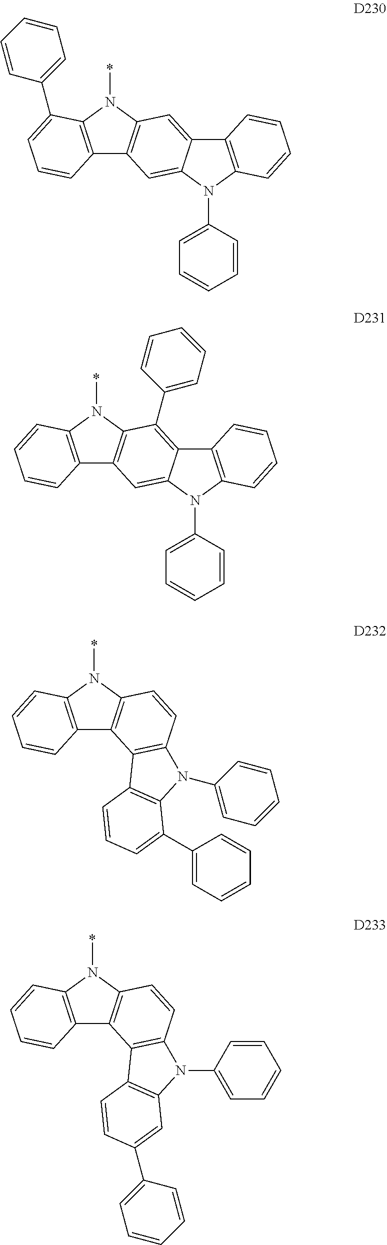

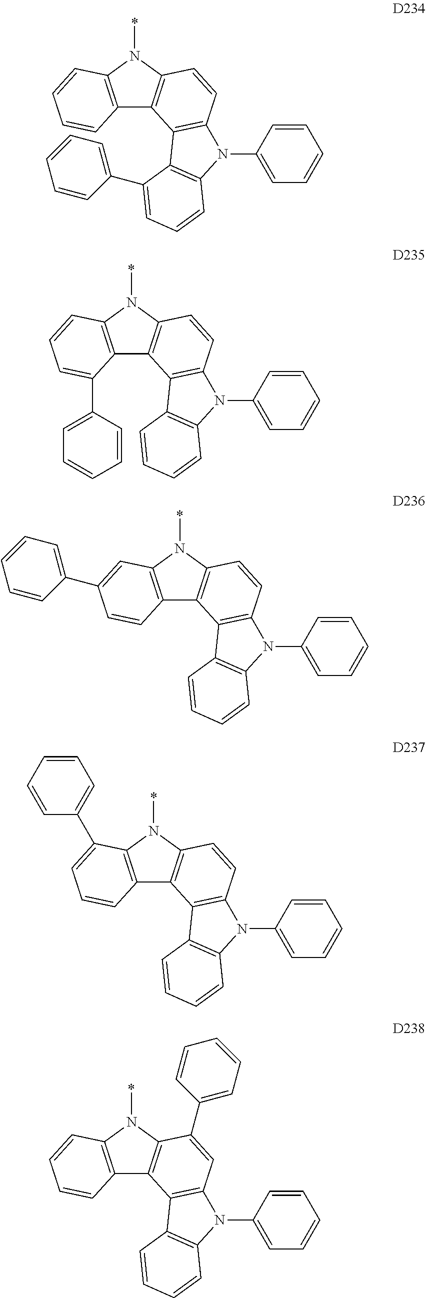

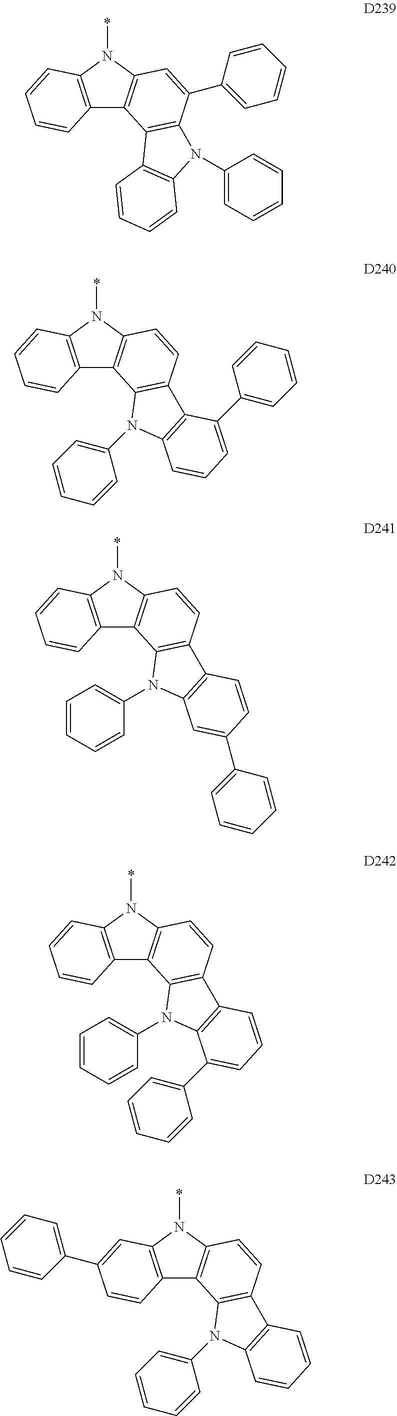

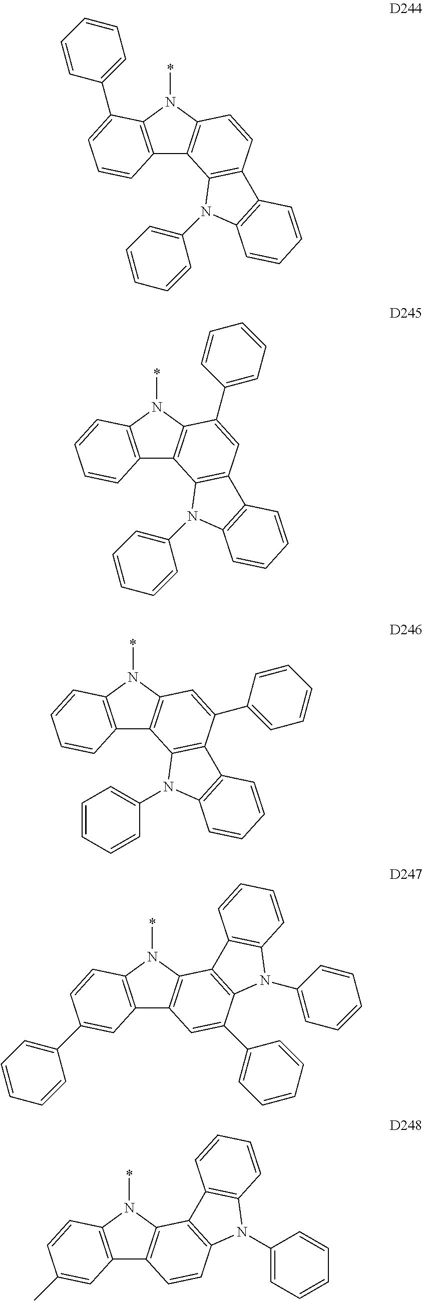

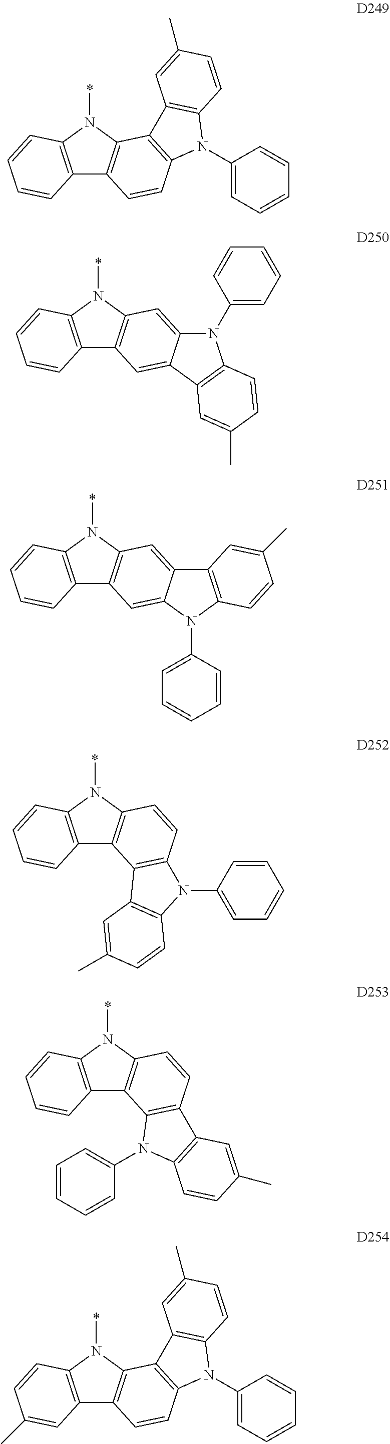

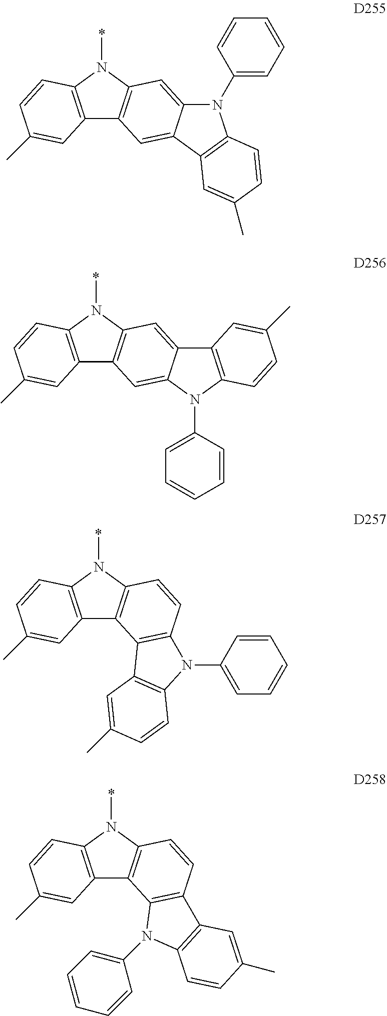

In addition to the above-mentioned specific examples, groups formed by substituting the methyl group (CH₃) of D75 to D92, D167 to D184 and D248 to D258 with a deuterated CD₃ are exemplified herein as D75(m) to D92(m), D167(m) to D184(m) and D248(m) to D258(m), respectively. Also, groups formed by substituting the phenyl group (C₆H₅) of D7 to D74, D99 to D166 and D185 to D258 with a deuterated C₆D₅ are exemplified herein as D7(p) to D74(p), D99(p) to D166(p) and D185(p) to D258(p), respectively. Further, groups formed by deuterating all the hydrogen atoms of D1 to D258 are exemplified herein as D1(D) to D258(D), respectively.

Hereinunder specific examples of D employable in the general formula (1) are further shown. Specific examples shown below are D not represented by the general formula (2). In the present invention, so far as at least one D is a group represented by the general formula (2), the other D can be a donor group such as the following specific examples. However, D that can be employed in the present invention should not be limitatively interpreted by the following specific examples. In the following specific examples, * indicates a bonding position. A methyl group is not shown. Consequently, D260, D261 and D263 represent structures substituted with a methyl group.

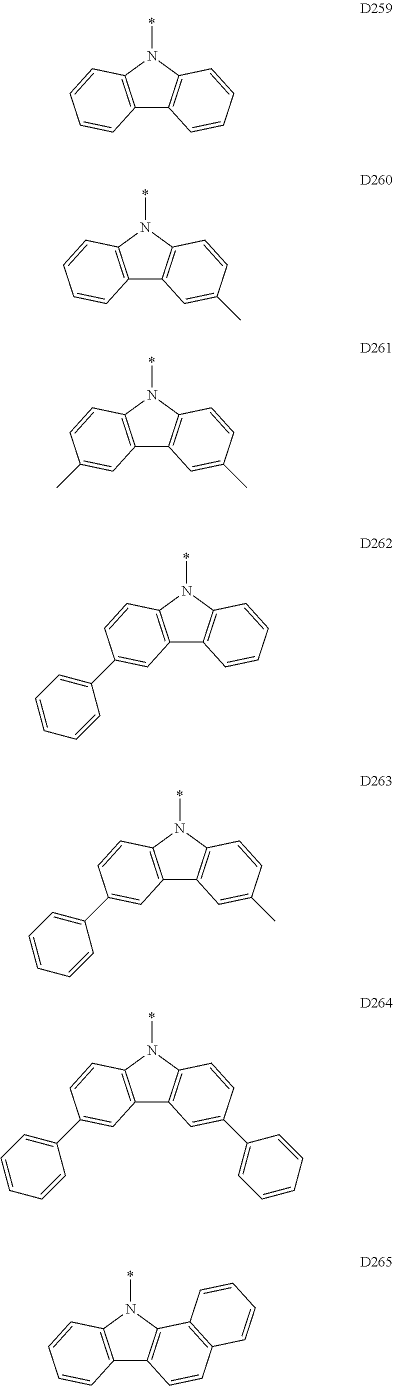

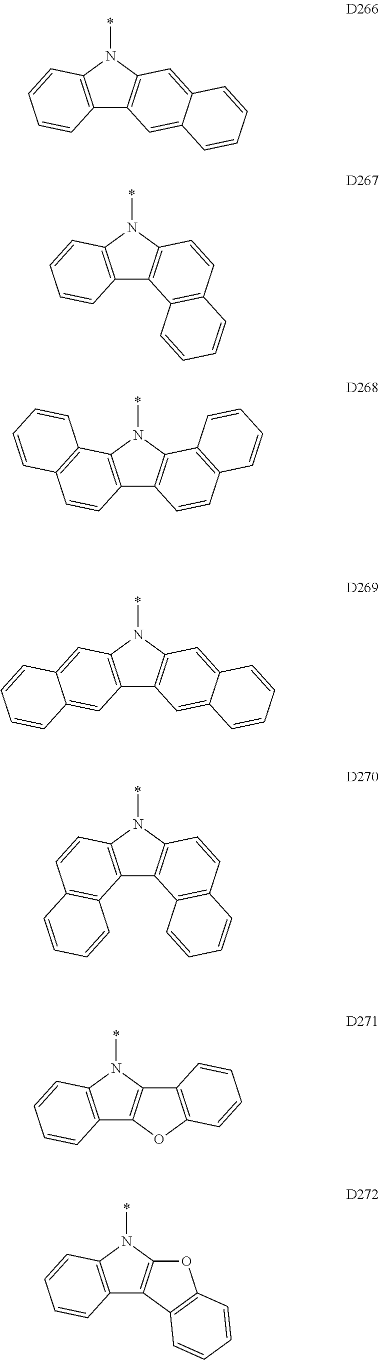

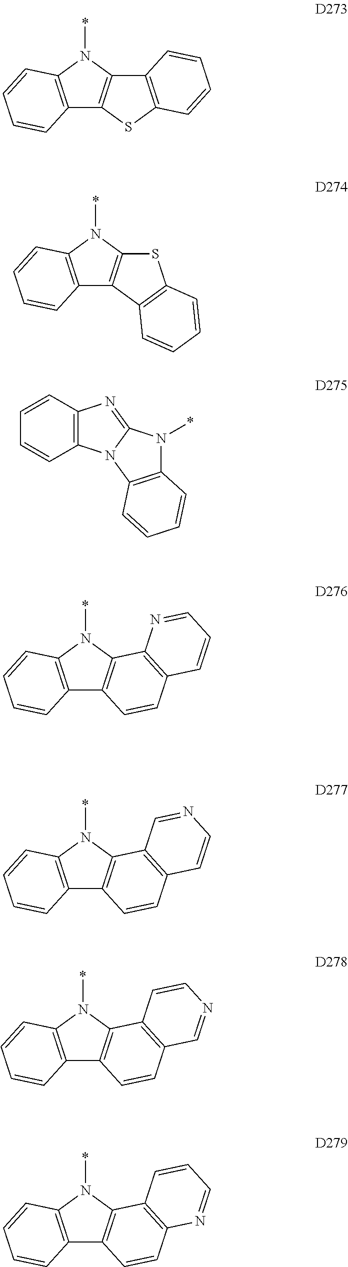

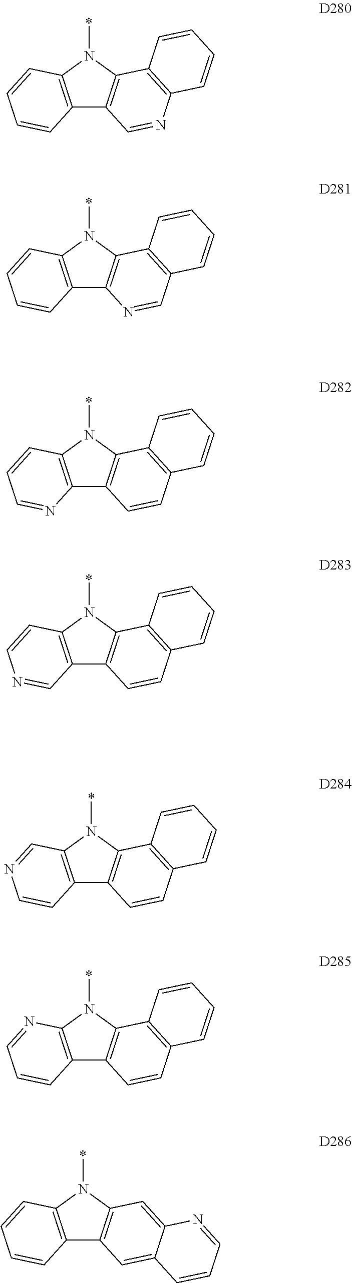

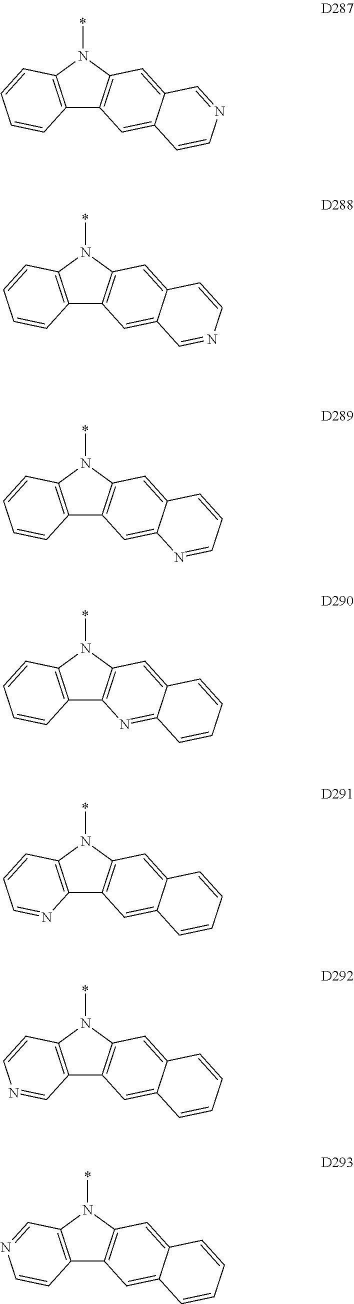

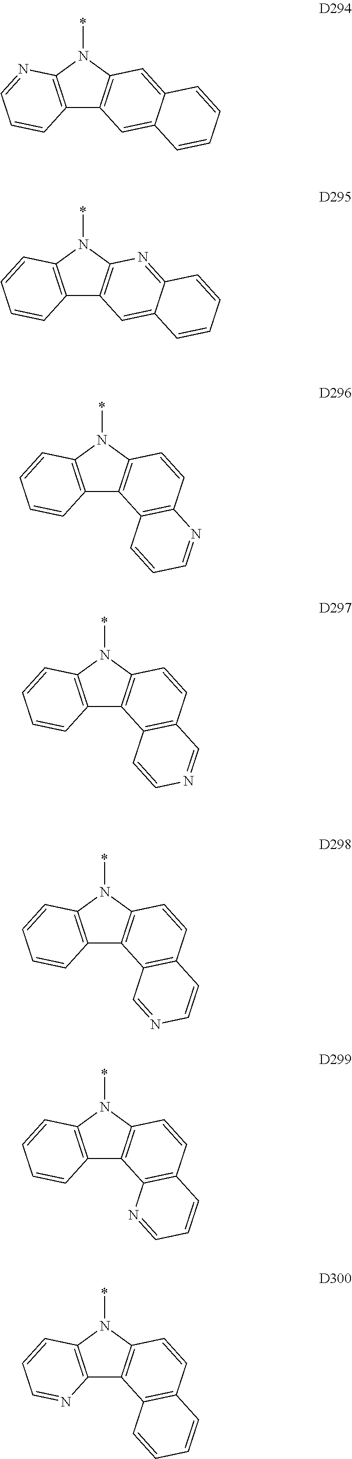

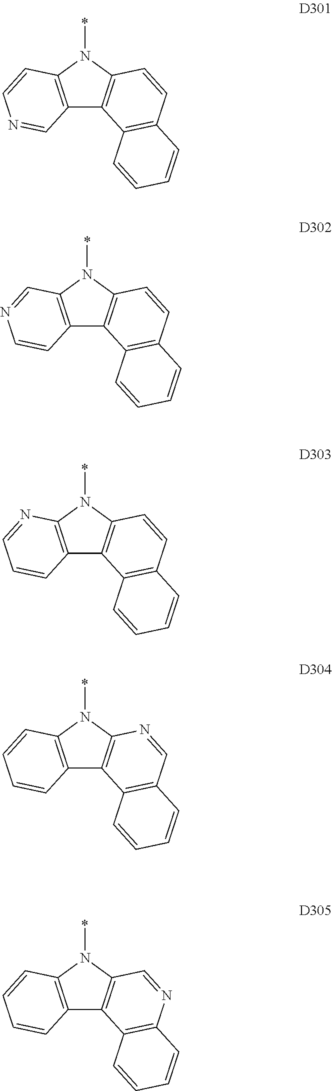 [SMILES: *n1c2ccncc2c2c3ccccc3ccc21 ; *n1c2cnccc2c2c3ccccc3ccc21 ; *n1c2ccc3ccccc3c2c2cccnc21 ; *n1c2ccccc2c2c3ccccc3cnc21 ; *n1c2ccccc2c2c3ccccc3ncc21]

In the general formula (1), A represents one or a combination of two or more groups selected from the group consisting of a cyano group, a phenyl group, a pyrimidyl group, a triazyl group and an alkyl group (except for a substituted alkyl group). Namely, A is a cyano group, a substituted or unsubstituted phenyl group, a substituted or unsubstituted pyrimidyl group, or a substituted or unsubstituted triazyl group, and the substituent for the phenyl group, the pyrimidyl group and the triazyl group includes one or a combination of two or more groups selected from the group consisting of a cyano group, a phenyl group, a pyrimidyl group, a triazyl group and an alkyl group, and the phenyl group and the pyrimidyl group can be fused with a benzene ring.

In a preferred embodiment of the present invention, A is a cyano group or a phenyl group substituted with a cyano group. In one embodiment of the present invention, A is a substituted or unsubstituted pyrimidyl group, or a substituted or unsubstituted triazyl group, preferably a pyrimidyl group substituted with a substituted or unsubstituted phenyl group, or a triazyl group substituted with a substituted or unsubstituted phenyl group. In one embodiment of the present invention, A is a phenyl group substituted with a substituted or unsubstituted pyrimidyl group, or a phenyl group substituted with a substituted or unsubstituted triazyl group.

Hereinunder, specific examples of A that can be employed in the general formula (1) are shown. A employable in the general formula (1) can also be a group containing any of the following structures. For example, A can be a phenyl group substituted with a group having any of the following structures, or a group of the following structure in which the benzene ring is fused with a ring (for example, a benzene ring). A that can be employed in the present invention should not be limitatively interpreted by the following specific examples. In the following specific examples, * indicates a bonding position. Methyl group is omitted. For example, A15 is a group having two 4-methylphenyl groups.

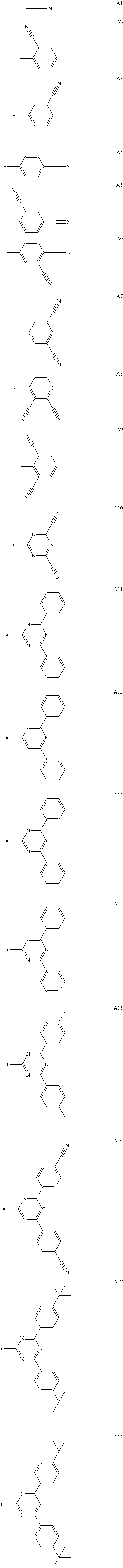

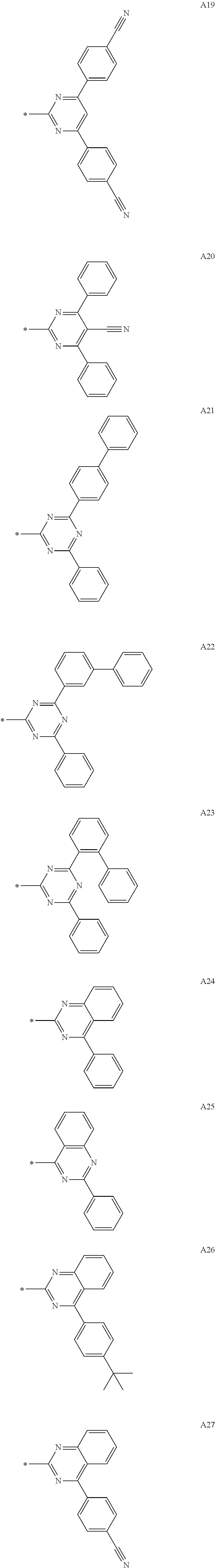

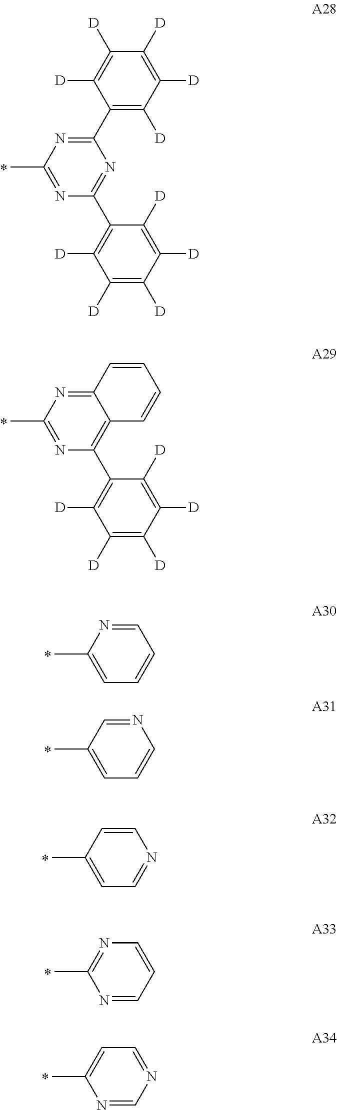

R¹ to R⁴ each independently represent a hydrogen atom, a deuterium atom, or one or a combination of two or more groups selected from the group consisting of an alkyl group, an aryl group, a heteroaryl group and a cyano group. Namely, R¹ to R⁴ each are independently a hydrogen atom, a deuterium atom, a substituted or unsubstituted alkyl group, a substituted or unsubstituted aryl group, a substituted or unsubstituted heteroaryl group, or a cyano group, the substituent for the alkyl group, the aryl group and the heteroaryl group includes one or a combination of two or more groups selected from the group consisting of an alkyl group, an aryl group, a heteroaryl group and a cyano group. In one embodiment of the present invention, the substituent is an alkyl group optionally substituted with an aryl group, or an aryl group optionally substituted with an alkyl group. In one embodiment of the present invention, the substituent is a cyano group, or an aryl group or a heteroaryl group substituted with a cyano group. When 2 or more of R¹ to R⁴ are substituents, these substituents can be the same or different. All R¹ to R⁴ can be hydrogen atoms or deuterium atoms.

In one preferred embodiment of the present invention, R¹ to R⁴ each are independently a hydrogen atom, a deuterium atom, or an alkyl group, an aryl group optionally substituted with a cyano group, or a pyridyl group, preferably a hydrogen atom, a deuterium atom, or an alkyl group, a phenyl group optionally substituted with a cyano group, or a pyridyl group. For example, a hydrogen atom, a deuterium atom, an alkylphenyl group, a cyanophenyl group, a phenyl group or a pyridyl group can be selected, and for example, a hydrogen atom, a deuterium atom, an alkylphenyl group or a phenyl group can be selected.

R¹ and R², and R³ and R⁴ each can bond to each other to form a cyclic structure selected from the group consisting of a benzene ring, a naphthalene ring and a pyridine ring, and the formed cyclic structure can be substituted with one or a combination of two or more groups selected from the group consisting of an alkyl group, an aryl group, and a cyano group. In one embodiment of the present invention, one pair of R¹ and R², and R³ and R⁴ bonds to each other to form a benzene ring, a naphthalene ring or a pyridine ring. In one embodiment of the present invention, both of R¹ and R², and R³ and R⁴ each bond to each other to form a benzene ring, a naphthalene ring or a pyridine ring. At that time, the ring formed by R¹ and R², and the ring formed by R³ and R⁴ can be the same or different. In one embodiment of the present invention, neither R¹ and R², nor R³ and R⁴ bond to each other to form a ring. In one embodiment of the present invention, the cyclic structure to be formed is a benzene ring or a naphthalene ring. In one embodiment of the present invention, the cyclic structure to be formed is a pyridine ring. The hydrogen atom bonding to the benzene ring, the naphthalene ring and the pyridine ring can be substituted with a deuterium atom or a substituent, and the substituent as referred to herein includes one or a combination of two or more groups selected from the group consisting of an alkyl group, an aryl group, a heteroaryl group and a cyano group. In one embodiment of the present invention, the substituent is an alkyl group optionally substituted with an aryl group, or an aryl group optionally substituted with an alkyl group. In one embodiment of the present invention, the substituent is a cyano group, or an aryl group substituted with a cyano group. The hydrogen atom bonding to the benzene ring, the naphthalene ring and the pyridine ring can be unsubstituted.

Hereinunder, specific examples of the aryl group optionally substituted with an alkyl group are shown. However, the aryl group optionally substituted with an alkyl group, which can be employed in the present invention, should not be limitatively interpreted by the following specific examples. In the following specific examples, * indicates a bonding position. Methyl group is omitted. For example, N4 is a 4-methylphenyl group.

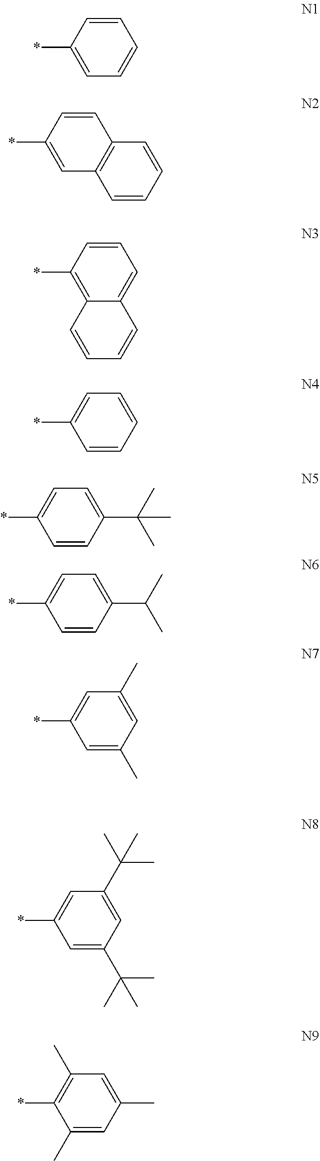

The compound represented by the general formula (1) can be a compound represented by the following general formula (3).

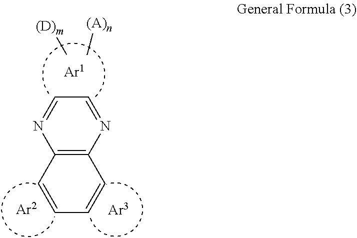

In the general formula (3), Ar¹ represents a cyclic structure, and represents a benzene ring, a naphthalene ring, an anthracene ring, or a phenanthrene ring. D represents a donor group, and at least one D is a group represented by the above general formula (2). A represents one or a combination of two or more groups selected from the group consisting of a cyano group, a phenyl group, a pyrimidyl group, a triazyl group and an alkyl group (except for a substituted alkyl group). m represents 1, 2 or 3, n represents 0, 1 or 2. When m is 2 or 3, plural D's can be the same or different. When n is 2, two A's can be the same or different. Ar² and Ar³ each can independently form a cyclic structure selected from the group consisting of a benzene ring, a naphthalene ring and a pyridine ring, and the formed cyclic structure can be substituted with one or a combination of two or more groups selected from the group consisting of an alkyl group, an aryl group, a heteroaryl group and a cyano group.

For the details and the preferred range of Ar¹, D, A, m and n in the general formula (3), reference can be made to the corresponding description of the above-mentioned general formula (1). For the details and the preferred range of the benzene ring, the naphthalene ring and the pyridine ring that Ar² and Ar³ represent, reference can be made to the description of the benzene ring, the naphthalene ring and the pyridine ring to be formed by R¹ and R², and R³ and R⁴ each bonding to each other in the general formula (1).

In one aspect of the present invention, D in the general formula (3) is a substituted or unsubstituted 5H-benzofuro[3,2-c]carbazol-5-yl group, A is a cyano group, a phenyl group, a pyrimidyl group, a triazyl group or a benzonitrile group, n is 0 or 1, Ar² and Ar³ each are independently a benzene ring, a naphthalene ring, a pyridine ring, or a benzene ring substituted with a cyano group.

The compound represented by the general formula (1) preferably has a ring skeleton of any of the following, for example. At least one hydrogen atom in the following skeletons can be substituted with a deuterium atom or a substituent falling within the range of the general formula (1). However, any other ring is not fused with the skeletons. The general formula (1) indispensably has D, and therefore one D alone is described in the following ring skeletons.

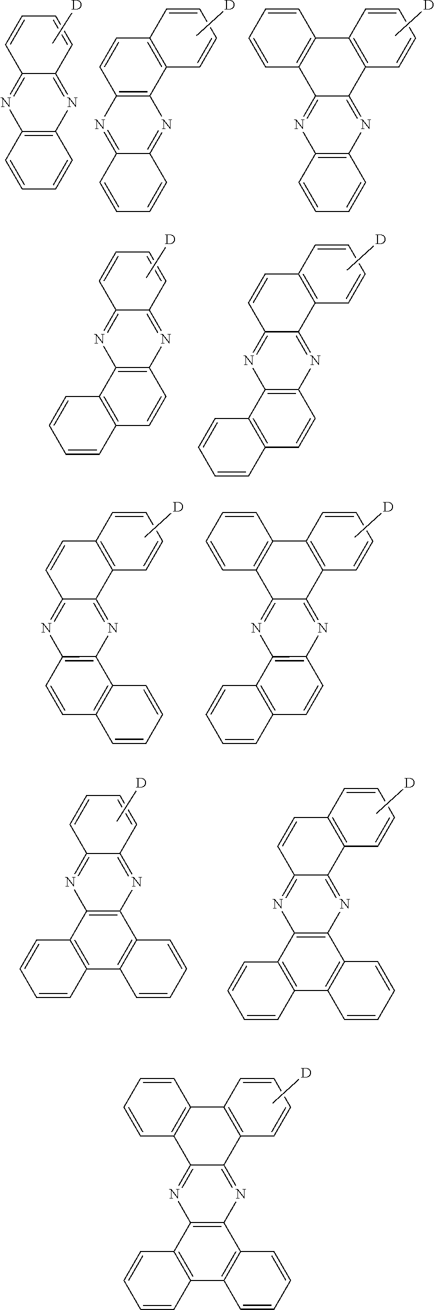

In one preferred embodiment of the present invention, the compound represented by the general formula (1) has a ring skeleton of any of the following Ring Skeleton Group 1.

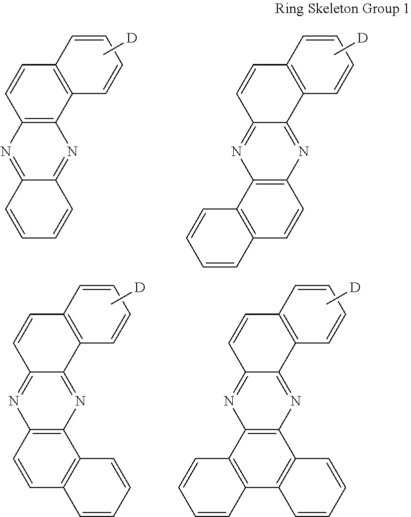

In one preferred embodiment of the present invention, the compound represented by the general formula (1) has a ring skeleton of any of the following Ring Skeleton Group 2.

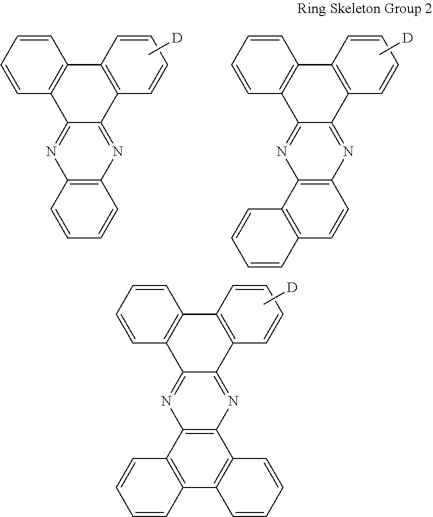

In Ring Skeleton Group 1 and Ring Skeleton Group 2, in one preferred embodiment, A does not exist in the molecule. In one embodiment of the present invention, a hydrogen atom, a deuterium atom, an unsubstituted alkyl group or an aryl group optionally substituted with an alkyl group bonds to the aromatic ring fused with the lower part of the pyrazine ring in Ring Skeleton Group 1 and Ring Skeleton Group 2. In one preferred embodiment of the present invention, a hydrogen atom, a deuterium atom or an unsubstituted alkyl group bonds to the aromatic ring fused with the lower part of the pyrazine ring in Ring Skeleton Group 1 and Ring Skeleton Group 2.

The compound represented by the general formula (1) can be a compound represented by any of the following general formulae (4a) to (4g).

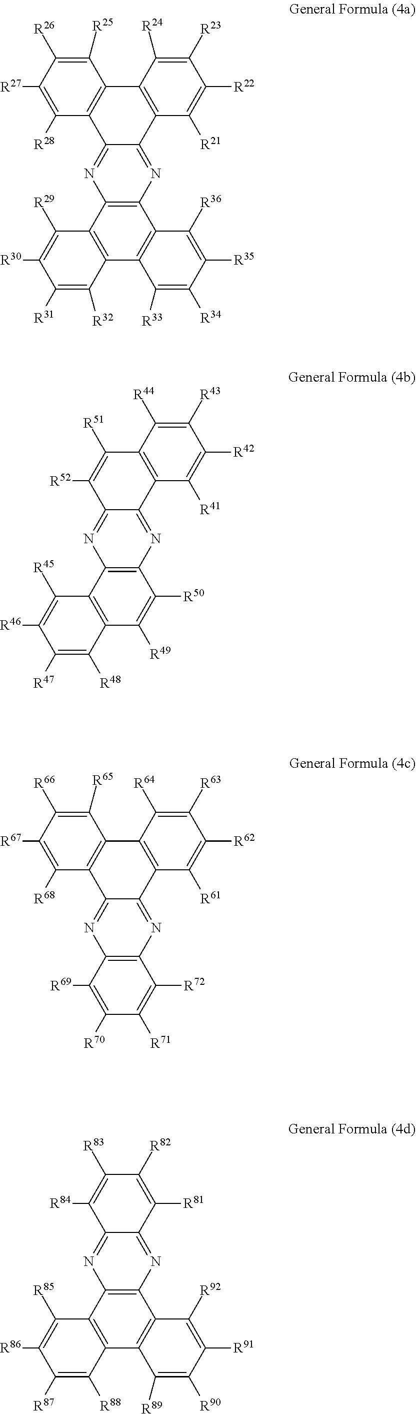

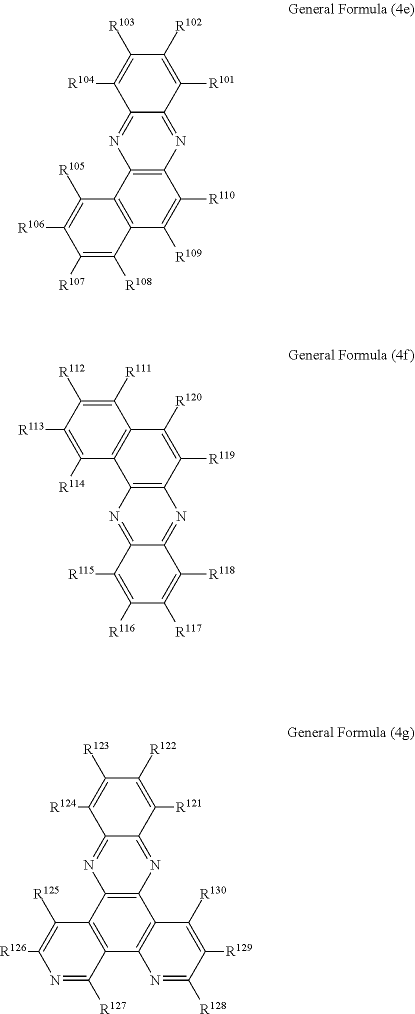

In the general formulae (4a) to (4g), R²¹ to R²⁸, R⁴¹ to R⁴⁴, R⁵¹, R⁵², R⁶¹ to R⁶⁸, R⁸¹ to R⁸⁴, R¹⁰¹ to R¹⁰⁴, R¹¹¹ to R¹¹⁴, R¹¹⁹, R¹²⁰ and R¹²¹ to R¹²⁴ each independently represent a hydrogen atom, a deuterium atom, D or A. Provided that 1 to 3 of R²¹ to R²⁸ are D, and 0 to 2 are A; 1 to 3 of R⁴¹ to R⁴⁴, R⁵¹ and R⁵² are D, and 0 to 2 are A; 1 to 3 of R⁶¹ to R⁶⁸ are D, and 0 to 2 are A; 1 to 3 of R⁸¹ to R⁸⁴ are D, and 0 to 2 are A; 1 to 3 of R¹⁰¹ to R¹⁰⁴ are D and 0 to 2 are A; 1 to 3 of R¹¹¹ to R¹¹⁴, R¹¹⁹ and R¹²⁰ are D, and 0 to 2 are A; 1 to 3 of R¹²¹ to R¹²⁴ are D, and 0 to 2 are A. R²⁹ to R³⁶, R⁴⁵ to R⁵⁰, R⁶⁹ to R⁷², R⁸⁵ to R⁹², R¹⁰⁵ to R¹¹⁰, R¹¹⁵ to R¹¹⁸, and R¹²⁵ to R¹³⁰ each independently represent a hydrogen atom, a deuterium atom, or one or a combination of two or more groups selected from the group consisting of an alkyl group, an aryl group and a cyano group. In the general formulae (4a) to (4g), the ring skeleton described is not further fused with any other ring.

For the details and the preferred range of the general formulae (4a) to (4g), reference can be made to the corresponding description of the general formula (1). In one aspect of the present invention, the compound represented by the general formula (4a) is selected. In one aspect of the present invention, the compound represented by the general formula (4b) is selected. In one aspect of the present invention, the compound represented by the general formula (4c) is selected. In one aspect of the present invention, the compound represented by the general formula (4d) is selected. In one aspect of the present invention, the compound represented by the general formula (4e) is selected. In one aspect of the present invention, the compound represented by the general formula (4f) is selected. In one aspect of the present invention, the compound represented by the general formula (4g) is selected.

Specific examples of the compound represented by the general formula (1) are shown in the following Tables 1 to 42. Specific examples of the compound represented by the general formula (4a′) are shown in Tables 1 to 6; specific examples of the compound represented by the general formula (4b′) are shown in Tables 7 to 12; specific examples of the compound represented by the general formula (4c′) are shown in Tables 13 to 18; specific examples of the compound represented by the general formula (4d′) are shown in Tables 19 to 24; specific examples of the compound represented by the general formula (4e′) are shown in Tables 25 to 30; specific examples of the compound represented by the general formula (4f) are shown in Tables 31 to 36; specific examples of the compound represented by the general formula (4g′) are shown in Tables 37 to 42. However, the compound represented by the general formula (1) employable in the present invention should not be limitatively interpreted by these specific examples.

TABLE 1
General Formula (4a′)

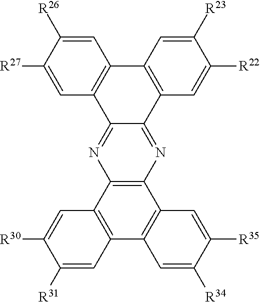

| | | | R²⁶, R²⁷, R³⁰, |
|---|---|---|---|
| No. | R²² | R²³ | R³¹, R³⁴, R³⁵ |
| 1 | D1 | H | H |
| 2 | D2 | H | H |
| 3 | D3 | H | H |
| 4 | D4 | H | H |
| 5 | D5 | H | H |
| 6 | D6 | H | H |
| 7 | D7 | H | H |
| 8 | D8 | H | H |
| 9 | D9 | H | H |
| 10 | D10 | H | H |
| 11 | D11 | H | H |
| 12 | D12 | H | H |
| 13 | D13 | H | H |
| 14 | D14 | H | H |
| 15 | D15 | H | H |
| 16 | D16 | H | H |
| 17 | D17 | H | H |
| 18 | D18 | H | H |
| 19 | D19 | H | H |
| 20 | D20 | H | H |
| 21 | D21 | H | H |
| 22 | D22 | H | H |
| 23 | D23 | H | H |
| 24 | D24 | H | H |
| 25 | D25 | H | H |
| 26 | D26 | H | H |
| 27 | D27 | H | H |
| 28 | D28 | H | H |
| 29 | D29 | H | H |
| 30 | D30 | H | H |
| 31 | D31 | H | H |
| 32 | D32 | H | H |
| 33 | D33 | H | H |
| 34 | D34 | H | H |
| 35 | D35 | H | H |
| 36 | D36 | H | H |
| 37 | D37 | H | H |
| 38 | D38 | H | H |
| 39 | D39 | H | H |
| 40 | D40 | H | H |
| 41 | D41 | H | H |
| 42 | D42 | H | H |
| 43 | D43 | H | H |
| 44 | D44 | H | H |
| 45 | D45 | H | H |
| 46 | D46 | H | H |
| 47 | D47 | H | H |
| 48 | D48 | H | H |
| 49 | D49 | H | H |
| 50 | D50 | H | H |
| 51 | D51 | H | H |
| 52 | D52 | H | H |
| 53 | D53 | H | H |
| 54 | D54 | H | H |
| 55 | D55 | H | H |
| 56 | D56 | H | H |
| 57 | D57 | H | H |
| 58 | D58 | H | H |
| 59 | D59 | H | H |
| 60 | D60 | H | H |
| 61 | D61 | H | H |
| 62 | D62 | H | H |
| 63 | D63 | H | H |
| 64 | D64 | H | H |
| 65 | D65 | H | H |
| 66 | D66 | H | H |
| 67 | D67 | H | H |
| 68 | D68 | H | H |
| 69 | D69 | H | H |
| 70 | D70 | H | H |
| 71 | D71 | H | H |
| 72 | D72 | H | H |
| 73 | D73 | H | H |
| 74 | D74 | H | H |
| 75 | D75 | H | H |
| 76 | D76 | H | H |
| 77 | D77 | H | H |
| 78 | D78 | H | H |
| 79 | D79 | H | H |
| 80 | D80 | H | H |
| 81 | D81 | H | H |
| 82 | D82 | H | H |
| 83 | D83 | H | H |
| 84 | D84 | H | H |
| 85 | D85 | H | H |
| 86 | D86 | H | H |
| 87 | D87 | H | H |
| 88 | D88 | H | H |
| 89 | D89 | H | H |
| 90 | D90 | H | H |
| 91 | D91 | H | H |
| 92 | D92 | H | H |
| 93 | D93 | H | H |
| 94 | D94 | H | H |
| 95 | D95 | H | H |
| 96 | D96 | H | H |
| 97 | D97 | H | H |
| 98 | D98 | H | H |
| 99 | D99 | H | H |
| 100 | D100 | H | H |
| 101 | D101 | H | H |
| 102 | D102 | H | H |
| 103 | D103 | H | H |
| 104 | D104 | H | H |
| 105 | D105 | H | H |
| 106 | D106 | H | H |
| 107 | D107 | H | H |
| 108 | D108 | H | H |
| 109 | D109 | H | H |
| 110 | D110 | H | H |
| 111 | D111 | H | H |
| 112 | D112 | H | H |
| 113 | D113 | H | H |
| 114 | D114 | H | H |
| 115 | D115 | H | H |
| 116 | D116 | H | H |
| 117 | D117 | H | H |
| 118 | D118 | H | H |
| 119 | D119 | H | H |
| 120 | D120 | H | H |

TABLE 2

| No. | R²² | R²³ | R²⁶, R²⁷, R³⁰, R³¹, R³⁴, R³⁵ |
|---|---|---|---|
| 121 | D121 | H | H |
| 122 | D122 | H | H |
| 123 | D123 | H | H |
| 124 | D124 | H | H |
| 125 | D125 | H | H |
| 126 | D126 | H | H |
| 127 | D127 | H | H |
| 128 | D128 | H | H |
| 129 | D129 | H | H |
| 130 | D130 | H | H |
| 131 | D131 | H | H |
| 132 | D132 | H | H |
| 133 | D133 | H | H |
| 134 | D134 | H | H |
| 135 | D135 | H | H |
| 136 | D136 | H | H |
| 137 | D137 | H | H |
| 138 | D138 | H | H |
| 139 | D139 | H | H |
| 140 | D140 | H | H |
| 141 | D141 | H | H |
| 142 | D142 | H | H |
| 143 | D143 | H | H |
| 144 | D144 | H | H |
| 145 | D145 | H | H |
| 146 | D146 | H | H |
| 147 | D147 | H | H |
| 148 | D148 | H | H |
| 149 | D149 | H | H |
| 150 | D150 | H | H |
| 151 | D151 | H | H |
| 152 | D152 | H | H |
| 153 | D153 | H | H |
| 154 | D154 | H | H |
| 155 | D155 | H | H |
| 156 | D156 | H | H |
| 157 | D157 | H | H |
| 158 | D158 | H | H |
| 159 | D159 | H | H |
| 160 | D160 | H | H |
| 161 | D161 | H | H |
| 162 | D162 | H | H |
| 163 | D163 | H | H |
| 164 | D164 | H | H |
| 165 | D165 | H | H |
| 166 | D166 | H | H |
| 167 | D167 | H | H |
| 168 | D168 | H | H |
| 169 | D169 | H | H |
| 170 | D170 | H | H |
| 171 | D171 | H | H |
| 172 | D172 | H | H |
| 173 | D173 | H | H |
| 174 | D174 | H | H |
| 175 | D175 | H | H |
| 176 | D176 | H | H |
| 177 | D177 | H | H |
| 178 | D178 | H | H |
| 179 | D179 | H | H |
| 180 | D180 | H | H |
| 181 | D181 | H | H |
| 182 | D182 | H | H |
| 183 | D183 | H | H |
| 184 | D184 | H | H |
| 185 | D185 | H | H |
| 186 | D186 | H | H |
| 187 | D187 | H | H |
| 188 | D188 | H | H |
| 189 | D189 | H | H |
| 190 | D190 | H | H |
| 191 | D191 | H | H |
| 192 | D192 | H | H |
| 193 | D193 | H | H |
| 194 | D194 | H | H |
| 195 | D195 | H | H |
| 196 | D196 | H | H |
| 197 | D197 | H | H |
| 198 | D198 | H | H |
| 199 | D199 | H | H |
| 200 | D200 | H | H |
| 201 | D201 | H | H |
| 202 | D202 | H | H |
| 203 | D203 | H | H |
| 204 | D204 | H | H |
| 205 | D205 | H | H |
| 206 | D206 | H | H |
| 207 | D207 | H | H |
| 208 | D208 | H | H |
| 209 | D209 | H | H |
| 210 | D210 | H | H |
| 211 | D211 | H | H |
| 212 | D212 | H | H |
| 213 | D213 | H | H |
| 214 | D214 | H | H |
| 215 | D215 | H | H |
| 216 | D216 | H | H |
| 217 | D217 | H | H |
| 218 | D218 | H | H |
| 219 | D219 | H | H |
| 220 | D220 | H | H |
| 221 | D221 | H | H |
| 222 | D222 | H | H |
| 223 | D223 | H | H |
| 224 | D224 | H | H |
| 225 | D225 | H | H |
| 226 | D226 | H | H |
| 227 | D227 | H | H |
| 228 | D228 | H | H |
| 229 | D229 | H | H |
| 230 | D230 | H | H |
| 231 | D231 | H | H |
| 232 | D232 | H | H |
| 233 | D233 | H | H |
| 234 | D234 | H | H |
| 235 | D235 | H | H |
| 236 | D236 | H | H |
| 237 | D237 | H | H |
| 238 | D238 | H | H |
| 239 | D239 | H | H |
| 240 | D240 | H | H |

TABLE 3

| No. | R²² | R²³ | R²⁶, R²⁷, R³⁰, R³¹, R³⁴, R³⁵ |
|---|---|---|---|
| 241 | D241 | H | H |
| 242 | D242 | H | H |
| 243 | D243 | H | H |
| 244 | D244 | H | H |
| 245 | D245 | H | H |
| 246 | D246 | H | H |
| 247 | D247 | H | H |
| 248 | D248 | H | H |
| 249 | D249 | H | H |
| 250 | D250 | H | H |
| 251 | D251 | H | H |
| 252 | D252 | H | H |
| 253 | D253 | H | H |
| 254 | D254 | H | H |
| 255 | D255 | H | H |
| 256 | D256 | H | H |
| 257 | D257 | H | H |
| 258 | D258 | H | H |
| 259 | H | D1 | H |
| 260 | H | D2 | H |
| 261 | H | D3 | H |
| 262 | H | D4 | H |
| 263 | H | D5 | H |
| 264 | H | D6 | H |
| 265 | H | D7 | H |
| 266 | H | D8 | H |
| 267 | H | D9 | H |
| 268 | H | D10 | H |
| 269 | H | D11 | H |
| 270 | H | D12 | H |
| 271 | H | D13 | H |
| 272 | H | D14 | H |
| 273 | H | D15 | H |
| 274 | H | D16 | H |
| 275 | H | D17 | H |
| 276 | H | D18 | H |
| 277 | H | D19 | H |
| 278 | H | D20 | H |
| 279 | H | D21 | H |
| 280 | H | D22 | H |
| 281 | H | D23 | H |
| 282 | H | D24 | H |
| 283 | H | D25 | H |
| 284 | H | D26 | H |
| 285 | H | D27 | H |
| 286 | H | D28 | H |
| 287 | H | D29 | H |
| 288 | H | D30 | H |
| 289 | H | D31 | H |
| 290 | H | D32 | H |
| 291 | H | D33 | H |
| 292 | H | D34 | H |
| 293 | H | D35 | H |
| 294 | H | D36 | H |
| 295 | H | D37 | H |
| 296 | H | D38 | H |
| 297 | H | D39 | H |
| 298 | H | D40 | H |
| 299 | H | D41 | H |
| 300 | H | D42 | H |
| 301 | H | D43 | H |
| 302 | H | D44 | H |
| 303 | H | D45 | H |
| 304 | H | D46 | H |
| 305 | H | D47 | H |
| 306 | H | D48 | H |
| 307 | H | D49 | H |
| 308 | H | D50 | H |
| 309 | H | D51 | H |
| 310 | H | D52 | H |
| 311 | H | D53 | H |
| 312 | H | D54 | H |
| 313 | H | D55 | H |
| 314 | H | D56 | H |
| 315 | H | D57 | H |
| 316 | H | D58 | H |
| 317 | H | D59 | H |
| 318 | H | D60 | H |
| 319 | H | D61 | H |
| 320 | H | D62 | H |
| 321 | H | D63 | H |
| 322 | H | D64 | H |
| 323 | H | D65 | H |
| 324 | H | D66 | H |
| 325 | H | D67 | H |
| 326 | H | D68 | H |
| 327 | H | D69 | H |
| 328 | H | D70 | H |
| 329 | H | D71 | H |
| 330 | H | D72 | H |
| 331 | H | D73 | H |
| 332 | H | D74 | H |
| 333 | H | D75 | H |
| 334 | H | D76 | H |
| 335 | H | D77 | H |
| 336 | H | D78 | H |
| 337 | H | D79 | H |
| 338 | H | D80 | H |
| 339 | H | D81 | H |
| 340 | H | D82 | H |
| 341 | H | D83 | H |
| 342 | H | D84 | H |
| 343 | H | D85 | H |
| 344 | H | D86 | H |
| 345 | H | D87 | H |
| 346 | H | D88 | H |
| 347 | H | D89 | H |
| 348 | H | D90 | H |
| 349 | H | D91 | H |
| 350 | H | D92 | H |
| 351 | H | D93 | H |
| 352 | H | D94 | H |
| 353 | H | D95 | H |
| 354 | H | D96 | H |
| 355 | H | D97 | H |
| 356 | H | D98 | H |
| 357 | H | D99 | H |
| 358 | H | D100 | H |
| 359 | H | D101 | H |
| 360 | H | D102 | H |

TABLE 4

| No. | R²² | R²³ | R²⁶, R²⁷, R³⁰, R³¹, R³⁴, R³⁵ |
|---|---|---|---|
| 361 | H | D103 | H |
| 362 | H | D104 | H |
| 363 | H | D105 | H |
| 364 | H | D106 | H |
| 365 | H | D107 | H |
| 366 | H | D108 | H |
| 367 | H | D109 | H |
| 368 | H | D110 | H |
| 369 | H | D111 | H |
| 370 | H | D112 | H |
| 371 | H | D113 | H |
| 372 | H | D114 | H |
| 373 | H | D115 | H |
| 374 | H | D116 | H |
| 375 | H | D117 | H |
| 376 | H | D118 | H |
| 377 | H | D119 | H |
| 378 | H | D120 | H |
| 379 | H | D121 | H |
| 380 | H | D122 | H |
| 381 | H | D123 | H |
| 382 | H | D124 | H |
| 383 | H | D125 | H |
| 384 | H | D126 | H |
| 385 | H | D127 | H |
| 386 | H | D128 | H |
| 387 | H | D129 | H |
| 388 | H | D130 | H |
| 389 | H | D131 | H |
| 390 | H | D132 | H |
| 391 | H | D133 | H |
| 392 | H | D134 | H |
| 393 | H | D135 | H |
| 394 | H | D136 | H |
| 395 | H | D137 | H |
| 396 | H | D138 | H |
| 397 | H | D139 | H |
| 398 | H | D140 | H |
| 399 | H | D141 | H |
| 400 | H | D142 | H |
| 401 | H | D143 | H |
| 402 | H | D144 | H |
| 403 | H | D145 | H |
| 404 | H | D146 | H |
| 405 | H | D147 | H |
| 406 | H | D148 | H |
| 407 | H | D149 | H |
| 408 | H | D150 | H |
| 409 | H | D151 | H |
| 410 | H | D152 | H |
| 411 | H | D153 | H |
| 412 | H | D154 | H |
| 413 | H | D155 | H |
| 414 | H | D156 | H |
| 415 | H | D157 | H |
| 416 | H | D158 | H |
| 417 | H | D159 | H |
| 418 | H | D160 | H |
| 419 | H | D161 | H |
| 420 | H | D162 | H |
| 421 | H | D163 | H |
| 422 | H | D164 | H |
| 423 | H | D165 | H |
| 424 | H | D166 | H |
| 425 | H | D167 | H |
| 426 | H | D168 | H |
| 427 | H | D169 | H |
| 428 | H | D170 | H |
| 429 | H | D171 | H |
| 430 | H | D172 | H |
| 431 | H | D173 | H |
| 432 | H | D174 | H |
| 433 | H | D175 | H |
| 434 | H | D176 | H |
| 435 | H | D177 | H |
| 436 | H | D178 | H |
| 437 | H | D179 | H |
| 438 | H | D180 | H |
| 439 | H | D181 | H |
| 440 | H | D182 | H |
| 441 | H | D183 | H |
| 442 | H | D184 | H |
| 443 | H | D185 | H |
| 444 | H | D186 | H |
| 445 | H | D187 | H |
| 446 | H | D188 | H |
| 447 | H | D189 | H |
| 448 | H | D190 | H |
| 449 | H | D191 | H |
| 450 | H | D192 | H |
| 451 | H | D193 | H |
| 452 | H | D194 | H |
| 453 | H | D195 | H |
| 454 | H | D196 | H |
| 455 | H | D197 | H |
| 456 | H | D198 | H |
| 457 | H | D199 | H |
| 458 | H | D200 | H |
| 459 | H | D201 | H |
| 460 | H | D202 | H |
| 461 | H | D203 | H |
| 462 | H | D204 | H |
| 463 | H | D205 | H |
| 464 | H | D206 | H |
| 465 | H | D207 | H |
| 466 | H | D208 | H |
| 467 | H | D209 | H |
| 468 | H | D210 | H |
| 469 | H | D211 | H |
| 470 | H | D212 | H |
| 471 | H | D213 | H |
| 472 | H | D214 | H |
| 473 | H | D215 | H |
| 474 | H | D216 | H |
| 475 | H | D217 | H |
| 476 | H | D218 | H |
| 477 | H | D219 | H |
| 478 | H | D220 | H |
| 479 | H | D221 | H |
| 480 | H | D222 | H |

TABLE 5

| | | | R²⁶, R²⁷, R³⁰, |
|---|---|---|---|
| No. | R²² | R²³ | R³¹, R³⁴, R³⁵ |
| 481 | H | D223 | H |
| 482 | H | D224 | H |
| 483 | H | D225 | H |
| 484 | H | D226 | H |
| 485 | H | D227 | H |
| 486 | H | D228 | H |
| 487 | H | D229 | H |
| 488 | H | D230 | H |
| 489 | H | D231 | H |
| 490 | H | D232 | H |
| 491 | H | D233 | H |
| 492 | H | D234 | H |
| 493 | H | D235 | H |
| 494 | H | D236 | H |
| 495 | H | D237 | H |
| 496 | H | D238 | H |
| 497 | H | D239 | H |
| 498 | H | D240 | H |
| 499 | H | D241 | H |
| 500 | H | D242 | H |
| 501 | H | D243 | H |
| 502 | H | D244 | H |
| 503 | H | D245 | H |
| 504 | H | D246 | H |
| 505 | H | D247 | H |
| 506 | H | D248 | H |
| 507 | H | D249 | H |
| 508 | H | D250 | H |
| 509 | H | D251 | H |
| 510 | H | D252 | H |
| 511 | H | D253 | H |
| 512 | H | D254 | H |
| 513 | H | D255 | H |
| 514 | H | D256 | H |
| 515 | H | D257 | H |
| 516 | H | D258 | H |

The following Table 6 further exemplifies the compound represented by the general formula (4a′) in a table form. In Table 6, structures formed by further substituting a part of the structure specified by the compound number are further given compound numbers. For example, in Table 6, Compounds 517 to 774 (expressed as Nos. 517 to 774 in the table) are compounds formed by further substituting R²⁷ (expressed as R²⁷ in the table) of Compounds 1 to 258 with a substituent of R²² (expressed as R²² in the table) of Compounds 1 to 258. Compound 517 is a compound formed by further substituting R²⁷ of Compound 1 with D1 of R²² of Compound 1; Compound 518 is a compound formed by further substituting R²⁷ of Compound 2 with D2 of R²² of Compound 2. The structures of the compounds listed in Table 6 and the compounds listed in Table 12, Table 18, Table 24, Table 30, Table 36 and Table 42 are identified in such a manner. In Table 6, Table 12, Table 18, Table 24, Table 30, Table 36 and Table 42, every numbered compound is individually identified for the structure thereof, and is specifically disclosed one by one in the present description.

TABLE 6

| | | Substituting |
|---|---|---|
| New Compound Nos. | Substituted Groups | Groups |
| Nos. 517 to 774 are | by substituting R27 of | with the same |
| compounds obtained | Nos. 1 to 258 | group as R22. |
| Nos. 775 to 1032 are | by substituting R26 of | with the same |
| compounds obtained | Nos. 259 to 516 | group as R23. |
| Nos. 1033 to 2064 are | by substituting R30 of | with N1. |
| compounds obtained | Nos. 1 to 1032 | |
| Nos. 2065 to 3096 are | by substituting R31 of | with N1. |
| compounds obtained | Nos. 1 to 1032 | |
| Nos. 3097 to 3612 are | by substituting R34 of | with N1. |
| compounds obtained | Nos. 1 to 516 | |
| Nos. 3613 to 4128 are | by substituting R35 of | with N1. |
| compounds obtained | Nos. 1 to 516 | |
| Nos. 4129 to 5160 are | by substituting R30 and | with N1. |
| compounds obtained | R35 of Nos. 1 to 1032 | |
| Nos. 5161 to 6192 are | by substituting R31 and | with N1. |
| compounds obtained | R34 of Nos. 1 to 1032 | |
| Nos. 6193 to 7224 are | by substituting R30 of | with A1. |
| compounds obtained | Nos. 1 to 1032 | |
| Nos. 7225 to 8256 are | by substituting R31 of | with A1. |
| compounds obtained | Nos. 1 to 1032 | |
| Nos. 8257 to 8772 are | by substituting R34 of | with A1. |
| compounds obtained | Nos. 1 to 516 | |
| Nos. 8773 to 9288 are | by substituting R35 of | with A1. |
| compounds obtained | Nos. 1 to 516 | |
| Nos. 9289 to 10320 are | by substituting R30 and | with A1. |
| compounds obtained | R35 of Nos. 1 to 1032 | |
| Nos. 10321 to 11352 are | by substituting R31 and | with A1. |
| compounds obtained | R34 of Nos. 1 to 1032 | |
| Nos. 11353 to 12384 are | by substituting R30 of | with N5. |
| compounds obtained | Nos. 1 to 1032 | |
| Nos. 12385 to 13416 are | by substituting R31 of | with N5. |
| compounds obtained | Nos. 1 to 1032 | |
| Nos. 13417 to 13932 are | by substituting R34 of | with N5. |
| compounds obtained | Nos. 1 to 516 | |
| Nos. 13933 to 14448 are | by substituting R35 of | with N5. |
| compounds obtained | Nos. 1 to 516 | |
| Nos. 14449 to 15480 are | by substituting R30 and | with N5. |
| compounds obtained | R35 of Nos. 1 to 1032 | |
| Nos. 15481 to 15738 are | by substituting R23 of | with the same |
| compounds obtained | Nos. 1 to 258 | group as R22. |
| Nos. 15739 to 15996 are | by substituting R31 of | with A1. |
| compounds obtained | Nos. 15481 to 15738 | |
| Nos. 15997 to 16512 are | by substituting R34 of | with A1. |
| compounds obtained | Nos. 15481 to 15996 | |

TABLE 7
General Formula (4b′)

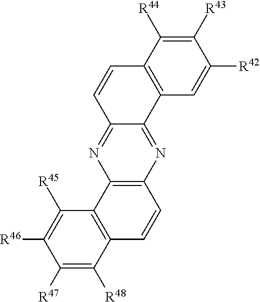

| No. | R⁴² | R⁴³ | R⁴⁴~R⁴⁸ |
|---|---|---|---|
| 16513 | D1 | H | H |
| 16514 | D2 | H | H |
| 16515 | D3 | H | H |
| 16516 | D4 | H | H |
| 16517 | D5 | H | H |
| 16518 | D6 | H | H |
| 16519 | D7 | H | H |
| 16520 | D8 | H | H |
| 16521 | D9 | H | H |
| 16522 | D10 | H | H |
| 16523 | D11 | H | H |
| 16524 | D12 | H | H |
| 16525 | D13 | H | H |
| 16526 | D14 | H | H |
| 16527 | D15 | H | H |
| 16528 | D16 | H | H |
| 16529 | D17 | H | H |
| 16530 | D18 | H | H |
| 16531 | D19 | H | H |
| 16532 | D20 | H | H |
| 16533 | D21 | H | H |
| 16534 | D22 | H | H |
| 16535 | D23 | H | H |
| 16536 | D24 | H | H |
| 16537 | D25 | H | H |
| 16538 | D26 | H | H |
| 16539 | D27 | H | H |
| 16540 | D28 | H | H |
| 16541 | D29 | H | H |
| 16542 | D30 | H | H |
| 16543 | D31 | H | H |
| 16544 | D32 | H | H |
| 16545 | D33 | H | H |
| 16546 | D34 | H | H |
| 16547 | D35 | H | H |
| 16548 | D36 | H | H |
| 16549 | D37 | H | H |
| 16550 | D38 | H | H |
| 16551 | D39 | H | H |
| 16552 | D40 | H | H |
| 16553 | D41 | H | H |
| 16554 | D42 | H | H |
| 16555 | D43 | H | H |
| 16556 | D44 | H | H |
| 16557 | D45 | H | H |
| 16558 | D46 | H | H |
| 16559 | D47 | H | H |
| 16560 | D48 | H | H |
| 16561 | D49 | H | H |
| 16562 | D50 | H | H |
| 16563 | D51 | H | H |
| 16564 | D52 | H | H |
| 16565 | D53 | H | H |
| 16566 | D54 | H | H |
| 16567 | D55 | H | H |
| 16568 | D56 | H | H |
| 16569 | D57 | H | H |
| 16570 | D58 | H | H |
| 16571 | D59 | H | H |
| 16572 | D60 | H | H |
| 16573 | D61 | H | H |
| 16574 | D62 | H | H |
| 16575 | D63 | H | H |
| 16576 | D64 | H | H |
| 16577 | D65 | H | H |
| 16578 | D66 | H | H |
| 16579 | D67 | H | H |
| 16580 | D68 | H | H |
| 16581 | D69 | H | H |
| 16582 | D70 | H | H |
| 16583 | D71 | H | H |
| 16584 | D72 | H | H |
| 16585 | D73 | H | H |
| 16586 | D74 | H | H |
| 16587 | D75 | H | H |
| 16588 | D76 | H | H |
| 16589 | D77 | H | H |
| 16590 | D78 | H | H |
| 16591 | D79 | H | H |
| 16592 | D80 | H | H |
| 16593 | D81 | H | H |
| 16594 | D82 | H | H |
| 16595 | D83 | H | H |
| 16596 | D84 | H | H |
| 16597 | D85 | H | H |
| 16598 | D86 | H | H |
| 16599 | D87 | H | H |
| 16600 | D88 | H | H |
| 16601 | D89 | H | H |
| 16602 | D90 | H | H |
| 16603 | D91 | H | H |
| 16604 | D92 | H | H |
| 16605 | D93 | H | H |
| 16606 | D94 | H | H |
| 16607 | D95 | H | H |
| 16608 | D96 | H | H |
| 16609 | D97 | H | H |
| 16610 | D98 | H | H |
| 16611 | D99 | H | H |
| 16612 | D100 | H | H |
| 16613 | D101 | H | H |
| 16614 | D102 | H | H |
| 16615 | D103 | H | H |
| 16616 | D104 | H | H |
| 16617 | D105 | H | H |
| 16618 | D106 | H | H |
| 16619 | D107 | H | H |
| 11661 | D108 | H | H |
| 16621 | D109 | H | H |
| 16622 | D110 | H | H |
| 16623 | D111 | H | H |
| 16624 | D112 | H | H |
| 16625 | D113 | H | H |
| 16626 | D114 | H | H |
| 16627 | D115 | H | H |
| 16628 | D116 | H | H |
| 16629 | D117 | H | H |
| 16630 | D118 | H | H |
| 16631 | D119 | H | H |
| 16632 | D120 | H | H |

TABLE 8

| No. | R⁴² | R⁴³ | R⁴⁴~R⁴⁸ |
|---|---|---|---|
| 16633 | D121 | H | H |
| 16634 | D122 | H | H |
| 16635 | D123 | H | H |
| 16636 | D124 | H | H |
| 16637 | D125 | H | H |
| 16638 | D126 | H | H |
| 16639 | D127 | H | H |
| 16640 | D128 | H | H |
| 16641 | D129 | H | H |
| 16642 | D130 | H | H |
| 16643 | D131 | H | H |
| 16644 | D132 | H | H |
| 16645 | D133 | H | H |
| 16646 | D134 | H | H |
| 16647 | D135 | H | H |
| 16648 | D136 | H | H |
| 16649 | D137 | H | H |
| 16650 | D138 | H | H |
| 16651 | D139 | H | H |
| 16652 | D140 | H | H |
| 16653 | D141 | H | H |
| 16654 | D142 | H | H |
| 16655 | D143 | H | H |
| 16656 | D144 | H | H |
| 16657 | D145 | H | H |
| 16658 | D146 | H | H |
| 16659 | D147 | H | H |
| 16660 | D148 | H | H |
| 16661 | D149 | H | H |
| 16662 | D150 | H | H |
| 16663 | D151 | H | H |
| 16664 | D152 | H | H |
| 16665 | D153 | H | H |
| 16666 | D154 | H | H |
| 16667 | D155 | H | H |
| 16668 | D156 | H | H |
| 16669 | D157 | H | H |
| 16670 | D158 | H | H |
| 16671 | D159 | H | H |
| 16672 | D160 | H | H |
| 16673 | D161 | H | H |
| 16674 | D162 | H | H |
| 16675 | D163 | H | H |
| 16676 | D164 | H | H |
| 16677 | D165 | H | H |
| 16678 | D166 | H | H |
| 16679 | D167 | H | H |
| 16680 | D168 | H | H |
| 16681 | D169 | H | H |
| 16682 | D170 | H | H |
| 16683 | D171 | H | H |
| 16684 | D172 | H | H |
| 16685 | D173 | H | H |
| 16686 | D174 | H | H |
| 16687 | D175 | H | H |
| 16688 | D176 | H | H |
| 16689 | D177 | H | H |
| 16690 | D178 | H | H |
| 16691 | D179 | H | H |
| 16692 | D180 | H | H |
| 16693 | D181 | H | H |
| 16694 | D182 | H | H |
| 16695 | D183 | H | H |
| 16696 | D184 | H | H |
| 16697 | D185 | H | H |
| 16698 | D186 | H | H |
| 16699 | D187 | H | H |
| 16700 | D188 | H | H |
| 16701 | D189 | H | H |
| 16702 | D190 | H | H |
| 16703 | D191 | H | H |
| 16704 | D192 | H | H |
| 16705 | D193 | H | H |
| 16706 | D194 | H | H |
| 16707 | D195 | H | H |
| 16708 | D196 | H | H |
| 16709 | D197 | H | H |
| 16710 | D198 | H | H |
| 16711 | D199 | H | H |
| 16712 | D200 | H | H |
| 16713 | D201 | H | H |
| 16714 | D202 | H | H |
| 16715 | D203 | H | H |
| 16716 | D204 | H | H |
| 16717 | D205 | H | H |
| 16718 | D206 | H | H |
| 16719 | D207 | H | H |
| 16720 | D208 | H | H |
| 16721 | D209 | H | H |
| 16722 | D210 | H | H |
| 16723 | D211 | H | H |
| 16724 | D212 | H | H |
| 16725 | D213 | H | H |
| 16726 | D214 | H | H |
| 16727 | D215 | H | H |
| 16728 | D216 | H | H |
| 16729 | D217 | H | H |
| 16730 | D218 | H | H |
| 16731 | D219 | H | H |
| 16732 | D220 | H | H |
| 16733 | D221 | H | H |
| 16734 | D222 | H | H |
| 16735 | D223 | H | H |
| 16736 | D224 | H | H |
| 16737 | D225 | H | H |
| 16738 | D226 | H | H |
| 16739 | D227 | H | H |
| 16740 | D228 | H | H |
| 16741 | D229 | H | H |
| 16742 | D230 | H | H |
| 16743 | D231 | H | H |
| 16744 | D232 | H | H |
| 16745 | D233 | H | H |
| 16746 | D234 | H | H |
| 16747 | D235 | H | H |
| 16748 | D236 | H | H |
| 16749 | D237 | H | H |
| 16750 | D238 | H | H |
| 16751 | D239 | H | H |
| 16752 | D240 | H | H |

TABLE 9

| No. | R⁴² | R⁴³ | R⁴⁴~R⁴⁸ |
|---|---|---|---|
| 16753 | D241 | H | H |
| 16754 | D242 | H | H |
| 16755 | D243 | H | H |
| 16756 | D244 | H | H |
| 16757 | D245 | H | H |
| 16758 | D246 | H | H |
| 16759 | D247 | H | H |
| 16760 | D248 | H | H |
| 16761 | D249 | H | H |
| 16762 | D250 | H | H |
| 16763 | D251 | H | H |
| 16764 | D252 | H | H |
| 16765 | D253 | H | H |
| 16766 | D254 | H | H |
| 16767 | D255 | H | H |
| 16768 | D256 | H | H |
| 16769 | D257 | H | H |
| 16770 | D258 | H | H |
| 16771 | H | D1 | H |
| 16772 | H | D2 | H |
| 16773 | H | D3 | H |
| 16774 | H | D4 | H |
| 16775 | H | D5 | H |
| 16776 | H | D6 | H |
| 16777 | H | D7 | H |
| 16778 | H | D8 | H |
| 16779 | H | D9 | H |
| 16780 | H | D10 | H |
| 16781 | H | D11 | H |
| 16782 | H | D12 | H |
| 16783 | H | D13 | H |
| 16784 | H | D14 | H |
| 16785 | H | D15 | H |
| 16786 | H | D16 | H |
| 16787 | H | D17 | H |
| 16788 | H | D18 | H |
| 16789 | H | D19 | H |
| 16790 | H | D20 | H |
| 16791 | H | D21 | H |
| 16792 | H | D22 | H |
| 16793 | H | D23 | H |
| 16794 | H | D24 | H |
| 16795 | H | D25 | H |
| 16796 | H | D26 | H |
| 16797 | H | D27 | H |
| 16798 | H | D28 | H |
| 16799 | H | D29 | H |
| 16800 | H | D30 | H |
| 16801 | H | D31 | H |
| 16802 | H | D32 | H |
| 16803 | H | D33 | H |
| 16804 | H | D34 | H |
| 16805 | H | D35 | H |
| 16806 | H | D36 | H |
| 16807 | H | D37 | H |
| 16808 | H | D38 | H |
| 16809 | H | D39 | H |
| 16810 | H | D40 | H |
| 16811 | H | D41 | H |
| 16812 | H | D42 | H |
| 16813 | H | D43 | H |
| 16814 | H | D44 | H |
| 16815 | H | D45 | H |
| 16816 | H | D46 | H |
| 16817 | H | D47 | H |
| 16818 | H | D48 | H |
| 16819 | H | D49 | H |
| 16820 | H | D50 | H |
| 16821 | H | D51 | H |
| 16822 | H | D52 | H |
| 16823 | H | D53 | H |
| 16824 | H | D54 | H |
| 16825 | H | D55 | H |
| 16826 | H | D56 | H |
| 16827 | H | D57 | H |
| 16828 | H | D58 | H |
| 16829 | H | D59 | H |
| 16830 | H | D60 | H |
| 16831 | H | D61 | H |
| 16832 | H | D62 | H |
| 16833 | H | D63 | H |
| 16834 | H | D64 | H |
| 16835 | H | D65 | H |
| 16836 | H | D66 | H |
| 16837 | H | D67 | H |
| 16838 | H | D68 | H |
| 16839 | H | D69 | H |
| 16840 | H | D70 | H |
| 16841 | H | D71 | H |
| 16842 | H | D72 | H |
| 16843 | H | D73 | H |
| 16844 | H | D74 | H |
| 16845 | H | D75 | H |
| 16846 | H | D76 | H |
| 16847 | H | D77 | H |
| 16848 | H | D78 | H |
| 16849 | H | D79 | H |
| 16850 | H | D80 | H |
| 16851 | H | D81 | H |
| 16852 | H | D82 | H |
| 16853 | H | D83 | H |
| 16854 | H | D84 | H |
| 16855 | H | D85 | H |
| 16856 | H | D86 | H |
| 16857 | H | D87 | H |
| 16858 | H | D88 | H |
| 16859 | H | D89 | H |
| 16860 | H | D90 | H |
| 16861 | H | D91 | H |
| 16862 | H | D92 | H |
| 16863 | H | D93 | H |
| 16864 | H | D94 | H |
| 16865 | H | D95 | H |
| 16866 | H | D96 | H |
| 16867 | H | D97 | H |
| 16868 | H | D98 | H |
| 16869 | H | D99 | H |
| 16870 | H | D100 | H |
| 16871 | H | D101 | H |
| 16872 | H | D102 | H |

TABLE 10

| No. | R⁴² | R⁴³ | R⁴⁴~R⁴⁸ |
|---|---|---|---|
| 16873 | H | D103 | H |
| 16874 | H | D104 | H |
| 16875 | H | D105 | H |
| 16876 | H | D106 | H |
| 16877 | H | D107 | H |
| 16878 | H | D108 | H |
| 16879 | H | D109 | H |
| 16880 | H | D110 | H |
| 16881 | H | D111 | H |
| 16882 | H | D112 | H |
| 16883 | H | D113 | H |
| 16884 | H | D114 | H |
| 16885 | H | D115 | H |
| 16886 | H | D116 | H |
| 16887 | H | D117 | H |
| 16888 | H | D118 | H |
| 16889 | H | D119 | H |
| 16890 | H | D120 | H |
| 16891 | H | D121 | H |
| 16892 | H | D122 | H |
| 16893 | H | D123 | H |
| 16894 | H | D124 | H |
| 16895 | H | D125 | H |
| 16896 | H | D126 | H |
| 16897 | H | D127 | H |
| 16898 | H | D128 | H |
| 16899 | H | D129 | H |
| 16900 | H | D130 | H |
| 16901 | H | D131 | H |
| 16902 | H | D132 | H |
| 16903 | H | D133 | H |
| 16904 | H | D134 | H |
| 16905 | H | D135 | H |
| 16906 | H | D136 | H |
| 16907 | H | D137 | H |
| 16908 | H | D138 | H |
| 16909 | H | D139 | H |
| 16910 | H | D140 | H |
| 16911 | H | D141 | H |
| 16912 | H | D142 | H |
| 16913 | H | D143 | H |
| 16914 | H | D144 | H |
| 16915 | H | D145 | H |
| 16916 | H | D146 | H |
| 16917 | H | D147 | H |
| 16918 | H | D148 | H |
| 16919 | H | D149 | H |
| 16920 | H | D150 | H |
| 16921 | H | D151 | H |
| 16922 | H | D152 | H |
| 16923 | H | D153 | H |
| 16924 | H | D154 | H |
| 16925 | H | D155 | H |
| 16926 | H | D156 | H |
| 16927 | H | D157 | H |
| 16928 | H | D158 | H |
| 16929 | H | D159 | H |
| 16930 | H | D160 | H |
| 16931 | H | D161 | H |
| 16932 | H | D162 | H |
| 16933 | H | D163 | H |
| 16934 | H | D164 | H |
| 16935 | H | D165 | H |
| 16936 | H | D166 | H |
| 16937 | H | D167 | H |
| 16938 | H | D168 | H |
| 16939 | H | D169 | H |
| 16940 | H | D170 | H |
| 16941 | H | D171 | H |
| 16942 | H | D172 | H |
| 16943 | H | D173 | H |
| 16944 | H | D174 | H |
| 16945 | H | D175 | H |
| 16946 | H | D176 | H |
| 16947 | H | D177 | H |
| 16948 | H | D178 | H |
| 16949 | H | D179 | H |
| 16950 | H | D180 | H |
| 16951 | H | D181 | H |
| 16952 | H | D182 | H |
| 16953 | H | D183 | H |
| 16954 | H | D184 | H |
| 16955 | H | D185 | H |
| 16956 | H | D186 | H |
| 16957 | H | D187 | H |
| 16958 | H | D188 | H |
| 16959 | H | D189 | H |
| 16960 | H | D190 | H |
| 16961 | H | D191 | H |
| 16962 | H | D192 | H |
| 16963 | H | D193 | H |
| 16964 | H | D194 | H |
| 16965 | H | D195 | H |
| 16966 | H | D196 | H |
| 16967 | H | D197 | H |
| 16968 | H | D198 | H |
| 16969 | H | D199 | H |
| 16970 | H | D200 | H |
| 16971 | H | D201 | H |
| 16972 | H | D202 | H |
| 16973 | H | D203 | H |
| 16974 | H | D204 | H |
| 16975 | H | D205 | H |
| 16976 | H | D206 | H |
| 16977 | H | D207 | H |
| 16978 | H | D208 | H |
| 16979 | H | D209 | H |
| 16980 | H | D210 | H |
| 16981 | H | D211 | H |
| 16982 | H | D212 | H |
| 16983 | H | D213 | H |
| 16984 | H | D214 | H |
| 16985 | H | D215 | H |
| 16986 | H | D216 | H |
| 16987 | H | D217 | H |
| 16988 | H | D218 | H |
| 16989 | H | D219 | H |
| 16990 | H | D220 | H |
| 16991 | H | D221 | H |
| 16992 | H | D222 | H |

TABLE 11

| No. | R⁴² | R⁴³ | R⁴⁴~R⁴⁸ |
|---|---|---|---|
| 16993 | H | D223 | H |
| 16994 | H | D224 | H |
| 16995 | H | D225 | H |
| 16996 | H | D226 | H |
| 16997 | H | D227 | H |
| 16998 | H | D228 | H |
| 16999 | H | D229 | H |
| 17000 | H | D230 | H |
| 17001 | H | D231 | H |
| 17002 | H | D232 | H |
| 17003 | H | D233 | H |
| 17004 | H | D234 | H |
| 17005 | H | D235 | H |
| 17006 | H | D236 | H |
| 17007 | H | D237 | H |
| 17008 | H | D238 | H |
| 17009 | H | D239 | H |
| 17010 | H | D240 | H |
| 17011 | H | D241 | H |
| 17012 | H | D242 | H |
| 17013 | H | D243 | H |
| 17014 | H | D244 | H |
| 17015 | H | D245 | H |
| 17016 | H | D246 | H |
| 17017 | H | D247 | H |
| 17018 | H | D248 | H |
| 17019 | H | D249 | H |
| 17020 | H | D250 | H |
| 17021 | H | D251 | H |
| 17022 | H | D252 | H |
| 17023 | H | D253 | H |
| 17024 | H | D254 | H |
| 17025 | H | D255 | H |
| 17026 | H | D256 | H |
| 17027 | H | D257 | H |
| 17028 | H | D258 | H |

TABLE 12

| | | Substituting |
|---|---|---|
| New Compound Nos. | Substituted Groups | Groups |
| Nos. 17029 to 17286 are | by substituting R43 of | with the same |
| compounds obtained | Nos. 16513 to 16770 | group as R42. |
| Nos. 17287 to 17544 are | by substituting R44 of | with the same |
| compounds obtained | Nos. 16771 to 17028 | group as R43. |
| Nos. 17545 to 18576 are | by substituting R45 of | with N1. |
| compounds obtained | Nos. 16513 to 17544 | |
| Nos. 18577 to 19608 are | by substituting R46 of | with N1. |
| compounds obtained | Nos. 16513 to 17544 | |
| Nos. 19609 to 20640 are | by substituting R47 of | with N1. |
| compounds obtained | Nos. 16513 to 17544 | |
| Nos. 20641 to 21672 are | by substituting R48 of | with N1. |
| compounds obtained | Nos. 16513 to 17544 | |
| Nos. 21673 to 22704 are | by substituting R45 of | with A1. |
| compounds obtained | Nos. 16513 to 17544 | |
| Nos. 22705 to 23736 are | by substituting R46 of | with A1. |
| compounds obtained | Nos. 16513 to 17544 | |
| Nos. 23737 to 24768 are | by substituting R47 of | with A1. |
| compounds obtained | Nos. 16513 to 17544 | |
| Nos. 24769 to 25800 are | by substituting R48 of | with A1. |
| compounds obtained | Nos. 16513 to 17544 | |
| Nos. 25801 to 26832 are | by substituting R45 of | with N5. |
| compounds obtained | Nos. 16513 to 17544 | |
| Nos. 26833 to 27864 are | by substituting R46 of | with N5. |
| compounds obtained | Nos. 16513 to 17544 | |
| Nos. 27865 to 28896 are | by substituting R47 of | with N5. |
| compounds obtained | Nos. 16513 to 17544 | |
| Nos. 28897 to 29928 are | by substituting R48 of | with N5. |
| compounds obtained | Nos. 16513 to 17544 | |

TABLE 13
General Formula (4c′)

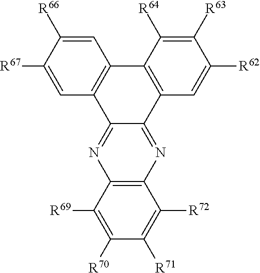

| | | | R⁶⁶, R⁶⁷, |
|---|---|---|---|
| No. | R⁶² | R⁶³ | R⁶⁹~R⁷² |
| 29929 | D1 | H | H |
| 29930 | D2 | H | H |
| 29931 | D3 | H | H |
| 29932 | D4 | H | H |
| 29933 | DS | H | H |
| 29934 | D6 | H | H |
| 29935 | D7 | H | H |
| 29936 | D8 | H | H |
| 29937 | D9 | H | H |
| 29938 | D10 | H | H |
| 29939 | D11 | H | H |
| 29940 | D12 | H | H |
| 29941 | D13 | H | H |
| 29942 | D14 | H | H |
| 29943 | D15 | H | H |
| 29944 | D16 | H | H |
| 29945 | D17 | H | H |
| 29946 | D18 | H | H |
| 29947 | D19 | H | H |
| 29948 | D20 | H | H |
| 29949 | D21 | H | H |
| 29950 | D22 | H | H |
| 29951 | D23 | H | H |
| 29952 | D24 | H | H |
| 29953 | D25 | H | H |
| 29954 | D26 | H | H |
| 29955 | D27 | H | H |
| 29956 | D28 | H | H |
| 29957 | D29 | H | H |
| 29958 | D30 | H | H |
| 29959 | D31 | H | H |
| 29960 | D32 | H | H |
| 29961 | D33 | H | H |
| 29962 | D34 | H | H |
| 29963 | D35 | H | H |
| 29964 | D36 | H | H |
| 29965 | D37 | H | H |
| 29966 | D38 | H | H |
| 29967 | D39 | H | H |
| 29968 | D40 | H | H |
| 29969 | D41 | H | H |
| 29970 | D42 | H | H |
| 29971 | D43 | H | H |
| 29972 | D44 | H | H |
| 29973 | D45 | H | H |
| 29974 | D46 | H | H |
| 29975 | D47 | H | H |
| 29976 | D48 | H | H |
| 29977 | D49 | H | H |
| 29978 | D50 | H | H |
| 29979 | D51 | H | H |
| 29980 | D52 | H | H |
| 29981 | D53 | H | H |
| 29982 | D54 | H | H |
| 29983 | D55 | H | H |
| 29984 | D56 | H | H |
| 29985 | D57 | H | H |
| 29986 | D58 | H | H |
| 29987 | D59 | H | H |
| 29988 | D60 | H | H |
| 29989 | D61 | H | H |
| 29990 | D62 | H | H |
| 29991 | D63 | H | H |
| 29992 | D64 | H | H |
| 29993 | D65 | H | H |
| 29994 | D66 | H | H |
| 29995 | D67 | H | H |
| 29996 | D68 | H | H |
| 29997 | D69 | H | H |
| 29998 | D70 | H | H |
| 29999 | D71 | H | H |
| 30000 | D72 | H | H |
| 30001 | D73 | H | H |
| 30002 | D74 | H | H |
| 30003 | D75 | H | H |
| 30004 | D76 | H | H |
| 30005 | D77 | H | H |
| 30006 | D78 | H | H |
| 30007 | D79 | H | H |
| 30008 | D80 | H | H |
| 30009 | D81 | H | H |
| 30010 | D82 | H | H |
| 30011 | D83 | H | H |
| 30012 | D84 | H | H |
| 30013 | D85 | H | H |
| 30014 | D86 | H | H |
| 30015 | D87 | H | H |
| 30016 | D88 | H | H |
| 30017 | D89 | H | H |
| 30018 | D90 | H | H |
| 30019 | D91 | H | H |
| 30020 | D92 | H | H |
| 30021 | D93 | H | H |
| 30022 | D94 | H | H |
| 30023 | D95 | H | H |
| 30024 | D96 | H | H |
| 30025 | D97 | H | H |
| 30026 | D98 | H | H |
| 30027 | D99 | H | H |
| 30028 | D100 | H | H |
| 30029 | D101 | H | H |
| 30030 | D102 | H | H |
| 30031 | D103 | H | H |
| 30032 | D104 | H | H |
| 30033 | D105 | H | H |
| 30034 | D106 | H | H |
| 30035 | D107 | H | H |
| 30036 | D108 | H | H |
| 30037 | D109 | H | H |
| 30038 | D110 | H | H |
| 30039 | D111 | H | H |
| 30040 | D112 | H | H |
| 30041 | D113 | H | H |
| 30042 | D114 | H | H |
| 30043 | D115 | H | H |
| 30044 | D116 | H | H |
| 30045 | D117 | H | H |
| 30046 | D118 | H | H |
| 30047 | D119 | H | H |
| 30048 | D120 | H | H |

TABLE 14

| No. | R⁶² | R⁶³ | R⁶⁶, R⁶⁷, R⁶⁹~R⁷² |
|---|---|---|---|
| 30049 | D121 | H | H |
| 30050 | D122 | H | H |
| 30051 | D123 | H | H |
| 30052 | D124 | H | H |
| 30053 | D125 | H | H |
| 30054 | D126 | H | H |
| 30055 | D127 | H | H |
| 30056 | D128 | H | H |
| 30057 | D129 | H | H |
| 30058 | D130 | H | H |
| 30059 | D131 | H | H |
| 30060 | D132 | H | H |
| 30061 | D133 | H | H |
| 30062 | D134 | H | H |
| 30063 | D135 | H | H |
| 30064 | D136 | H | H |
| 30065 | D137 | H | H |
| 30066 | D138 | H | H |
| 30067 | D139 | H | H |
| 30068 | D140 | H | H |
| 30069 | D141 | H | H |
| 30070 | D142 | H | H |
| 30071 | D143 | H | H |
| 30072 | D144 | H | H |
| 30073 | D145 | H | H |
| 30074 | D146 | H | H |
| 30075 | D147 | H | H |
| 30076 | D148 | H | H |
| 30077 | D149 | H | H |
| 30078 | D150 | H | H |
| 30079 | D151 | H | H |
| 30080 | D152 | H | H |
| 30081 | D153 | H | H |
| 30082 | D154 | H | H |
| 30083 | D155 | H | H |
| 30084 | D156 | H | H |
| 30085 | D157 | H | H |
| 30086 | D158 | H | H |
| 30087 | D159 | H | H |
| 30088 | D160 | H | H |
| 30089 | D161 | H | H |
| 30090 | D162 | H | H |
| 30091 | D163 | H | H |
| 30092 | D164 | H | H |
| 30093 | D165 | H | H |
| 30094 | D166 | H | H |
| 30095 | D167 | H | H |
| 30096 | D168 | H | H |
| 30097 | D169 | H | H |
| 30098 | D170 | H | H |
| 30099 | D171 | H | H |
| 30100 | D172 | H | H |
| 30101 | D173 | H | H |
| 30102 | D174 | H | H |
| 30103 | D175 | H | H |
| 30104 | D176 | H | H |
| 30105 | D177 | H | H |
| 30106 | D178 | H | H |
| 30107 | D179 | H | H |
| 30108 | D180 | H | H |
| 30109 | D181 | H | H |
| 30110 | D182 | H | H |
| 30111 | D183 | H | H |
| 30112 | D184 | H | H |
| 30113 | D185 | H | H |
| 30114 | D186 | H | H |
| 30115 | D187 | H | H |
| 30116 | D188 | H | H |
| 30117 | D189 | H | H |
| 30118 | D190 | H | H |
| 30119 | D191 | H | H |
| 30120 | D192 | H | H |
| 30121 | D193 | H | H |
| 30122 | D194 | H | H |
| 30123 | D195 | H | H |
| 30124 | D196 | H | H |
| 30125 | D197 | H | H |
| 30126 | D198 | H | H |
| 30127 | D199 | H | H |
| 30128 | D200 | H | H |
| 30129 | D201 | H | H |
| 30130 | D202 | H | H |
| 30131 | D203 | H | H |
| 30132 | D204 | H | H |
| 30133 | D205 | H | H |
| 30134 | D206 | H | H |
| 30135 | D207 | H | H |
| 30136 | D208 | H | H |
| 30137 | D209 | H | H |
| 30138 | D210 | H | H |
| 30139 | D211 | H | H |
| 30140 | D212 | H | H |
| 30141 | D213 | H | H |
| 30142 | D214 | H | H |
| 30143 | D215 | H | H |
| 30144 | D216 | H | H |
| 30145 | D217 | H | H |
| 30146 | D218 | H | H |
| 30147 | D219 | H | H |
| 30148 | D220 | H | H |
| 30149 | D221 | H | H |
| 30150 | D222 | H | H |
| 30151 | D223 | H | H |
| 30152 | D224 | H | H |
| 30153 | D225 | H | H |
| 30154 | D226 | H | H |
| 30155 | D227 | H | H |
| 30156 | D228 | H | H |
| 30157 | D229 | H | H |
| 30158 | D230 | H | H |
| 30159 | D231 | H | H |
| 30160 | D232 | H | H |
| 30161 | D233 | H | H |
| 30162 | D234 | H | H |
| 30163 | D235 | H | H |
| 30164 | D236 | H | H |
| 30165 | D237 | H | H |
| 30166 | D238 | H | H |
| 30167 | D239 | H | H |
| 30168 | D240 | H | H |

TABLE 15

| | | | R⁶⁶, R⁶⁷ |
|---|---|---|---|
| No. | R⁶² | R⁶³ | R⁶⁹~R⁷² |
| 30169 | D241 | H | H |
| 30170 | D242 | H | H |
| 30171 | D243 | H | H |
| 30172 | D244 | H | H |
| 30173 | D245 | H | H |
| 30174 | D246 | H | H |
| 30175 | D247 | H | H |
| 30176 | D248 | H | H |
| 30177 | D249 | H | H |
| 30178 | D250 | H | H |
| 30179 | D251 | H | H |
| 30180 | D252 | H | H |
| 30181 | D253 | H | H |
| 30182 | D254 | H | H |
| 30183 | D255 | H | H |
| 30184 | D256 | H | H |
| 30185 | D257 | H | H |
| 30186 | D258 | H | H |
| 30187 | H | D1 | H |
| 30188 | H | D2 | H |
| 30189 | H | D3 | H |
| 30190 | H | D4 | H |
| 30191 | H | D5 | H |
| 30192 | H | D6 | H |
| 30193 | H | D7 | H |
| 30194 | H | D8 | H |
| 30195 | H | D9 | H |
| 30196 | H | D10 | H |
| 30197 | H | D11 | H |
| 30198 | H | D12 | H |
| 30199 | H | D13 | H |
| 30200 | H | D14 | H |
| 30201 | H | D15 | H |
| 30202 | H | D16 | H |
| 30203 | H | D17 | H |
| 30204 | H | D18 | H |
| 30205 | H | D19 | H |
| 30206 | H | D20 | H |
| 30207 | H | D21 | H |
| 30208 | H | D22 | H |
| 30209 | H | D23 | H |
| 30210 | H | D24 | H |
| 30211 | H | D25 | H |
| 30212 | H | D26 | H |
| 30213 | H | D27 | H |
| 30214 | H | D28 | H |
| 30215 | H | D29 | H |
| 30216 | H | D30 | H |
| 30217 | H | D31 | H |
| 30218 | H | D32 | H |
| 30219 | H | D33 | H |
| 30220 | H | D34 | H |
| 30221 | H | D35 | H |
| 30222 | H | D36 | H |
| 30223 | H | D37 | H |
| 30224 | H | D38 | H |
| 30225 | H | D39 | H |
| 30226 | H | D40 | H |
| 30227 | H | D41 | H |
| 30228 | H | D42 | H |
| 30229 | H | D43 | H |
| 30230 | H | D44 | H |
| 30231 | H | D45 | H |
| 30232 | H | D46 | H |
| 30233 | H | D47 | H |
| 30234 | H | D48 | H |
| 30235 | H | D49 | H |
| 30236 | H | D50 | H |
| 30237 | H | D51 | H |
| 30238 | H | D52 | H |
| 30239 | H | D53 | H |
| 30240 | H | D54 | H |
| 30241 | H | D55 | H |
| 30242 | H | D56 | H |
| 30243 | H | D57 | H |
| 30244 | H | D58 | H |
| 30245 | H | D59 | H |
| 30246 | H | D60 | H |
| 30247 | H | D61 | H |
| 30248 | H | D62 | H |
| 30249 | H | D63 | H |
| 30250 | H | D64 | H |
| 30251 | H | D65 | H |
| 30252 | H | D66 | H |
| 30253 | H | D67 | H |
| 30254 | H | D68 | H |
| 30255 | H | D69 | H |
| 30256 | H | D70 | H |
| 30257 | H | D71 | H |
| 30258 | H | D72 | H |
| 30259 | H | D73 | H |
| 30260 | H | D74 | H |
| 30261 | H | D75 | H |
| 30262 | H | D76 | H |
| 30263 | H | D77 | H |
| 30264 | H | D78 | H |
| 30265 | H | D79 | H |
| 30266 | H | D80 | H |
| 30267 | H | D81 | H |
| 30268 | H | D82 | H |
| 30269 | H | D83 | H |
| 30270 | H | D84 | H |
| 30271 | H | D85 | H |
| 30272 | H | D86 | H |
| 30273 | H | D87 | H |
| 30274 | H | D88 | H |
| 30275 | H | D89 | H |
| 30276 | H | D90 | H |
| 30277 | H | D91 | H |
| 30278 | H | D92 | H |
| 30279 | H | D93 | H |
| 30280 | H | D94 | H |
| 30281 | H | D95 | H |
| 30282 | H | D96 | H |
| 30283 | H | D97 | H |
| 30284 | H | D98 | H |
| 30285 | H | D99 | H |
| 30286 | H | D100 | H |
| 30287 | H | D101 | H |
| 30288 | H | D102 | H |

TABLE 16

| | | | R⁶⁶, R⁶⁷ |
|---|---|---|---|
| No. | R⁶² | R⁶³ | R⁶⁹~R⁷² |
| 30289 | H | D103 | H |
| 30290 | H | D104 | H |
| 30291 | H | D105 | H |
| 30292 | H | D106 | H |
| 30293 | H | D107 | H |
| 30294 | H | D108 | H |
| 30295 | H | D109 | H |
| 30296 | H | D110 | H |
| 30297 | H | D111 | H |
| 30298 | H | D112 | H |
| 30299 | H | D113 | H |
| 30300 | H | D114 | H |
| 30301 | H | D115 | H |
| 30302 | H | D116 | H |
| 30303 | H | D117 | H |
| 30304 | H | D118 | H |
| 30305 | H | D119 | H |
| 30306 | H | D120 | H |
| 30307 | H | D121 | H |
| 30308 | H | D122 | H |
| 30309 | H | D123 | H |
| 30310 | H | D124 | H |
| 30311 | H | D125 | H |
| 30312 | H | D126 | H |
| 30313 | H | D127 | H |
| 30314 | H | D128 | H |
| 30315 | H | D129 | H |
| 30316 | H | D130 | H |
| 30317 | H | D131 | H |
| 30318 | H | D132 | H |
| 30319 | H | D133 | H |
| 30320 | H | D134 | H |
| 30321 | H | D135 | H |
| 30322 | H | D136 | H |
| 30323 | H | D137 | H |
| 30324 | H | D138 | H |
| 30325 | H | D139 | H |
| 30326 | H | D140 | H |
| 30327 | H | D141 | H |
| 30328 | H | D142 | H |
| 30329 | H | D143 | H |
| 30330 | H | D144 | H |
| 30331 | H | D145 | H |
| 30332 | H | D146 | H |
| 30333 | H | D147 | H |
| 30334 | H | D148 | H |
| 30335 | H | D149 | H |
| 30336 | H | D150 | H |
| 30337 | H | D151 | H |
| 30338 | H | D152 | H |
| 30339 | H | D153 | H |
| 30340 | H | D154 | H |
| 30341 | H | D155 | H |
| 30342 | H | D156 | H |
| 30343 | H | D157 | H |
| 30344 | H | D158 | H |
| 30345 | H | D159 | H |
| 30346 | H | D160 | H |
| 30347 | H | D161 | H |
| 30348 | H | D162 | H |
| 30349 | H | D163 | H |
| 30350 | H | D164 | H |
| 30351 | H | D165 | H |
| 30352 | H | D166 | H |
| 30353 | H | D167 | H |
| 30354 | H | D168 | H |
| 30355 | H | D169 | H |
| 30356 | H | D170 | H |
| 30357 | H | D171 | H |
| 30358 | H | D172 | H |
| 30359 | H | D173 | H |
| 30360 | H | D174 | H |
| 30361 | H | D175 | H |
| 30362 | H | D176 | H |
| 30363 | H | D177 | H |
| 30364 | H | D178 | H |
| 30365 | H | D179 | H |
| 30366 | H | D180 | H |
| 30367 | H | D181 | H |
| 30368 | H | D182 | H |
| 30369 | H | D183 | H |
| 30370 | H | D184 | H |
| 30371 | H | D185 | H |
| 30372 | H | D186 | H |
| 30373 | H | D187 | H |
| 30374 | H | D188 | H |
| 30375 | H | D189 | H |
| 30376 | H | D190 | H |
| 30377 | H | D191 | H |
| 30378 | H | D192 | H |
| 30379 | H | D193 | H |
| 30380 | H | D194 | H |
| 30381 | H | D195 | H |
| 30382 | H | D196 | H |
| 30383 | H | D197 | H |
| 30384 | H | D198 | H |
| 30385 | H | D199 | H |
| 30386 | H | D200 | H |
| 30387 | H | D201 | H |
| 30388 | H | D202 | H |
| 30389 | H | D203 | H |
| 30390 | H | D204 | H |
| 30391 | H | D205 | H |
| 30392 | H | D206 | H |
| 30393 | H | D207 | H |
| 30394 | H | D208 | H |
| 30395 | H | D209 | H |
| 30396 | H | D210 | H |
| 30397 | H | D211 | H |
| 30398 | H | D212 | H |
| 30399 | H | D213 | H |
| 30400 | H | D214 | H |
| 30401 | H | D215 | H |
| 30402 | H | D216 | H |
| 30403 | H | D217 | H |
| 30404 | H | D218 | H |
| 30405 | H | D219 | H |
| 30406 | H | D220 | H |
| 30407 | H | D221 | H |
| 30408 | H | D222 | H |

TABLE 17

| | | | R⁶⁶, R⁶⁷ |
|---|---|---|---|
| No. | R⁶² | R⁶³ | R⁶⁹~R⁷² |
| 30409 | H | D223 | H |
| 30410 | H | D224 | H |
| 30411 | H | D225 | H |
| 30412 | H | D226 | H |
| 30413 | H | D227 | H |
| 30414 | H | D228 | H |
| 30415 | H | D229 | H |
| 30416 | H | D230 | H |
| 30417 | H | D231 | H |
| 30418 | H | D232 | H |
| 30419 | H | D233 | H |
| 30420 | H | D234 | H |
| 30421 | H | D235 | H |
| 30422 | H | D236 | H |
| 30423 | H | D237 | H |
| 30424 | H | D238 | H |
| 30425 | H | D239 | H |
| 30426 | H | D240 | H |
| 30427 | H | D241 | H |
| 30428 | H | D242 | H |
| 30429 | H | D243 | H |
| 30430 | H | D244 | H |
| 30431 | H | D245 | H |
| 30432 | H | D246 | H |
| 30433 | H | D247 | H |
| 30434 | H | D248 | H |
| 30435 | H | D249 | H |
| 30436 | H | D250 | H |
| 30437 | H | D251 | H |
| 30438 | H | D252 | H |
| 30439 | H | D253 | H |
| 30440 | H | D254 | H |
| 30441 | H | D255 | H |
| 30442 | H | D256 | H |
| 30443 | H | D257 | H |
| 30444 | H | D258 | H |

TABLE 18

| | | Substituting |
|---|---|---|
| New Compound Nos. | Substituted Groups | Groups |
| Nos. 30445 to 30702 are | by substituting R67 of | with the same |
| compounds obtained | Nos. 29929 to 30186 | group as R62. |
| Nos. 30703 to 30960 are | by substituting R66 of | with the same |
| compounds obtained | Nos. 30187 to 30444 | group as R63. |
| Nos. 30961 to 31992 are | by substituting R69 of | with N1. |
| compounds obtained | Nos. 29929 to 30960 | |
| Nos. 31993 to 33024 are | by substituting R70 of | with N1. |
| compounds obtained | Nos. 29929 to 30960 | |
| Nos. 33025 to 33540 are | by substituting R71 of | with N1. |
| compounds obtained | Nos. 29929 to 30444 | |
| Nos. 33541 to 34056 are | by substituting R72 of | with N1. |
| compounds obtained | Nos. 29929 to 30444 | |
| Nos. 34057 to 34314 are | by substituting R62 of | with A1. |
| compounds obtained | Nos. 30187 to 30444 | |
| Nos. 34315 to 34572 are | by substituting R62 of | with A1. |
| compounds obtained | Nos. 30703 to 30960 | |
| Nos. 34573 to 35346 are | by substituting R66 of | with A1. |
| compounds obtained | Nos. 29929 to 30702 | |
| Nos. 35347 to 35862 are | by substituting R71 of | with N5. |
| compounds obtained | Nos. 29929 to 30444 | |
| Nos. 35863 to 36120 are | by substituting R63 and | with the same |
| compounds obtained | R64 of Nos. 29929 to 30186 | group as R62. |
| Nos. 36121 to 36378 are | by substituting R64 of | with the same |
| compounds obtained | Nos. 29929 to 30186 | group as R62 |
| | substituting R63 of | and with R259. |
| | Nos. 29929 to 30186 | |
| Nos. 36379 to 36636 are | by substituting R62 and | with the same |
| compounds obtained | R64 of Nos. 30187 to 30444 | group as R259. |
| Nos. 36637 to 37410 are | by substituting R69 of | with A1. |
| compounds obtained | Nos. 36895 to 37668 | |
| Nos. 37411 to 38184 are | by substituting R70 of | with A1. |
| compounds obtained | Nos. 36895 to 37668 | |
| Nos. 38185 to 38958 are | by substituting R71 of | with A1. |
| compounds obtained | Nos. 36895 to 37668 | |
| Nos. 38959 to 39732 are | by substituting R72 of | with A1. |
| compounds obtained | Nos. 36895 to 37668 | |
| Nos. 39733 to 40506 are | by substituting R70 and | with N1. |
| compounds obtained | R71 of Nos. 36895 to 37668 | |
| Nos. 40507 to 41022 are | by substituting R70 and | with N1. |
| compounds obtained | R71 of Nos. 30445 to 30960 | |
| Nos. 41023 to 41280 are | by substituting R63 of | with the same |
| compounds obtained | Nos. 29929 to 30186 | group as R62. |
| Nos. 41281 to 41538 are | by substituting R66 of | with A1. |
| compounds obtained | Nos. 41023 to 41280 | |

TABLE 19
General Formula (4d′)

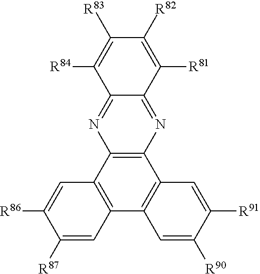

| | | | R⁸³, R⁸⁴, R⁸⁶, |
|---|---|---|---|
| No. | R⁸¹ | R³² | R⁸⁷, R⁹⁰, R⁹¹ |
| 41539 | D1 | H | H |
| 41540 | D2 | H | H |
| 41541 | D3 | H | H |
| 41542 | D4 | H | H |
| 41543 | D5 | H | H |
| 41544 | D6 | H | H |
| 41545 | D7 | H | H |
| 41546 | D8 | H | H |
| 41547 | D9 | H | H |
| 41548 | D10 | H | H |
| 41549 | D11 | H | H |
| 41550 | D12 | H | H |
| 41551 | D13 | H | H |
| 41552 | D14 | H | H |
| 41553 | D15 | H | H |
| 41554 | D16 | H | H |
| 41555 | D17 | H | H |
| 41556 | D18 | H | H |
| 41557 | D19 | H | H |
| 41558 | D20 | H | H |
| 41559 | D21 | H | H |
| 41560 | D22 | H | H |
| 41561 | D23 | H | H |
| 41562 | D24 | H | H |
| 41563 | D25 | H | H |
| 41564 | D26 | H | H |
| 41565 | D27 | H | H |
| 41566 | D28 | H | H |
| 41567 | D29 | H | H |
| 41568 | D30 | H | H |
| 41569 | D31 | H | H |
| 41570 | D32 | H | H |
| 41571 | D33 | H | H |
| 41572 | D34 | H | H |
| 41573 | D35 | H | H |
| 41574 | D36 | H | H |
| 41575 | D37 | H | H |
| 41576 | D38 | H | H |
| 41577 | D39 | H | H |
| 41578 | D40 | H | H |
| 41579 | D41 | H | H |
| 41580 | D42 | H | H |
| 41581 | D43 | H | H |
| 41582 | D44 | H | H |
| 41583 | D45 | H | H |
| 41584 | D46 | H | H |
| 41585 | D47 | H | H |
| 41586 | D48 | H | H |
| 41587 | D49 | H | H |
| 41588 | D50 | H | H |
| 41589 | D51 | H | H |
| 41590 | D52 | H | H |
| 41591 | D53 | H | H |
| 41592 | D54 | H | H |
| 41593 | D55 | H | H |
| 41594 | D56 | H | H |
| 41595 | D57 | H | H |
| 41596 | D58 | H | H |
| 41597 | D59 | H | H |
| 41598 | D60 | H | H |
| 41599 | D61 | H | H |
| 41600 | D62 | H | H |
| 41601 | D63 | H | H |
| 41602 | D64 | H | H |
| 41603 | D65 | H | H |
| 41604 | D66 | H | H |
| 41605 | D67 | H | H |
| 41606 | D68 | H | H |
| 41607 | D69 | H | H |
| 41608 | D70 | H | H |
| 41609 | D71 | H | H |
| 41610 | D72 | H | H |
| 41611 | D73 | H | H |
| 41612 | D74 | H | H |
| 41613 | D75 | H | H |
| 41614 | D76 | H | H |
| 41615 | D77 | H | H |
| 41616 | D78 | H | H |
| 41617 | D79 | H | H |
| 41618 | D80 | H | H |
| 41619 | D81 | H | H |
| 41620 | D82 | H | H |
| 41621 | D83 | H | H |
| 41622 | D84 | H | H |
| 41623 | D85 | H | H |
| 41624 | D86 | H | H |
| 41625 | D87 | H | H |
| 41626 | D88 | H | H |
| 41627 | D89 | H | H |
| 41628 | D90 | H | H |
| 41629 | D91 | H | H |
| 41630 | D92 | H | H |
| 41631 | D93 | H | H |
| 41632 | D94 | H | H |
| 41633 | D95 | H | H |
| 41634 | D96 | H | H |
| 41635 | D97 | H | H |
| 41636 | D98 | H | H |
| 41637 | D99 | H | H |
| 41638 | D100 | H | H |
| 41639 | D101 | H | H |
| 41640 | D102 | H | H |
| 41641 | D103 | H | H |
| 41642 | D104 | H | H |
| 41643 | D105 | H | H |
| 41644 | D106 | H | H |
| 41645 | D107 | H | H |
| 41646 | D108 | H | H |
| 41647 | D109 | H | H |
| 41648 | D110 | H | H |
| 41649 | D111 | H | H |
| 41650 | D112 | H | H |
| 41651 | D113 | H | H |
| 41652 | D114 | H | H |
| 41653 | D115 | H | H |
| 41654 | D116 | H | H |
| 41655 | D117 | H | H |
| 41656 | D118 | H | H |
| 41857 | D119 | H | H |
| 41668 | D120 | H | H |

TABLE 20

| | | | R⁸³, R⁸⁴, R⁸⁶ |
|---|---|---|---|
| No. | R⁸¹ | R⁸² | R⁸⁷, R⁹⁰, R⁹¹ |
| 41659 | D121 | H | H |
| 41660 | D122 | H | H |
| 41661 | D123 | H | H |
| 41662 | D124 | H | H |
| 41663 | D125 | H | H |
| 41664 | D126 | H | H |
| 41665 | D127 | H | H |
| 41666 | D128 | H | H |
| 41667 | D129 | H | H |
| 41668 | D130 | H | H |
| 41669 | D131 | H | H |
| 41670 | D132 | H | H |
| 41671 | D133 | H | H |
| 41672 | D134 | H | H |
| 41673 | D135 | H | H |
| 41674 | D136 | H | H |
| 41675 | D137 | H | H |
| 41676 | D138 | H | H |
| 41677 | D139 | H | H |
| 41678 | D140 | H | H |
| 41679 | D141 | H | H |
| 41680 | D142 | H | H |
| 41681 | D143 | H | H |
| 41682 | D144 | H | H |
| 41683 | D145 | H | H |
| 41684 | D146 | H | H |
| 41685 | D147 | H | H |
| 41686 | D148 | H | H |
| 41687 | D149 | H | H |
| 41688 | D150 | H | H |
| 41689 | D151 | H | H |
| 41690 | D152 | H | H |
| 41691 | D153 | H | H |
| 41692 | D154 | H | H |
| 41693 | D155 | H | H |
| 41694 | D156 | H | H |
| 41695 | D157 | H | H |
| 41696 | D158 | H | H |
| 41697 | D159 | H | H |
| 41698 | D160 | H | H |
| 41699 | D161 | H | H |
| 41700 | D162 | H | H |
| 41701 | D163 | H | H |
| 41702 | D164 | H | H |
| 41703 | D165 | H | H |
| 41704 | D166 | H | H |
| 41705 | D167 | H | H |
| 41706 | D168 | H | H |
| 41707 | D169 | H | H |
| 41708 | D170 | H | H |
| 41709 | D171 | H | H |
| 41710 | D172 | H | H |
| 41711 | D173 | H | H |
| 41712 | D174 | H | H |
| 41713 | D175 | H | H |
| 41714 | D176 | H | H |
| 41715 | D177 | H | H |
| 41716 | D178 | H | H |
| 41717 | D179 | H | H |
| 41718 | D180 | H | H |
| 41719 | D181 | H | H |
| 41720 | D182 | H | H |
| 41721 | D183 | H | H |
| 41722 | D184 | H | H |
| 41723 | D185 | H | H |
| 41724 | D186 | H | H |
| 41725 | D187 | H | H |
| 41726 | D188 | H | H |
| 41727 | D189 | H | H |
| 41728 | D190 | H | H |
| 41729 | D191 | H | H |
| 41730 | D192 | H | H |
| 41731 | D193 | H | H |
| 41732 | D194 | H | H |
| 41733 | D195 | H | H |
| 41734 | D196 | H | H |
| 41735 | D197 | H | H |
| 41736 | D198 | H | H |
| 41737 | D199 | H | H |
| 41738 | D200 | H | H |
| 41739 | D201 | H | H |
| 41740 | D202 | H | H |
| 41741 | D203 | H | H |
| 41742 | D204 | H | H |
| 41743 | D205 | H | H |
| 41744 | D206 | H | H |
| 41745 | D207 | H | H |
| 41746 | D208 | H | H |
| 41747 | D209 | H | H |
| 41748 | D210 | H | H |
| 41749 | D211 | H | H |
| 41750 | D212 | H | H |
| 41751 | D213 | H | H |
| 41752 | D214 | H | H |
| 41753 | D215 | H | H |
| 41754 | D216 | H | H |
| 41755 | D217 | H | H |
| 41756 | D218 | H | H |
| 41757 | D219 | H | H |
| 41758 | D220 | H | H |
| 41759 | D221 | H | H |
| 41760 | D222 | H | H |
| 41761 | D223 | H | H |
| 41762 | D224 | H | H |
| 41763 | D225 | H | H |
| 41764 | D226 | H | H |
| 41765 | D227 | H | H |
| 41766 | D228 | H | H |
| 41767 | D229 | H | H |
| 41768 | D230 | H | H |
| 41769 | D231 | H | H |
| 41770 | D232 | H | H |
| 41771 | D233 | H | H |
| 41772 | D234 | H | H |
| 41773 | D235 | H | H |
| 41774 | D236 | H | H |
| 41775 | D237 | H | H |
| 41776 | D238 | H | H |
| 41777 | D239 | H | H |
| 41778 | D240 | H | H |

TABLE 21

| | | | R⁸³, R⁸⁴, R⁸⁶ |
|---|---|---|---|
| No. | R⁸¹ | R⁸² | R⁸⁷, R⁹⁰, R⁹¹ |
| 41779 | D241 | H | H |
| 41780 | D242 | H | H |
| 41781 | D243 | H | H |
| 41782 | D244 | H | H |
| 41783 | D245 | H | H |
| 41784 | D246 | H | H |
| 41785 | D247 | H | H |
| 41786 | D248 | H | H |
| 41787 | D249 | H | H |
| 41788 | D250 | H | H |
| 41789 | D251 | H | H |
| 41790 | D252 | H | H |
| 41791 | D253 | H | H |
| 41792 | D254 | H | H |
| 41793 | D255 | H | H |
| 41794 | D256 | H | H |
| 41795 | D257 | H | H |
| 41796 | D258 | H | H |
| 41797 | H | D1 | H |
| 41798 | H | D2 | H |
| 41799 | H | D3 | H |
| 41800 | H | D4 | H |
| 41801 | H | D5 | H |
| 41802 | H | D6 | H |
| 41803 | H | D7 | H |
| 41804 | H | D8 | H |
| 41805 | H | D9 | H |
| 41806 | H | D10 | H |
| 41807 | H | D11 | H |
| 41808 | H | D12 | H |
| 41809 | H | D13 | H |
| 41810 | H | D14 | H |
| 41811 | H | D15 | H |
| 41812 | H | D16 | H |
| 41813 | H | D17 | H |
| 41814 | H | D18 | H |
| 41815 | H | D19 | H |
| 41816 | H | D20 | H |
| 41817 | H | D21 | H |
| 41818 | H | D22 | H |
| 41819 | H | D23 | H |
| 41820 | H | D24 | H |
| 41821 | H | D25 | H |
| 41822 | H | D26 | H |
| 41823 | H | D27 | H |
| 41824 | H | D28 | H |
| 41825 | H | D29 | H |
| 41826 | H | D30 | H |
| 41827 | H | D31 | H |
| 41828 | H | D32 | H |
| 41829 | H | D33 | H |
| 41830 | H | D34 | H |
| 41831 | H | D35 | H |
| 41832 | H | D36 | H |
| 41833 | H | D37 | H |
| 41834 | H | D38 | H |
| 41835 | H | D39 | H |
| 41836 | H | D40 | H |
| 41837 | H | D41 | H |
| 41838 | H | D42 | H |
| 41839 | H | D43 | H |
| 41840 | H | D44 | H |
| 41841 | H | D45 | H |
| 41842 | H | D46 | H |
| 41843 | H | D47 | H |
| 41844 | H | D48 | H |
| 41845 | H | D49 | H |
| 41846 | H | D50 | H |
| 41847 | H | D51 | H |
| 41848 | H | D52 | H |
| 41849 | H | D53 | H |
| 41850 | H | D54 | H |
| 41851 | H | D55 | H |
| 41852 | H | D56 | H |
| 41853 | H | D57 | H |
| 41854 | H | D58 | H |
| 41855 | H | D59 | H |
| 41856 | H | D60 | H |
| 41857 | H | D61 | H |
| 41858 | H | D62 | H |
| 41859 | H | D63 | H |
| 41860 | H | D64 | H |
| 41861 | H | D65 | H |
| 41862 | H | D66 | H |
| 41863 | H | D67 | H |
| 41864 | H | D68 | H |
| 41865 | H | D69 | H |
| 41866 | H | D70 | H |
| 41867 | H | D71 | H |
| 41868 | H | D72 | H |
| 41869 | H | D73 | H |
| 41870 | H | D74 | H |
| 41871 | H | D75 | H |
| 41872 | H | D76 | H |
| 41873 | H | D77 | H |
| 41874 | H | D78 | H |
| 41875 | H | D79 | H |
| 41876 | H | D80 | H |
| 41877 | H | D81 | H |
| 41878 | H | D82 | H |
| 41879 | H | D83 | H |
| 41880 | H | D84 | H |
| 41881 | H | D85 | H |
| 41882 | H | D86 | H |
| 41883 | H | D87 | H |
| 41884 | H | D88 | H |
| 41885 | H | D89 | H |
| 41886 | H | D90 | H |
| 41887 | H | D91 | H |
| 41888 | H | D92 | H |
| 41889 | H | D93 | H |
| 41890 | H | D94 | H |
| 41891 | H | D95 | H |
| 41892 | H | D96 | H |
| 41893 | H | D97 | H |
| 41894 | H | D98 | H |
| 41895 | H | D99 | H |
| 41896 | H | D100 | H |
| 41897 | H | D101 | H |
| 41898 | H | D102 | H |

TABLE 22

| | | | R⁸³, R⁸⁴, R⁸⁶ |
|---|---|---|---|
| No. | R⁸¹ | R⁸² | R⁸⁷, R⁹⁰, R⁹¹ |
| 41899 | H | D103 | H |
| 41900 | H | D104 | H |
| 41901 | H | D105 | H |
| 41902 | H | D106 | H |
| 41903 | H | D107 | H |
| 41904 | H | D108 | H |
| 41905 | H | D109 | H |
| 41906 | H | D110 | H |
| 41907 | H | D111 | H |
| 41908 | H | D112 | H |
| 41909 | H | D113 | H |
| 41910 | H | D114 | H |
| 41911 | H | D115 | H |
| 41912 | H | D116 | H |
| 41913 | H | D117 | H |
| 41914 | H | D118 | H |
| 41915 | H | D119 | H |
| 41916 | H | D120 | H |
| 41917 | H | D121 | H |
| 41918 | H | D122 | H |
| 41919 | H | D123 | H |
| 41920 | H | D124 | H |
| 41921 | H | D125 | H |
| 41922 | H | D126 | H |
| 41923 | H | D127 | H |
| 41924 | H | D128 | H |
| 41925 | H | D129 | H |
| 41926 | H | D130 | H |
| 41927 | H | D131 | H |
| 41928 | H | D132 | H |
| 41929 | H | D133 | H |
| 41930 | H | D134 | H |
| 41931 | H | D135 | H |
| 41932 | H | D136 | H |
| 41933 | H | D137 | H |
| 41934 | H | D138 | H |
| 41935 | H | D139 | H |
| 41936 | H | D140 | H |
| 41937 | H | D141 | H |
| 41938 | H | D142 | H |
| 41939 | H | D143 | H |
| 41940 | H | D144 | H |
| 41941 | H | D145 | H |
| 41942 | H | D146 | H |
| 41943 | H | D147 | H |
| 41944 | H | D148 | H |
| 41945 | H | D149 | H |
| 41946 | H | D150 | H |
| 41947 | H | D151 | H |
| 41948 | H | D152 | H |
| 41949 | H | D153 | H |
| 41950 | H | D154 | H |
| 41951 | H | D155 | H |
| 41952 | H | D156 | H |
| 41953 | H | D157 | H |
| 41954 | H | D158 | H |
| 41955 | H | D159 | H |
| 41956 | H | D160 | H |
| 41957 | H | D161 | H |
| 41958 | H | D162 | H |
| 41959 | H | D163 | H |
| 41960 | H | D164 | H |
| 41961 | H | D165 | H |
| 41962 | H | D166 | H |
| 41963 | H | D167 | H |
| 41964 | H | D168 | H |
| 41965 | H | D169 | H |
| 41966 | H | D170 | H |
| 41967 | H | D171 | H |
| 41968 | H | D172 | H |
| 41969 | H | D173 | H |
| 41970 | H | D174 | H |
| 41971 | H | D175 | H |
| 41972 | H | D176 | H |
| 41973 | H | D177 | H |
| 41974 | H | D178 | H |
| 41975 | H | D179 | H |
| 41976 | H | D180 | H |
| 41977 | H | D181 | H |
| 41978 | H | D182 | H |
| 41979 | H | D183 | H |
| 41980 | H | D184 | H |
| 41981 | H | D185 | H |
| 41982 | H | D186 | H |
| 41983 | H | D187 | H |
| 41984 | H | D188 | H |
| 41985 | H | D189 | H |
| 41986 | H | D190 | H |
| 41987 | H | D191 | H |
| 41988 | H | D192 | H |
| 41989 | H | D193 | H |
| 41990 | H | D194 | H |
| 41991 | H | D195 | H |
| 41992 | H | D196 | H |
| 41993 | H | D197 | H |
| 41994 | H | D198 | H |
| 41995 | H | D199 | H |
| 41996 | H | D200 | H |
| 41997 | H | D201 | H |
| 41998 | H | D202 | H |
| 41999 | H | D203 | H |
| 42000 | H | D204 | H |
| 42001 | H | D205 | H |
| 42002 | H | D206 | H |
| 42003 | H | D207 | H |
| 42004 | H | D208 | H |
| 42005 | H | D209 | H |
| 42006 | H | D210 | H |
| 42007 | H | D211 | H |
| 42008 | H | D212 | H |
| 42009 | H | D213 | H |
| 42010 | H | D214 | H |
| 42011 | H | D215 | H |
| 42012 | H | D216 | H |
| 42013 | H | D217 | H |
| 42014 | H | D218 | H |
| 42015 | H | D219 | H |
| 42016 | H | D220 | H |
| 42017 | H | D221 | H |
| 42018 | H | D222 | H |

TABLE 23

| | | | R⁸³, R⁸⁴, R⁸⁶, |
|---|---|---|---|
| No. | R⁸¹ | R⁸² | R⁸⁷, R⁹⁰, R⁹¹ |
| 42019 | H | D223 | H |
| 42020 | H | D224 | H |
| 42021 | H | D225 | H |
| 42022 | H | D226 | H |
| 42023 | H | D227 | H |
| 42024 | H | D228 | H |
| 42025 | H | D229 | H |
| 42026 | H | D230 | H |
| 42027 | H | D231 | H |
| 42028 | H | D232 | H |
| 42029 | H | D233 | H |
| 42030 | H | D234 | H |
| 42031 | H | D235 | H |
| 42032 | H | D236 | H |
| 42033 | H | D237 | H |
| 42034 | H | D238 | H |
| 42035 | H | D239 | H |
| 42036 | H | D240 | H |
| 42037 | H | D241 | H |
| 42038 | H | D242 | H |
| 42039 | H | D243 | H |
| 42040 | H | D244 | H |
| 42041 | H | D245 | H |
| 42042 | H | D246 | H |
| 42043 | H | D247 | H |
| 42044 | H | D248 | H |
| 42045 | H | D249 | H |
| 42046 | H | D250 | H |
| 42047 | H | D251 | H |
| 42048 | H | D252 | H |
| 42049 | H | D253 | H |
| 42050 | H | D254 | H |
| 42051 | H | D255 | H |
| 42052 | H | D256 | H |
| 42053 | H | D257 | H |
| 42054 | H | D258 | H |

TABLE 24

| New Compound Nos. | Substituted Groups | Substituting Groups |
|---|---|---|
| Nos. 42055 to 42312 are compounds obtained | by substituting R84 of Nos. 41539 to 41796 | with the same group as R81. |
| Nos. 42313 to 42570 are compounds obtained | by substituting R83 of Nos. 41797 to 42054 | with the same group as R82. |
| Nos. 42571 to 42828 are compounds obtained | by substituting R83 of Nos. 41539 to 41796 | with the same group as R81. |
| Nos. 42829 to 43860 are compounds obtained | by substituting R90 of Nos. 41539 to 42570 | with N1. |
| Nos. 43861 to 44892 are compounds obtained | by substituting R91 of Nos. 41539 to 42570 | with N1. |
| Nos. 44893 to 45408 are compounds obtained | by substituting R87 of Nos. 41539 to 42054 | with N1. |
| Nos. 45409 to 45924 are compounds obtained | by substituting R86 of Nos. 41539 to 42054 | with N1. |
| Nos. 45925 to 46956 are compounds obtained | by substituting R91 of Nos. 41539 to 42570 | with A1. |
| Nos. 46957 to 47472 are compounds obtained | by substituting R87 of Nos. 41539 to 42054 | with A1. |
| Nos. 47473 to 47988 are compounds obtained | by substituting R86 of Nos. 41539 to 42054 | with A1. |
| Nos. 47989 to 49020 are compounds obtained | by substituting R90 and R87 of Nos. 41539 to 42570 | with A1. |
| Nos. 49021 to 50052 are compounds obtained | by substituting R91 and R86 of Nos. 41539 to 42570 | with A1. |
| Nos. 50053 to 51084 are compounds obtained | by substituting R90 of Nos. 41539 to 42570 | with N5. |
| Nos. 51085 to 52116 are compounds obtained | by substituting R91 of Nos. 41539 to 42570 | with N5. |
| Nos. 52117 to 52632 are compounds obtained | by substituting R87 of Nos. 41539 to 42054 | with N5. |
| Nos. 52633 to 53148 are compounds obtained | by substituting R86 of Nos. 41539 to 42054 | with N5. |
| Nos. 53149 to 54180 are compounds obtained | by substituting R90 and R87 of Nos. 41539 to 42570 | with N5. |
| Nos. 54181 to 54696 are compounds obtained | by substituting R91 and R86 of Nos. 41539 to 42054 | with N5. |
| Nos. 54697 to 54954 are compounds obtained | by substituting R82 and R83 of Nos. 41539 to 41796 | with the same group as R81. |
| Nos. 54955 to 55212 are compounds obtained | by substituting R83 of Nos. 41539 to 41796 | with the same group as R81 and |
| | substituting R82 of Nos. 41539 to 41796 | with R259. |
| Nos. 55213 to 55470 are compounds obtained | by substituting R81 and R83 of Nos. 41797 to 42054 | with R259. |
| Nos. 55471 to 56244 are compounds obtained | by substituting R86 of Nos. 54697 to 55470 | with A1. |
| Nos. 56245 to 57018 are compounds obtained | by substituting R87 of Nos. 54697 to 55470 | with A1. |
| Nos. 57019 to 57792 are compounds obtained | by substituting R90 of Nos. 54697 to 55470 | with A1. |
| Nos. 57793 to 58566 are compounds obtained | by substituting R91 of Nos. 54697 to 55470 | with A1. |

TABLE 25
General Formula (4e′)

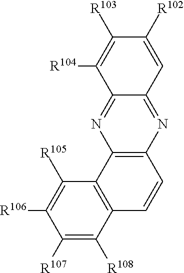

| No. | R¹⁰² | R¹⁰³ | R¹⁰⁴~R¹⁰⁸ |
|---|---|---|---|
| 58567 | D1 | H | H |
| 58568 | D2 | H | H |
| 58569 | D3 | H | H |
| 58570 | D4 | H | H |
| 58571 | D5 | H | H |
| 58572 | D6 | H | H |
| 58573 | D7 | H | H |
| 58574 | D8 | H | H |
| 58575 | D9 | H | H |
| 58576 | D10 | H | H |
| 58577 | D11 | H | H |
| 58578 | D12 | H | H |
| 58579 | D13 | H | H |
| 58580 | D14 | H | H |
| 58581 | D15 | H | H |
| 58582 | D16 | H | H |
| 58583 | D17 | H | H |
| 58584 | D18 | H | H |
| 58585 | D19 | H | H |
| 58586 | D20 | H | H |
| 58587 | D21 | H | H |
| 58588 | D22 | H | H |
| 58589 | D23 | H | H |
| 58590 | D24 | H | H |
| 58591 | D25 | H | H |
| 58592 | D26 | H | H |
| 58593 | D27 | H | H |
| 58594 | D28 | H | H |
| 58595 | D29 | H | H |
| 58596 | D30 | H | H |
| 58597 | D31 | H | H |
| 58598 | D32 | H | H |
| 58599 | D33 | H | H |
| 58600 | D34 | H | H |
| 58601 | D35 | H | H |
| 58602 | D36 | H | H |
| 58603 | D37 | H | H |
| 58604 | D38 | H | H |
| 53605 | D39 | H | H |
| 58606 | D40 | H | H |
| 58607 | D41 | H | H |
| 58608 | D42 | H | H |
| 58609 | D43 | H | H |
| 58610 | D44 | H | H |
| 58611 | D45 | H | H |
| 58612 | D46 | H | H |
| 58613 | D47 | H | H |
| 58614 | D48 | H | H |
| 58615 | D49 | H | H |
| 58616 | D50 | H | H |
| 58617 | D51 | H | H |
| 58618 | D52 | H | H |
| 58619 | D53 | H | H |
| 58620 | D54 | H | H |
| 58621 | D55 | H | H |
| 58622 | D56 | H | H |
| 58623 | D57 | H | H |
| 58624 | D58 | H | H |
| 58625 | D59 | H | H |
| 58626 | D60 | H | H |
| 58627 | D61 | H | H |
| 58628 | D62 | H | H |
| 58629 | D63 | H | H |
| 58630 | D64 | H | H |
| 58631 | D65 | H | H |
| 58632 | D66 | H | H |
| 58833 | D67 | H | H |
| 58634 | D68 | H | H |
| 58635 | D69 | H | H |
| 58636 | D70 | H | H |
| 58637 | D71 | H | H |
| 58638 | D72 | H | H |
| 58639 | D73 | H | H |
| 58640 | D74 | H | H |
| 58641 | D75 | H | H |
| 58642 | D76 | H | H |
| 58643 | D77 | H | H |
| 58644 | D78 | H | H |
| 58645 | D79 | H | H |
| 58646 | D80 | H | H |
| 58647 | D81 | H | H |
| 58648 | D82 | H | H |
| 58649 | D83 | H | H |
| 58650 | D84 | H | H |
| 58651 | D85 | H | H |
| 58652 | D86 | H | H |
| 58653 | D87 | H | H |
| 58654 | D88 | H | H |
| 58655 | D89 | H | H |
| 58656 | D90 | H | H |
| 58657 | D91 | H | H |
| 58658 | D92 | H | H |
| 58659 | D93 | H | H |
| 58660 | D94 | H | H |
| 58661 | D95 | H | H |
| 58662 | D96 | H | H |
| 58663 | D97 | H | H |
| 58664 | D98 | H | H |
| 58665 | D99 | H | H |
| 58666 | D100 | H | H |
| 58667 | D101 | H | H |
| 58668 | D102 | H | H |
| 58869 | D103 | H | H |
| 58670 | D104 | H | H |
| 58671 | D105 | H | H |
| 58672 | D106 | H | H |
| 58673 | D107 | H | H |
| 58674 | D108 | H | H |
| 58675 | D109 | H | H |
| 58676 | D110 | H | H |
| 58677 | D111 | H | H |
| 58678 | D112 | H | H |
| 58679 | D113 | H | H |
| 58680 | D114 | H | H |
| 58681 | D115 | H | H |
| 58682 | D116 | H | H |
| 58683 | D117 | H | H |
| 58684 | D118 | H | H |
| 58685 | D119 | H | H |
| 58686 | D120 | H | H |

TABLE 26

| No. | R¹⁰² | R¹⁰³ | R¹⁰⁴~R¹⁰⁸ |
|---|---|---|---|
| 58687 | D121 | H | H |
| 58688 | D122 | H | H |
| 58689 | D123 | H | H |
| 58690 | D124 | H | H |
| 58691 | D125 | H | H |
| 58692 | D126 | H | H |
| 58693 | D127 | H | H |
| 58694 | D128 | H | H |
| 58695 | D129 | H | H |
| 58696 | D130 | H | H |
| 58697 | D131 | H | H |
| 58698 | D132 | H | H |
| 58699 | D133 | H | H |
| 58700 | D134 | H | H |
| 58701 | D135 | H | H |
| 58702 | D136 | H | H |
| 58703 | D137 | H | H |
| 58704 | D138 | H | H |
| 58705 | D139 | H | H |
| 58706 | D140 | H | H |
| 58707 | D141 | H | H |
| 58708 | D142 | H | H |
| 58709 | D143 | H | H |
| 58710 | D144 | H | H |
| 58711 | D145 | H | H |
| 58712 | D146 | H | H |
| 58713 | D147 | H | H |
| 58714 | D148 | H | H |
| 58715 | D149 | H | H |
| 58716 | D150 | H | H |
| 58717 | D151 | H | H |
| 58718 | D152 | H | H |
| 58719 | D153 | H | H |
| 58720 | D154 | H | H |
| 58721 | D155 | H | H |
| 58722 | D156 | H | H |
| 58723 | D157 | H | H |
| 58724 | D158 | H | H |
| 58725 | D159 | H | H |
| 58726 | D160 | H | H |
| 58727 | D161 | H | H |
| 58728 | D162 | H | H |
| 58729 | D163 | H | H |
| 58730 | D164 | H | H |
| 58731 | D165 | H | H |
| 58732 | D166 | H | H |
| 58733 | D167 | H | H |
| 58734 | D168 | H | H |
| 58735 | D169 | H | H |
| 58736 | D170 | H | H |
| 58737 | D171 | H | H |
| 58738 | D172 | H | H |
| 58739 | D173 | H | H |
| 58740 | D174 | H | H |
| 58741 | D175 | H | H |
| 58742 | D176 | H | H |
| 58743 | D177 | H | H |
| 58744 | D178 | H | H |
| 58745 | D179 | H | H |
| 58746 | D180 | H | H |
| 58747 | D181 | H | H |
| 58748 | D182 | H | H |
| 58749 | D183 | H | H |
| 58750 | D184 | H | H |
| 58751 | D185 | H | H |
| 58752 | D186 | H | H |
| 58753 | D187 | H | H |
| 58754 | D188 | H | H |
| 58755 | D189 | H | H |
| 58756 | D190 | H | H |
| 58757 | D191 | H | H |
| 58758 | D192 | H | H |
| 58759 | D193 | H | H |
| 58760 | D194 | H | H |
| 58761 | D195 | H | H |
| 58762 | D196 | H | H |
| 58763 | D197 | H | H |
| 58764 | D198 | H | H |
| 58765 | D199 | H | H |
| 58766 | D200 | H | H |
| 58767 | D201 | H | H |
| 58768 | D202 | H | H |
| 58769 | D203 | H | H |
| 58770 | D204 | H | H |
| 58771 | D205 | H | H |
| 58772 | D206 | H | H |
| 58773 | D207 | H | H |
| 58774 | D208 | H | H |
| 58775 | D209 | H | H |
| 58776 | D210 | H | H |
| 58777 | D211 | H | H |
| 58778 | D212 | H | H |
| 58779 | D213 | H | H |
| 58780 | D214 | H | H |
| 58781 | D215 | H | H |
| 58782 | D216 | H | H |
| 58783 | D217 | H | H |
| 58784 | D218 | H | H |
| 58785 | D219 | H | H |
| 58786 | D220 | H | H |
| 58787 | D221 | H | H |
| 58788 | D222 | H | H |
| 58789 | D223 | H | H |
| 58790 | D224 | H | H |
| 58791 | D225 | H | H |
| 58792 | D226 | H | H |
| 58793 | D227 | H | H |
| 58794 | D228 | H | H |
| 58795 | D229 | H | H |
| 58796 | D230 | H | H |
| 58797 | D231 | H | H |
| 58798 | D232 | H | H |
| 58799 | D233 | H | H |
| 58800 | D234 | H | H |
| 58801 | D235 | H | H |
| 58802 | D236 | H | H |
| 58803 | D237 | H | H |
| 58804 | D238 | H | H |
| 58805 | D239 | H | H |
| 58806 | D240 | H | H |

TABLE 27

| No. | R¹⁰² | R¹⁰³ | R¹⁰⁴~R¹⁰⁸ |
|---|---|---|---|
| 58807 | D241 | H | H |
| 58808 | D242 | H | H |
| 58809 | D243 | H | H |
| 58810 | D244 | H | H |
| 58811 | D245 | H | H |
| 58812 | D246 | H | H |
| 58813 | D247 | H | H |
| 58814 | D248 | H | H |
| 58815 | D249 | H | H |
| 58816 | D250 | H | H |
| 58817 | D251 | H | H |
| 58818 | D252 | H | H |
| 58819 | D253 | H | H |
| 58820 | D254 | H | H |
| 58821 | D255 | H | H |
| 58822 | D256 | H | H |
| 58823 | D257 | H | H |
| 58824 | D258 | H | H |
| 58825 | H | D1 | H |
| 58826 | H | D2 | H |
| 58827 | H | D3 | H |
| 58828 | H | D4 | H |
| 58829 | H | D5 | H |
| 58830 | H | D6 | H |
| 58831 | H | D7 | H |
| 58832 | H | D8 | H |
| 58833 | H | D9 | H |
| 58834 | H | D10 | H |
| 58835 | H | D11 | H |
| 58836 | H | D12 | H |
| 58837 | H | D13 | H |
| 58838 | H | D14 | H |
| 58839 | H | D15 | H |
| 58840 | H | D16 | H |
| 58841 | H | D17 | H |
| 58842 | H | D18 | H |
| 58843 | H | D19 | H |
| 58844 | H | D20 | H |
| 58845 | H | D21 | H |
| 58846 | H | D22 | H |
| 58847 | H | D23 | H |
| 58848 | H | D24 | H |
| 58849 | H | D25 | H |
| 58850 | H | D26 | H |
| 58851 | H | D27 | H |
| 58852 | H | D28 | H |
| 58853 | H | D29 | H |
| 58854 | H | D30 | H |
| 58855 | H | D31 | H |
| 58856 | H | D32 | H |
| 58857 | H | D33 | H |
| 58858 | H | D34 | H |
| 58859 | H | D35 | H |
| 58860 | H | D36 | H |
| 58861 | H | D37 | H |
| 58862 | H | D38 | H |
| 58863 | H | D39 | H |
| 58864 | H | D40 | H |
| 58865 | H | D41 | H |
| 58866 | H | D42 | H |
| 58867 | H | D43 | H |
| 58868 | H | D44 | H |
| 58869 | H | D45 | H |
| 58870 | H | D46 | H |
| 58871 | H | D47 | H |
| 58872 | H | D48 | H |
| 58873 | H | D49 | H |
| 58874 | H | D50 | H |
| 58875 | H | D51 | H |
| 58876 | H | D52 | H |
| 58877 | H | D53 | H |
| 58878 | H | D54 | H |
| 58879 | H | D55 | H |
| 58880 | H | D56 | H |
| 58881 | H | D57 | H |
| 58882 | H | D58 | H |
| 58883 | H | D59 | H |
| 58884 | H | D60 | H |
| 58885 | H | D61 | H |
| 58886 | H | D62 | H |
| 58887 | H | D63 | H |
| 58888 | H | D64 | H |
| 58889 | H | D65 | H |
| 58890 | H | D66 | H |
| 58891 | H | D67 | H |
| 58892 | H | D68 | H |
| 58893 | H | D69 | H |
| 58894 | H | D70 | H |
| 58895 | H | D71 | H |
| 58896 | H | D72 | H |
| 58897 | H | D73 | H |
| 58898 | H | D74 | H |
| 58899 | H | D75 | H |
| 58900 | H | D76 | H |
| 58901 | H | D77 | H |
| 58902 | H | D78 | H |
| 58903 | H | D79 | H |
| 58904 | H | D80 | H |
| 58905 | H | D81 | H |
| 58906 | H | D82 | H |
| 58907 | H | D83 | H |
| 58908 | H | D84 | H |
| 58909 | H | D85 | H |
| 58910 | H | D86 | H |
| 58911 | H | D87 | H |
| 58912 | H | D88 | H |
| 58913 | H | D89 | H |
| 58914 | H | D90 | H |
| 58915 | H | D91 | H |
| 58916 | H | D92 | H |
| 58917 | H | D93 | H |
| 58918 | H | D94 | H |
| 58919 | H | D95 | H |
| 58920 | H | D96 | H |
| 58921 | H | D97 | H |
| 58922 | H | D98 | H |
| 58923 | H | D99 | H |
| 58924 | H | D100 | H |
| 58925 | H | D101 | H |
| 58926 | H | D102 | H |

TABLE 28

| No. | R¹⁰² | R¹⁰³ | R¹⁰⁴~R¹⁰⁸ |
|---|---|---|---|
| 58927 | H | D103 | H |
| 58928 | H | D104 | H |
| 58929 | H | D105 | H |
| 58930 | H | D106 | H |
| 58931 | H | D107 | H |
| 58932 | H | D108 | H |
| 58933 | H | D109 | H |
| 58934 | H | D110 | H |
| 58935 | H | D111 | H |
| 58936 | H | D112 | H |
| 58937 | H | D113 | H |
| 58938 | H | D114 | H |
| 58939 | H | D115 | H |
| 58940 | H | D116 | H |
| 58941 | H | D117 | H |
| 58942 | H | D118 | H |
| 58943 | H | D119 | H |
| 58944 | H | D120 | H |
| 58945 | H | D121 | H |
| 58946 | H | D122 | H |
| 58947 | H | D123 | H |
| 58948 | H | D124 | H |
| 58949 | H | D125 | H |
| 58950 | H | D126 | H |
| 58951 | H | D127 | H |
| 58952 | H | D128 | H |
| 58953 | H | D129 | H |
| 58954 | H | D130 | H |
| 58955 | H | D131 | H |
| 58956 | H | D132 | H |
| 58957 | H | D133 | H |
| 58958 | H | D134 | H |
| 58959 | H | D135 | H |
| 58960 | H | D136 | H |
| 58961 | H | D137 | H |
| 58962 | H | D138 | H |
| 58963 | H | D139 | H |
| 58964 | H | D140 | H |
| 58965 | H | D141 | H |
| 58966 | H | D142 | H |
| 58967 | H | D143 | H |
| 58968 | H | D144 | H |
| 58969 | H | D145 | H |
| 58970 | H | D146 | H |
| 58971 | H | D147 | H |
| 58972 | H | D148 | H |
| 58973 | H | D149 | H |
| 58974 | H | D150 | H |
| 58975 | H | D151 | H |
| 58976 | H | D152 | H |
| 58977 | H | D153 | H |
| 58978 | H | D154 | H |
| 58979 | H | D155 | H |
| 58980 | H | D156 | H |
| 58981 | H | D157 | H |
| 58982 | H | D158 | H |
| 58983 | H | D159 | H |
| 58984 | H | D160 | H |
| 58985 | H | D161 | H |
| 58986 | H | D162 | H |
| 58987 | H | D163 | H |
| 58988 | H | D164 | H |
| 58989 | H | D165 | H |
| 58990 | H | D166 | H |
| 58991 | H | D167 | H |
| 58992 | H | D168 | H |
| 58993 | H | D169 | H |
| 58994 | H | D170 | H |
| 58995 | H | D171 | H |
| 58996 | H | D172 | H |
| 58997 | H | D173 | H |
| 58998 | H | D174 | H |
| 58999 | H | D175 | H |
| 59000 | H | D176 | H |
| 59001 | H | D177 | H |
| 59002 | H | D178 | H |
| 59003 | H | D179 | H |
| 59004 | H | D180 | H |
| 59005 | H | D181 | H |
| 59006 | H | D182 | H |
| 59007 | H | D183 | H |
| 59008 | H | D184 | H |
| 59009 | H | D185 | H |
| 59010 | H | D186 | H |
| 59011 | H | D187 | H |
| 59012 | H | D188 | H |
| 59013 | H | D189 | H |
| 59014 | H | D190 | H |
| 59015 | H | D191 | H |
| 59016 | H | D192 | H |
| 59017 | H | D193 | H |
| 59018 | H | D194 | H |
| 59019 | H | D195 | H |
| 59020 | H | D196 | H |
| 59021 | H | D197 | H |
| 59022 | H | D198 | H |
| 59023 | H | D199 | H |
| 59024 | H | D200 | H |
| 59025 | H | D201 | H |
| 59026 | H | D202 | H |
| 59027 | H | D203 | H |
| 59028 | H | D204 | H |
| 59029 | H | D205 | H |
| 59030 | H | D206 | H |
| 59031 | H | D207 | H |
| 59032 | H | D208 | H |
| 59033 | H | D209 | H |
| 59034 | H | D210 | H |
| 59035 | H | D211 | H |
| 59036 | H | D212 | H |
| 59037 | H | D213 | H |
| 59038 | H | D214 | H |
| 59039 | H | D215 | H |
| 59040 | H | D216 | H |
| 59041 | H | D217 | H |
| 59042 | H | D218 | H |
| 59043 | H | D219 | H |
| 59044 | H | D220 | H |
| 59045 | H | D221 | H |
| 59046 | H | D222 | H |

TABLE 29

| No. | R¹⁰² | R¹⁰³ | R¹⁰⁴~R¹⁰⁸ |
|---|---|---|---|
| 59047 | H | D223 | H |
| 59048 | H | D224 | H |
| 59049 | H | D225 | H |
| 59050 | H | D226 | H |
| 59051 | H | D227 | H |
| 59052 | H | D228 | H |
| 59053 | H | D229 | H |
| 59054 | H | D230 | H |
| 59055 | H | D231 | H |
| 59056 | H | D232 | H |
| 59057 | H | D233 | H |
| 59058 | H | D234 | H |
| 59059 | H | D235 | H |
| 59060 | H | D236 | H |
| 59061 | H | D237 | H |
| 59062 | H | D238 | H |
| 59063 | H | D239 | H |
| 59064 | H | D240 | H |
| 59065 | H | D241 | H |
| 59066 | H | D242 | H |
| 59067 | H | D243 | H |
| 59068 | H | D244 | H |
| 59069 | H | D245 | H |
| 59070 | H | D246 | H |
| 59071 | H | D247 | H |
| 59072 | H | D248 | H |
| 59073 | H | D249 | H |
| 59074 | H | D250 | H |
| 59075 | H | D251 | H |
| 59076 | H | D252 | H |
| 59077 | H | D253 | H |
| 59078 | H | D254 | H |
| 59079 | H | D255 | H |
| 59080 | H | D256 | H |
| 59081 | H | D257 | H |
| 59082 | H | D258 | H |

TABLE 30

| | | Substituting |
|---|---|---|
| New Compound Nos. | Substituted Groups | Groups |
| Nos. 59083 to 59340 are | by substituting R103 of | with the same |
| compounds obtained | Nos. 58567 to 58824 | group as R102. |
| Nos. 59341 to 59598 are | by substituting R104 of | with the same |
| compounds obtained | Nos. 58825 to 59082 | group as R103. |
| Nos. 59599 to 60630 are | by substituting R105 of | with N1. |
| compounds obtained | Nos. 58567 to 59598 | |
| Nos. 60631 to 61662 are | by substituting R106 of | with N1. |
| compounds obtained | Nos. 58567 to 59598 | |
| Nos. 61663 to 62694 are | by substituting R107 of | with N1. |
| compounds obtained | Nos. 58567 to 59598 | |
| Nos. 62695 to 63726 are | by substituting R108 of | with N1. |
| compounds obtained | Nos. 58567 to 59598 | |
| Nos. 63727 to 64758 are | by substituting R105 of | with A1. |
| compounds obtained | Nos. 58567 to 59598 | |
| Nos. 64759 to 65790 are | by substituting R106 of | with A1. |
| compounds obtained | Nos. 58567 to 59598 | |
| Nos. 65791 to 66822 are | by substituting R107 of | with A1. |
| compounds obtained | Nos. 58567 to 59598 | |
| Nos. 66823 to 67854 are | by substituting R108 of | with A1. |
| compounds obtained | Nos. 58567 to 59598 | |
| Nos. 67855 to 68886 are | by substituting R105 of | with N5. |
| compounds obtained | Nos. 58567 to 59598 | |
| Nos. 68887 to 69918 are | by substituting R106 of | with N5. |
| compounds obtained | Nos. 58567 to 59598 | |
| Nos. 69919 to 70950 are | by substituting R107 of | with N5. |
| compounds obtained | Nos. 58567 to 59598 | |
| Nos. 70951 to 71982 are | by substituting R108 of | with N5. |
| compounds obtained | Nos. 58567 to 59598 | |

TABLE 31
General Formula (4f′)

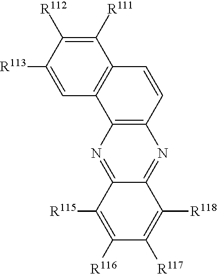

| | | | R¹¹⁴, |
|---|---|---|---|
| No. | R¹¹² | R¹¹³ | R¹¹⁵~R¹¹⁶ |
| 71983 | D1 | H | H |
| 71984 | D2 | H | H |
| 71985 | D3 | H | H |
| 71986 | D4 | H | H |
| 71987 | D5 | H | H |
| 71988 | D6 | H | H |
| 71989 | D7 | H | H |
| 71990 | D8 | H | H |
| 71991 | D9 | H | H |
| 71992 | D10 | H | H |
| 71993 | D11 | H | H |
| 71994 | D12 | H | H |
| 71995 | D13 | H | H |
| 71996 | D14 | H | H |
| 71997 | D15 | H | H |
| 71998 | D16 | H | H |
| 71999 | D17 | H | H |
| 72000 | D18 | H | H |
| 72001 | D19 | H | H |
| 72002 | D20 | H | H |
| 72003 | D21 | H | H |
| 72004 | D22 | H | H |
| 72005 | D23 | H | H |
| 72006 | D24 | H | H |
| 72007 | D25 | H | H |
| 72008 | D26 | H | H |
| 72009 | D27 | H | H |
| 72010 | D28 | H | H |
| 72011 | D29 | H | H |
| 72012 | D30 | H | H |
| 72013 | D31 | H | H |
| 72014 | D32 | H | H |
| 72015 | D33 | H | H |
| 72016 | D34 | H | H |
| 72017 | D35 | H | H |
| 72018 | D36 | H | H |
| 72019 | D37 | H | H |
| 72020 | D38 | H | H |
| 72021 | D39 | H | H |
| 72022 | D40 | H | H |
| 72023 | D41 | H | H |
| 72024 | D42 | H | H |
| 72025 | D43 | H | H |
| 72026 | D44 | H | H |
| 72027 | D45 | H | H |
| 72028 | D46 | H | H |
| 72029 | D47 | H | H |
| 72030 | D48 | H | H |
| 72031 | D49 | H | H |
| 72032 | D50 | H | H |
| 72033 | D51 | H | H |
| 72034 | D52 | H | H |
| 72035 | D53 | H | H |
| 72036 | D54 | H | H |
| 72037 | D55 | H | H |
| 72038 | D56 | H | H |
| 72039 | D57 | H | H |
| 72040 | D58 | H | H |
| 72041 | D59 | H | H |
| 72042 | D60 | H | H |
| 72043 | D61 | H | H |
| 72044 | D62 | H | H |
| 72045 | D63 | H | H |
| 72046 | D64 | H | H |
| 72047 | D65 | H | H |
| 72048 | D66 | H | H |
| 72049 | D67 | H | H |
| 72050 | D68 | H | H |
| 72051 | D69 | H | H |
| 72052 | D70 | H | H |
| 72053 | D71 | H | H |
| 72054 | D72 | H | H |
| 72055 | D73 | H | H |
| 72056 | D74 | H | H |
| 72057 | D75 | H | H |
| 72058 | D76 | H | H |
| 72059 | D77 | H | H |
| 72060 | D78 | H | H |
| 72061 | D79 | H | H |
| 72062 | D80 | H | H |
| 72063 | D81 | H | H |
| 72064 | D82 | H | H |
| 72065 | D83 | H | H |
| 72066 | D84 | H | H |
| 72067 | D85 | H | H |
| 72068 | D86 | H | H |
| 72069 | D87 | H | H |
| 72070 | D88 | H | H |
| 72071 | D89 | H | H |
| 72072 | D90 | H | H |
| 72073 | D91 | H | H |
| 72074 | D92 | H | H |
| 72075 | D93 | H | H |
| 72076 | D94 | H | H |
| 72077 | D95 | H | H |
| 72078 | D96 | H | H |
| 72079 | D97 | H | H |
| 72080 | D98 | H | H |
| 72081 | D99 | H | H |
| 72082 | D100 | H | H |
| 72083 | D101 | H | H |
| 72084 | D102 | H | H |
| 72085 | D103 | H | H |
| 72086 | D104 | H | H |
| 72087 | D105 | H | H |
| 72088 | D106 | H | H |
| 72089 | D107 | H | H |
| 72090 | D108 | H | H |
| 72091 | D109 | H | H |
| 72092 | D110 | H | H |
| 72093 | D111 | H | H |
| 72094 | D112 | H | H |
| 72095 | D113 | H | H |
| 72096 | D114 | H | H |
| 72097 | D115 | H | H |
| 72098 | D116 | H | H |
| 72099 | D117 | H | H |
| 72100 | D118 | H | H |
| 72101 | D119 | H | H |
| 72102 | D120 | H | H |

TABLE 32

| | | | R¹¹¹, |
|---|---|---|---|
| No. | R¹¹² | R¹¹³ | R¹¹⁵~R¹¹⁸ |
| 72103 | D121 | H | H |
| 72104 | D122 | H | H |
| 72105 | D123 | H | H |
| 72106 | D124 | H | H |
| 72107 | D125 | H | H |
| 72108 | D126 | H | H |
| 72109 | D127 | H | H |
| 72110 | D128 | H | H |
| 72111 | D129 | H | H |
| 72112 | D130 | H | H |
| 72113 | D131 | H | H |
| 72114 | D132 | H | H |
| 72115 | D133 | H | H |
| 72116 | D134 | H | H |
| 72117 | D135 | H | H |
| 72118 | D136 | H | H |
| 72119 | D137 | H | H |
| 72120 | D138 | H | H |
| 72121 | D139 | H | H |
| 72122 | D140 | H | H |
| 72123 | D141 | H | H |
| 72124 | D142 | H | H |
| 72125 | D143 | H | H |
| 72126 | D144 | H | H |
| 72127 | D145 | H | H |
| 72128 | D146 | H | H |
| 72129 | D147 | H | H |
| 72130 | D148 | H | H |
| 72131 | D149 | H | H |
| 72132 | D150 | H | H |
| 72133 | D151 | H | H |
| 72134 | D152 | H | H |
| 72135 | D153 | H | H |
| 72136 | D154 | H | H |
| 72137 | D155 | H | H |
| 72138 | D156 | H | H |
| 72139 | D157 | H | H |
| 72140 | D158 | H | H |
| 72141 | D159 | H | H |
| 72142 | D160 | H | H |
| 72143 | D161 | H | H |
| 72144 | D162 | H | H |
| 72145 | D163 | H | H |
| 72146 | D164 | H | H |
| 72147 | D165 | H | H |
| 72148 | D166 | H | H |
| 72149 | D167 | H | H |
| 72150 | D168 | H | H |
| 72151 | D169 | H | H |
| 72152 | D170 | H | H |
| 72153 | D171 | H | H |
| 72154 | D172 | H | H |
| 72155 | D173 | H | H |
| 72156 | D174 | H | H |
| 72157 | D175 | H | H |
| 72158 | D176 | H | H |
| 72159 | D177 | H | H |
| 72160 | D178 | H | H |
| 72161 | D179 | H | H |
| 72162 | D180 | H | H |
| 72163 | D181 | H | H |
| 72164 | D182 | H | H |
| 72165 | D183 | H | H |
| 72166 | D184 | H | H |
| 72167 | D185 | H | H |
| 72168 | D186 | H | H |
| 72169 | D187 | H | H |
| 72170 | D188 | H | H |
| 72171 | D189 | H | H |
| 72172 | D190 | H | H |
| 72173 | D191 | H | H |
| 72174 | D192 | H | H |
| 72175 | D193 | H | H |
| 72176 | D194 | H | H |
| 72177 | D195 | H | H |
| 72178 | D196 | H | H |
| 72179 | D197 | H | H |
| 72180 | D198 | H | H |
| 72181 | D199 | H | H |
| 72182 | D200 | H | H |
| 72183 | D201 | H | H |
| 72184 | D202 | H | H |
| 72185 | D203 | H | H |
| 72186 | D204 | H | H |
| 72187 | D205 | H | H |
| 72188 | D206 | H | H |
| 72189 | D207 | H | H |
| 72190 | D208 | H | H |
| 72191 | D209 | H | H |
| 72192 | D210 | H | H |
| 72193 | D211 | H | H |
| 72194 | D212 | H | H |
| 72195 | D213 | H | H |
| 72196 | D214 | H | H |
| 72197 | D215 | H | H |
| 72198 | D216 | H | H |
| 72199 | D217 | H | H |
| 72200 | D218 | H | H |
| 72201 | D219 | H | H |
| 72202 | D220 | H | H |
| 72203 | D221 | H | H |
| 72204 | D222 | H | H |
| 72205 | D223 | H | H |
| 72206 | D224 | H | H |
| 72207 | D225 | H | H |
| 72208 | D226 | H | H |
| 72209 | D227 | H | H |
| 72210 | D228 | H | H |
| 72211 | D229 | H | H |
| 72212 | D230 | H | H |
| 72213 | D231 | H | H |
| 72214 | D232 | H | H |
| 72215 | D233 | H | H |
| 72216 | D234 | H | H |
| 72217 | D235 | H | H |
| 72218 | D236 | H | H |
| 72219 | D237 | H | H |
| 72220 | D238 | H | H |
| 72221 | D239 | H | H |
| 72222 | D240 | H | H |

TABLE 33

| | | | R¹¹¹, |
|---|---|---|---|
| No. | R¹¹² | R¹¹³ | R¹¹⁵~R¹¹⁸ |
| 72223 | D241 | H | H |
| 72224 | D242 | H | H |
| 72225 | D243 | H | H |
| 72226 | D244 | H | H |
| 72227 | D245 | H | H |
| 72228 | D246 | H | H |
| 72229 | D247 | H | H |
| 72230 | D248 | H | H |
| 72231 | D249 | H | H |
| 72232 | D250 | H | H |
| 72233 | D251 | H | H |
| 72234 | D252 | H | H |
| 72235 | D253 | H | H |
| 72236 | D254 | H | H |
| 72237 | D255 | H | H |
| 72238 | D256 | H | H |
| 72239 | D257 | H | H |
| 72240 | D258 | H | H |
| 72241 | H | D1 | H |
| 72242 | H | D2 | H |
| 72243 | H | D3 | H |
| 72244 | H | D4 | H |
| 72245 | H | D5 | H |
| 72246 | H | D6 | H |
| 72247 | H | D7 | H |
| 72248 | H | D8 | H |
| 72249 | H | D9 | H |
| 72250 | H | D10 | H |
| 72251 | H | D11 | H |
| 72252 | H | D12 | H |
| 72253 | H | D13 | H |
| 72254 | H | D14 | H |
| 72255 | H | D15 | H |
| 72256 | H | D16 | H |
| 72257 | H | D17 | H |
| 72258 | H | D18 | H |
| 72259 | H | D19 | H |
| 72260 | H | D20 | H |
| 72261 | H | D21 | H |
| 72262 | H | D22 | H |
| 72263 | H | D23 | H |
| 72264 | H | D24 | H |
| 72265 | H | D25 | H |
| 72266 | H | D26 | H |
| 72267 | H | D27 | H |
| 72268 | H | D28 | H |
| 72269 | H | D29 | H |
| 72270 | H | D30 | H |
| 72271 | H | D31 | H |
| 72272 | H | D32 | H |
| 72273 | H | D33 | H |
| 72274 | H | D34 | H |
| 72275 | H | D35 | H |
| 72276 | H | D36 | H |
| 72277 | H | D37 | H |
| 72278 | H | D38 | H |
| 72279 | H | D39 | H |
| 72280 | H | D40 | H |
| 72281 | H | D41 | H |
| 72282 | H | D42 | H |
| 72283 | H | D43 | H |
| 72284 | H | D44 | H |
| 72285 | H | D45 | H |
| 72286 | H | D46 | H |
| 72287 | H | D47 | H |
| 72288 | H | D48 | H |
| 72289 | H | D49 | H |
| 72290 | H | D50 | H |
| 72291 | H | D51 | H |
| 72292 | H | D52 | H |
| 72293 | H | D53 | H |
| 72294 | H | D54 | H |
| 72295 | H | D55 | H |
| 72296 | H | D56 | H |
| 72297 | H | D57 | H |
| 72298 | H | D58 | H |
| 72299 | H | D59 | H |
| 72300 | H | D60 | H |
| 72301 | H | D61 | H |
| 72302 | H | D62 | H |
| 72303 | H | D63 | H |
| 72304 | H | D64 | H |
| 72305 | H | D65 | H |
| 72306 | H | D66 | H |
| 72307 | H | D67 | H |
| 72308 | H | D68 | H |
| 72309 | H | D69 | H |
| 72310 | H | D70 | H |
| 72311 | H | D71 | H |
| 72312 | H | D72 | H |
| 72313 | H | D73 | H |
| 72314 | H | D74 | H |
| 72315 | H | D75 | H |
| 72316 | H | D76 | H |
| 72317 | H | D77 | H |
| 72318 | H | D78 | H |
| 72319 | H | D79 | H |
| 72320 | H | D80 | H |
| 72321 | H | D81 | H |
| 72322 | H | D82 | H |
| 72323 | H | D83 | H |
| 72324 | H | D84 | H |
| 72325 | H | D85 | H |
| 72326 | H | D86 | H |
| 72327 | H | D87 | H |
| 72328 | H | D88 | H |
| 72329 | H | D89 | H |
| 72330 | H | D90 | H |
| 72331 | H | D91 | H |
| 72332 | H | D92 | H |
| 72333 | H | D93 | H |
| 72334 | H | D94 | H |
| 72335 | H | D95 | H |
| 72336 | H | D96 | H |
| 72337 | H | D97 | H |
| 72338 | H | D98 | H |
| 72339 | H | D99 | H |
| 72340 | H | D100 | H |
| 72341 | H | D101 | H |
| 72342 | H | D102 | H |

TABLE 34

| | | | R¹¹¹, |
|---|---|---|---|
| No. | R¹¹² | R¹¹³ | R¹¹⁵~R¹¹⁸ |
| 72343 | H | D103 | H |
| 72344 | H | D104 | H |
| 72345 | H | D105 | H |
| 72346 | H | D106 | H |
| 72347 | H | D107 | H |
| 72348 | H | D108 | H |
| 72349 | H | D109 | H |
| 72350 | H | D110 | H |
| 72351 | H | D111 | H |
| 72352 | H | D112 | H |
| 72353 | H | D113 | H |
| 72354 | H | Dt14 | H |
| 72355 | H | D115 | H |
| 72356 | H | D116 | H |
| 72357 | H | D117 | H |
| 72358 | H | D118 | H |
| 72359 | H | D119 | H |
| 72360 | H | D120 | H |
| 72361 | H | D121 | H |
| 72362 | H | D122 | H |
| 72363 | H | D123 | H |
| 72364 | H | D124 | H |
| 72365 | H | D125 | H |
| 72366 | H | D126 | H |
| 72367 | H | D127 | H |
| 72368 | H | D128 | H |
| 72369 | H | D129 | H |
| 72370 | H | D130 | H |
| 72371 | H | D131 | H |
| 72372 | H | DI32 | H |
| 72373 | H | D133 | H |
| 72374 | H | D134 | H |
| 72375 | H | D135 | H |
| 72376 | H | D136 | H |
| 72377 | H | D137 | H |
| 72378 | H | D138 | H |
| 72379 | H | D139 | H |
| 72380 | H | D140 | H |
| 72381 | H | D141 | H |
| 72382 | H | D142 | H |
| 72383 | H | D143 | H |
| 72384 | H | D144 | H |
| 72385 | H | D145 | H |
| 72386 | H | D146 | H |
| 72387 | H | D147 | H |
| 72388 | H | D148 | H |
| 72389 | H | D149 | H |
| 72390 | H | D150 | H |
| 72391 | H | D151 | H |
| 72392 | H | D152 | H |
| 72393 | H | D153 | H |
| 72394 | H | D154 | H |
| 72395 | H | D155 | H |
| 72396 | H | D156 | H |
| 72397 | H | D157 | H |
| 72398 | H | D158 | H |
| 72399 | H | D159 | H |
| 72400 | H | D160 | H |
| 72401 | H | D161 | H |
| 72402 | H | D162 | H |
| 72403 | H | D163 | H |
| 72404 | H | D164 | H |
| 72405 | H | D165 | H |
| 72406 | H | D166 | H |
| 72407 | H | D167 | H |
| 72408 | H | D168 | H |
| 72409 | H | D169 | H |
| 72410 | H | D170 | H |
| 72411 | H | D171 | H |
| 72412 | H | D172 | H |
| 72413 | H | D173 | H |
| 72414 | H | D174 | H |
| 72415 | H | D175 | H |
| 72416 | H | D176 | H |
| 72417 | H | D177 | H |
| 72418 | H | D178 | H |
| 72419 | H | D179 | H |
| 72420 | H | D180 | H |
| 72421 | H | D181 | H |
| 72422 | H | D182 | H |
| 72423 | H | D183 | H |
| 72424 | H | D184 | H |
| 72425 | H | D185 | H |
| 72426 | H | D186 | H |
| 72427 | H | D187 | H |
| 72428 | H | D188 | H |
| 72429 | H | D189 | H |
| 72430 | H | D190 | H |
| 72431 | H | D191 | H |
| 72432 | H | D192 | H |
| 72433 | H | D193 | H |
| 72434 | H | D194 | H |
| 72435 | H | D195 | H |
| 72436 | H | D196 | H |
| 72437 | H | D197 | H |
| 72438 | H | D198 | H |
| 72439 | H | D199 | H |
| 72440 | H | D200 | H |
| 72441 | H | D201 | H |
| 72442 | H | D202 | H |
| 72443 | H | D203 | H |
| 72444 | H | D204 | H |
| 72445 | H | D205 | H |
| 72446 | H | D206 | H |
| 72447 | H | D207 | H |
| 72448 | H | D208 | H |
| 72449 | H | D209 | H |
| 72450 | H | D210 | H |
| 72451 | H | D211 | H |
| 72452 | H | D212 | H |
| 72453 | H | D213 | H |
| 72454 | H | D214 | H |
| 72455 | H | D215 | H |
| 72456 | H | D216 | H |
| 72457 | H | D217 | H |
| 72458 | H | D218 | H |
| 72459 | H | D219 | H |
| 72460 | H | D220 | H |
| 72461 | H | D221 | H |
| 72462 | H | D222 | H |

TABLE 35

| | | | R¹¹¹, |
|---|---|---|---|
| No. | R¹¹² | R¹¹³ | R¹¹⁵~R¹¹⁸ |
| 72463 | H | D223 | H |
| 72464 | H | D224 | H |
| 72465 | H | D225 | H |
| 72466 | H | D226 | H |
| 72467 | H | D227 | H |
| 72468 | H | D228 | H |
| 72469 | H | D229 | H |
| 72470 | H | D230 | H |
| 72471 | H | D231 | H |
| 72472 | H | D232 | H |
| 72473 | H | D233 | H |
| 72474 | H | D234 | H |
| 72475 | H | D235 | H |
| 72476 | H | D236 | H |
| 72477 | H | D237 | H |
| 72478 | H | D238 | H |
| 72479 | H | D239 | H |
| 72480 | H | D240 | H |
| 72481 | H | D241 | H |
| 72482 | H | D242 | H |
| 72483 | H | D243 | H |
| 72484 | H | D244 | H |
| 72485 | H | D245 | H |
| 72486 | H | D246 | H |
| 72487 | H | D247 | H |
| 72488 | H | D248 | H |
| 72489 | H | D249 | H |
| 72490 | H | D250 | H |
| 72491 | H | D251 | H |
| 72492 | H | D252 | H |
| 72493 | H | D253 | H |
| 72494 | H | D254 | H |
| 72495 | H | D255 | H |
| 72496 | H | D256 | H |
| 72497 | H | D257 | H |
| 72498 | H | D258 | H |

TABLE 36

| | | Substituting |
|---|---|---|
| New Compound Nos. | Substituted Groups | Groups |
| Nos. 72499 to 72756 are | by substituting R113 of | with the same |
| compounds obtained | Nos. 71983 to 72240 | group as R112. |
| Nos. 72757 to 73014 are | by substituting R111 of | with the same |
| compounds obtained | Nos. 72241 to 72498 | group as R113. |
| Nos. 73015 to 74046 are | by substituting R115 of | with N1. |
| compounds obtained | Nos. 71983 to 73014 | |
| Nos. 74047 to 75078 are | by substituting R116 of | with N1. |
| compounds obtained | Nos. 71983 to 73014 | |
| Nos. 75079 to 76110 are | by substituting R117 of | with N1. |
| compounds obtained | Nos. 71983 to 73014 | |
| Nos. 76111 to 77142 are | by substituting R118 of | with N1. |
| compounds obtained | Nos. 71983 to 73014 | |
| Nos. 77143 to 78174 are | by substituting R115 of | with A1. |
| compounds obtained | Nos. 71983 to 73014 | |
| Nos. 78175 to 79206 are | by substituting R116 of | with A1. |
| compounds obtained | Nos. 71983 to 73014 | |
| Nos. 79207 to 80238 are | by substituting R117 of | with A1. |
| compounds obtained | Nos. 71983 to 73014 | |
| Nos. 80239 to 81270 are | by substituting R118 of | with A1. |
| compounds obtained | Nos. 71983 to 73014 | |
| Nos. 81271 to 82302 are | by substituting R115 of | with N5. |
| compounds obtained | Nos. 71983 to 73014 | |
| Nos. 82303 to 83334 are | by substituting R116 of | with N5. |
| compounds obtained | Nos. 71983 to 73014 | |
| Nos. 83335 to 84366 are | by substituting R117 of | with N5. |
| compounds obtained | Nos. 71983 to 73014 | |
| Nos. 84367 to 85398 are | by substituting R118 of | with N5. |
| compounds obtained | Nos. 71983 to 73014 | |

TABLE 37
General Formula (4g′)

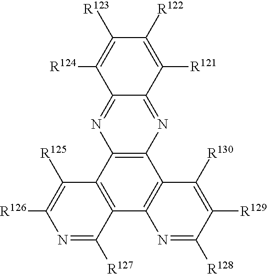

| No. | R¹²¹ | R¹²² | R¹²³~R¹³⁰ |
|---|---|---|---|
| 85399 | D1 | H | H |
| 85400 | D2 | H | H |
| 85401 | D3 | H | H |
| 85402 | D4 | H | H |
| 85403 | D5 | H | H |
| 85404 | D6 | H | H |
| 85405 | D7 | H | H |
| 85406 | D8 | H | H |
| 85407 | D9 | H | H |
| 85408 | D10 | H | H |
| 85409 | D11 | H | H |
| 85410 | D12 | H | H |
| 85411 | D13 | H | H |
| 85412 | D14 | H | H |
| 85413 | D15 | H | H |
| 85414 | D16 | H | H |
| 85415 | D17 | H | H |
| 85416 | D18 | H | H |
| 85417 | D19 | H | H |
| 85418 | D20 | H | H |
| 85419 | D21 | H | H |
| 85420 | D22 | H | H |
| 85421 | D23 | H | H |
| 85422 | D24 | H | H |
| 85423 | D25 | H | H |
| 85424 | D26 | H | H |
| 85425 | D27 | H | H |
| 85426 | D28 | H | H |
| 85427 | D29 | H | H |
| 85428 | D30 | H | H |
| 85429 | D31 | H | H |
| 85430 | D32 | H | H |
| 85431 | D33 | H | H |
| 85432 | D34 | H | H |
| 85433 | D35 | H | H |
| 85434 | D36 | H | H |
| 85435 | D37 | H | H |
| 85436 | D38 | H | H |
| 85437 | D39 | H | H |
| 85438 | D40 | H | H |
| 85439 | D41 | H | H |
| 85440 | D42 | H | H |
| 85441 | D43 | H | H |
| 85442 | D44 | H | H |
| 85443 | D45 | H | H |
| 85444 | D46 | H | H |
| 85445 | D47 | H | H |
| 85446 | D48 | H | H |
| 85447 | D49 | H | H |
| 85448 | D50 | H | H |
| 85449 | D51 | H | H |
| 85450 | D52 | H | H |
| 85451 | D53 | H | H |
| 85452 | D54 | H | H |
| 85453 | D55 | H | H |
| 85454 | D56 | H | H |
| 85455 | D57 | H | H |
| 85456 | D58 | H | H |
| 85457 | D59 | H | H |
| 85458 | D60 | H | H |
| 85459 | D61 | H | H |
| 85460 | D62 | H | H |
| 85461 | D63 | H | H |
| 85462 | D64 | H | H |
| 85463 | D65 | H | H |
| 85464 | D66 | H | H |
| 85465 | D67 | H | H |
| 85466 | D68 | H | H |
| 85467 | D69 | H | H |
| 85468 | D70 | H | H |
| 85469 | D71 | H | H |
| 85470 | D72 | H | H |
| 85471 | D73 | H | H |
| 85472 | D74 | H | H |
| 85473 | D75 | H | H |
| 85474 | D76 | H | H |
| 85475 | D77 | H | H |
| 85476 | D78 | H | H |
| 85477 | D79 | H | H |
| 85478 | D80 | H | H |
| 85479 | D81 | H | H |
| 85480 | D82 | H | H |
| 85481 | D83 | H | H |
| 85482 | D84 | H | H |
| 85483 | D85 | H | H |
| 85484 | D86 | H | H |
| 85485 | D87 | H | H |
| 85486 | D88 | H | H |
| 85487 | D89 | H | H |
| 85488 | D90 | H | H |
| 85489 | D91 | H | H |
| 85490 | D92 | H | H |
| 85491 | D93 | H | H |
| 85492 | D94 | H | H |
| 85493 | D95 | H | H |
| 85494 | D96 | H | H |
| 85495 | D97 | H | H |
| 85496 | D98 | H | H |
| 85497 | D99 | H | H |
| 85498 | D100 | H | H |
| 85499 | D101 | H | H |
| 85500 | D102 | H | H |
| 85501 | D103 | H | H |
| 85502 | D104 | H | H |
| 85503 | D105 | H | H |
| 85504 | D106 | H | H |
| 85505 | D107 | H | H |
| 85506 | D108 | H | H |
| 85507 | D109 | H | H |
| 85508 | D110 | H | H |
| 85509 | D111 | H | H |
| 85510 | D112 | H | H |
| 85511 | D113 | H | H |
| 85512 | D114 | H | H |
| 85513 | D115 | H | H |
| 85514 | D116 | H | H |
| 85515 | D117 | H | H |
| 85516 | DI18 | H | H |
| 85517 | D119 | H | H |
| 85518 | D120 | H | H |

TABLE 38

| No. | R¹²¹ | R¹²² | R¹²³~R¹³⁰ |
|---|---|---|---|
| 85519 | D121 | H | H |
| 85520 | D122 | H | H |
| 85521 | D123 | H | H |
| 85522 | D124 | H | H |
| 85523 | D125 | H | H |
| 85524 | D126 | H | H |
| 85525 | D127 | H | H |
| 85526 | D128 | H | H |
| 85527 | D129 | H | H |
| 85528 | D130 | H | H |
| 85529 | D131 | H | H |
| 85530 | D132 | H | H |
| 85531 | D133 | H | H |
| 85532 | D134 | H | H |
| 85533 | D135 | H | H |
| 85534 | D136 | H | H |
| 85535 | D137 | H | H |
| 85536 | D138 | H | H |
| 85537 | D139 | H | H |
| 85538 | D140 | H | H |
| 85539 | D141 | H | H |
| 85540 | D142 | H | H |
| 85541 | D143 | H | H |
| 85542 | D144 | H | H |
| 85543 | D145 | H | H |
| 85544 | D146 | H | H |
| 85545 | D147 | H | H |
| 85546 | D148 | H | H |
| 85547 | D149 | H | H |
| 85548 | D150 | H | H |
| 85549 | D151 | H | H |
| 85550 | D152 | H | H |
| 85551 | D153 | H | H |
| 85552 | D154 | H | H |
| 85553 | D155 | H | H |
| 85554 | D156 | H | H |
| 85555 | D157 | H | H |
| 85556 | D158 | H | H |
| 85557 | D159 | H | H |
| 85558 | D160 | H | H |
| 85559 | D161 | H | H |
| 85560 | D162 | H | H |
| 85561 | D163 | H | H |
| 85562 | D164 | H | H |
| 85563 | D165 | H | H |
| 85564 | D166 | H | H |
| 85565 | D167 | H | H |
| 85566 | D168 | H | H |
| 85567 | D169 | H | H |
| 85568 | D170 | H | H |
| 85569 | D171 | H | H |
| 85570 | D172 | H | H |
| 85571 | D173 | H | H |
| 85572 | D174 | H | H |
| 85573 | D175 | H | H |
| 85574 | D176 | H | H |
| 85575 | D177 | H | H |
| 85576 | D178 | H | H |
| 85577 | D179 | H | H |
| 85578 | D180 | H | H |
| 85579 | D181 | H | H |
| 85580 | D182 | H | H |
| 85581 | D183 | H | H |
| 85582 | D184 | H | H |
| 85583 | D185 | H | H |
| 85584 | D186 | H | H |
| 85585 | D187 | H | H |
| 85586 | D188 | H | H |
| 85587 | D189 | H | H |
| 85588 | D190 | H | H |
| 85589 | D191 | H | H |
| 85590 | D192 | H | H |
| 85591 | D193 | H | H |
| 85592 | D194 | H | H |
| 85593 | D195 | H | H |
| 85594 | D196 | H | H |
| 85595 | D197 | H | H |
| 85596 | D198 | H | H |
| 85597 | D199 | H | H |
| 85598 | D200 | H | H |
| 85599 | D201 | H | H |
| 85600 | D202 | H | H |
| 85601 | D203 | H | H |
| 85602 | D204 | H | H |
| 85603 | D205 | H | H |
| 85604 | D206 | H | H |
| 85605 | D207 | H | H |
| 85606 | D208 | H | H |
| 85607 | D209 | H | H |
| 85608 | D210 | H | H |
| 85609 | D211 | H | H |
| 85610 | D212 | H | H |
| 85611 | D213 | H | H |
| 85612 | D214 | H | H |
| 85613 | D215 | H | H |
| 85614 | D216 | H | H |
| 85615 | D217 | H | H |
| 85616 | D218 | H | H |
| 85617 | D219 | H | H |
| 85618 | D220 | H | H |
| 85619 | D221 | H | H |
| 85620 | D222 | H | H |
| 85621 | D223 | H | H |
| 85622 | D224 | H | H |
| 85623 | D225 | H | H |
| 85624 | D226 | H | H |
| 85625 | D227 | H | H |
| 85626 | D228 | H | H |
| 85627 | D229 | H | H |
| 85628 | D230 | H | H |
| 85629 | D231 | H | H |
| 85630 | D232 | H | H |
| 85631 | D233 | H | H |
| 85632 | D234 | H | H |
| 85633 | D235 | H | H |
| 85634 | D236 | H | H |
| 85635 | D237 | H | H |
| 85636 | D238 | H | H |
| 85637 | D239 | H | H |
| 85638 | D240 | H | H |

TABLE 39

| No. | R¹²¹ | R¹²² | R¹²³~R¹³⁰ |
|---|---|---|---|
| 85639 | D241 | H | H |
| 85640 | D242 | H | H |
| 85641 | D243 | H | H |
| 85642 | D244 | H | H |
| 85643 | D245 | H | H |
| 85644 | D246 | H | H |
| 85645 | D247 | H | H |
| 85646 | D248 | H | H |
| 85647 | D249 | H | H |
| 85648 | D250 | H | H |
| 85649 | D251 | H | H |
| 85650 | D252 | H | H |
| 85651 | D253 | H | H |
| 85652 | D254 | H | H |
| 85653 | D255 | H | H |
| 85654 | D256 | H | H |
| 85655 | D257 | H | H |
| 85656 | D258 | H | H |
| 85657 | H | D1 | H |
| 85658 | H | D2 | H |
| 85659 | H | D3 | H |
| 85660 | H | D4 | H |
| 85661 | H | D5 | H |
| 85662 | H | D6 | H |
| 85663 | H | D7 | H |
| 85664 | H | D8 | H |
| 85665 | H | D9 | H |
| 85666 | H | D10 | H |
| 85667 | H | D11 | H |
| 85668 | H | D12 | H |
| 85669 | H | D13 | H |
| 85670 | H | D14 | H |
| 85671 | H | D15 | H |
| 85672 | H | D16 | H |
| 85673 | H | D17 | H |
| 85674 | H | D18 | H |
| 85675 | H | D19 | H |
| 85676 | H | D20 | H |
| 85677 | H | D21 | H |
| 85678 | H | D22 | H |
| 85679 | H | D23 | H |
| 85680 | H | D24 | H |
| 85681 | H | D25 | H |
| 85682 | H | D26 | H |
| 85683 | H | D27 | H |
| 85684 | H | D28 | H |
| 85685 | H | D29 | H |
| 85686 | H | D30 | H |
| 85687 | H | D31 | H |
| 85688 | H | D32 | H |
| 85689 | H | D33 | H |
| 85690 | H | D34 | H |
| 85691 | H | D35 | H |
| 85692 | H | D36 | H |
| 85693 | H | D37 | H |
| 85694 | H | D38 | H |
| 85695 | H | D39 | H |
| 85696 | H | D40 | H |
| 85697 | H | D41 | H |
| 85698 | H | D42 | H |
| 85699 | H | D43 | H |
| 85700 | H | D44 | H |
| 85701 | H | D45 | H |
| 85702 | H | D46 | H |
| 85703 | H | D47 | H |
| 85704 | H | D48 | H |
| 85705 | H | D49 | H |
| 85706 | H | D50 | H |
| 85707 | H | D51 | H |
| 85708 | H | D52 | H |
| 85709 | H | D53 | H |
| 85710 | H | D54 | H |
| 85711 | H | D55 | H |
| 85712 | H | D56 | H |
| 85713 | H | D57 | H |
| 85714 | H | D58 | H |
| 85715 | H | D59 | H |
| 85716 | H | D60 | H |
| 85717 | H | D61 | H |
| 85718 | H | D62 | H |
| 85719 | H | D63 | H |
| 85720 | H | D64 | H |
| 85721 | H | D65 | H |
| 85722 | H | D66 | H |
| 85723 | H | D67 | H |
| 85724 | H | D68 | H |
| 85725 | H | D69 | H |
| 85726 | H | D70 | H |
| 85727 | H | D71 | H |
| 85728 | H | D72 | H |
| 85729 | H | D73 | H |
| 85730 | H | D74 | H |
| 85731 | H | D75 | H |
| 85732 | H | D76 | H |
| 85733 | H | D77 | H |
| 85734 | H | D78 | H |
| 85735 | H | D79 | H |
| 85736 | H | D80 | H |
| 85737 | H | D81 | H |
| 85738 | H | D82 | H |
| 85739 | H | D83 | H |
| 85740 | H | D84 | H |
| 85741 | H | D85 | H |
| 85742 | H | D86 | H |
| 85743 | H | D87 | H |
| 85744 | H | D88 | H |
| 85745 | H | D89 | H |
| 85746 | H | D90 | H |
| 85747 | H | D91 | H |
| 85748 | H | D92 | H |
| 85749 | H | D93 | H |
| 85750 | H | D94 | H |
| 85751 | H | D95 | H |
| 85752 | H | D96 | H |
| 85753 | H | D97 | H |
| 85754 | H | D98 | H |
| 85755 | H | D99 | H |
| 85756 | H | D100 | H |
| 85757 | H | D101 | H |
| 85758 | H | D102 | H |

TABLE 40

| No. | R¹²¹ | R¹²² | R¹²³~R¹³⁰ |
|---|---|---|---|
| 85759 | H | D103 | H |
| 85760 | H | D104 | H |
| 85761 | H | D105 | H |
| 85762 | H | D106 | H |
| 85763 | H | D107 | H |
| 85764 | H | D108 | H |
| 85765 | H | D109 | H |
| 85766 | H | D110 | H |
| 85767 | H | D111 | H |
| 85768 | H | D112 | H |
| 85769 | H | D113 | H |
| 85770 | H | D114 | H |
| 85771 | H | D115 | H |
| 85772 | H | D116 | H |
| 85773 | H | D117 | H |
| 85774 | H | D118 | H |
| 85775 | H | D119 | H |
| 85776 | H | D120 | H |
| 85777 | H | D121 | H |
| 85778 | H | D122 | H |
| 85779 | H | D123 | H |
| 85780 | H | D124 | H |
| 85781 | H | D125 | H |
| 85782 | H | D126 | H |
| 85783 | H | D127 | H |
| 85784 | H | D128 | H |
| 85785 | H | D129 | H |
| 85786 | H | D130 | H |
| 85787 | H | D131 | H |
| 85788 | H | D132 | H |
| 85789 | H | D133 | H |
| 85790 | H | D134 | H |
| 85791 | H | D135 | H |
| 85792 | H | D136 | H |
| 85793 | H | D137 | H |
| 85794 | H | D138 | H |
| 85795 | H | D139 | H |
| 85796 | H | D140 | H |
| 85797 | H | D141 | H |
| 85798 | H | D142 | H |
| 85799 | H | D143 | H |
| 85800 | H | D144 | H |
| 85801 | H | D145 | H |
| 85802 | H | D146 | H |
| 85803 | H | D147 | H |
| 85804 | H | D148 | H |
| 85805 | H | D149 | H |
| 85806 | H | D150 | H |
| 85807 | H | D151 | H |
| 85808 | H | D152 | H |
| 85809 | H | D153 | H |
| 85810 | H | D154 | H |
| 85811 | H | D155 | H |
| 85812 | H | D156 | H |
| 85813 | H | D157 | H |
| 85814 | H | D158 | H |
| 85815 | H | D159 | H |
| 85816 | H | D160 | H |
| 85817 | H | D161 | H |
| 85818 | H | D162 | H |
| 85819 | H | D163 | H |
| 85820 | H | D164 | H |
| 85821 | H | D165 | H |
| 85822 | H | D166 | H |
| 85823 | H | D167 | H |
| 85824 | H | D168 | H |
| 85825 | H | D169 | H |
| 85826 | H | D170 | H |
| 85827 | H | D171 | H |
| 85828 | H | D172 | H |
| 85829 | H | D173 | H |
| 85830 | H | D174 | H |
| 85831 | H | D175 | H |
| 85832 | H | D176 | H |
| 85833 | H | D177 | H |
| 85834 | H | D178 | H |
| 85835 | H | D179 | H |
| 85836 | H | D180 | H |
| 85837 | H | D181 | H |
| 85838 | H | D182 | H |
| 85839 | H | D183 | H |
| 85840 | H | D184 | H |
| 85841 | H | D185 | H |
| 85842 | H | D186 | H |
| 85843 | H | D187 | H |
| 85844 | H | D188 | H |
| 85845 | H | D189 | H |
| 85846 | H | D190 | H |
| 85847 | H | D191 | H |
| 85848 | H | D192 | H |
| 85849 | H | D193 | H |
| 85850 | H | D194 | H |
| 85851 | H | D195 | H |
| 85852 | H | D196 | H |
| 85853 | H | D197 | H |
| 85854 | H | D198 | H |
| 85855 | H | D199 | H |
| 85856 | H | D200 | H |
| 85857 | H | D201 | H |
| 85858 | H | D202 | H |
| 85859 | H | D203 | H |
| 85860 | H | D204 | H |
| 85861 | H | D205 | H |
| 85862 | H | D206 | H |
| 85863 | H | D207 | H |
| 85864 | H | D208 | H |
| 85865 | H | D209 | H |
| 85866 | H | D210 | H |
| 85867 | H | D211 | H |
| 85868 | H | D212 | H |
| 85869 | H | D213 | H |
| 85870 | H | D214 | H |
| 85871 | H | D215 | H |
| 85872 | H | D216 | H |
| 85873 | H | D217 | H |
| 85874 | H | D218 | H |
| 85875 | H | D219 | H |
| 85876 | H | D220 | H |
| 85877 | H | D221 | H |
| 85878 | H | D222 | H |

TABLE 41

| No. | R¹²¹ | R¹²² | R¹²³~R¹³⁰ |
|---|---|---|---|
| 85879 | H | D223 | H |
| 85880 | H | D224 | H |
| 85881 | H | D225 | H |
| 85882 | H | D226 | H |
| 85883 | H | D227 | H |
| 85884 | H | D228 | H |
| 85885 | H | D229 | H |
| 85886 | H | D230 | H |
| 85887 | H | D231 | H |
| 85888 | H | D232 | H |
| 85889 | H | D233 | H |
| 85890 | H | D234 | H |
| 85891 | H | D235 | H |
| 85892 | H | D236 | H |
| 85893 | H | D237 | H |
| 85894 | H | D238 | H |
| 85895 | H | D239 | H |
| 85896 | H | D240 | H |
| 85897 | H | D241 | H |
| 85898 | H | D242 | H |
| 85899 | H | D243 | H |
| 85900 | H | D244 | H |
| 85901 | H | D245 | H |
| 85902 | H | D246 | H |
| 85903 | H | D247 | H |
| 85904 | H | D248 | H |
| 85905 | H | D249 | H |
| 85906 | H | D250 | H |
| 85907 | H | D251 | H |
| 85908 | H | D252 | H |
| 85909 | H | D253 | H |
| 85910 | H | D254 | H |
| 85911 | H | D255 | H |
| 85912 | H | D256 | H |
| 85913 | H | D257 | H |
| 85914 | H | D258 | H |

TABLE 42

| | | Substituting |
|---|---|---|
| New Compound Nos. | Substituted Groups | Groups |
| Nos. 85915 to 86172 are | by substituting R123 of | with the same |
| compounds obtained | Nos. 85399 to 85656 | group as R102. |
| Nos. 86173 to 86430 are | by substituting R124 of | with the same |
| compounds obtained | Nos. 85657 to 85914 | group as R103. |
| Nos. 86431 to 87462 are | by substituting R125 of | with N1. |
| compounds obtained | Nos. 85399 to 86430 | |
| Nos. 87463 to 88494 are | by substituting R126 of | with N1. |
| compounds obtained | Nos. 85399 to 86430 | |
| Nos. 88495 to 89526 are | by substituting R127 of | with N1. |
| compounds obtained | Nos. 85399 to 86430 | |
| Nos. 89527 to 90558 are | by substituting R128 of | with N1. |
| compounds obtained | Nos. 85399 to 86430 | |
| Nos. 90559 to 91590 are | by substituting R129 of | with N1. |
| compounds obtained | Nos. 85399 to 86430 | |
| Nos. 91591 to 92622 are | by substituting R130 of | with N1. |
| compounds obtained | Nos. 85399 to 86430 | |
| Nos. 92623 to 93654 are | by substituting R125 and | with N1. |
| compounds obtained | R130 of Nos. 85399 to | |
| | 86430 | |
| Nos. 93655 to 94686 are | by substituting R126 and | with N1. |
| compounds obtained | R129 of Nos. 85399 to | |
| | 86430 | |
| Nos. 94687 to 95718 are | by substituting R127 and | with N1. |
| compounds obtained | R128 of Nos. 85399 to | |
| | 86430 | |
| Nos. 95719 to 96750 are | by substituting R125 of | with A1. |
| compounds obtained | Nos. 85399 to 86430 | |
| Nos. 96751 to 97782 are | by substituting R126 of | with A1. |
| compounds obtained | Nos. 85399 to 86430 | |
| Nos. 97783 to 98814 are | by substituting R127 of | with A1. |
| compounds obtained | Nos. 85399 to 86430 | |
| Nos. 98815 to 99846 are | by substituting R128 of | with A1. |
| compounds obtained | Nos. 85399 to 86430 | |
| Nos. 99847 to 100878 are | by substituting R129 of | with A1. |
| compounds obtained | Nos. 85399 to 86430 | |
| Nos. 100879 to 101910 are | by substituting R130 of | with A1. |
| compounds obtained | Nos. 85399 to 86430 | |
| Nos. 101911 to 102942 are | by substituting R125 and | with A1. |
| compounds obtained | R130 of Nos. 85399 to | |
| | 86430 | |
| Nos. 102943 to 103974 are | by substituting R126 and | with A1. |
| compounds obtained | R129 of Nos. 85399 to | |
| | 86430 | |
| Nos. 103975 to 105006 are | by substituting R127 and | with A1. |
| compounds obtained | R128 of Nos. 85399 to | |
| | 86430 | |
| Nos. 105007 to 106038 are | by substituting R125 of | with N5. |
| compounds obtained | Nos. 85399 to 86430 | |
| Nos. 106039 to 107070 are | by substituting R126 of | with N5. |
| compounds obtained | Nos. 85399 to 86430 | |
| Nos. 107071 to 108102 are | by substituting R127 of | with N5. |
| compounds obtained | Nos. 85399 to 86430 | |
| Nos. 108103 to 109134 are | by substituting R128 of | with N5. |
| compounds obtained | Nos. 85399 to 86430 | |
| Nos. 109135 to 110166 are | by substituting R129 of | with N5. |
| compounds obtained | Nos. 85399 to 86430 | |
| Nos. 110167 to 111198 are | by substituting R130 of | with N5. |
| compounds obtained | Nos. 85399 to 86430 | |
| Nos. 111199 to 112230 are | by substituting R125 and | with N5. |
| compounds obtained | R130 of Nos. 85399 to | |
| | 86430 | |
| Nos. 112231 to 113262 are | by substituting R126 and | with N5. |
| compounds obtained | R129 of Nos. 85399 to | |
| | 86430 | |
| Nos. 113263 to 114294 are | by substituting R127 and | with N5. |
| compounds obtained | R128 of Nos. 85399 to | |
| | 86430 | |

Compounds formed by substituting all the hydrogen atoms existing in the molecule of Compounds 1 to 114294 with a deuterium atoms are disclosed as Compound 1d to 114294d. In the case where the above-exemplified compounds have rotational isomers, mixtures of rotational isomers and each separated rotational isomer are considered to be disclosed in the present description.

In one embodiment of the present invention, a compound having an axisymmetric structure is selected as the compound represented by the general formula (1). In one embodiment of the present invention, a compound having an asymmetric structure is selected as the compound represented by the general formula (1).

In the compound represented by the general formula (1), an acceptor group may not bond to the skeleton of the general formula (1). The acceptor group as referred to herein is a group having a positive Hammett's σp value. The compound represented by the general formula (1) may be one not having a Hammett's σp value of 0.2 or more.

The molecular weight of the compound represented by the general formula (1) is, for example, when an organic layer containing the compound represented by the general formula (1) is intended to be formed by an evaporation method and used, preferably 1500 or less, more preferably 1200 or less, even more preferably 1000 or less, and further more preferably 900 or less. The lower limit of the molecular weight is a molecular weight of the smallest compound of the compound group represented by the general formula (1).

The compound represented by the general formula (1) can be formed into a layer by a coating method, irrespective of the molecular weight thereof. According to a coating method, the compound having a relatively large molecular weight can be formed into a layer. The compound represented by the general formula (1) has an advantage that the compound is readily soluble in an organic solvent. Consequently, a coating method is readily applicable to the compound represented by the general formula (1) and, in addition, the compound can be purified to have an increased purity.

By applying the present invention, it is considered that a compound containing plural number of structures represented by the general formula (1) in the molecule can be used as a light emitting material.

For example, it is considered that a polymerizable group is previously introduced into the structure represented by the general formula (1), and the polymer formed by polymerizing the polymerizable group is used as a light emitting material. Specifically, it is considered that a monomer containing a polymerizable functional group in any structure represented by the general formula (1) (for example, in any of Ar¹, D, A, and R¹ to R⁴) is prepared, and this is homo-polymerized, or is copolymerized with any other monomer to give a polymer having a repeating unit, and the polymer is used as a light emitting material. Or it is also considered that compounds represented by the general formula (1) are coupled to give a dimer or a trimer, and these are used as a light emitting material.

Examples of the polymer having a repeating unit that contains the structure represented by the general formula (1) include polymers having a structure represented by any of the following two general formulae.

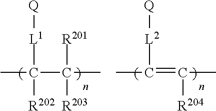

In the above general formulae, Q represents a group containing the structure represented by the general formula (1), L¹ and L² each represent a linking group. The carbon number of the linking group is preferably 0 to 20, more preferably 1 to 15, and further preferably 2 to 10. The linking group preferably has a structure represented by —X¹¹-L¹¹-. Here, X¹¹ represents an oxygen atom or a sulfur atom, and is preferably an oxygen atom. L¹¹ represents a linking group, and is preferably a substituted or unsubstituted alkylene group, or a substituted or unsubstituted arylene group, more preferably a substituted or unsubstituted alkylene group having 1 to 10 carbon atoms, or a substituted or unsubstituted phenylene group.

R²⁰¹, R²⁰², R²⁰³ and R²⁰⁴ each independently represent a substituent. Preferably, they each are a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom or a chlorine atom, and further preferably an unsubstituted alkyl group having 1 to 3 carbon atoms or an unsubstituted alkoxy group having 1 to 3 carbon atoms.

The linking group represented by L¹ and L² can bond to any position (for example, any of Ar¹, D, A, and R¹ to R⁴) of the structure represented by the general formula (1) that constitutes Q. Two or more linking groups can bond to one Q to form a crosslinked structure or a network structure.

Examples of specific structures of the repeating unit include structures represented by the following formulae.

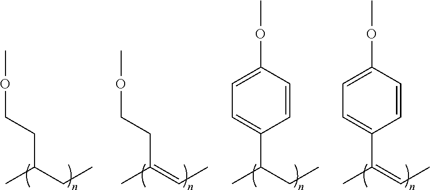

Polymers having a repeating unit that contains any of these formulae can be synthesized by previously introducing a hydroxy group into any structure represented by the general formula (1) (for example, into any of Ar¹, D, A, and R¹ to R⁴), then reacting the group serving as a linker with the following compound to thereby introduce a polymerizable group, and polymerizing the polymerizable group.

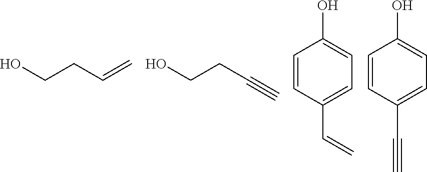

The polymer having a structure represented by the general formula (1) in the molecule can be a polymer having only a repeating unit that has the structure represented by the general formula (1), or can be a polymer containing a repeating unit that has any other structure. The repeating unit having the structure represented by the general formula (1) to be contained in the polymer may be a single kind or two or more kinds. The repeating unit not having the structure represented by the general formula (1) includes those derived from monomers used in general copolymerization. For example, it includes repeating units derived from monomers having an ethylenically unsaturated bond, such as ethylene or styrene.

Preferably, the compound represented by the general formula (1) does not contain a metal atom. For example, as the compound represented by the general formula (1), a compound formed of atoms selected from the group consisting of a carbon atom, a hydrogen atom, a deuterium atom, a nitrogen atom, an oxygen atom and a sulfur atom can be selected. For example, as the compound represented by the general formula (1), a compound formed of atoms selected from the group consisting of a carbon atom, a hydrogen atom, a deuterium atom, a nitrogen atom, and an oxygen atom can be selected. For example, as the compound represented by the general formula (1), a compound formed of atoms selected from the group consisting of a carbon atom, a hydrogen atom, a deuterium atom, a nitrogen atom, and a sulfur atom can be selected. For example, as the compound represented by the general formula (1), a compound formed of atoms selected from the group consisting of a carbon atom, a hydrogen atom, a deuterium atom, and a nitrogen atom can be selected. For example, as the compound represented by the general formula (1), a compound formed of atoms selected from the group consisting of a carbon atom, a hydrogen atom and a nitrogen atom can be selected.

In the present description, the “alkyl group” can be linear, branched or cyclic. Two or more of a linear moiety, a cyclic moiety and a branched moiety can exist therein as combined. The carbon number of the alkyl group can be, for example 1 or more, 2 or more, or 4 or more. The carbon number can also be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, an isohexyl group, a 2-ethylhexyl group, an n-heptyl group, an isoheptyl group, an n-octyl group, an isooctyl group, an n-nonyl group, an isononyl group, an n-decanyl group, an isodecanyl group, a cyclopentyl group, a cyclohexyl group, and a cycloheptyl group. The alkyl group as a substituent can be further substituted with an aryl group.

The “alkenyl group” can be linear, branched or cyclic. Two or more of a linear moiety, a cyclic moiety and a branched moiety can exist therein as combined. The carbon number of the alkenyl group can be, for example 2 or more, or 4 or more. The carbon number can also be 30 or less, 20 or less, 10 or less, 6 or less, or 4 or less. Specific examples of the alkenyl group include an ethenyl group, an n-propenyl group, an isopropenyl group, an n-butenyl group, an isobutenyl group, an n-pentenyl group, an isopentenyl group, an n-hexenyl group, an isohexenyl group, and a 2-ethylhexenyl group. The alkenyl group which is the substituent can be further substituted with a substituent.

The “aryl group” and the “heteroaryl group” each may be a monocyclic ring or may be a fused ring of two or more kinds of rings. In the case of a fused ring, the number of fused rings is preferably 2 to 6, and can be selected from, for example, 2 to 4. Specific examples of the ring include a benzene ring, a pyridine ring, a pyrimidine ring, a triazine ring, a naphthalene ring, an anthracene ring, a phenanthrene ring, a triphenylene ring, a quinoline ring, a pyrazine ring, a quinoxaline ring, and a naphthyridine ring, and the ring can be a fused ring of these rings. Specific examples of the aryl group or the heteroaryl group include a phenyl group, a 1-naphthyl group, a 2-naphthyl group, a 1-anthracenyl group, a 2-anthracenyl group, a 9-anthracenyl group, a 2-pyridyl group, a 3-pyridyl group, and a 4-pyridyl group. The ring skeleton-constituting atom number of the aryl group is preferably 6 to 40, more preferably 6 to 20, and can be selected within a range of 6 to 14, or can be selected within a range of 6 to 10. The number of the ring skeleton-constituting atoms of the heteroaryl group is preferably 4 to 40, more preferably 5 to 20, and can be selected from a range of 5 to 14, or can be selected from a range of 5 to 10. For the “arylene group” and the “heteroaryl group”, the valency number in the description of the aryl group and the heteroaryl group is changed from 1 to 2.

In the present description, “Substituent Group A” means one or a combination of two or more groups selected from the group consisting of a hydroxyl group, a halogen atom (for example, a fluorine atom, a chlorine atom, a bromine atom, an iodine atom), an alkyl group (for example, having 1 to 40 carbon atoms), an alkoxy group (for example, having 1 to 40 carbon atoms), an alkylthio group (for example, having 1 to 40 carbon atoms), an aryl group (for example, having 6 to 30 carbon atoms), an aryloxy group (for example, having 6 to 30 carbon atoms), an arylthio group (for example, having 6 to 30 carbon atoms), a heteroaryl group (for example, having a ring skeleton-constituting atom number of 5 to 30), a heteroaryloxy group (for example, having a ring skeleton-constituting atom number of 5 to 30), a heteroarylthio group (for example, having a ring skeleton-constituting atom number of 5 to 30), an acyl group (for example, having 1 to 40 carbon atoms), an alkenyl group (for example, having 1 to 40 carbon atoms), an alkynyl group (for example, having 1 to 40 carbon atoms), an alkoxycarbonyl group (for example, having 1 to 40 carbon atoms), an aryloxycarbonyl group (for example, having 1 to 40 carbon atoms), a heteroaryloxycarbonyl group (for example, having 1 to 40 carbon atoms), a silyl group (for example, a trialkylsilyl group having 1 to 40 carbon atoms) and a nitro group.

In the present description, “Substituent Group B” means one or a combination of two or more groups selected from the group consisting of an alkyl group (for example, having 1 to 40 carbon atoms), an alkoxy group (for example, having 1 to 40 carbon atoms), an aryl group (for example, having 6 to 30 carbon atoms), an aryloxy group (for example, having 6 to 30 carbon atoms), a heteroaryl group (for example, having a ring skeleton-constituting atom number of 5 to 30), a heteroaryloxy group (for example, having a ring skeleton-constituting atom number of 5 to 30), a diarylamino group (for example, having 0 to 20 carbon atoms).

In the present description, “Substituent Group C” means one or a combination of two or more groups selected from the group consisting of an alkyl group (for example, having 1 to 20 carbon atoms), an aryl group (for example, having 6 to 22 carbon atoms), a heteroaryl group (for example, having a ring skeleton-constituting atom number of 5 to 20), and a diarylamino group (for example, having 12 to 20 carbon atoms).

In the present description, “Substituent Group D” means one or a combination of two or more groups selected from the group consisting of an alkyl group (for example, having 1 to 20 carbon atoms), an aryl group (for example, having 6 to 22 carbon atoms), and a heteroaryl group (for example, having a ring skeleton-constituting atom number of 5 to 20).

In the present description, “Substituent Group E” means one or a combination of two or more groups selected from the group consisting of an alkyl group (for example, having 1 to 20 carbon atoms), and an aryl group (for example, having 6 to 22 carbon atoms).

A part or all of the hydrogen atoms existing in the group described in these Substituent Groups A to E can be substituted with deuterium atoms.

In the present description, the substituent in the case of a description “substituent” or “substituted or unsubstituted” can be selected, for example, from Substituent Group A, or from Substituent Group B, or from Substituent Group C, or from Substituent Group D, or from Substituent Group E.

In some embodiments, the compound represented by the general formula (1) is a light emitting material.

In some embodiments, the compound represented by the general formula (1) is a compound capable of emitting delayed fluorescence.

In some embodiments of the present disclosure, the compound represented by the general formula (1) is, when excited thermally or by an electronic means, able to emit light in a UV region, emit light of blue, green, yellow, orange or red in a visible spectral region (e.g., about 420 nm to about 500 nm, about 500 nm to about 600 nm, or about 600 nm to about 700 nm) or in a near IR region.

In some embodiments of the present disclosure, the compound represented by the general formula (1) is, when excited thermally or by an electronic means, able to emit light of red or orange in a visible spectral region (e.g., about 620 nm to about 780 nm, about 650 nm).

In some embodiments of the present disclosure, the compound represented by the general formula (1) is, when excited thermally or by an electronic means, able to emit light of orange or yellow in a visible spectral region (e.g., about 570 nm to about 620 nm, about 590 nm, about 570 nm).

In some embodiments of the present disclosure, the compound represented by the general formula (1) is, when excited thermally or by an electronic means, able to emit light of green in a visible spectral region (e.g., about 490 nm to about 575 nm, about 510 nm).

In some embodiments of the present disclosure, the compound represented by the general formula (1) is, when excited thermally or by an electronic means, able to emit light of blue in a visible spectral region (e.g., about 400 nm to about 490 nm, about 475 nm).

In some embodiments of the present disclosure, the compound represented by the general formula (1) is, when excited thermally or by an electronic means, able to emit light in a UV spectral region (e.g., about 280 to 400 nm).

In some embodiments of the present disclosure, the compound represented by the general formula (1) is, when excited thermally or by an electronic means, able to emit light in an IR spectral region (e.g., about 780 nm to 2 μm).

In some embodiments of the present disclosure, an organic semiconductor device using the compound represented by the general formula (1) can be produced. For example, CMOS (complementary metal-oxide semiconductor) using the compound represented by the general formula (1) can be produced. In some embodiments of the present disclosure, an organic optical device such as an organic electroluminescent device and a solid-state image sensing device (for example, CMOS image sensor) using the compound represented by the general formula (1) can be produced.

Electronic characteristics of small-molecule chemical substance libraries can be calculated by known ab initio quantum chemistry calculation. For example, according to time-dependent density functional theory calculation using 6-31G* as a base, and a functional group known as Becke's three parameters, Lee-Yang-Parr hybrid functionals, the Hartree-Fock equation (TD-DFT/B3LYP/6-31G*) is analyzed and molecular fractions (parts) having HOMO not lower than a specific threshold value and LUMO not higher than a specific threshold value can be screened.

With that, for example, in the presence of a HOMO energy (for example, ionization potential) of −6.5 eV or more, a donor part (“D”) can be selected. On the other hand, for example, in the presence of a LUMO energy (for example, electron affinity) of −0.5 eV or less, an acceptor part (“A”) can be selected. A bridge part (“B”) is a strong conjugated system, for example, capable of strictly limiting the acceptor part and the donor part in a specific three-dimensional configuration, and therefore prevents the donor part and the acceptor part from overlapping in the pai-conjugated system.

In some embodiments, a compound library is screened using at least one of the following characteristics.
1. Light emission around a specific wavelength
2. A triplet state over a calculated specific energy level
3. ΔE_{ST} value lower than a specific value
4. Quantum yield more than a specific value
5. HOMO level
6. LUMO level

In some embodiments, the difference (ΔE_{ST}) between the lowest singlet excited state and the lowest triplet excited state at 77 K is less than about 0.5 eV, less than about 0.4 eV, less than about 0.3 eV, less than about 0.2 eV, or less than about 0.1 eV. In some embodiments, ΔE_{ST} value is less than about 0.09 eV, less than about 0.08 eV, less than about 0.07 eV, less than about 0.06 eV, less than about 0.05 eV, less than about 0.04 eV, less than about 0.03 eV, less than about 0.02 eV, or less than about 0.01 eV.

In some embodiments, the compound represented by the general formula (1) shows a quantum yield of more than 25%, for example, about 30%, about 35%, about 40%, about 45%, about 50%, about 55%, about 60%, about 65%, about 70%, about 75%, about 80%, about 85%, about 90%, about 95% or more.

In some embodiments, the compound represented by the general formula (1) is a host material. For example, a light emitting material that exhibits a light emitting property, as a dopant, can be used in combination with the host material represented by the general formula (1). As a dopant, a fluorescent material and a phosphorescent material can be used widely, and a delayed fluorescent material can also be used. A typical use embodiment is an embodiment of using the compound as a host material in a light emitting layer of an organic electroluminescent device. The compound represented by the general formula (1) can be used in combination with any other host material. In that case, preferably, a compound having a higher lowest excited singlet state energy level than that of the compound represented by the general formula (1) is combined as the other host material.

### [Synthesis Method for Compound Represented by General Formula (1)]

The compound represented by the general formula (1) is a novel compound.

The compound represented by the general formula (1) can be synthesized by combining known reactions. For example, the compound can be synthesized by utilizing ring-closing reaction or by utilizing substitution reaction. Regarding the specific condition for synthesis, reference can be made to Synthesis Examples given hereinunder.

### [Structure Using the Compound Represented by the General Formula (1)]

In some embodiments, the compound represented by the general formula (1) is used along with one or more materials (e.g., small molecules, polymers, metals, metal complexes), by combining them, or by dispersing the compound, or by covalent-bonding with the compound, or by coating with the compound, or by carrying the compound, or by associating with the compound, and solid films or layers are formed. For example, by combining the compound represented by the general formula (1) with an electroactive material, a film can be formed. In some cases, the compound represented by the general formula (1) can be combined with a hole transporting polymer. In some cases, the compound represented by the general formula (1) can be combined with an electron transporting polymer. In some cases, the compound represented by the general formula (1) can be combined with a hole transporting polymer and an electron transporting polymer. In some cases, the compound represented by the general formula (1) can be combined with a copolymer having both a hole transporting moiety and an electron transporting moiety. In the embodiments mentioned above, the electrons and/or the holes formed in a solid film or layer can be interacted with the compound represented by the general formula (1).

### [Film Formation]

In some embodiments, a film containing the compound represented by the general formula (1) of the invention can be formed in a wet process. In a wet process, a solution prepared by dissolving a composition containing the compound of the present invention is applied onto a surface, and then the solvent is removed to form a film. The wet process includes a spin coating method, a slit coating method, an inkjet method (a spraying method), a gravure printing method, an offset printing method and flexographic printing method, which, however, are not limitative. In the wet process, an appropriate organic solvent capable of dissolving a composition containing the compound of the present invention is selected and used. In some embodiments, a substituent (e.g., an alkyl group) capable of increasing the solubility in an organic solvent can be introduced into the compound contained in the composition.

In some embodiments, a film containing the compound of the present invention can be formed in a dry process. In some embodiments, a vacuum evaporation method is employable as a dry process, which, however, is not limitative. In the case where a vacuum evaporation method is employed, compounds to constitute a film can be co-evaporated from individual evaporation sources, or can be co-evaporated from a single evaporation source formed by mixing the compounds. In the case where a single evaporation source is used, a mixed powder prepared by mixing compound powders can be used, or a compression molded body prepared by compression-molding the mixed powder can be used, or a mixture prepared by heating and melting the constituent compounds and cooling the resulting melt can be used. In some embodiments, by co-evaporation under the condition where the evaporation rate (weight reduction rate) of the plural compounds contained in a single evaporation source is the same or is nearly the same, a film having a compositional ratio corresponding to the compositional ratio of the plural compounds contained in the evaporation source can be formed. When plural compounds are mixed in the same compositional ratio as the compositional ratio of the film to be formed to prepare an evaporation source, a film having a desired compositional ratio can be formed in a simplified manner. In some embodiments, the temperature at which the compounds to be co-evaporated have the same weight reduction ratio is specifically defined, and the temperature can be employed as the temperature of co-evaporation.

### [Use Examples of Compound Represented by General Formula (1)]

The compound represented by the general formula (1) has excellent light emission characteristics, and is useful as a material for organic light emitting devices. The compound of the present invention includes a compound excellent in light emission characteristics, for example, having a high light emission efficiency. Further, the compound of the present invention includes a compound excellent in light emission characteristics and having a long lifetime. Consequently, the compound of the present invention is especially favorably used for organic light emitting diodes and the like.

### Organic Light Emitting Diode:

One embodiment of the present invention relates to use of the compound represented by the general formula (1) of the present invention as a light emitting material for organic light emitting devices. In some embodiments, the compound represented by the general formula (1) of the present invention can be effectively used as a light emitting material in a light emitting layer in an organic light emitting device. In some embodiments, the compound represented by the general formula (1) of the includes delayed fluorescence (delayed fluorescent material) that emits delayed fluorescence. In some embodiments, the present invention provides a delayed fluorescent material having a structure represented by the general formula (1). In some embodiments, the present invention relates to use of the compound represented by the general formula (1) as a delayed fluorescent material. In some embodiments, the compound represented by the general formula (1) of the present invention can be used as a host material, and can be used along with one or more light emitting materials, and the light emitting material can be a fluorescent material, a phosphorescent material or a TADF. In some embodiments, the compound represented by the general formula (1) can be used as a hole transporting material. In some embodiments, the compound represented by the general formula (1) can be used as an electron transporting material. In some embodiments, the present invention relates to a method of generating delayed fluorescence from the compound represented by the general formula (1). In some embodiments, the organic light emitting device containing the compound as a light emitting material emits delayed fluorescence and shows a high light emission efficiency.

In some embodiments, the light emitting layer contains the compound represented by the general formula (1), and the compound represented by the general formula (1) is aligned in parallel to the substrate. In some embodiments, the substrate is a film-forming surface. In some embodiment, the alignment of the compound represented by the general formula (1) relative to the film-forming surface can have some influence on the propagation direction of light emitted by the aligned compounds, or can determine the direction. In some embodiments, by aligning the propagation direction of light emitted by the compound represented by the general formula (1), the light extraction efficiency from the light emitting layer can be improved.

One embodiment of the present invention relates to an organic light emitting device. In some embodiments, the organic light emitting device includes a light emitting layer. In some embodiments, the light emitting layer contains, as a light emitting material, the compound represented by the general formula (1). In some embodiments, the organic light emitting device is an organic photoluminescent device (organic PL device). In some embodiments, the organic light emitting device is an organic electroluminescent device (organic EL device). In some embodiments, the compound represented by the general formula (1) assists light irradiation from the other light emitting materials contained in the light emitting layer (as a so-called assist dopant). In some embodiments, the compound represented by the general formula (1) contained in the light emitting layer is in a lowest excited energy level, and is contained between the lowest excited single energy level of the host material contained in the light emitting layer and the lowest excited singlet energy level of the other light emitting materials contained in the light emitting layer.

In some embodiments, the organic photoluminescent device comprises at least one light emitting layer. In some embodiments, the organic electroluminescent device comprises at least an anode, a cathode, and an organic layer between the anode and the cathode. In some embodiments, the organic layer comprises at least a light emitting layer. In some embodiments, the organic layer comprises only a light emitting layer. In some embodiments, the organic layer comprises one or more organic layers in addition to the light emitting layer. Examples of the organic layer include a hole transporting layer, a hole injection layer, an electron barrier layer, a hole barrier layer, an electron injection layer, an electron transporting layer and an exciton barrier layer. In some embodiments, the hole transporting layer may be a hole injection and transporting layer having a hole injection function, and the electron transporting layer may be an electron injection and transporting layer having an electron injection function.

### Light Emitting Layer:

In some embodiments, the light emitting layer is a layer where holes and electrons injected from the anode and the cathode, respectively, are recombined to form excitons. In some embodiments, the layer emits light.

In some embodiments, only a light emitting material is used as the light emitting layer. In some embodiments, the light emitting layer contains a light emitting material and a host material. In some embodiments, the light emitting material is a compound represented by the general formula (1). In some embodiments, for improving light emission efficiency of an organic electroluminescent device and an organic photoluminescence device, the singlet exciton and the triplet exciton generated in a light emitting material are confined inside the light emitting material. In some embodiments, a host material is used in the light emitting layer in addition to a light emitting material therein. In some embodiments, the host material is an organic compound. In some embodiments, the organic compound has an excited singlet energy and an excited triplet energy, and at least one of them is higher than those in the light emitting material of the present invention. In some embodiments, the singlet exciton and the triplet exciton generated in the light emitting material of the present invention are confined in the molecules of the light emitting material of the present invention. In some embodiments, the singlet and triplet excitons are fully confined for improving light emission efficiency. In some embodiments, although high light emission efficiency is still attained, singlet excitons and triplet excitons are not fully confined, that is, a host material capable of attaining high light emission efficiency can be used in the present invention with no specific limitation. In some embodiments, in the light emitting material in the light emitting layer of the device of the present invention, luminous radiation occurs. In some embodiments, radiated light includes both fluorescence and delayed fluorescence. In some embodiments, radiated light includes radiated light from a host material. In some embodiments, radiated light is composed of radiated light from a host material. In some embodiments, radiated light includes radiated light from the compound represented by the general formula (1) and radiated light from a host material. In some embodiment, a TADF molecule and a host material are used. In some embodiments, TADF is an assist dopant, of which the excited singlet energy is lower than that of the host material in the light emitting layer and is higher than that of the light emitting material in the light emitting layer.

In the case where the compound represented by the general formula (1) is used as an assist dopant, various compounds can be employed as a light emitting material (preferably a fluorescent material). As such light emitting materials, employable are an anthracene derivative, a tetracene derivative, a naphthacene derivative, a pyrene derivative, a perylene derivative, a chrysene derivative, a rubrene derivative, a coumarin derivative, a pyran derivative, a stilbene derivative, a fluorene derivative, an anthryl derivative, a pyrromethene derivative, a terphenyl derivative, a terphenylene derivative, a fluoranthene derivative, an amine derivative, a quinacridone derivative, an oxadiazole derivative, a malononitrile derivative, a carbazole derivative, a julolidine derivative, a thiazole derivative, and a metal (Al, Zn)-having derivative. These exemplified skeletons can have a substituent, or may not have a substituent. These exemplified skeletons can be combined.

Light emitting materials that can be used in combination with the assist dopant having a structure represented by the general formula (1) are shown below.

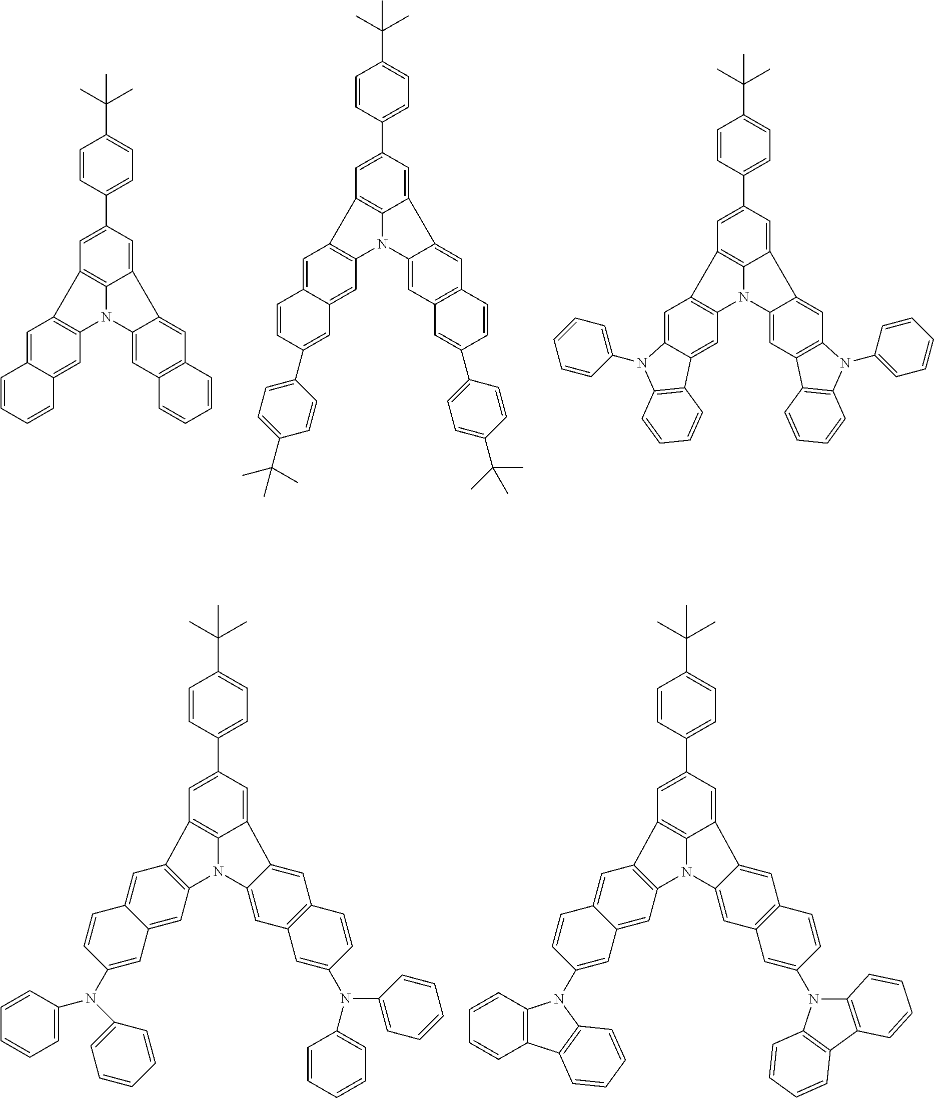

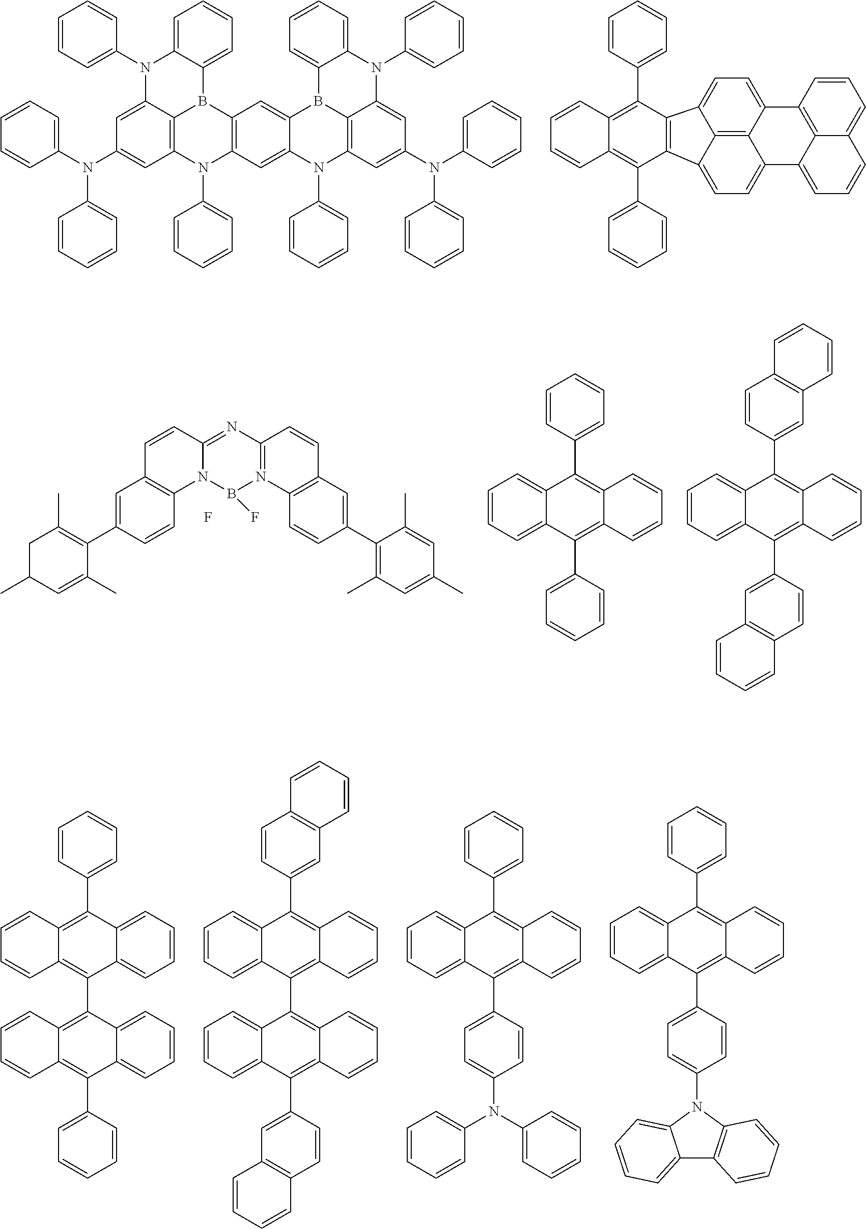

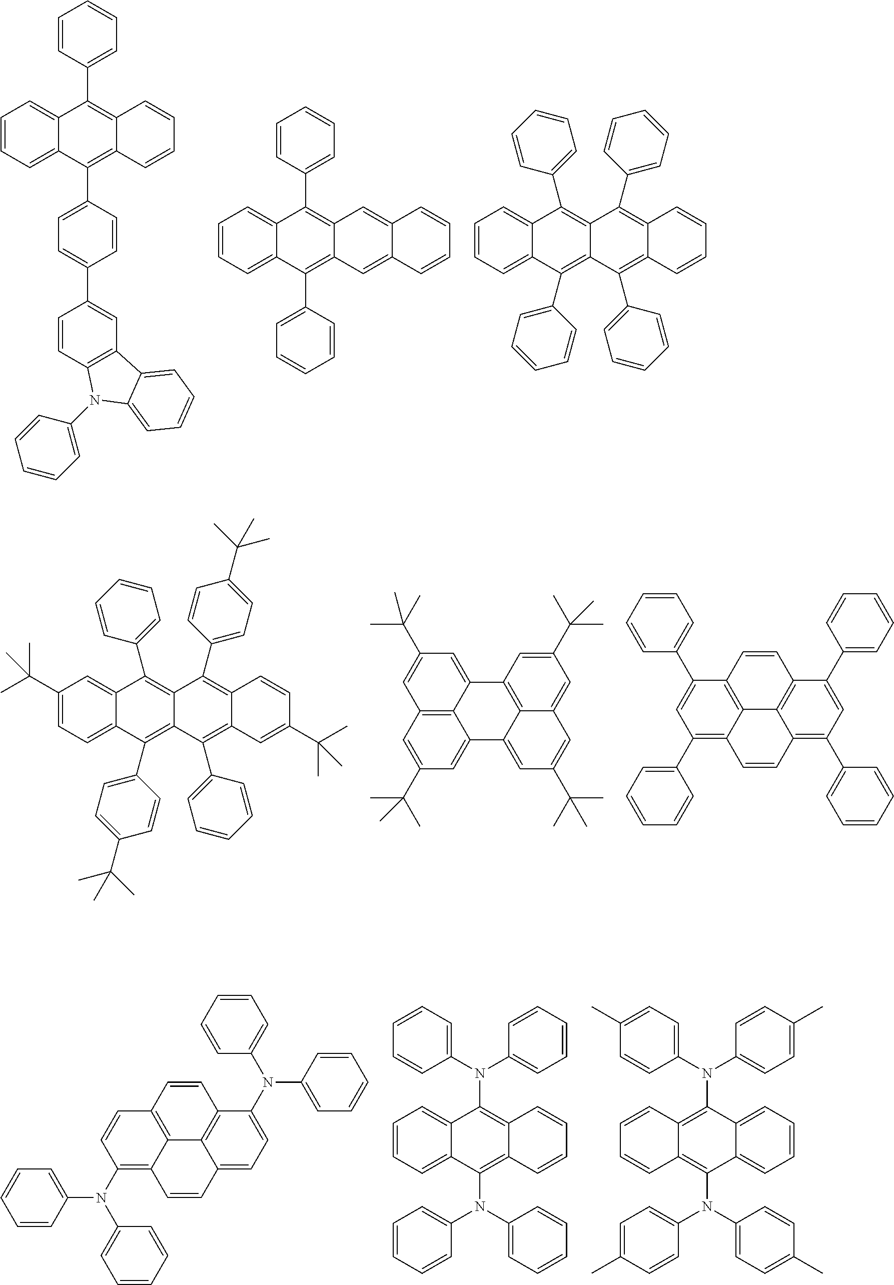

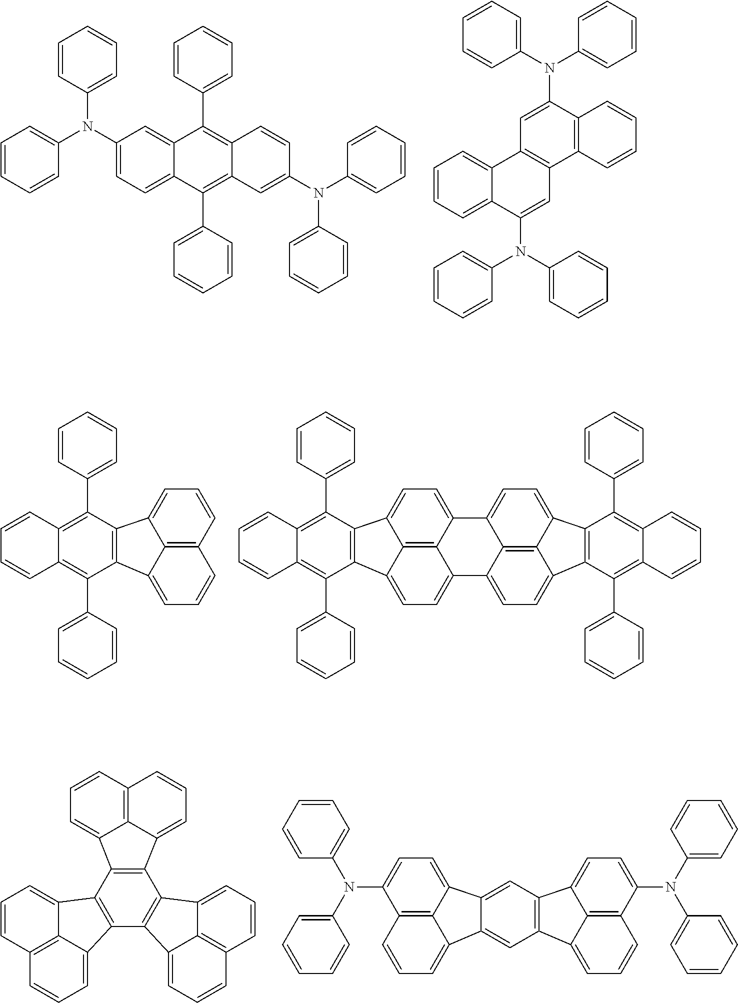

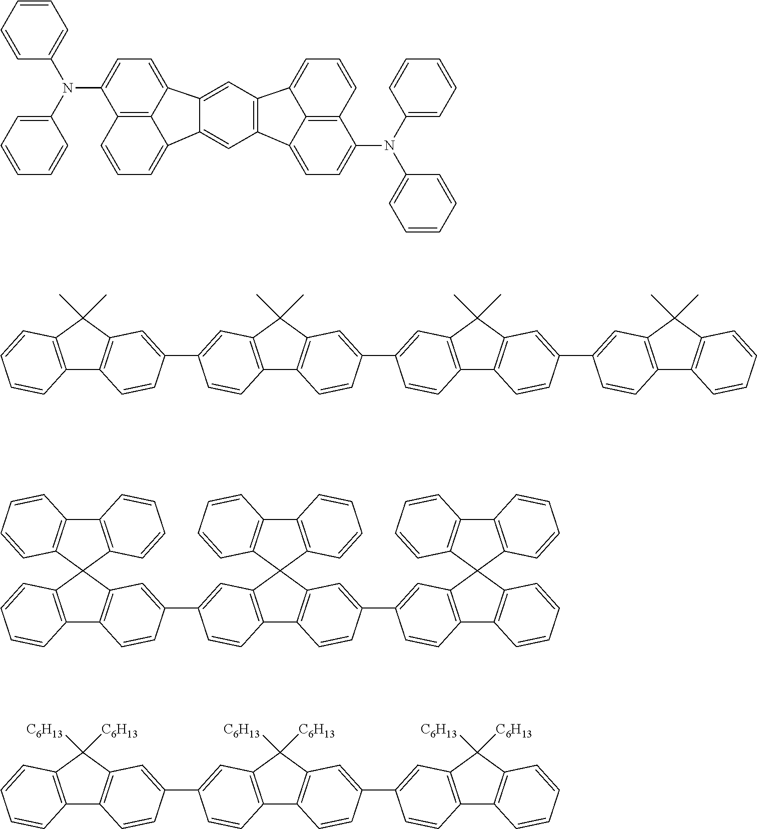

In addition, the compounds described in WO2015/022974, paragraphs 0220 to 0239 are also especially favorably employable as a light emitting material for use along with the assist dopant having a structure represented by the general formula (1).

In some embodiments where a host material is used, the amount of the compound of the present invention contained in a light emitting layer as a light emitting material is 0.1% by weight or more. In some embodiments where a host material is used, the amount of the compound of the present invention contained in a light emitting layer as a light emitting material is 1% by weight or more. In some embodiments where a host material is used, the amount of the compound of the present invention contained in a light emitting layer as a light emitting material is 50% by weight or less. In some embodiments where a host material is used, the amount of the compound of the present invention contained in a light emitting layer as a light emitting material is 20% by weight or less. In some embodiments where a host material is used, the amount of the compound of the present invention contained in a light emitting layer as a light emitting material is 10% by weight or less.

In some embodiments, the host material in a light emitting layer is an organic compound having a hole transporting capability and an electron transporting capability. In some embodiments, the host material in a light emitting layer is an organic compound that prevents increase in the wavelength of emitted light. In some embodiments, the host material in a light emitting layer is an organic compound having a high glass transition temperature.

In some embodiments, the host material is selected from the group consisting of the followings:

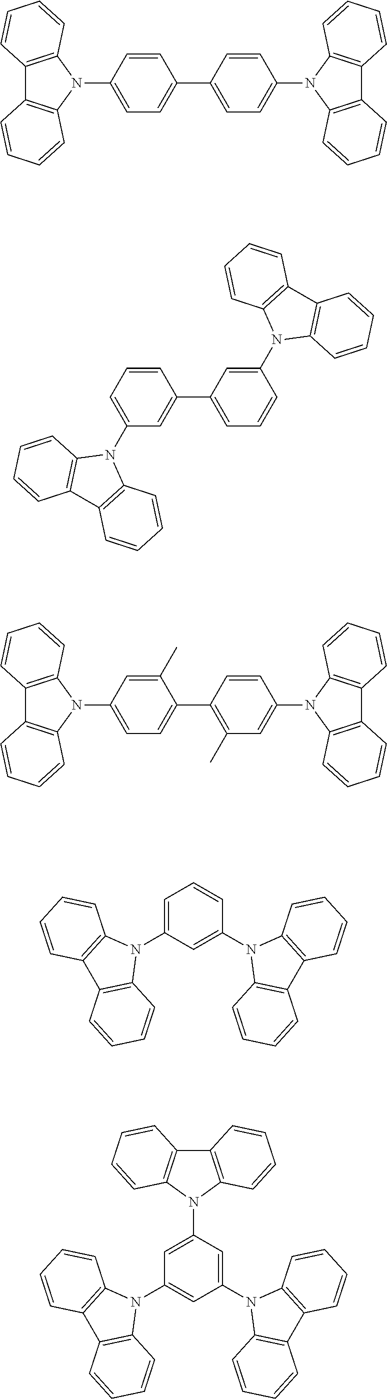 [SMILES: c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1 ; Cc1cc(-n2c3ccccc3c3ccccc32)ccc1-c1ccc(-n2c3ccccc3c3ccccc32)cc1C ; c1cc(-n2c3ccccc3c3ccccc32)cc(-n2c3ccccc3c3ccccc32)c1 ; c1ccc2c(c1)c1ccccc1n2-c1cc(-n2c3ccccc3c3ccccc32)cc(-n2c3ccccc3c3ccccc32)c1]

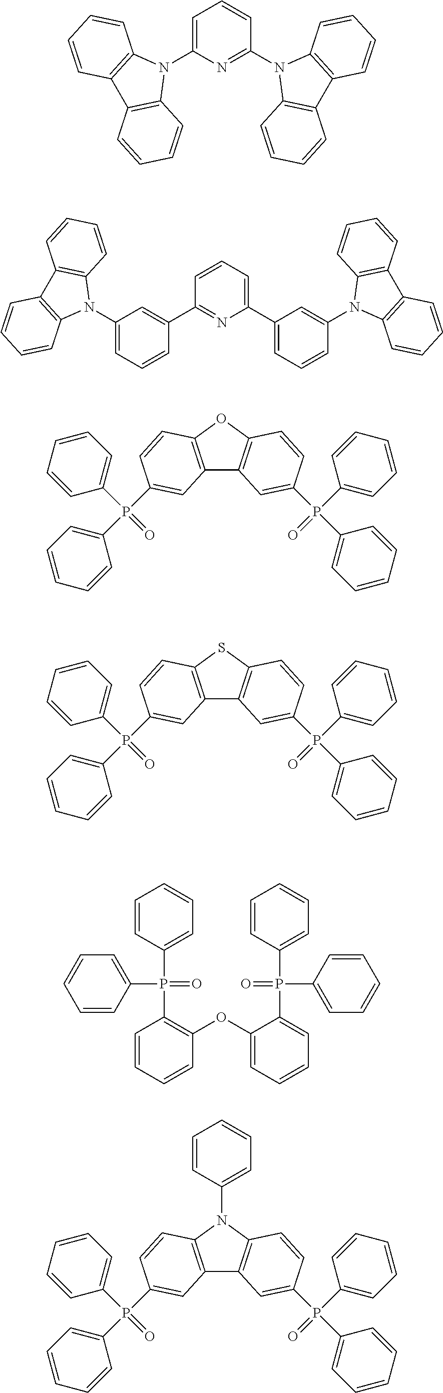 [SMILES: c1cc(-n2c3ccccc3c3ccccc32)nc(-n2c3ccccc3c3ccccc32)c1 ; c1cc(-c2cccc(-c3cccc(-n4c5ccccc5c5ccccc54)c3)n2)cc(-n2c3ccccc3c3ccccc32)c1 ; O=P(c1ccccc1)(c1ccccc1)c1ccc2oc3ccc(P(=O)(c4ccccc4)c4ccccc4)cc3c2c1 ; O=P(c1ccccc1)(c1ccccc1)c1ccc2sc3ccc(P(=O)(c4ccccc4)c4ccccc4)cc3c2c1 ; O=P(c1ccccc1)(c1ccccc1)c1ccccc1Oc1ccccc1P(=O)(c1ccccc1)c1ccccc1 ; O=P(c1ccccc1)(c1ccccc1)c1ccc2c(c1)c1cc(P(=O)(c3ccccc3)c3ccccc3)ccc1n2-c1ccccc1]

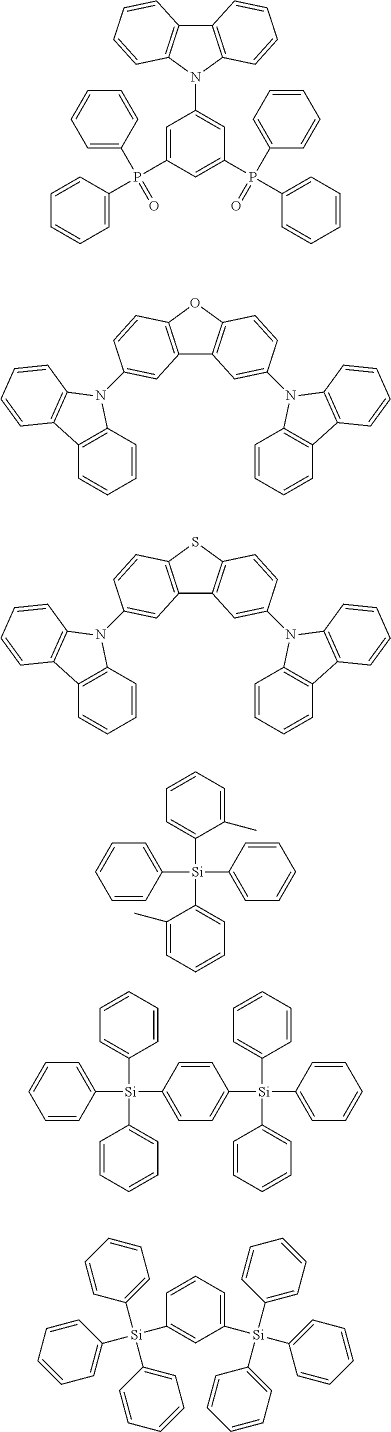 [SMILES: O=P(c1ccccc1)(c1ccccc1)c1cc(-n2c3ccccc3c3ccccc32)cc(P(=O)(c2ccccc2)c2ccccc2)c1 ; c1ccc2c(c1)c1ccccc1n2-c1ccc2oc3ccc(-n4c5ccccc5c5ccccc54)cc3c2c1 ; c1ccc2c(c1)c1ccccc1n2-c1ccc2sc3ccc(-n4c5ccccc5c5ccccc54)cc3c2c1 ; Cc1ccccc1[Si](c1ccccc1)(c1ccccc1)c1ccccc1C ; c1ccc([Si](c2ccccc2)(c2ccccc2)c2ccc([Si](c3ccccc3)(c3ccccc3)c3ccccc3)cc2)cc1 ; c1ccc([Si](c2ccccc2)(c2ccccc2)c2cccc([Si](c3ccccc3)(c3ccccc3)c3ccccc3)c2)cc1]

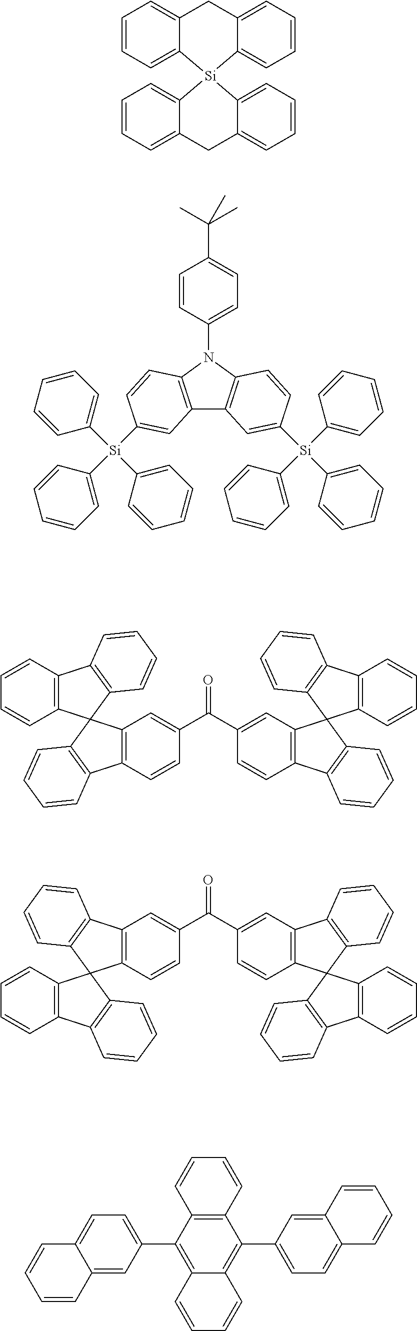

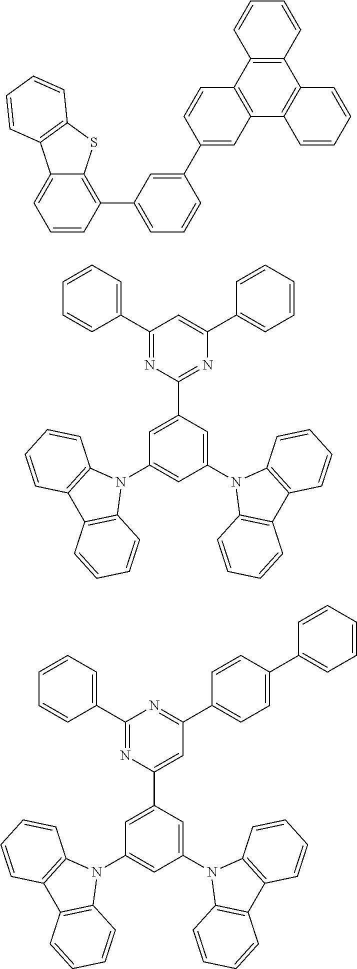

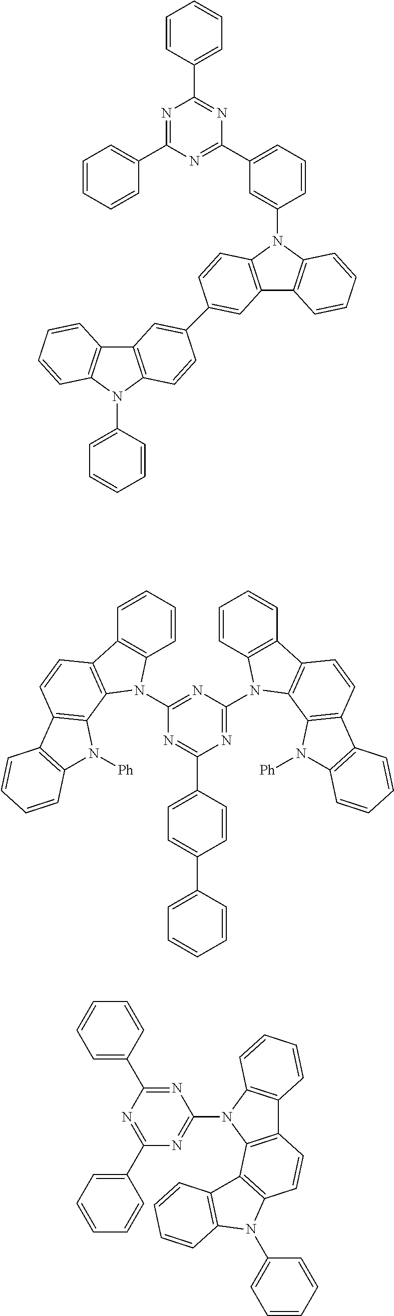 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

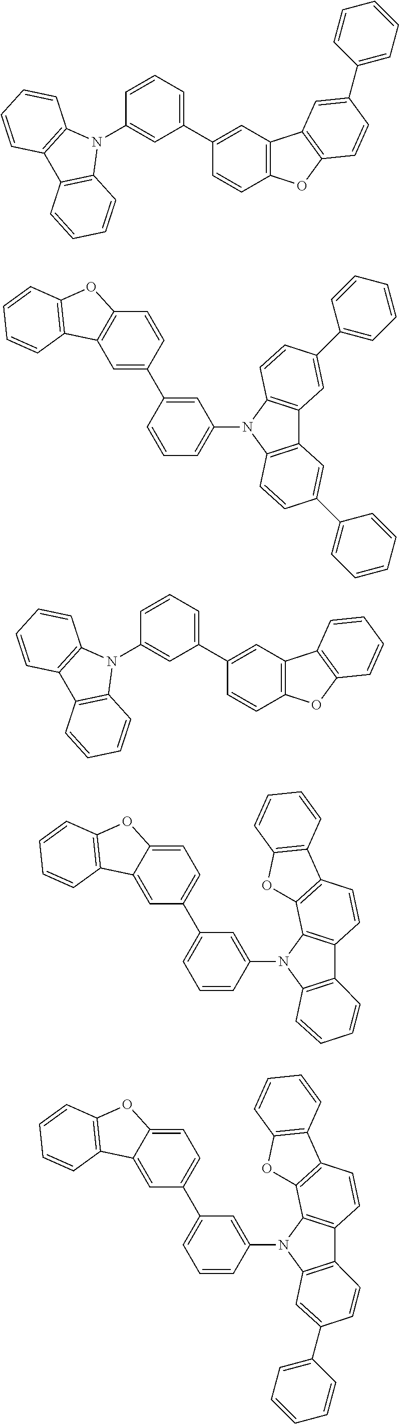

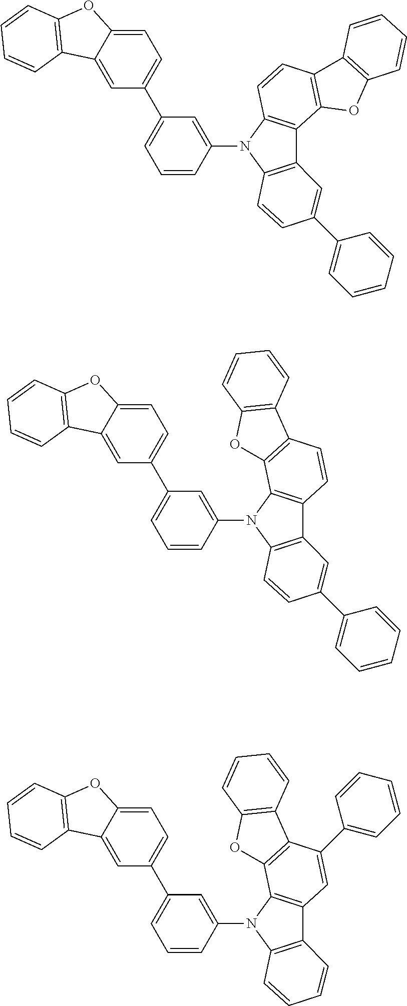 [SMILES: c1ccc(-c2ccc3c(c2)c2c4oc5ccccc5c4ccc2n3-c2cccc(-c3ccc4oc5ccccc5c4c3)c2)cc1 ; c1ccc(-c2ccc3c(c2)c2ccc4c5ccccc5oc4c2n3-c2cccc(-c3ccc4oc5ccccc5c4c3)c2)cc1 ; c1ccc(-c2cc3c4ccccc4n(-c4cccc(-c5ccc6oc7ccccc7c6c5)c4)c3c3oc4ccccc4c23)cc1]

In some embodiments, the light emitting layer contains two or more kinds of TADF molecules differing in the structure. For example, the light emitting layer can contain three kinds of materials of a host material, a first TADF molecule and a second TADF molecule whose excited singlet energy level is higher in that order. In that case, both the first TADF molecule and the second TADF molecule are preferably such that the difference ΔE_{ST} between the lowest excited singlet energy level and the lowest excited triplet energy level at 77 K is 0.3 eV or less, more preferably 0.25 eV or less, even more preferably 0.2 eV or less, further more preferably 0.15 eV or less, further more preferably 0.1 eV or less, further more preferably 0.07 eV or less, further more preferably 0.05 eV or less, further more preferably 0.03 eV or less, and particularly preferably 0.01 eV or less. The content of the first TADF molecule in the light emitting layer is preferably larger than the content of the second TADF molecule therein. The content of the host material in the light emitting layer is preferably larger than the content of the second TADF molecule therein. The content of the first TADF molecule in the light emitting layer can be larger than or can be smaller than or can be the same as the content of the host material therein. In some embodiments, the composition in the light emitting layer can be 10 to 70% by weight of a host material, 10 to 80% by weight of a first TADF molecule, and 0.1 to 30% by weight of a second TADF molecule. In some embodiments, the composition in the light emitting layer can be 20 to 45% by weight of a host material, 50 go 75% by weight of a first TADF molecule, and 5 to 20% by weight of a second TADF molecule. In some embodiments, the photoluminescence quantum yield φPL1 (A) by photo-excitation of a co-deposited film of a first TADF molecule and a host material (the content of the first TADF molecule in the co-deposited film=A % by weight) and the photoluminescence quantum yield φPL2 (A) by photo-excitation of a co-deposited film of a second TADF molecule and a host material (the content of the second TADF molecule in the co-deposited film=A % by weight) satisfy a relational formula φPL1 (A)>φPL2 (A). In some embodiments, the photoluminescence quantum yield φPL2 (B) by photo-excitation of a co-deposited film of a second TADF molecule and a host material (the content of the second TADF molecule in the co-deposited film=B % by weight) and the photoluminescence quantum yield φPL2 (100) by photo-excitation of a single film of a second TADF molecule satisfy a relational formula φPL2 (B)>φPL2 (100). In some embodiments, the light emitting layer can contain three kinds of TADF molecules differing in the structure. The compound of the present invention can be any of the plural TADF compounds contained in the light emitting layer.

In some embodiments, the light emitting layer can be composed of materials selected from the group consisting of a host material, an assist dopant and a light emitting material. In some embodiments, the light emitting layer does not contain a metal element. In some embodiments, the light emitting layer can be formed of a material composed of atoms alone selected from the group consisting of a carbon atom, a hydrogen atom, a deuterium atom, a nitrogen atom, an oxygen atom and a sulfur atom. Or the light emitting layer can be formed of a material composed of atoms alone selected from the group consisting of a carbon atom, a hydrogen atom, a deuterium atom, a nitrogen atom and an oxygen atom. Or the light emitting layer can be formed of a material composed of atoms alone selected from the group consisting of a carbon atom, a hydrogen atom, a nitrogen atom and an oxygen atom.

In the case where the light emitting layer contains any other TADF material than the compound of the present invention, the TADF material can be a known delayed fluorescent material. As preferred delayed fluorescent materials, there can be mentioned compounds included in the general formulae described in WO2013/154064, paragraphs 0008 to 0048 and 0095 to 0133; WO2013/011954, paragraphs 0007 to 0047 and 0073˜0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101˜0121; JP 2014-9352 A, paragraphs 0007 to 0041 and 0060 to 0069; and JP 2014-9224 A, paragraphs 0008 to 0048 and 0067 to 0076; JP 2017-119663 A, paragraphs 0013 to 0025; JP 2017-119664 A, paragraphs 0013 to 0026; JP 2017-222623 A, paragraphs 0012 to 0025; JP 2017-226838 A, paragraphs 0010 to 0050; JP 2018-100411 A, paragraphs 0012 to 0043; WO2018/047853, paragraphs 0016 to 0044; and especially, exemplary compounds therein capable of emitting delayed fluorescence. In addition, also preferably employable here are light emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541 and WO2015/159541. These patent publications described in this paragraph are hereby incorporated as a part of this description by reference.

In the following, the constituent members and the other layers than the light emitting layer of the organic electroluminescent device are described.

### Substrate:

In some embodiments, the organic electroluminescent device of the invention is supported by a substrate, wherein the substrate is not particularly limited and may be any of those that have been commonly used in an organic electroluminescent device, for example those formed of glass, transparent plastics, quartz and silicon.

### Anode:

In some embodiments, the anode of the organic electroluminescent device is made of a metal, an alloy, an electroconductive compound, or a combination thereof. In some embodiments, the metal, alloy, or electroconductive compound has a large work function (4 eV or more). In some embodiments, the metal is Au. In some embodiments, the electroconductive transparent material is selected from CuI, indium tin oxide (ITO), SnO₂, and ZnO. In some embodiments, an amorphous material capable of forming a transparent electroconductive film, such as IDIXO (In₂O₃—ZnO), is be used. In some embodiments, the anode is a thin film. In some embodiments, the thin film is made by vapor deposition or sputtering. In some embodiments, the film is patterned by a photolithography method. In some embodiments, where the pattern does not require high accuracy (for example, approximately 100 μm or more), the pattern may be formed with a mask having a desired shape on vapor deposition or sputtering of the electrode material. In some embodiments, when a coating material such as an organic electroconductive compound can be applied, a wet film forming method, such as a printing method and a coating method is used. In some embodiments, when the emitted light goes through the anode, the anode has a transmittance of more than 10%, and the anode has a sheet resistance of several hundred Ohm per unit area or less. In some embodiments, the thickness of the anode is from 10 to 1,000 nm. In some embodiments, the thickness of the anode is from 10 to 200 nm. In some embodiments, the thickness of the anode varies depending on the material used.

### Cathode

In some embodiments, the cathode is made of an electrode material such as a metal having a small work function (4 eV or less) (referred to as an electron injection metal), an alloy, an electroconductive compound, or a combination thereof. In some embodiments, the electrode material is selected from sodium, a sodium-potassium alloy, magnesium, lithium, a magnesium-copper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth element. In some embodiments, a mixture of an electron injection metal and a second metal that is a stable metal having a larger work function than the electron injection metal is used. In some embodiments, the mixture is selected from a magnesium-silver mixture, a magnesium-aluminum mixture, a magnesium-indium mixture, an aluminum-aluminum oxide (Al₂O₃) mixture, a lithium-aluminum mixture, and aluminum. In some embodiments, the mixture increases the electron injection property and the durability against oxidation. In some embodiments, the cathode is produced by forming the electrode material into a thin film by vapor deposition or sputtering. In some embodiments, the cathode has a sheet resistance of several hundred Ohm per unit area or less. In some embodiments, the thickness of the cathode ranges from 10 nm to 5 μm. In some embodiments, the thickness of the cathode ranges from 50 to 200 nm. In some embodiments, for transmitting the emitted light, any one of the anode and the cathode of the organic electroluminescent device is transparent or translucent. In some embodiments, the transparent or translucent electroluminescent device enhances the light emission luminance.

In some embodiments, the cathode is formed with an electroconductive transparent material, as described for the anode, to form a transparent or translucent cathode. In some embodiments, a device comprises an anode and a cathode, both being transparent or translucent.

### Injection Layer

An injection layer is a layer between the electrode and the organic layer. In some embodiments, the injection layer decreases the drive voltage and enhances the light emission luminance. In some embodiments, the injection layer includes a hole injection layer and an electron injection layer. The injection layer can be positioned between the anode and the light emitting layer or the hole transporting layer, and between the cathode and the light emitting layer or the electron transporting layer. In some embodiments, an injection layer is present. In some embodiments, no injection layer is present.

Preferred compound examples for use as a hole injection material are shown below.

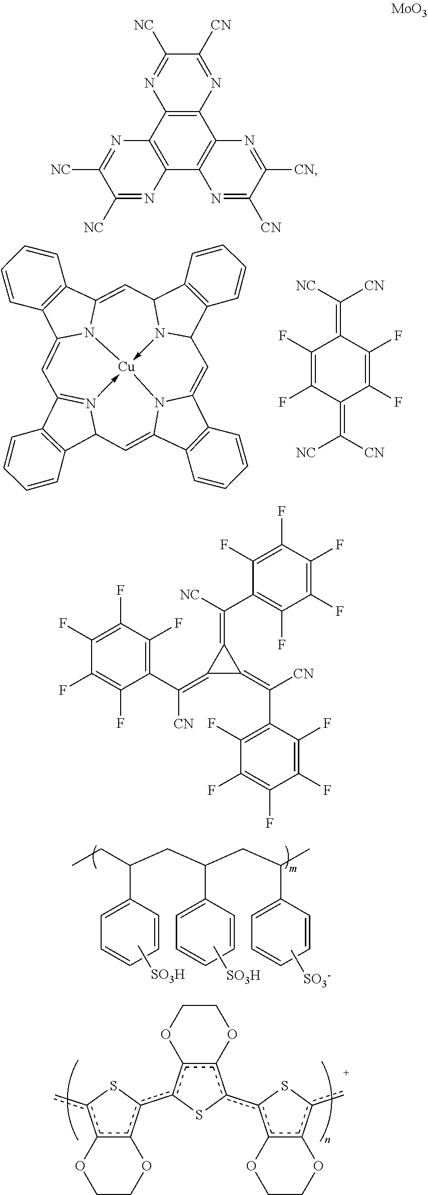

Next, preferred compound examples for use as an electron injection material are shown below.

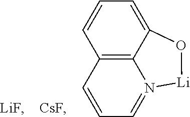

### Barrier Layer

A barrier layer is a layer capable of inhibiting charges (electrons or holes) and/or excitons present in the light emitting layer from being diffused outside the light emitting layer. In some embodiments, the electron barrier layer is between the light emitting layer and the hole transporting layer, and inhibits electrons from passing through the light emitting layer toward the hole transporting layer. In some embodiments, the hole barrier layer is between the light emitting layer and the electron transporting layer, and inhibits holes from passing through the light emitting layer toward the electron transporting layer. In some embodiments, the barrier layer inhibits excitons from being diffused outside the light emitting layer. In some embodiments, the electron barrier layer and the hole barrier layer are exciton barrier layers. As used in the present description, the term “electron barrier layer” or “exciton barrier layer” includes a layer that has the functions of both electron barrier layer and an exciton barrier layer.

### Hole Barrier Layer

A hole barrier layer acts as an electron transporting layer. In some embodiments, the hole barrier layer inhibits holes from reaching the electron transporting layer while transporting electrons. In some embodiments, the hole barrier layer enhances the recombination probability of electrons and holes in the light emitting layer. The material for the hole barrier layer may be the same materials as the ones described for the electron transporting layer.

Preferred compound examples for use for the hole barrier layer are shown below.

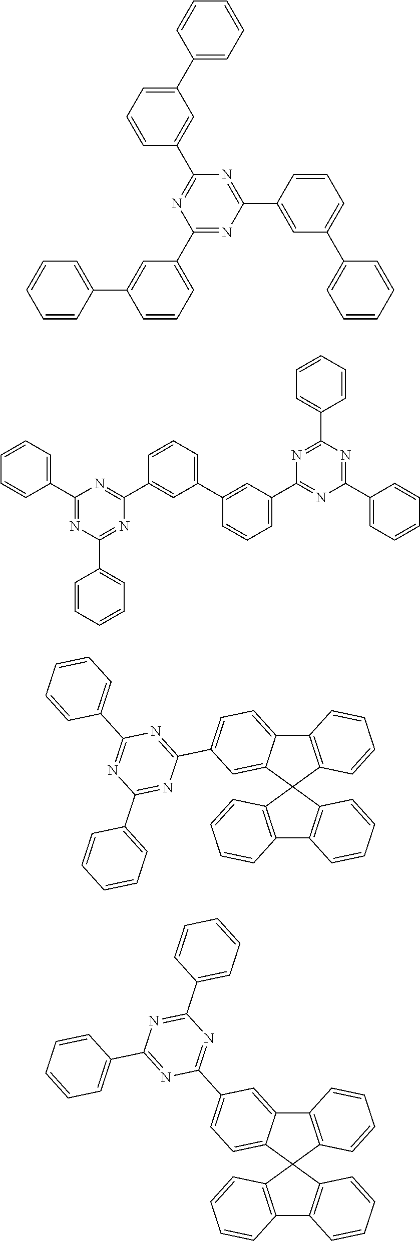

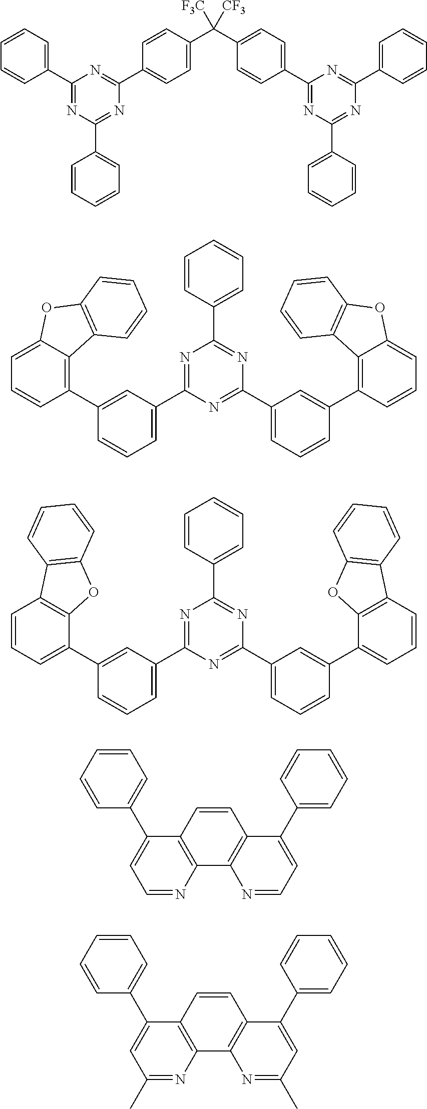

### Electron Barrier Layer

An electron barrier layer transports holes. In some embodiments, the electron barrier layer inhibits electrons from reaching the hole transporting layer while transporting holes. In some embodiments, the electron barrier layer enhances the recombination probability of electrons and holes in the light emitting layer. The material used for the electron barrier layer can be the same material as the ones described above for the hole transporting layer.

Preferred compound examples for use as the electron barrier material are shown below.

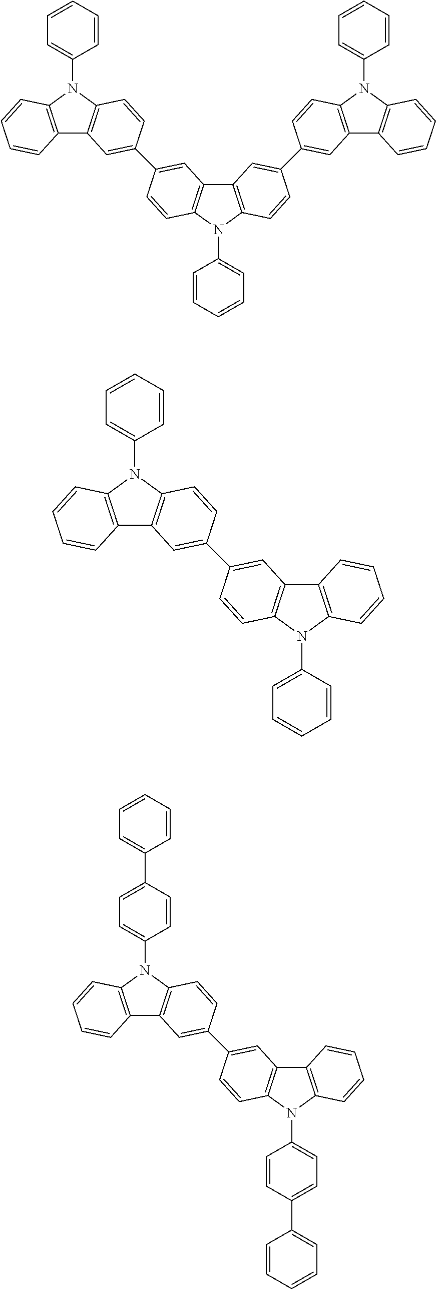 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

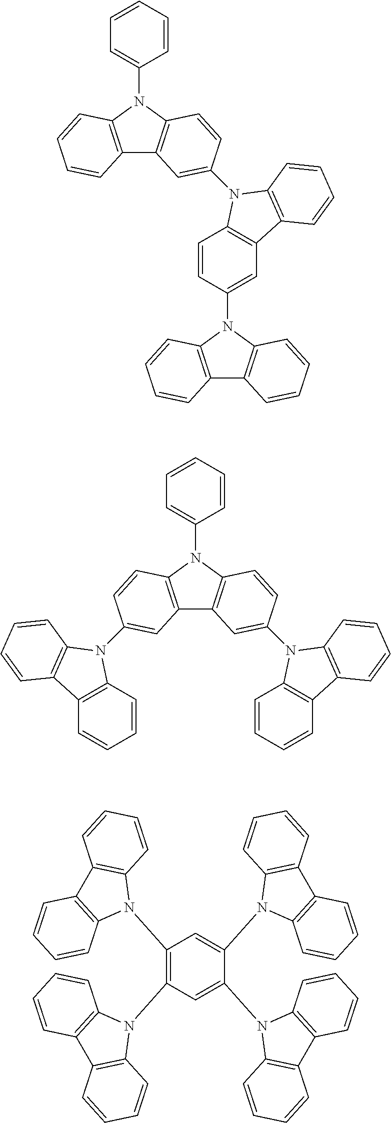 [SMILES: c1ccc(-n2c3ccccc3c3cc(-n4c5ccccc5c5cc(-n6c7ccccc7c7ccccc76)ccc54)ccc32)cc1 ; c1ccc(-n2c3ccc(-n4c5ccccc5c5ccccc54)cc3c3cc(-n4c5ccccc5c5ccccc54)ccc32)cc1 ; c1ccc2c(c1)c1ccccc1n2-c1cc(-n2c3ccccc3c3ccccc32)c(-n2c3ccccc3c3ccccc32)cc1-n1c2ccccc2c2ccccc21]

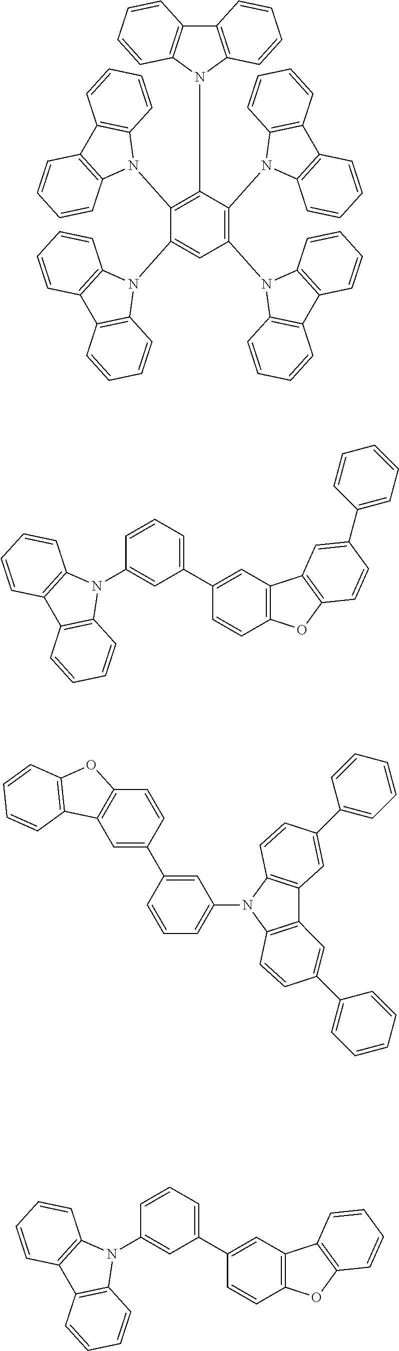 [SMILES: c1ccc2c(c1)c1ccccc1n2-c1cc(-n2c3ccccc3c3ccccc32)c(-n2c3ccccc3c3ccccc32)c(-n2c3ccccc3c3ccccc32)c1-n1c2ccccc2c2ccccc21 ; c1ccc(-c2ccc3oc4ccc(-c5cccc(-n6c7ccccc7c7ccccc76)c5)cc4c3c2)cc1 ; c1ccc(-c2ccc3c(c2)c2cc(-c4ccccc4)ccc2n3-c2cccc(-c3ccc4oc5ccccc5c4c3)c2)cc1 ; c1cc(-c2ccc3oc4ccccc4c3c2)cc(-n2c3ccccc3c3ccccc32)c1]

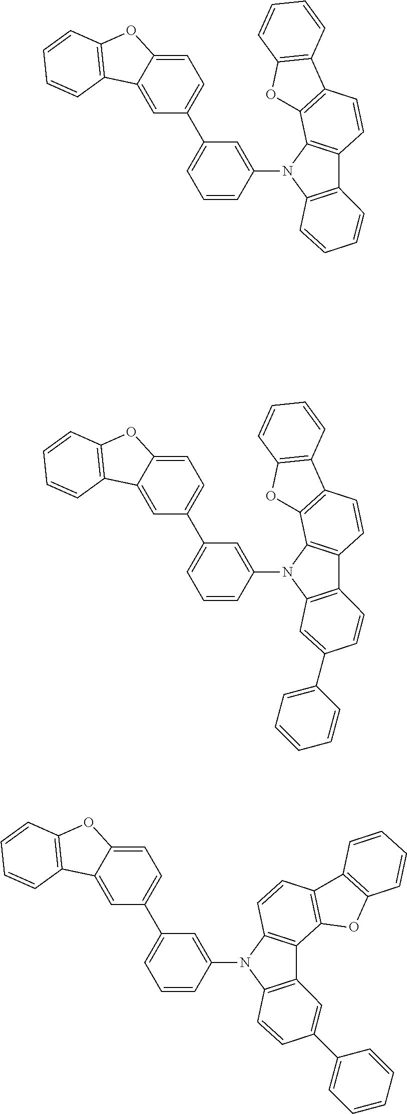 [SMILES: c1cc(-c2ccc3oc4ccccc4c3c2)cc(-n2c3ccccc3c3ccc4c5ccccc5oc4c32)c1 ; c1ccc(-c2ccc3c4ccc5c6ccccc6oc5c4n(-c4cccc(-c5ccc6oc7ccccc7c6c5)c4)c3c2)cc1 ; c1ccc(-c2ccc3c(c2)c2c4oc5ccccc5c4ccc2n3-c2cccc(-c3ccc4oc5ccccc5c4c3)c2)cc1]

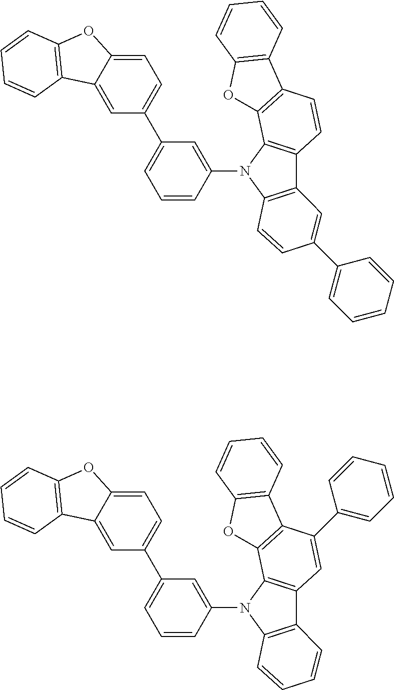 [SMILES: c1ccc(-c2ccc3c(c2)c2ccc4c5ccccc5oc4c2n3-c2cccc(-c3ccc4oc5ccccc5c4c3)c2)cc1 ; c1ccc(-c2cc3c4ccccc4n(-c4cccc(-c5ccc6oc7ccccc7c6c5)c4)c3c3oc4ccccc4c23)cc1]

### Exciton Barrier Layer

An exciton barrier layer inhibits excitons generated through recombination of holes and electrons in the light emitting layer from being diffused to the charge transporting layer. In some embodiments, the exciton barrier layer enables effective confinement of excitons in the light emitting layer. In some embodiments, the light emission efficiency of the device is enhanced. In some embodiments, the exciton barrier layer is adjacent to the light emitting layer on any of the side of the anode and the side of the cathode, and on both the sides. In some embodiments, where the exciton barrier layer is on the side of the anode, the layer can be between the hole transporting layer and the light emitting layer and adjacent to the light emitting layer. In some embodiments, where the exciton barrier layer is on the side of the cathode, the layer can be between the light emitting layer and the cathode and adjacent to the light emitting layer. In some embodiments, a hole injection layer, an electron barrier layer, or a similar layer is between the anode and the exciton barrier layer that is adjacent to the light emitting layer on the side of the anode. In some embodiments, a hole injection layer, an electron barrier layer, a hole barrier layer, or a similar layer is between the cathode and the exciton barrier layer that is adjacent to the light emitting layer on the side of the cathode. In some embodiments, the exciton barrier layer comprises excited singlet energy and excited triplet energy, at least one of which is higher than the excited singlet energy and the excited triplet energy of the light emitting material, respectively.

### Hole Transporting Layer

The hole transporting layer comprises a hole transporting material. In some embodiments, the hole transporting layer is a single layer. In some embodiments, the hole transporting layer comprises a plurality of layers.

In some embodiments, the hole transporting material has one of injection or transporting property of holes and barrier property of electrons. In some embodiments, the hole transporting material is an organic material. In some embodiments, the hole transporting material is an inorganic material. Examples of known hole transporting materials that may be used herein include but are not limited to a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an allylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer, or a combination thereof. In some embodiments, the hole transporting material is selected from a porphyrin compound, an aromatic tertiary amine compound, and a styrylamine compound. In some embodiments, the hole transporting material is an aromatic tertiary amine compound. Preferred compound examples for use as the hole transporting material are shown below.

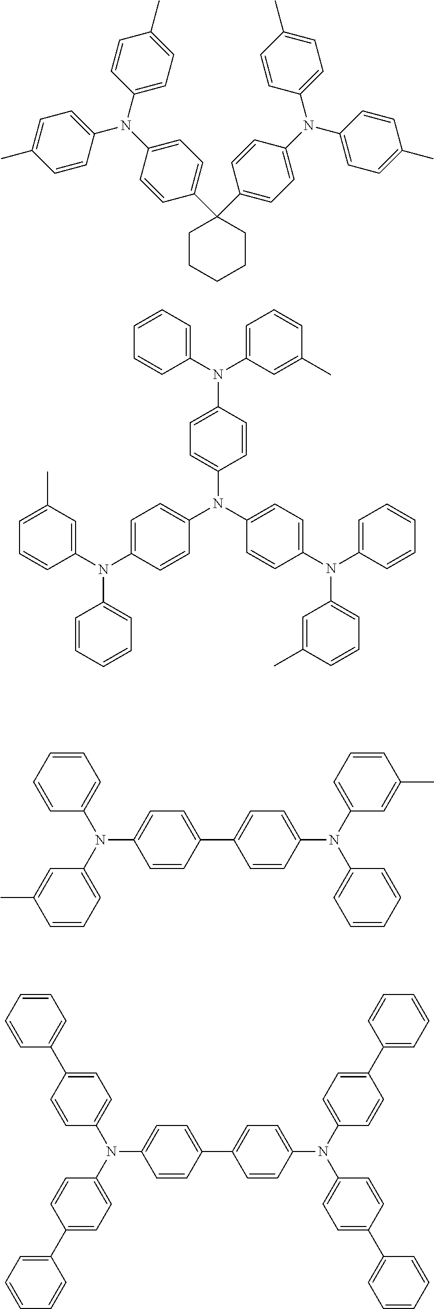

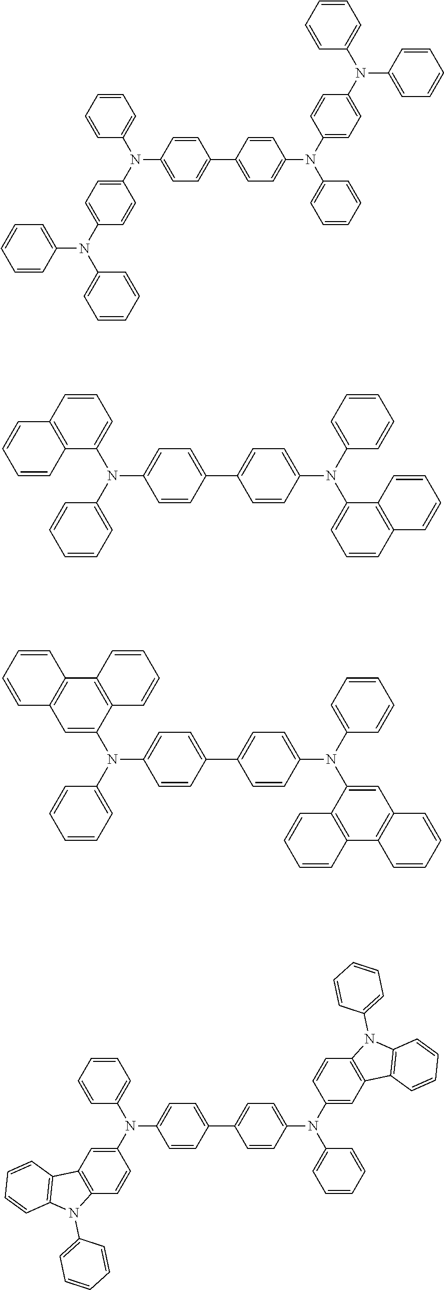 [SMILES: c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1 ; c1ccc(N(c2ccc(-c3ccc(N(c4ccccc4)c4cc5ccccc5c5ccccc45)cc3)cc2)c2cc3ccccc3c3ccccc23)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

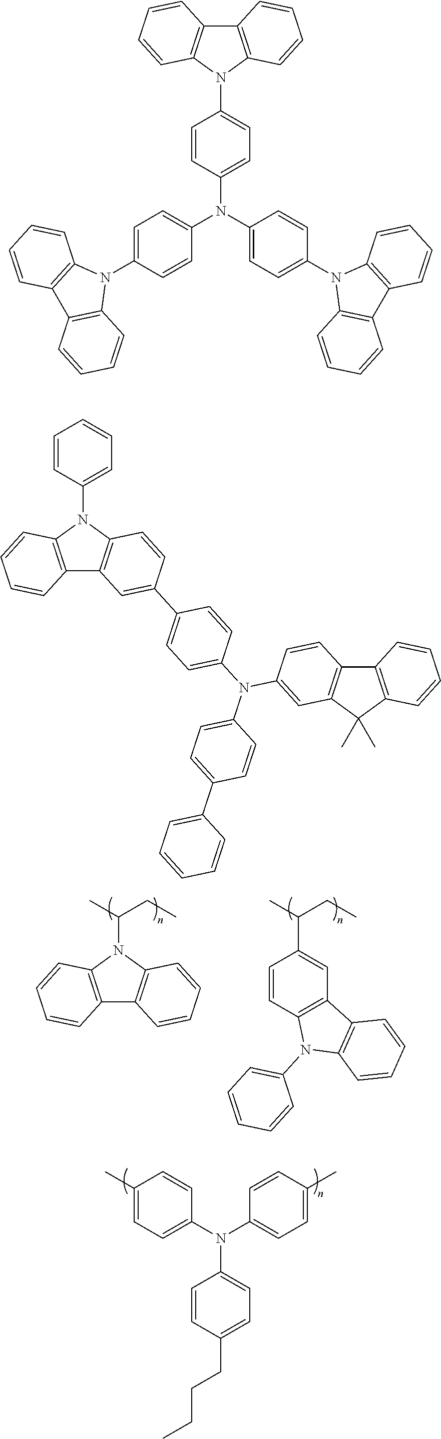

### Electron Transporting Layer

The electron transporting layer comprises an electron transporting material. In some embodiments, the electron transporting layer is a single layer. In some embodiments, the electron transporting layer comprises a plurality of layers.

In some embodiments, the electron transporting material needs only to have a function of transporting electrons, which are injected from the cathode, to the light emitting layer. In some embodiments, the electron transporting material also function as a hole barrier material. Examples of the electron transporting layer that may be used herein include but are not limited to a nitro-substituted fluorene derivative, a diphenylquinone derivative, a thiopyran dioxide derivative, carbodiimide, a fluorenylidene methane derivative, anthraquinodimethane, an anthrone derivatives, an oxadiazole derivative, an azole derivative, an azine derivative, or a combination thereof, or a polymer thereof. In some embodiments, the electron transporting material is a thiadiazole derivative, or a quinoxaline derivative. In some embodiments, the electron transporting material is a polymer material. Preferred compound examples for use as the electron transporting material are shown below.

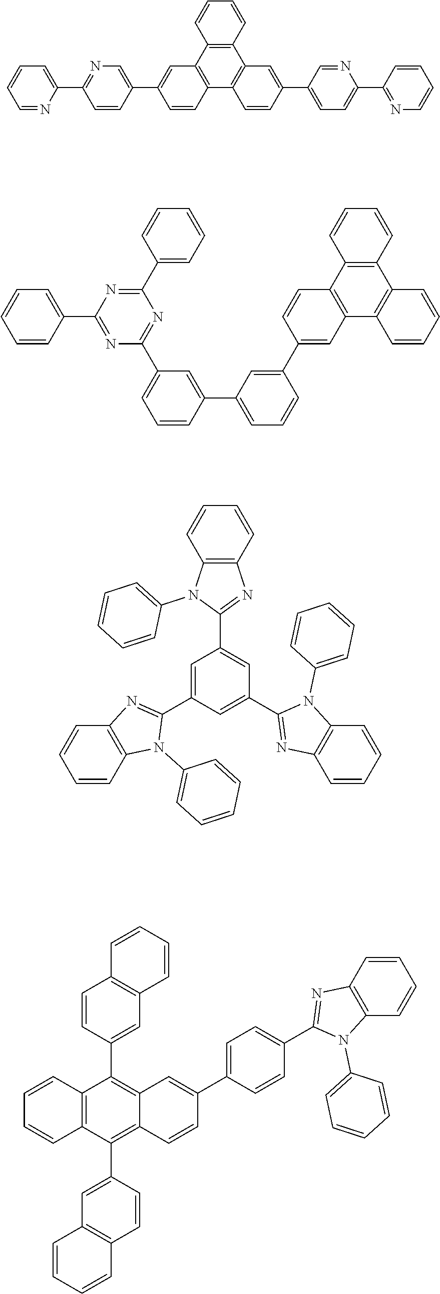 [SMILES: c1ccc(-c2ccc(-c3ccc4c5ccc(-c6ccc(-c7ccccn7)nc6)cc5c5ccccc5c4c3)cn2)nc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-n2c(-c3cc(-c4nc5ccccc5n4-c4ccccc4)cc(-c4nc5ccccc5n4-c4ccccc4)c3)nc3ccccc32)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

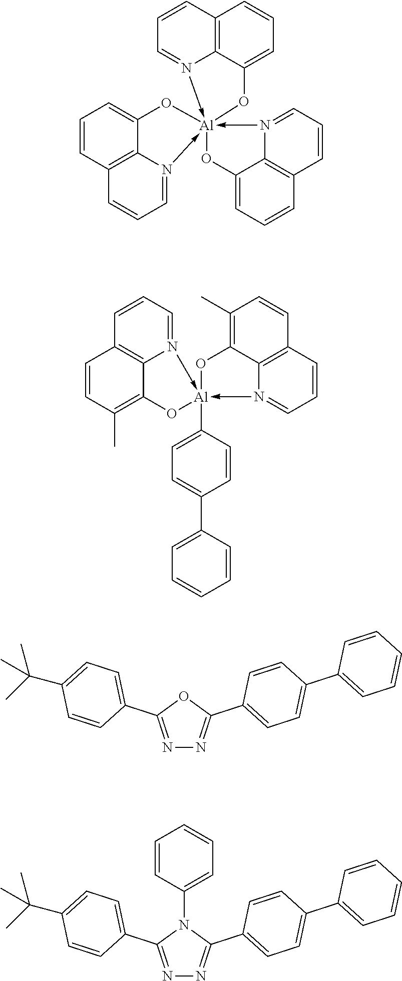

Hereinunder, compound examples preferred as a material that can be added to the organic layers are shown. For example, these can be added as a stabilization material.

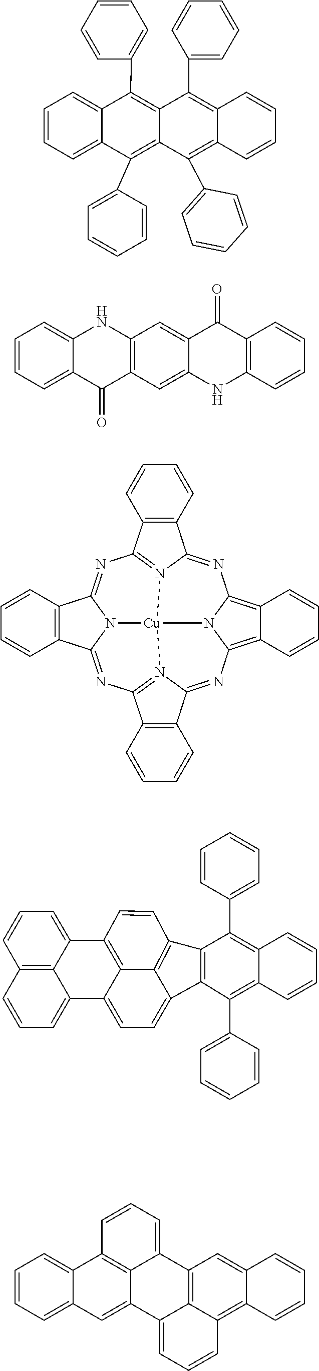

Preferred materials for use in the organic electroluminescent device are specifically shown. However, the materials usable in the invention should not be limitatively interpreted by the following exemplary compounds. Compounds that are exemplified as materials having a specific function can also be used as materials having any other function.

### Devices

In some embodiments, an light emitting layer is incorporated into a device. For example, the device includes, but is not limited to an OLED bulb, an OLED lamp, a television screen, a computer monitor, a mobile phone, and a tablet.

In some embodiments, an electronic device comprises an OLED comprising an anode, a cathode, and at least one organic layer comprising a light emitting layer between the anode and the cathode.

In some embodiments, compositions described herein may be incorporated into various light-sensitive or light-activated devices, such as a OLEDs or opto-electronic devices. In some embodiments, the composition may be useful in facilitating charge transfer or energy transfer within a device and/or as a hole transporting material. The device may be, for example, an organic light emitting diode (OLED), an organic integrated circuit (O—IC), an organic field-effect transistor (O-FET), an organic thin-film transistor (O-TFT), an organic light emitting transistor (O-LET), an organic solar cell (O—SC), an organic optical detector, an organic photoreceptor, an organic field-quench device (O-FQD), a light-emitting electrochemical cell (LEC) or an organic laser diode (O-laser).

### Bulbs or Lamps

In some embodiments, an electronic device comprises an OLED comprising an anode, a cathode, and at least one organic layer comprising a light emitting layer between the anode and the cathode.

In some embodiments, a device comprises OLEDs that differ in color. In some embodiments, a device comprises an array comprising a combination of OLEDs. In some embodiments, the combination of OLEDs is a combination of three colors (e.g., RGB). In some embodiments, the combination of OLEDs is a combination of colors that are not red, green, or blue (for example, orange and yellow green). In some embodiments, the combination of OLEDs is a combination of two, four, or more colors.

In some embodiments, a device is an OLED light comprising:
a circuit board having a first side with a mounting surface and an opposing second side, and defining at least one aperture;
at least one OLED on the mounting surface, the at least one OLED configured to emanate light, comprising:
an anode, a cathode, and at least one organic layer comprising a light emitting layer between the anode and the cathode;
a housing for the circuit board; and
at least one connector arranged at an end of the housing, the housing and the connector defining a package adapted for installation in a light fixture.

In some embodiments, the OLED light comprises a plurality of OLEDs mounted on a circuit board such that light emanates in a plurality of directions. In some embodiments, a portion of the light emanated in a first direction is deflected to emanate in a second direction. In some embodiments, a reflector is used to deflect the light emanated in a first direction.

### Displays or Screens

In some embodiments, the compounds of the invention can be used in a screen or a display. In some embodiments, the compounds of the invention are deposited onto a substrate using a process including, but not limited to, vacuum evaporation, deposition, vapor deposition, or chemical vapor deposition (CVD). In some embodiments, the substrate is a photoplate structure useful in a two-sided etching that provides a unique aspect ratio pixel. The screen (which may also be referred to as a mask) is used in a process in the manufacturing of OLED displays. The corresponding artwork pattern design facilitates an arrangement of a very steep and narrow tie-bar between the pixels in the vertical direction and a large, sweeping bevel opening in the horizontal direction. This allows the close patterning of pixels needed for high resolution displays while optimizing the chemical vapor deposition onto a TFT backplane.

The internal patterning of the pixel allows the construction of a three-dimensional pixel opening with varying aspect ratios in the horizontal and vertical directions. Additionally, the use of imaged “stripes” or halftone circles within the pixel area inhibits etching in specific areas until these specific patterns are undercut and fall off the substrate. At that point, the entire pixel area is processed at a similar etching rate but the depths are varying depending on the halftone pattern. Varying the size and spacing of the halftone pattern allows etching to be inhibited at different rates within the pixel, allowing for a localized deeper etch needed to create steep vertical bevels.

A preferred material for the deposition mask is invar. Invar is a metal alloy that is cold rolled into a long thin sheet in a steel mill. Invar cannot be electrodeposited onto a rotating mandrel as the nickel mask. A preferred and more cost feasible method for forming an open areas in the mask used for deposition is through a wet chemical etching.

In some embodiments, a screen or display pattern is a pixel matrix on a substrate. In some embodiments, a screen or display pattern is fabricated using lithography (e.g., photolithography and e-beam lithography). In some embodiments, a screen or display pattern is fabricated using a wet chemical etching. In further embodiments, a screen or display pattern is fabricated using plasma etching.

### Methods of Manufacturing Devices

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

In another aspect of the invention, provided herein is a method of manufacturing an organic light emitting diode (OLED) display, the method comprising:
forming a barrier layer on a base substrate of a mother panel;
forming a plurality of display units in units of cell panels on the barrier layer;
forming an encapsulation layer on each of the display units of the cell panels;
applying an organic film to an interface portion between the cell panels.

In some embodiments, the barrier layer is an inorganic film formed of, for example, SiNx, and an edge portion of the barrier layer is covered with an organic film formed of polyimide or acryl. In some embodiments, the organic film helps the mother panel to be softly cut in units of the cell panel.

In some embodiments, the thin film transistor (TFT) layer includes a light emitting layer, a gate electrode, and a source/drain electrode. Each of the plurality of display units may include a thin film transistor (TFT) layer, a planarization film formed on the TFT layer, and a light emitting unit formed on the planarization film, wherein the organic film applied to the interface portion is formed of a same material as a material of the planarization film and is formed at a same time as the planarization film is formed. In some embodiments, a light emitting unit is connected to the TFT layer with a passivation layer and a planarization film therebetween and an encapsulation layer that covers and protects the light emitting unit. In some embodiments of the method of manufacturing, the organic film contacts neither the display units nor the encapsulation layer.

Each of the organic film and the planarization film may include any one of polyimide and acryl. In some embodiments, the barrier layer may be an inorganic film. In some embodiments, the base substrate may be formed of polyimide. The method may further include, before the forming of the barrier layer on one surface of the base substrate formed of polyimide, attaching a carrier substrate formed of a glass material to another surface of the base substrate, and before the cutting along the interface portion, separating the carrier substrate from the base substrate. In some embodiments, the OLED display is a flexible display.

In some embodiments, the passivation layer is an organic film disposed on the TFT layer to cover the TFT layer. In some embodiments, the planarization film is an organic film formed on the passivation layer. In some embodiments, the planarization film is formed of polyimide or acryl, like the organic film formed on the edge portion of the barrier layer. In some embodiments, the planarization film and the organic film are simultaneously formed when the OLED display is manufactured. In some embodiments, the organic film may be formed on the edge portion of the barrier layer such that a portion of the organic film directly contacts the base substrate and a remaining portion of the organic film contacts the barrier layer while surrounding the edge portion of the barrier layer.

In some embodiments, the light emitting layer includes a pixel electrode, a counter electrode, and an organic light emitting layer disposed between the pixel electrode and the counter electrode. In some embodiments, the pixel electrode is connected to the source/drain electrode of the TFT layer.

In some embodiments, when a voltage is applied to the pixel electrode through the TFT layer, an appropriate voltage is formed between the pixel electrode and the counter electrode, and thus the organic light emitting layer emits light, thereby forming an image. Hereinafter, an image forming unit including the TFT layer and the light emitting unit is referred to as a display unit.

In some embodiments, the encapsulation layer that covers the display unit and prevents penetration of external moisture may be formed to have a thin film encapsulation structure in which an organic film and an inorganic film are alternately stacked. In some embodiments, the encapsulation layer has a thin film encapsulation structure in which a plurality of thin films are stacked. In some embodiments, the organic film applied to the interface portion is spaced apart from each of the plurality of display units. In some embodiments, the organic film is formed such that a portion of the organic film directly contacts the base substrate and a remaining portion of the organic film contacts the barrier layer while surrounding an edge portion of the barrier layer.

In one embodiment, the OLED display is flexible and uses the soft base substrate formed of polyimide. In some embodiments, the base substrate is formed on a carrier substrate formed of a glass material, and then the carrier substrate is separated.

In some embodiments, the barrier layer is formed on a surface of the base substrate opposite to the carrier substrate. In one embodiment, the barrier layer is patterned according to a size of each of the cell panels. For example, while the base substrate is formed over the entire surface of a mother panel, the barrier layer is formed according to a size of each of the cell panels, and thus a groove is formed at an interface portion between the barrier layers of the cell panels. Each of the cell panels can be cut along the groove.

In some embodiments, the method of manufacture further comprises cutting along the interface portion, wherein a groove is formed in the barrier layer, wherein at least a portion of the organic film is formed in the groove, and wherein the groove does not penetrate into the base substrate. In some embodiments, the TFT layer of each of the cell panels is formed, and the passivation layer which is an inorganic film and the planarization film which is an organic film are disposed on the TFT layer to cover the TFT layer. At the same time as the planarization film formed of, for example, polyimide or acryl is formed, the groove at the interface portion is covered with the organic film formed of, for example, polyimide or acryl. This is to prevent cracks from occurring by allowing the organic film to absorb an impact generated when each of the cell panels is cut along the groove at the interface portion. That is, if the entire barrier layer is entirely exposed without the organic film, an impact generated when each of the cell panels is cut along the groove at the interface portion is transferred to the barrier layer, thereby increasing the risk of cracks. However, in one embodiment, since the groove at the interface portion between the barrier layers is covered with the organic film and the organic film absorbs an impact that would otherwise be transferred to the barrier layer, each of the cell panels may be softly cut and cracks may be prevented from occurring in the barrier layer. In one embodiment, the organic film covering the groove at the interface portion and the planarization film are spaced apart from each other. For example, if the organic film and the planarization film are connected to each other as one layer, since external moisture may penetrate into the display unit through the planarization film and a portion where the organic film remains, the organic film and the planarization film are spaced apart from each other such that the organic film is spaced apart from the display unit.

In some embodiments, the display unit is formed by forming the light emitting unit, and the encapsulation layer is disposed on the display unit to cover the display unit. As such, once the mother panel is completely manufactured, the carrier substrate that supports the base substrate is separated from the base substrate. In some embodiments, when a laser beam is emitted toward the carrier substrate, the carrier substrate is separated from the base substrate due to a difference in a thermal expansion coefficient between the carrier substrate and the base substrate.

In some embodiments, the mother panel is cut in units of the cell panels. In some embodiments, the mother panel is cut along an interface portion between the cell panels by using a cutter. In some embodiments, since the groove at the interface portion along which the mother panel is cut is covered with the organic film, the organic film absorbs an impact during the cutting. In some embodiments, cracks may be prevented from occurring in the barrier layer during the cutting.

In some embodiments, the methods reduce a defect rate of a product and stabilize its quality.

Another aspect is an OLED display including: a barrier layer that is formed on a base substrate; a display unit that is formed on the barrier layer; an encapsulation layer that is formed on the display unit; and an organic film that is applied to an edge portion of the barrier layer.

### [Disclosure of Useful Material]

A compound represented by the following general formula (5) is disclosed hereinafter.

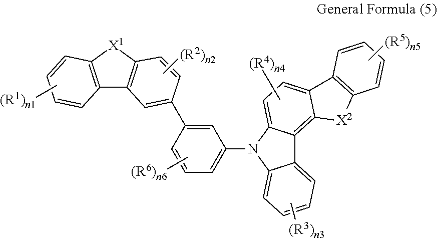

In the general formula (5), X¹ and X² each independently represent an oxygen atom or a sulfur atom. R¹ to R⁶ each independently represent an atom or a group selected from the group consisting of a deuterium atom, an alkyl group and an aryl group, or a group formed by combining at least two thereof. At least one R¹ is an aryl group optionally substituted with an atom or a group selected from the group consisting of a deuterium atom, an alkyl group and an aryl group or with a group formed by combining at least two thereof. R¹ to R⁶ do not bond to the other R¹ to R⁶ to form a cyclic structure, but the neighboring R³'s can bond to each other to form a benzofuro skeleton or a benzothieno skeleton. n1 represents an integer of 1 to 4, n3, n5 and n6 each independently represent an integer of 0 to 4, n2 represents an integer of 0 to 3, n4 represents an integer of 0 to 2.

In one preferred aspect of the present invention, X¹ and X² are the same. In one aspect of the present invention, X¹ and X² are different. In one preferred aspect of the present invention, X¹ and X² are both oxygen atoms. In one aspect of the present invention, X¹ and X² are both sulfur atoms. In one aspect of the present invention, X¹ is an oxygen atom, and X² is a sulfur atom. In one aspect of the present invention, X¹ is a sulfur atom, and X² is an oxygen atom.

R¹ to R⁶ are each independently an atom or a group selected from the group consisting of a deuterium atom, an alkyl group and an aryl group, or a group formed by combining at least two thereof, preferably, a deuterium atom, or an alkyl group optionally substituted with a deuterium atom, or an aryl group optionally substituted with an atom or a group selected from the group consisting of a deuterium atom, an alkyl group and an aryl group, or with a group formed by combining at least two thereof. In one preferred aspect of the present invention, R¹ to R⁶ are each independently a deuterium atom, or an aryl group optionally substituted with an atom or a group selected from the group consisting of a deuterium atom and an aryl group, or with a group formed by combining at least two thereof. The carbon number of the alkyl group in the description of the general formula (5) can be selected within a range of generally 1 to 40, preferably 1 to 15, more preferably 1 to 6, for example, 1 to 3. The carbon number of the aryl group can be selected within a range of generally 6 to 30, preferably 6 to 18, more preferably 6 to 14, for example, 6 to 10. In one aspect of the present invention, R³'s can bond to each other to form a benzofuro skeleton. In one aspect of the present invention, R³'s can bond to each other to form a benzothieno skeleton. The benzofuro skeleton and the benzothieno skeleton can be substituted with an atom or a group selected from the group consisting of a deuterium atom, an alkyl group and an aryl group or with a group formed by combining at least two thereof. In one aspect of the present invention, R³'s do not bond to each other to form a cyclic structure.

At least one R¹ is an aryl group optionally substituted with an atom or a group selected from the group consisting of a deuterium atom, an alkyl group and an aryl group, or with a group formed by combining at least two thereof, more preferably a phenyl group optionally substituted with an atom or a group selected from the group consisting of a deuterium atom, an alkyl group and an aryl group, or with a group formed by combining at least two thereof, further preferably a phenyl group optionally substituted with an atom or a group selected from the group consisting of a deuterium atom and a phenyl group, or with a group formed by combining at least two thereof. In one aspect of the present invention, R¹ of the kind is an unsubstituted aryl group, or an aryl group in which all the hydrogen atoms are substituted with deuterium atoms, preferably an unsubstituted phenyl group, or a phenyl group in which all the hydrogen atoms are substituted with deuterium atoms. In one preferred aspect of the present invention, only one R¹ is an aryl group optionally substituted with an atom or a group selected from the group consisting of a deuterium atom, an alkyl group and an aryl group or with a group formed by combining at least two thereof. In one aspect of the present invention, two R¹'s can be an aryl group optionally substituted with an atom or a group selected from the group consisting of a deuterium atom, an alkyl group and an aryl group or with a group formed by combining at least two thereof. Specific examples of the group that at least one R¹ can represent are shown below, but R¹ which the present invention can employ should not be limitatively interpreted by the following specific example. D in the following specific examples represents a deuterium atom.

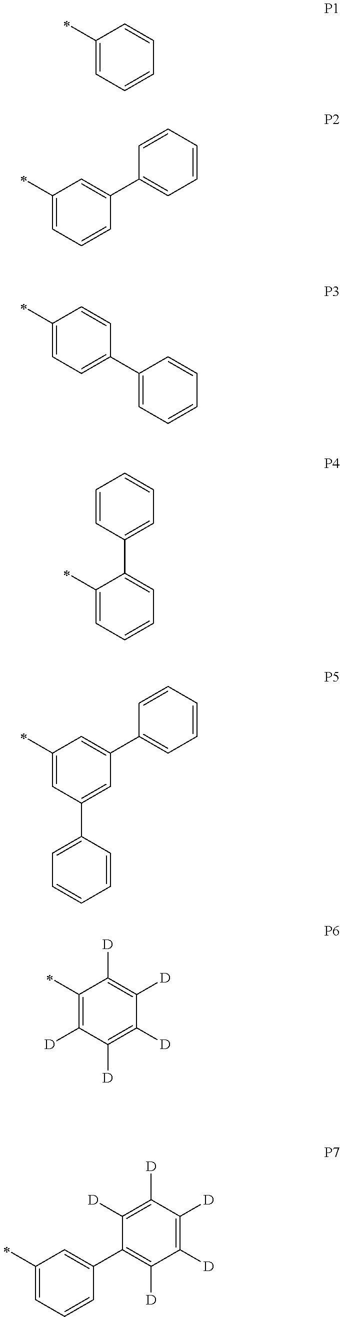

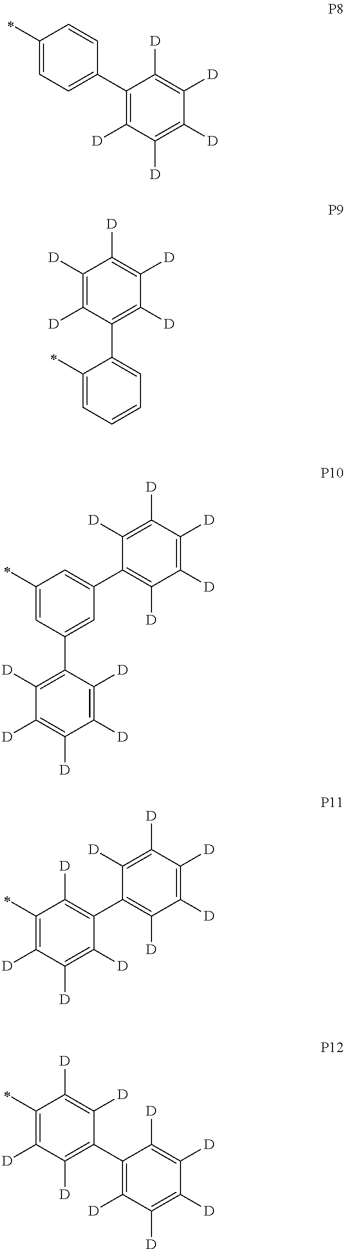

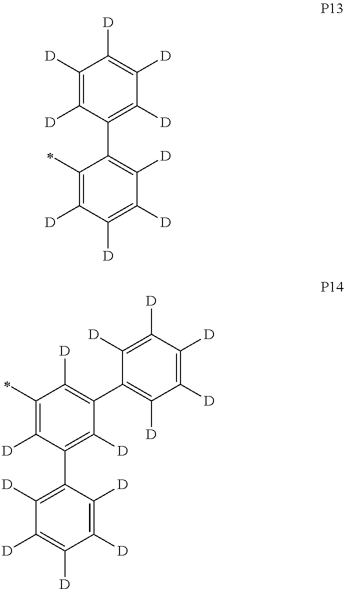

In one aspect of the present invention, n1 is 1 or 2, n2 to no are each independently an integer of 0 to 2, preferably n1 is 1, n2 to n6 are each independently 0 or 1, and further preferably n1 is 1, n2 and n3 are 0 and n4 to n6 are each independently 0 or 1. In one aspect of the present invention, n4+n5+n6 is 0 to 2, preferably 0 or 1, for example, 1, for example 0. In one aspect of the present invention, n1 is 1, n2 to n6 are 0. In one aspect of the present invention, n1 and n3 are 1, n2, n4, n5 and n6 are 0. In one aspect of the present invention, n1 and n5 are 1, n2, n3, n4 and n6 are 0.

Specific examples of the compound represented by the general formula (5) are shown below, but the compound that can be used in the present invention should not be construed as being limited by these specific examples.

TABLE 43

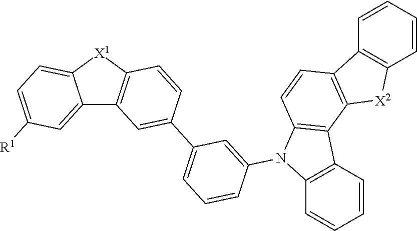

| No. | R¹ | X¹ | X² |
|---|---|---|---|
| M1 | P1 | O | O |
| M2 | P2 | O | O |
| M3 | P3 | O | O |
| M4 | P4 | O | O |
| M5 | P5 | O | O |
| M6 | P6 | O | O |
| M7 | P7 | O | O |
| M8 | P8 | O | O |
| M9 | P9 | O | O |
| M10 | P10 | O | O |
| M11 | P11 | O | O |
| M12 | P12 | O | O |
| M13 | P13 | O | O |
| M14 | P14 | O | O |
| M15 | P1 | O | S |
| M16 | P2 | O | S |
| M17 | P3 | O | S |
| M18 | P4 | O | S |
| M19 | P5 | O | S |
| M20 | P6 | O | S |
| M21 | P7 | O | S |
| M22 | P8 | O | S |
| M23 | P9 | O | S |
| M24 | P10 | O | S |
| M25 | P11 | O | S |
| M26 | P12 | O | S |
| M27 | P13 | O | S |
| M28 | P14 | O | S |
| M29 | P1 | S | S |
| M30 | P2 | S | S |
| M31 | P3 | S | S |
| M32 | P4 | S | S |
| M33 | P5 | S | S |
| M34 | P6 | S | S |
| M35 | P7 | S | S |
| M36 | P8 | S | S |
| M37 | P9 | S | S |
| M38 | P10 | S | S |
| M39 | P11 | S | S |
| M40 | P12 | S | S |
| M41 | P13 | S | S |
| M42 | P14 | S | S |
| M43 | P1 | S | O |
| M44 | P2 | S | O |
| M45 | P3 | S | O |
| M46 | P4 | S | O |
| M47 | P5 | S | O |
| M48 | P6 | S | O |
| M49 | P7 | S | O |
| M50 | P8 | S | O |
| M51 | P9 | S | O |
| M52 | P10 | S | O |

The compound represented by the general formula (5) is useful as a host material. Consequently, for example, as combined with a light emitting material, the compound can form a light emitting layer. The content of the compound represented by the general formula (5) in a light emitting layer can be more than 50% by weight, preferably more than 80% by weight, and can be, for example, more than 90% by weight. The light emitting material is selected from compounds whose lowest excited singlet energy is smaller than that of the compound represented by the general formula (5). The light emitting material can be a fluorescent material or a phosphorescent material, but is preferably a fluorescent material, and for example, a delayed fluorescent material can be used. In one aspect of the present invention, the light emitting layer is formed of two components, a compound represented by the general formula (5) as a host material, and a light emitting material. The light emitting layer can contain, in addition to the compound represented by the general formula (5) as a host material, and the light emitting material, an assist dopant whose lowest excited singlet energy is smaller than that of the host material and higher than that of the light emitting material. It is preferable to use a delayed fluorescent material as the assist dopant. A delayed fluorescent material can also be used as the light emitting material. In one aspect of the present invention, the light emitting layer is formed of three components, a compound represented by the general formula (5) as a host material, an assist dopant and a light emitting material.

As the light emitting material that can be combined with the compound represented by the general formula (5), for example, a compound represented by the following general formula (6) can be preferably used.

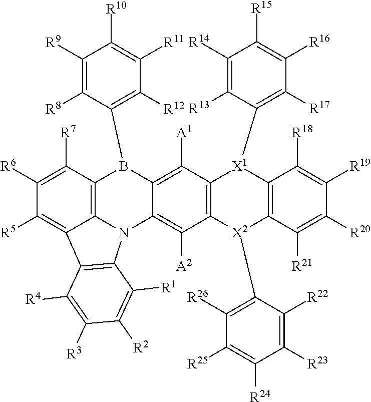

In the general formula (6), one of X¹ and X² is a nitrogen atom, and the other is a boron atom. R¹ to R²⁶, A¹ and A² each independently represent a hydrogen atom, a deuterium atom, or a substituent. R¹ and R², R² and R³, R³ and R⁴, R⁴ and R⁵, R⁵ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ each can bond to each other to form a cyclic structure. However, when X¹ is a nitrogen atom, R¹⁷ and R¹⁸ bond to each other to be a single bond to form a pyrrole ring, and when X² is a nitrogen atom, R²¹ and R²² bond to each other to be a single bond to form a pyrrole ring. However, in the case where X¹ is a nitrogen atom, and where R⁷ and R⁸ and R²¹ and R²² each bond to each other via a nitrogen atom to form a 6-membered ring, and R¹⁷ and R¹⁸ bond to each other to form a single bond, at least one of R¹ to R⁶ is a substituted or unsubstituted aryl group, or any of R¹ and R², R² and R³, R³ and R⁴, R⁴ and R⁵, and R⁵ and R⁶ bond to each other to form an aromatic ring or a heteroaromatic ring.

As the light emitting material, any other multiple resonance delayed fluorescent material than the compound represented by the general formula (6) or a non-multiple resonance delayed fluorescent material can also be used in the light emitting layer. Also as the assist dopant, a delayed fluorescent material can be favorably used. Regarding general formulae and specific examples of the delayed fluorescent material that can be employed as a light emitting material and an assist dopant, for example, reference can be made to WO2020/111205, [0028] to [0056], and WO2019/191665, pp. 62-159, which are incorporated herein by reference as a part of the present description. In one aspect of the present invention, a compound with a benzene ring having a donor group and an acceptor group bonding thereto can be favorably used as an assist dopant. Examples of the donor group include a substituted or unsubstituted carbazol-9-yl group, and examples of the acceptor group include a cyano group-having group and a triazine ring-having group, which, however, are not limitative.

Examples of preferred combinations of a host material, an assist dopant and a light emitting material usable in the light emitting layer are shown below, but the combinations employable in the present invention should not be limitatively interpreted by the following examples.

TABLE 44

| | Host Material | Assist Dopant | Light Emitting Material |
|---|---|---|---|
| 1 | 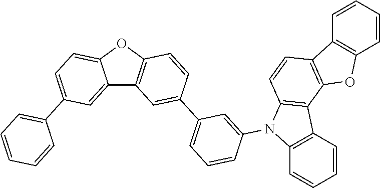 [SMILES: c1ccc(-c2ccc3oc4ccc(-c5cccc(-n6c7ccccc7c7c8oc9ccccc9c8ccc76)c5)cc4c3c2)cc1] | 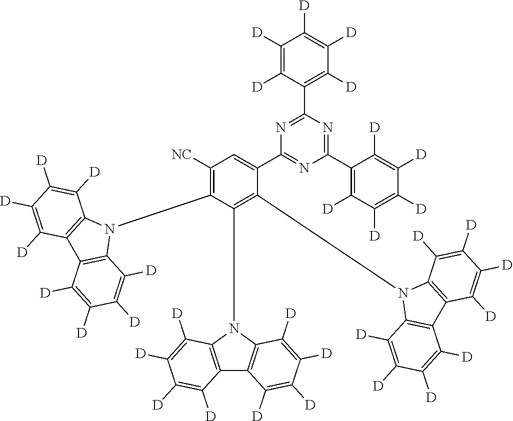 [SMILES: [2H]c1c([2H])c([2H])c(-c2nc(-c3cc(C#N)c(-n4c5c([2H])c([2H])c([2H])c([2H])c5c5c([2H])c([2H])c([2H])c([2H])c54)c(-n4c5c([2H])c([2H])c([2H])c([2H])c5c5c([2H])c([2H])c([2H])c([2H])c54)c3-n3c4c([2H])c([2H])c([2H])c([2H])c4c4c([2H])c([2H])c([2H])c([2H])c43)nc(-c3c([2H])c([2H])c([2H])c([2H])c3[2H])n2)c([2H])c1[2H]] | 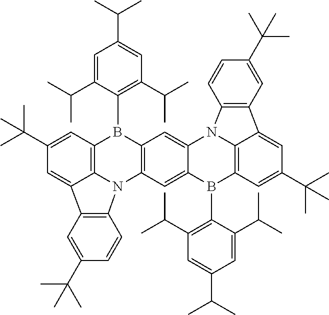 [SMILES: CC(C)c1cc(C(C)C)c(B2c3cc4c(cc3-n3c5ccc(C(C)(C)C)cc5c5cc(C(C)(C)C)cc2c53)B(c2c(C(C)C)cc(C(C)C)cc2C(C)C)c2cc(C(C)(C)C)cc3c5cc(C(C)(C)C)ccc5n-4c23)c(C(C)C)c1] |
| 2 | 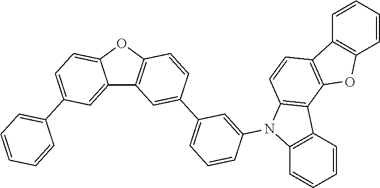 [SMILES: c1ccc(-c2ccc3oc4ccc(-c5cccc(-n6c7ccccc7c7c8oc9ccccc9c8ccc76)c5)cc4c3c2)cc1] | 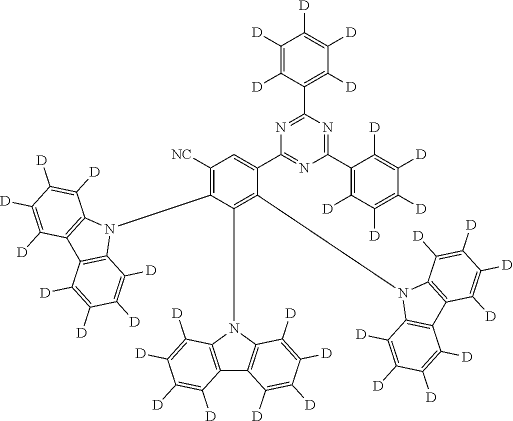 [SMILES: [2H]c1c([2H])c([2H])c(-c2nc(-c3cc(C#N)c(-n4c5c([2H])c([2H])c([2H])c([2H])c5c5c([2H])c([2H])c([2H])c([2H])c54)c(-n4c5c([2H])c([2H])c([2H])c([2H])c5c5c([2H])c([2H])c([2H])c([2H])c54)c3-n3c4c([2H])c([2H])c([2H])c([2H])c4c4c([2H])c([2H])c([2H])c([2H])c43)nc(-c3c([2H])c([2H])c([2H])c([2H])c3[2H])n2)c([2H])c1[2H]] | 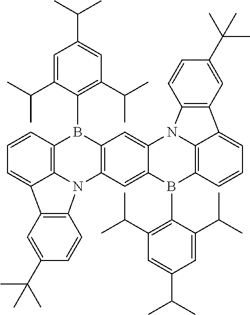 [SMILES: CC(C)c1cc(C(C)C)c(B2c3cc4c(cc3-n3c5ccc(C(C)(C)C)cc5c5cccc2c53)B(c2c(C(C)C)cc(C(C)C)cc2C(C)C)c2cccc3c5cc(C(C)(C)C)ccc5n-4c23)c(C(C)C)c1] |

Other examples of the light emitting layer include a combination in which the assist dopant in the above Table is replaced with any of the following assist dopants, a combination in which the light emitting material in the above Table is replaced with any of the following light emitting materials, and a combination in which the assist dopant and the light emitting material in the above Table are replaced with any of the following assist dopants and any of the following light emitting materials, respectively.

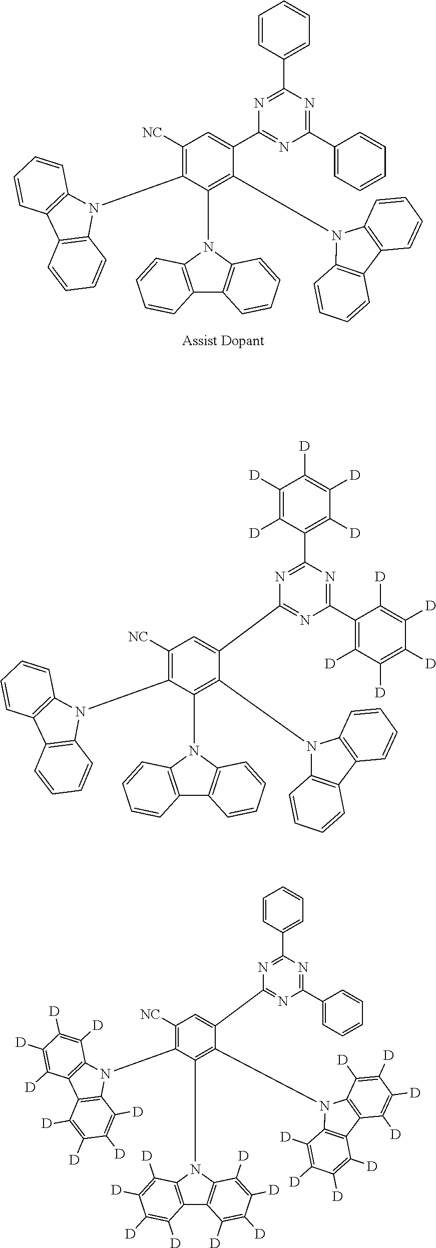

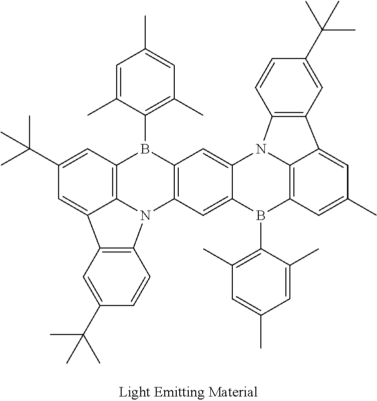 [SMILES: Cc1cc(C)c(B2c3cc4c(cc3-n3c5ccc(C(C)(C)C)cc5c5cc(C)cc2c53)B(c2c(C)cc(C)cc2C)c2cc(C(C)(C)C)cc3c5cc(C(C)(C)C)ccc5n-4c23)c(C)c1]

Also the compound represented by the general formula (5) is useful as an electron barrier material. Consequently, for example, a compound represented by the general formula (5) can be contained in a layer formed on the anode side of the light emitting layer of an organic electroluminescent device, preferably in a layer adjacent to the anode side of the light emitting layer. For example, the content of the compound represented by the general formula (5) in the layer can be more than 50% by weight, preferably more than 80% by weight, for example, 100% by weight.

By using the compound represented by the general formula (5), the light emission characteristics can be stabilized. Also, the light emitting device using the compound represented by the general formula (5) can exhibit better characteristics than other cases using similar compounds.

### EXAMPLES

The features of the present invention will be described more specifically with reference to Synthesis Examples and Examples given below. The materials, processes, procedures and the like shown below may be appropriately modified unless they deviate from the substance of the invention. Accordingly, the scope of the invention is not construed as being limited to the specific examples shown below. Hereinunder, the light emission characteristics were evaluated using a source meter (available from Keithley Instruments, Inc., 2400 series), a semiconductor parameter analyzer (available from Agilent Technologies, Inc., E5273A), a light power meter apparatus (available from Newport Corporation, 1930C), an optical spectroscope (available from Ocean Optics Corporation, USB 2000), a spectroradiometer (available from Topcon Corporation, SR-3) and a streak camera (available from Hamamatsu Photonics K.K., C4334).

### (Synthesis Example 1) Synthesis of Compound C1

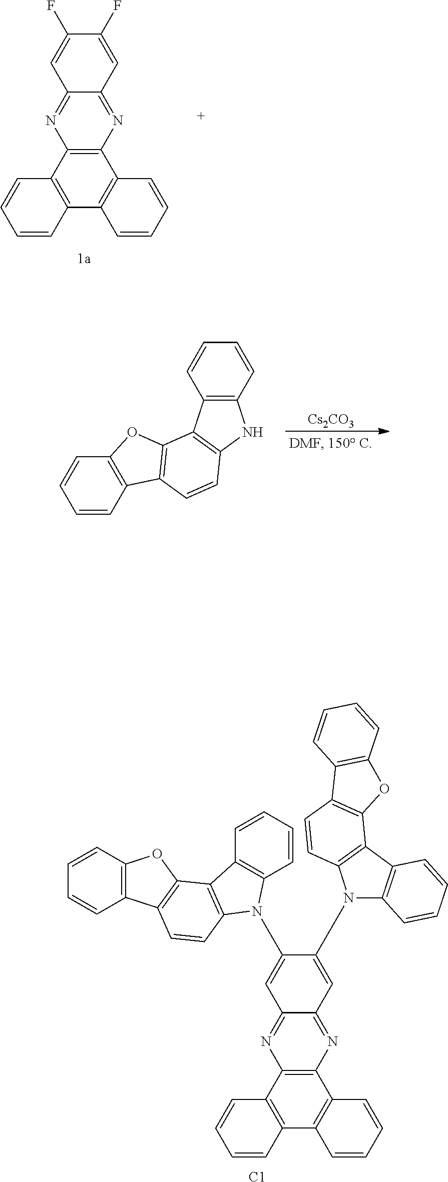

Under a nitrogen stream, a solution of Compound 1a (0.5 g, 1.58 mmol), 5H-benzofuro[3,2-c]carbazole (0.85 g, 3.3 mmol) and cesium carbonate (1.54 g, 4.8 mmol) dissolved in N,N-dimethylformamide (40 mL) was reacted by stirring under heat at 150° C. for 2 hours. The reaction mixture was restored to room temperature, the reaction was stopped with water, and then the precipitated sediment was filtered out. The residue was washed with methanol, and purified by silica gel column chromatography to give the intended Compound C1 (1.20 g, 1.51 mmol, yield 95%).

1H NMR (400 MHz, CDCl₃) δ 9.36 (dd, J=8.0 Hz, J=1.6 Hz, 2H), 8.89 (s, 2H), 8.54 (d, J=8.4 Hz, 2H), 8.15 (d, J=7.2 Hz, 2H), 7.85-7.78 (m, 4H), 7.74-7.70 (m, 2H), 7.58-7.50 (m, 4H), 7.38-7.25 (m, 8H), 7.19-7.05 (m, 4H). MS: m/z: calculated for C₅₆H₃₀N₄O₂[M+H]+: 790.24, found: 791.41.

### (Synthesis Example 2) Synthesis of Compound C2

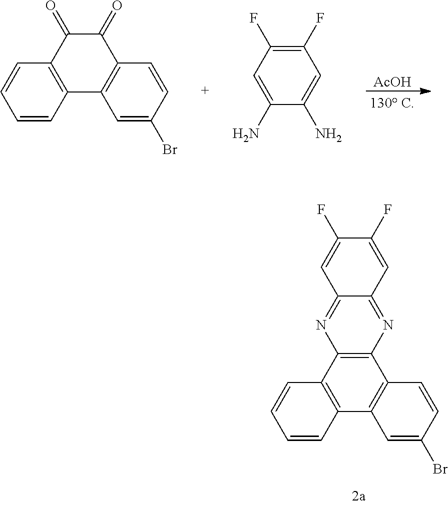 [SMILES: "AcOH" ; O=C1C(=O)c2ccc(Br)cc2-c2ccccc21 ; Nc1cc(F)c(F)cc1N ; Fc1cc2nc3c4ccccc4c4cc(Br)ccc4c3nc2cc1F]

Under a nitrogen stream, a solution of 3-bromophenanthrene-9,10-dione (1.0 g, 3.48 mmol) and 4,5-difluoro-1,2-phenylenediamine (0.6 g, 4.18 mmol) dissolved in acetic acid (100 mL) was reacted by stirring under heat at 130° C. for 24 hours. The reaction mixture was restored to room temperature, the reaction was stopped with water, and then the precipitated sediment was filtered out. The residue was washed with methanol, and purified by recrystallization with a mixed solvent of chloroform/methanol to give the Compound 2a (1.30 g, 3.30 mmol, yield 95%).

MS: m/z: calculated for C₂₀H₉BrF₂N₂[M+H]⁺: 393.99, found: 395.73.

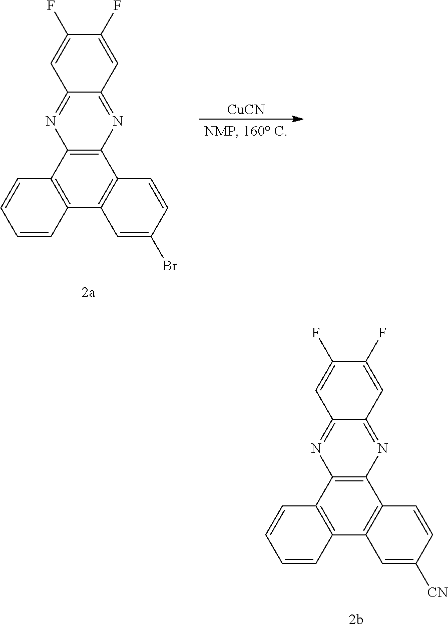 [SMILES: Fc1cc2nc3c4ccccc4c4cc(Br)ccc4c3nc2cc1F ; "CuCN" ; N#Cc1ccc2c(c1)c1ccccc1c1nc3cc(F)c(F)cc3nc21]

Under a nitrogen stream, a solution of Compound 2a (1.0 g, 3.48 mmol) and copper(I) cyanide (0.89 g, 9.8 mmol) dissolved in N-methyl-2-pyrrolidone (130 mL) was reacted by stirring under heat at 160° C. for 48 hours. The reaction mixture was restored to room temperature, processed with aqueous ammonia to stop the reaction, and extracted with chloroform. The solvent was evaporated away from the resultant organic layer with an evaporator, and the remaining crude product was purified by silica gel column chromatography to give Compound 2b (0.58 g, 1.70 mmol, yield 48%).

MS: m/z: calculated for C₂₁H₉F₂N₃[M+H]+: 341.08, found: 342.63.

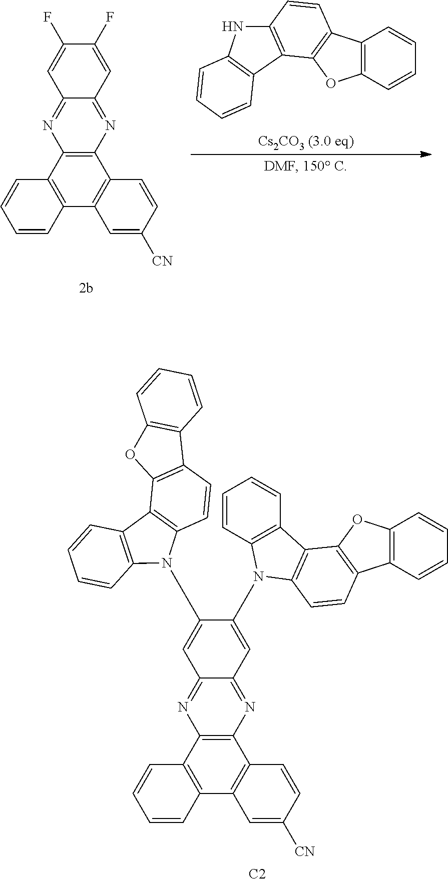 [SMILES: N#Cc1ccc2c(c1)c1ccccc1c1nc3cc(F)c(F)cc3nc21 ; c1ccc2c(c1)[nH]c1ccc3c4ccccc4oc3c12 ; N#Cc1ccc2c(c1)c1ccccc1c1nc3cc(-n4c5ccccc5c5c6oc7ccccc7c6ccc54)c(-n4c5ccccc5c5c6oc7ccccc7c6ccc54)cc3nc21 ; "C2"]

Under a nitrogen stream, a solution of Compound 2b (0.43 g, 1.25 mmol), 5H-benzofuro[3,2-c]carbazole (0.68 g, 2.6 mmol) and cesium carbonate (1.22 g, 3.75 mmol) dissolved in N,N-dimethylformamide (40 mL) was reacted by stirring under heat at 150° C. for 2 hours. The reaction mixture was restored to room temperature, the reaction was stopped with water, and then the precipitated sediment was filtered out. The residue was washed with methanol, and purified by silica gel column chromatography to give the intended Compound C2 (0.37 g, 0.45 mmol, yield 36%).

1H NMR (400 MHz, CDCl₃) δ 9.25-9.17 (m, 2H), 8.92 (s, 2H), 8.64 (s, 1H), 8.33 (d, J=8.0 Hz, 1H), 8.01-7.97 (m, 2H), 7.85 (d, J=7.6 Hz, 1H), 7.78-7.71 (m, 3H), 7.64 (t, J=7.6 Hz, 1H), 7.57-7.51 (m, 3H), 7.43-7.07 (m, 12H), 7.01-6.98 (m, 1H). MS: m/z: calculated for C₅₇H₂₉N₅O₂[M+H]⁺: 815.23, found: 816.42

### (Synthesis Example 3) Synthesis of Compound C3

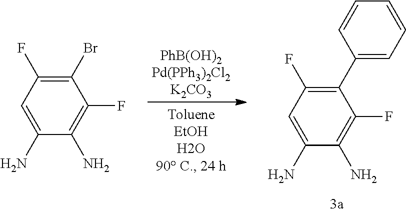 [SMILES: Nc1cc(F)c(Br)c(F)c1N ; Nc1cc(F)c(-c2ccccc2)c(F)c1N]

Under a nitrogen stream, a solution of 4-bromo-3,5-difluoro-1,2-benzenediamine (3.0 g, 13.5 mmol), phenylboronic acid (2.05 g, 17.0 mmol), bis(triphenylphosphine)palladium(II) dichloride (0.236 g, 0.3 mmol) and potassium carbonate (3.72 g, 27.0 mmol) dissolved in a mixed solvent of toluene/ethanol/water=60 mL/5 mL/10 mL was reacted by stirring at 90° C. for 24 hours. The reaction solution was restored to room temperature, given chloroform added thereto, and filtered through Celite. The resultant filtrate was washed with water, the organic layer was dewatered with anhydrous magnesium sulfate, filtered, and the solvent was evaporated away from the filtrate. The remaining crude product was purified by silica gel column chromatography to give Compound 3a (2.50 g, 11.3 mmol, yield 83%).

1H NMR (400 MHz, DMSO-d6) δ 7.38-7.26 (m, 5H), 6.24 (s, 1H), 5.20 (s, 2H), 4.24 (s, 2H).

MS: m/z: calculated for C₅₆H₃₀N₄O₂[M+H]+: 220.08, found: 220.22.

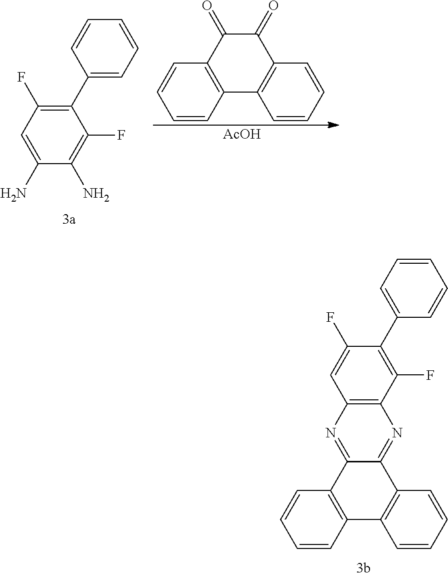

Under a nitrogen stream, a solution of Compound 3a (2.65 g, 12.0 mmol) and phenanthroline-9.10-dione (2.53 g, 12.2 mmol) dissolved in acetic acid (200 L) was reacted by stirring at 130° C. for 24 hours. The reaction mixture was restored to room temperature, given methanol added thereto, and the precipitated sediment was filtered out. The resultant solid was dried under reduced pressure, again washed with methanol, and then purified by silica gel column chromatography and recrystallization to give Compound 3b (4.5 g, 11.4 mmol, yield 95%).

1H NMR (400 MHz, CDCl₃) δ 9.36 (q, J=8.0 Hz, 2H), 8.55 (t, J=6.0 Hz, 2H), 7.84-7.74 (m, 5H), 7.66 (d, J=8.0 Hz, 2H), 7.58-7.50 (m, 3H).

MS: m/z: calculated for C₂₀H₉BrF₂N₂[M+H]+: 392.41, found: 393.67.

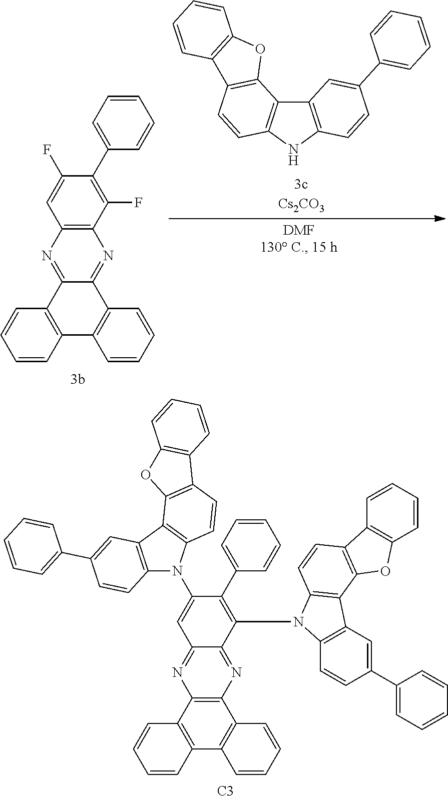

Under a nitrogen stream, a solution of Compound 3b (0.39 g, 1.0 mmol), Compound 3c (0.732 g, 2.2 mmol) and cesium carbonate (0.975 g, 3.0 mmol) dissolved in N,N-dimethylformamide (35 mL) was reacted by stirring at 130° C. for 15 hours. The reaction mixture was restored to room temperature, given methanol added thereto, and the precipitated sediment was filtered out. The resultant solid was purified by silica gel column chromatography, and then reprecipitated in a mixed solvent of toluene and methanol to give an orange solid of the intended Compound C3 (0.61 g, 0.60 mmol, yield 60%).

1H NMR (400 MHz, CDCl₃): δ 9.30 (s, 1H), 9.26 (d, J=7.6 Hz, 1H), 8.84 (s, 1H), 8.45 (s, 1H), 8.42-8.36 (m, 2H), 7.94-7.80 (m, 6H), 7.74-7.31 (m, 21H), 7.24-7.21 (m, 2H), 7.14 (t, J=8.0 Hz, 1H), 7.08 (t, J=8.0 Hz, 1H), 6.98 (d, J=7.2 Hz, 2H), 6.60-6.52 (m, 3H)

MS: m/z: calculated for C₇₄H₄₂N₄O₂[M+H]⁺: 1018.33, found: 1019.35.

### (Synthesis Example 4) Synthesis of Compound C4

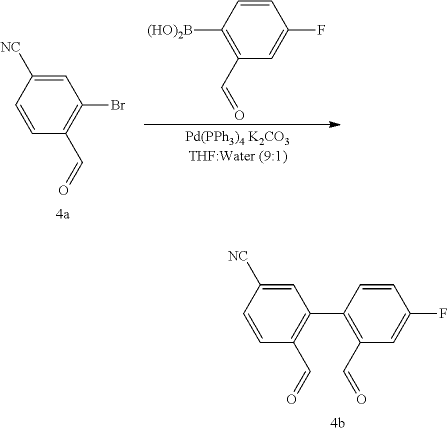 [SMILES: N#Cc1ccc(C=O)c(Br)c1 ; O=Cc1cc(F)ccc1B(O)O ; N#Cc1ccc(C=O)c(-c2ccc(F)cc2C=O)c1]

Under a nitrogen stream, a solution of Compound 4a (4.2 g, 20.0 mmol), 4-fluoro-2-formylphenylboronic acid (4.03 g. 24.0 mmol), tetrakis (triphenylphosphine)palladium (0) (1.15 g, 1.0 mmol) and sodium carbonate (5.52 g, 40.0 mmol) dissolved in a mixed solvent of tetrahydrofuran/water=75 mL/25 mL was reacted by stirring at 90° C. for 24 hours. The reaction solution was restored to room temperature, given chloroform added thereto, and filtered through Celite. The resultant filtrate was washed with water, the organic layer was dewatered with anhydrous magnesium sulfate, filtered, and the solvent was evaporated away from the filtrate. The remaining crude product was purified by silica gel column chromatography to give Compound 4b (4.30 g, 17.0 mmol, yield 85%).

1H NMR (400 MHz, CDCl3) δ 9.82 (s, 1H), 9.77 (s, 1H), 8.12 (d, J=8.0 Hz, 1H), 7.88 (d, J=8.0 Hz, 1H), 7.73 (d, J=8.0 Hz, 1H), 7.61 (s, 1H), 7.45-7.40 (m, 1H), 7.33-7.24 (m, 1H).

MS: m/z: calculated for C₅₆H₃₀N₄O₂[M+H]+: 253.23, found: 254.71.

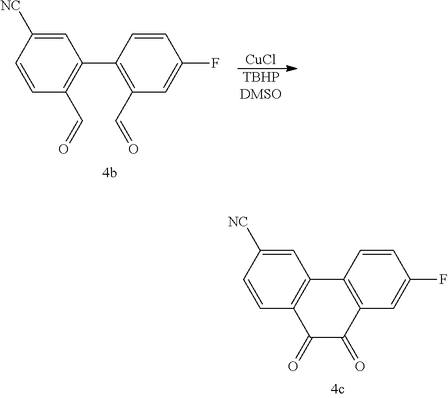

Under a nitrogen stream at room temperature, tert-butyl peroxide (70% aqueous solution, 3.8 mL, 39.5 mmol) was added to a solution of Compound 4b (2.0 g, 7.9 mmol) and copper(I) chloride (0.078 g, 0.79 mmol) dissolved in dimethyl sulfoxide (70 mL), and reacted by stirring for 2 hours. Ethyl acetate was added to the reaction solution, which was then washed with water. The organic layer was dewatered with anhydrous magnesium sulfate, then filtered, and the solvent was evaporated away from the filtrate. The remaining crude product was purified by silica gel column chromatography using a mixed solvent of ethyl acetate/chloroform/hexane=0.3/1/1 as an eluent to give a yellow solid of Compound 4c (0.250 g, yield 12.5%).

1H NMR (400 MHz, CDCl₃): δ 8.28 (d, J=8.0 Hz, 1H), 8.23 (s, 1H), 8.02 (q, J=4.0 Hz, 1H), 7.90 (dd, J=16.0 Hz, J=2.8 Hz 1H), 7.75-7.72 (m, 1H), 7.52-47 (m, 1H).

MS: m/z: calculated for C₅₆H₃₀N₄O₂[M+H]+: 251.22, found: 252.45.

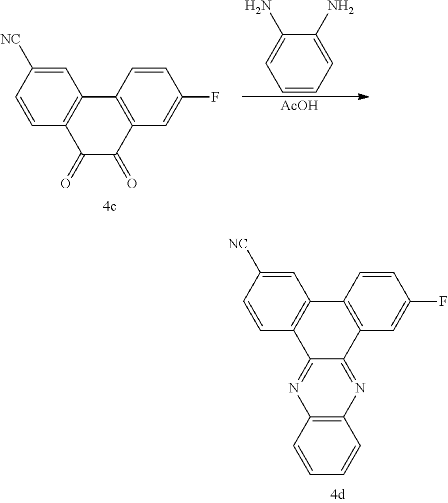 [SMILES: "AcOH" ; N#Cc1ccc2c(c1)-c1ccc(F)cc1C(=O)C2=O ; Nc1ccccc1N ; N#Cc1ccc2c(c1)c1ccc(F)cc1c1nc3ccccc3nc21]

Under a nitrogen stream, a solution of Compound 4c (0.25 g, 1.0 mmol) and 1,2-diaminobenzene (0.13 g, 1.2 mmol) dissolved in acetic acid (25 mL) was reacted by stirring at 130° C. for 24 hours. The reaction mixture was restored to room temperature, given methanol added thereto, and the precipitated sediment was filtered out. The resultant solid was reprecipitated in a mixed solvent of chloroform and methanol to give a pale yellow solid of Compound 4d (0.275 g, 0.85 mmol, yield 85%).

MS: m/z: calculated for C₅₆H₃₀N₄O₂[M+H]+: 323.33, found: 324.22.

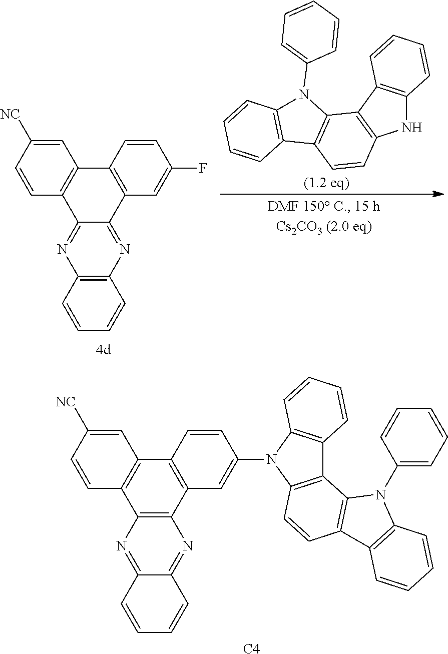

Under a nitrogen stream, a solution of Compound 4d (0.25 g, 0.77 mmol), 5,12-dihydro-12-phenylindolo[3,2-a]carbazole (0.308 g, 0.93 mmol) and cesium carbonate (0.504 g, 1.5 mmol) dissolved in N,N-dimethylformamide (25 mL) was reacted by stirring at 150° C. for 12 hours. The reaction mixture was restored to room temperature, given methanol added thereto, and the precipitated sediment was filtered out. The resultant solid was purified by silica gel column chromatography using toluene as an eluent to give a yellow solid of the intended Compound C4 (0.275 g, 1.14 mmol, yield 56%).

1H NMR (400 MHz, CDCl₃) δ 9.72 (s, 1H), 9.54 (d, J=8.0 Hz, 1H), 8.91 (s, 1H), 8.76 (d, J=8.0 Hz, 1H), 8.32 (d, J=8.0 Hz, 1H), 8.22 (d, J=8.0 Hz, 1H), 8.15-8.10 (m, 3H), 8.98 (d, J=8.0 Hz, 1H), 7.86 (t, J=8.0 Hz, 2H), 7.69 (s, 5H), 7.44-7.23 (m, 5H), 6.80 (t, J=8.0 Hz, 1H), 5.94 (d, J=8.0 Hz, 1H).

MS: m/z: calculated for C₅₆H₃₀N₄O₂[M+H]+: 635.73, found: 636.65.

### (Synthesis Example 5) Synthesis of Compound C5

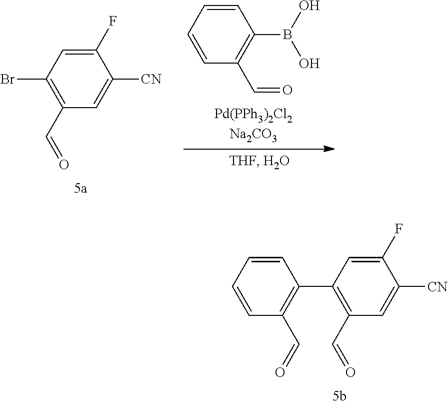 [SMILES: N#Cc1cc(C=O)c(Br)cc1F ; O=Cc1ccccc1B(O)O ; N#Cc1cc(C=O)c(-c2ccccc2C=O)cc1F]

Under a nitrogen stream, a solution of Compound 5a (3.75 g, 16.5 mmol), 2-formylphenylboronic acid (2.71 g, 18.1 mmol), bistriphenylphosphinepalladium(II) dichloride (1.16 g, 1.65 mmol) and sodium carbonate (2.62 g, 24.7 mmol) dissolved in a mixed solvent of tetrahydrofuran/water=50 mL/17 mL was reacted by stirring at 75° C. for 21 hours. The reaction solution was restored to room temperature, given chloroform added thereto, and filtered through Celite. The resultant filtrate was washed with water, the organic layer was dewatered with anhydrous magnesium sulfate, filtered, and the solvent was evaporated away from the filtrate. The remaining crude product was purified by silica gel column chromatography using a mixed solvent of ethyl acetate/chloroform/hexane=0.2/1/1 as an eluent to give a pale pink oil of Compound 5b (1.80 g, 7.12 mmol, yield 43%).

¹H NMR (400 MHz, CDCl₃): δ 9.92 (s, 1H), 9.66 (s, 1H), 8.32 (d, J=6.4 Hz, 1H), 8.05-8.02 (m, 1H), 7.78-7.73 (m, 2H), 7.34-7.31 (m, 1H), 7.19 (d, J=9.2 Hz, 1H)

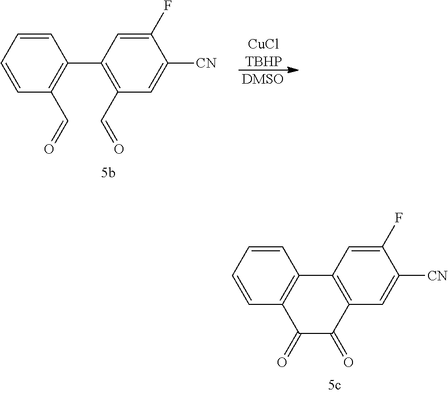

Under a nitrogen stream at room temperature, tert-butyl peroxide (70% aqueous solution, 4.4 mL, 32.0 mmol) was added to a solution of Compound 5b (1.80 g, 7.12 mmol) and copper(I) chloride (0.0705 g, 0.712 mmol) dissolved in dimethyl sulfoxide (55 mL), and reacted by stirring for 24 hours. Ethyl acetate was added to the reaction solution, which was then washed with water. The organic layer was dewatered with anhydrous magnesium sulfate, then filtered, and the solvent was evaporated away from the filtrate. The remaining crude product was purified by silica gel column chromatography using a mixed solvent of ethyl acetate/chloroform/hexane=0.3/1/1 as an eluent to give a yellow solid of Compound 5c (0.965 g, 3.84 mmol, yield 54%).

¹H NMR (400 MHz, CDCl₃): δ 8.51 (d, J=6.0 Hz, 1H), 8.29 (d, J=7.6 Hz, 1H), 7.99 (d, J=8.0 Hz, 1H), 7.89-7.81 (m, 2H), 7.66 (t, J=7.6 Hz, 1H)

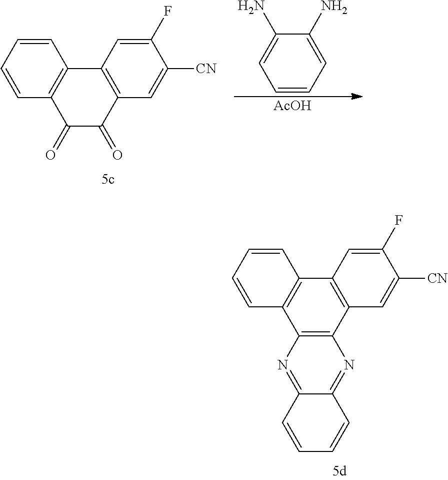 [SMILES: "AcOH" ; N#Cc1cc2c(cc1F)-c1ccccc1C(=O)C2=O ; Nc1ccccc1N ; N#Cc1cc2c(cc1F)c1ccccc1c1nc3ccccc3nc21]

Under a nitrogen stream, a solution of Compound 5c (1.06 g, 4.21 mmol) and o-phenylenediamine (0.501 g, 4.64 mmol) dissolved in acetic acid (100 mL) was reacted by stirring at 125° C. for 23 hours. The reaction mixture was restored to room temperature, given methanol added thereto, and the precipitated sediment was filtered out. The resultant solid was reprecipitated in a mixed solvent of chloroform and methanol to give a pale yellow solid of Compound 5d (0.963 g, 2.98 mmol, yield 71%).

¹H NMR (400 MHz, CDCl₃): δ 9.76 (d, J=7.2 Hz, 1H), 9.48-9.45 (m, 1H), 8.48-8.45 (m, 1H), 8.38-8.32 (m, 3H), 7.94-7.87 (m, 4H)

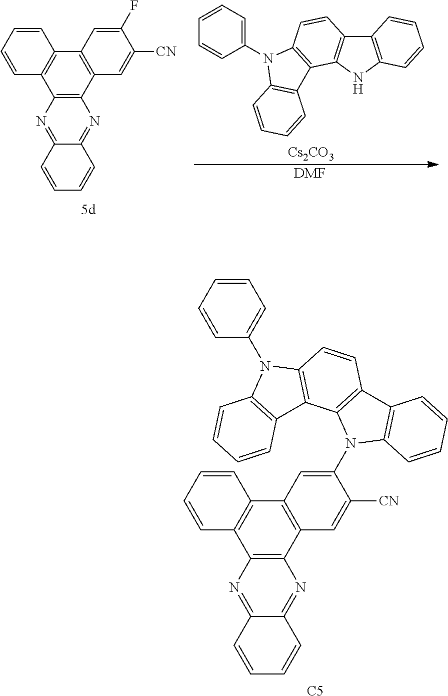

Under a nitrogen stream, a solution of Compound 5d (0.763 g, 2.36 mmol), 5-phenyl-5,12-dihydroindolo[3,2-a]carbazole (0.941 g, 2.83 mmol) and cesium carbonate (1.54 g, 4.72 mmol) dissolved in N,N-dimethylformamide (60 mL) was reacted by stirring at 150° C. for 1 hour. The reaction mixture was restored to room temperature, given methanol added thereto, and the precipitated sediment was filtered out. The resultant solid was purified by silica gel column chromatography using a mixed solvent of chloroform/toluene=1/1 as an eluent, and then recrystallized with a mixed solvent of chloroform, toluene and methanol to give a yellow solid of the intended Compound C5 (0.727 g, 1.14 mmol, yield 48%).

¹H NMR (400 MHz, CDCl₃): δ 10.16 (s, 1H), 9.50 (d, J=8.4 Hz, 1H), 8.80 (s, 1H), 8.48-8.42 (m, 1H), 8.30-8.20 (m, 3H), 8.02-7.97 (m, 2H), 7.84 (t, J=6.8 Hz, 1H), 7.74-7.60 (m, 5H), 7.57-7.51 (m, 1H), 7.44-7.39 (m, 3H), 7.34-7.24 (m, 2H), 7.09-7.04 (m, 1H), 6.52-6.47 (m, 1H), 6.09 (d, J=8.0 Hz, 1H)

MS: m/z: calculated for C₄₅H₂₅N₅[M+H]⁺: 635.2, found: 635.3.

### (Synthesis Example 6) Synthesis of Compound C6

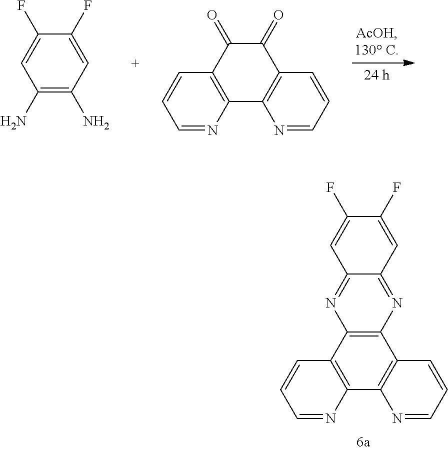 [SMILES: Nc1cc(F)c(F)cc1N ; O=C1C(=O)c2cccnc2-c2ncccc21 ; Fc1cc2nc3c4cccnc4c4ncccc4c3nc2cc1F]

Under a nitrogen stream, a solution of 1,10-phenanthroline-5,6-dione (1.0 g, 5.0 mmol) and 4,5-difluoro-1,2-phenylenediamine (0.87 g, 6.5 mmol) dissolved in acetic acid (100 mL) was reacted by stirring under heat at 130° C. for 24 hours. The reaction mixture was restored to room temperature, given methanol added thereto, and the precipitated sediment was filtered out. The resultant solid was dried under reduced pressure, again washed with methanol, and then purified by silica gel column chromatography and recrystallization to give Compound 6a (1.49 g, 4.68 mmol, yield 93%).

1H NMR (400 MHz, CDCl3) δ 9.58 (dd, J=8.0 Hz, J=1.6 Hz, 2H), 9.28 (s, 2H), 8.07 (t, J=8.0 Hz, 2H), 7.80 (q, J=4.0 Hz, 2H).

MS: m/z: calculated for C₂₀H₉BrF₂N₂[M+H]+: 318.29, found: 319.45.

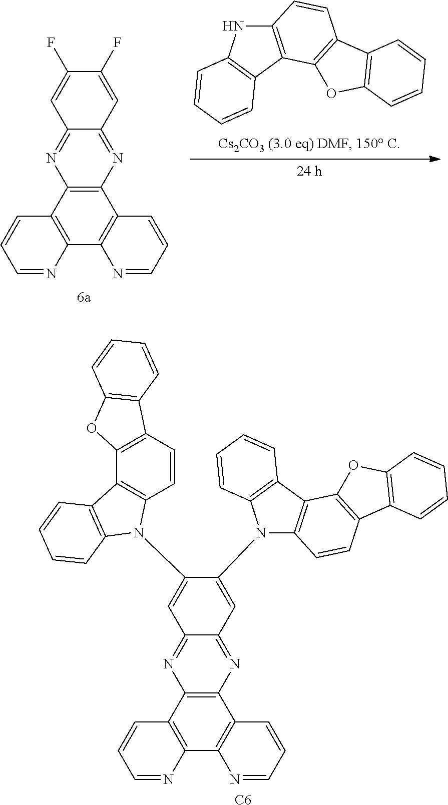

Under a nitrogen stream, a solution of Compound 6a (0.756 g, 2.37 mmol), 5H-benzofuro[3,2-c]carbazole (1.28 g, 5.0 mmol) and cesium carbonate (3.12 g, 10.0 mmol) dissolved in N,N-dimethylformamide (75 mL) was reacted by stirring under heat at 150° C. for 2 hours. The reaction mixture was restored to room temperature, the reaction was stopped with water, the precipitated sediment was filtered out, and the residue was washed with methanol. The resulting crude product was purified by silica gel column chromatography to give the intended Compound C6 (1.69 g, 2.13 mmol, yield 90%).

1H NMR (400 MHz, CDCl3) δ 9.55 (m, 2H), 9.70 (d, J=8.0 Hz, 2H), 8.87 (s, 2H), 8.16 (t, J=8.0 Hz, 2H), 7.83 (t, J=8.0 Hz, 2H), 7.72 (m, 2H), 7.55 (m, 4H), 7.30 (m, 8H), 7.13 (m, 4H).

MS: m/z: calculated for C₅₆H₃₀N₄O₂[M+H]+: 792.86, found: 793.41.

### (Synthesis Example 7) Synthesis of Compound C7

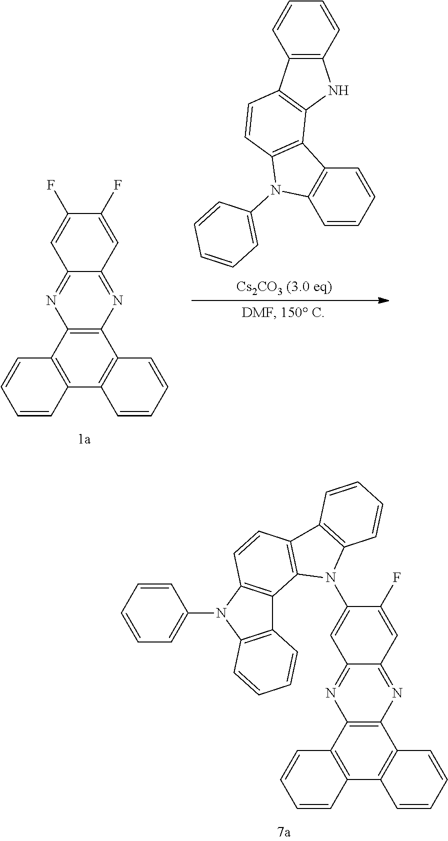 [SMILES: Fc1cc2nc3c4ccccc4c4ccccc4c3nc2cc1F ; c1ccc(-n2c3ccccc3c3c4[nH]c5ccccc5c4ccc32)cc1 ; Fc1cc2nc3c4ccccc4c4ccccc4c3nc2cc1-n1c2ccccc2c2ccc3c(c4ccccc4n3-c3ccccc3)c21]

Under a nitrogen stream, a solution of Compound 1a (1.0 g, 3.16 mmol), 5,12-dihydro-5-phenylindolo[3,2-a]carbazole (1.05 g, 3.16 mmol) and cesium carbonate (1.54 g, 4.74 mmol) dissolved in N,N-dimethylformamide (60 mL) was reacted by stirring under heat at 120° C. for 24 hours. The reaction mixture was restored to room temperature, the reaction was stopped with water, and then the precipitated sediment was filtered out. The residue was washed with methanol, and purified by silica gel column chromatography to give Compound 7a (1.3 g, 2.06 mmol, yield 65%).

¹H NMR (400 MHz, CDCl₃) δ 9.33 (d, J=8.0 Hz, 1H), 9.16 (d, J=8.4 Hz, 1H), 8.83-8.78 (m, 3H), 8.55 (d, J=10.8 Hz, 1H), 8.36-8.26 (m, 2H), 7.95-7.87 (m, 3H), 7.77-7.56 (m, 6H), 7.38-7.29 (m, 4H), 7.18 (d, J=8.0 Hz, 2H), 7.06 (t, J=7.8 Hz, 1H), 6.45 (t, J=7.4 Hz, 1H), 6.01 (d, J=8.4 Hz, 1H).

MS: m/z: calculated for C₄₄H₂₅FN₄[M+H]⁺: 628.21, found: 629.38.

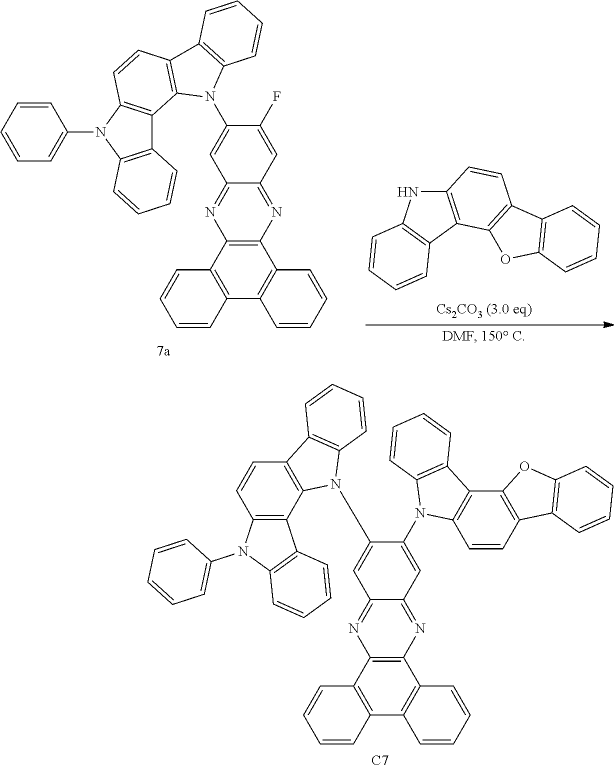

Under a nitrogen stream, a solution of Compound 7a (1.2 g, 1.9 mmol), 5H-benzofuro[3,2-c]carbazole (0.75 g, 2.9 mmol) and cesium carbonate (1.85 g, 5.7 mmol) dissolved in N,N-dimethylformamide (250 mL) was reacted by stirring under heat at 150° C. for 24 hours. The reaction mixture was restored to room temperature, the reaction was stopped with water, and then the precipitated sediment was filtered out. The residue was washed with methanol, and purified by silica gel column chromatography to give the intended Compound C7 (1.5 g, 1.73 mmol, yield 91%).

¹H NMR (400 MHz, CDCl₃) δ 9.59-9.55 (m, 1H), 9.48-9.45 (m, 1H), 9.28-9.22 (m, 1H), 8.73-8.65 (m, 3H), 8.07-7.73 (m, 6H), 7.68-7.57 (m, 2H), 7.50-6.94 (m, 14H), 6.81-6.30 (m, 7H).

MS: m/z: calculated for C₆₂H₃₅N₅O[M+H]⁺: 865.28, found: 866.44.

### (Synthesis Example 8) Synthesis of Compound C8

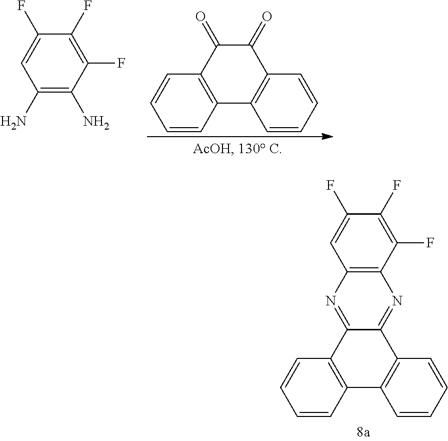 [SMILES: Nc1cc(F)c(F)c(F)c1N ; O=C1C(=O)c2ccccc2-c2ccccc21 ; Fc1cc2nc3c4ccccc4c4ccccc4c3nc2c(F)c1F]

Under a nitrogen stream, a solution of 3,4,5-trifluoro-1,2-phenylenediamine (3.4 g, 21.0 mmol) and phenanthroline-9,10-dione (4.36 g, 21.0 mmol) in acetic acid (250 mL) was stirred at 130° C. for 24 hours. The reaction mixture was restored to room temperature, given methanol added thereto, and the precipitated sediment was filtered out. The resultant solid was dried under reduced pressure, again washed with methanol, and then purified by recrystallization to give Compound 8a (6.5 g, 19.4 mmol, yield 92%).

¹H NMR (400 MHz, CDCl₃) δ 9.38 (d, J=8.0 Hz, 1H), 9.30 (d, J=8.0 Hz, 1H), 8.56 (t, J=8.0 Hz, 1H), 7.85-7.74 (m, 5H).

MS: m/z: calculated for C₂₀H₉F₃N₂[M+H]⁺: 334.30, found: 335.40.

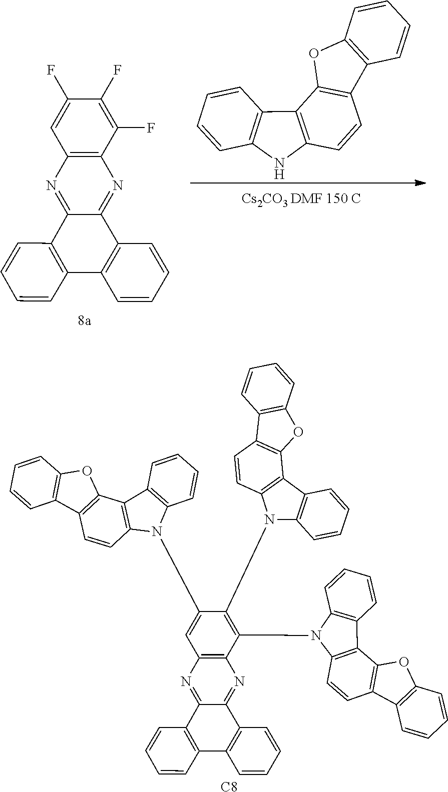 [SMILES: Fc1cc2nc3c4ccccc4c4ccccc4c3nc2c(F)c1F ; c1ccc2c(c1)[nH]c1ccc3c4ccccc4oc3c12 ; c1ccc2c(c1)oc1c2ccc2c1c1ccccc1n2-c1cc2nc3c4ccccc4c4ccccc4c3nc2c(-n2c3ccccc3c3c4oc5ccccc5c4ccc32)c1-n1c2ccccc2c2c3oc4ccccc4c3ccc21 ; "C8"]

Under a nitrogen stream, a solution of Compound 8a (0.3 g, 0.898 mmol), 5H-benzofuro[3,2-c]carbazole (1.15 g, 4.5 mmol) and cesium carbonate (0.097 g, 4.05 mmol) dissolved in N,N-dimethylformamide (50 mL) was reacted by stirring under heat at 150° C. for 2 hours. The reaction mixture was restored to room temperature, the reaction was stopped with water, and then the precipitated sediment was filtered out. The residue was washed with methanol, and purified by silica gel column chromatography to give the intended Compound C8 (0.85 g, 0.81 mmol, yield 90.2%).

¹H NMR (400 MHz, CDCl₃) δ 9.39 (d, J=8.0 Hz, 1H), 9.24 (d, J=4.0 Hz, 1H), 8.47 (d, J=8.0 Hz, 1H), 8.39 (d, J=8.0 Hz, 1H), 8.32 (d, J=8.0 Hz, 1H), 8.12 (t, J=8.0 Hz, 1H), 7.98 (d, J=8.0 Hz, 1H), 7.64-6.62 (m, 32H).

MS: m/z: calculated for C₅₇H₂₉N₅O₂[M+H]⁺: 1045.31, found: 1046.32

### (Synthesis Example 9) Synthesis of Compound C9

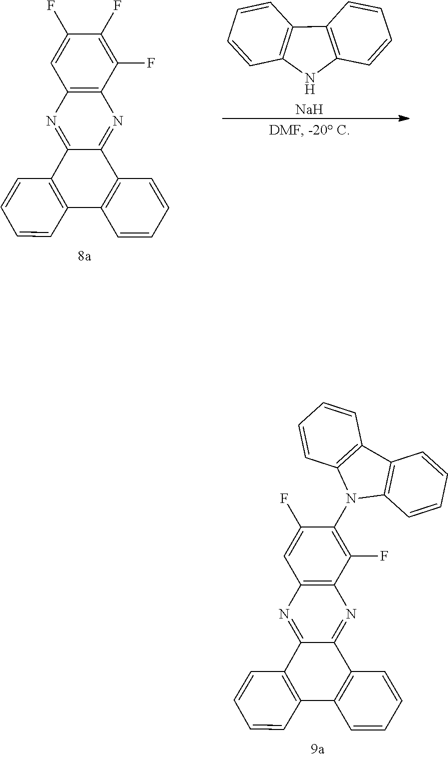

Under a nitrogen stream, 9H-carbazole (1.67 g, 10.0 mmol) was added to an N,N-dimethylformamide solution (800 mL) of sodium hydride (60% mineral oil dispersed, 0.24 g, 10.0 mmol). The mixture was cooled down to −20° C., Compound 8a (3.5 g, 10.5 mmol) was added thereto, and stirred for 24 hours. The reaction mixture was restored to room temperature, quenched with water added thereto, extracted with chloroform, the organic layer was washed with saturated saline water, and dried with anhydrous magnesium sulfate. This was concentrated under reduced pressure, and the resultant mixture was purified by silica gel column chromatography to give Compound 9a (0.48 g, 1.0 mmol, yield 9.5%).

¹H NMR (400 MHz, CDCl₃) δ9.29 (dd, J=8.0 Hz, J=1.6 Hz, 1H), 9.22 (dd, J=8.4 Hz, J=1.6 Hz, 1H), 8.85-8.82 (m, 2H), 8.38 (dd, J=10.4 Hz, J=1.2 Hz, 1H), 8.29 (d, J=7.6 Hz, 2H), 7.97-7.80 (m, 4H), 7.49-7.45 (m, 2H), 7.40-7.33 (m, 4H).

MS: m/z: calculated for C₃₂H₁₇F₂N₃[M+H]⁺: 481.14, found: 482.15.

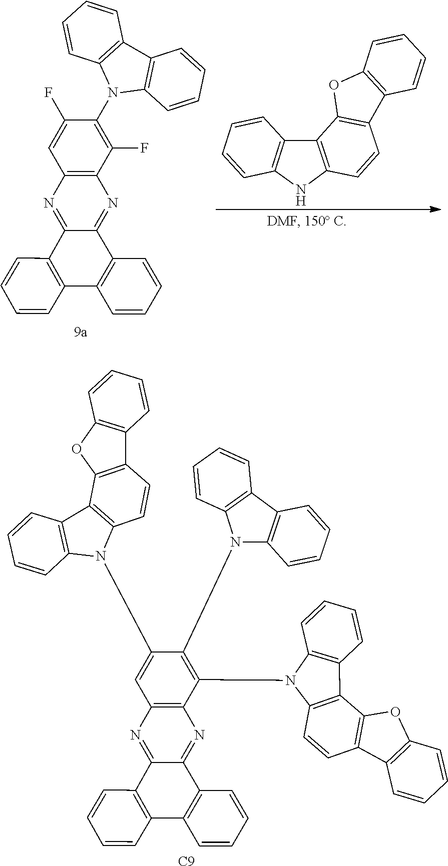

Under a nitrogen stream, 5H-benzofuro[3,2-d]carbazole (0.64 g, 2.5 mmol) was added to an N,N-dimethylformamide solution (100 mL) of sodium hydride (60% mineral oil dispersed, 0.054 g, 2.25 mmol). Compound 9a (0.45 g, 0.93 mmol) was added thereto, and stirred at 150° C. for 3 hours. The reaction mixture was restored to room temperature, the reaction was stopped with water, and then the precipitated sediment was filtered out. The residue was washed with methanol, and purified by silica gel column chromatography to give Compound C9 (0.8 g, 0.83 mmol, yield 89.2%).

MS: m/z: calculated for C₆₈H₃₇N₅O₂[M+H]⁺: 955.29, found: 956.44

### (Synthesis Example 10) Synthesis of Compound C10

The following Compound C10 is synthesized according to the same method as in Synthesis Example 8.

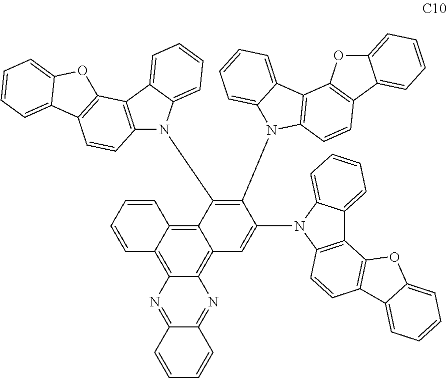 [SMILES: c1ccc2nc3c4cc(-n5c6ccccc6c6c7oc8ccccc8c7ccc65)c(-n5c6ccccc6c6c7oc8ccccc8c7ccc65)c(-n5c6ccccc6c6c7oc8ccccc8c7ccc65)c4c4ccccc4c3nc2c1]

### (Synthesis Example 11) Synthesis of Compound C11

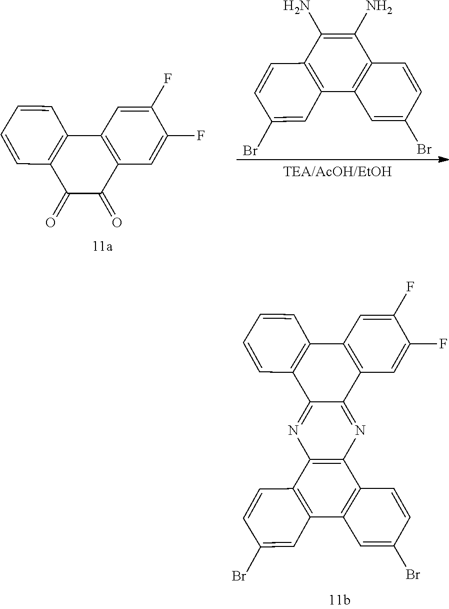 [SMILES: O=C1C(=O)c2cc(F)c(F)cc2-c2ccccc21 ; Nc1c(N)c2ccc(Br)cc2c2cc(Br)ccc12 ; Fc1cc2c3ccccc3c3nc4c5ccc(Br)cc5c5cc(Br)ccc5c4nc3c2cc1F]

Under a nitrogen stream, Compound 11a (0.660 g, 2.70 mmol) and 3,6-dibromophenanthrene-9,10-diamine (1.01 g, 2.76 mmol) were added to a mixed solvent of acetic acid/ethanol=100 mL/25 mL, and stirred at 100° C. for 30 minutes. Triethylamine (3 mL) was added to the reaction solution, and under a nitrogen stream, stirred at 130° C. for 15 hours. The reaction solution was restored to room temperature, given methanol added thereto, and the precipitated sediment was filtered out. The resultant solid was reprecipitated in a mixed solvent of chloroform and methanol to give a pale yellow solid of Compound 11b (1.40 g, 2.44 mmol, yield 90%).

MS: m/z: calculated for C₃₀H₁₂F₂N₄[M+H]+: 571.9, found: 572.0

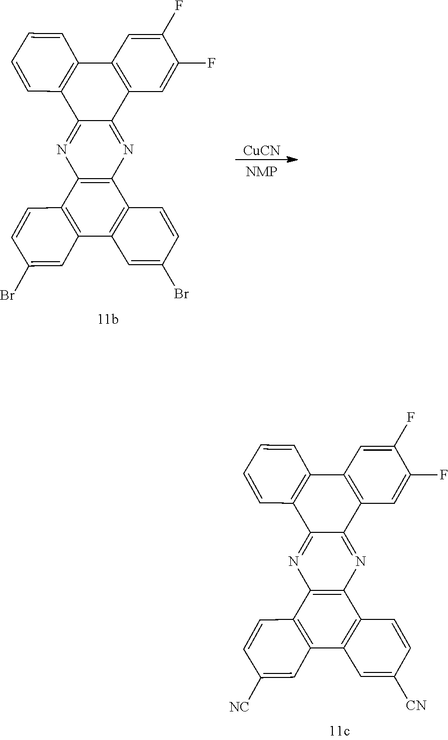 [SMILES: Fc1cc2c3ccccc3c3nc4c5ccc(Br)cc5c5cc(Br)ccc5c4nc3c2cc1F ; "CuCN" ; N#Cc1ccc2c(c1)c1cc(C#N)ccc1c1nc3c4cc(F)c(F)cc4c4ccccc4c3nc21]

Under a nitrogen stream, a solution of Compound 11b (0.68 g, 1.2 mmol) and copper(I) cyanide (0.636 g, 7.11 mmol) dissolved in N-methyl-2-pyrrolidone (70 mL) was reacted by stirring under heat at 180° C. for 48 hours. The reaction mixture was restored to room temperature, the reaction was stopped with aqueous ammonia, then water was added, and the precipitated sediment was filtered out. The resultant solid was reprecipitated in a mixed solvent of chloroform and methanol to give Compound 11c (0.55 g, 1.2 mmol, yield 99%).

MS: m/z: calculated for C₅₆H₃₀N₄O₂[M+H]+: 466.1, found: 466.1

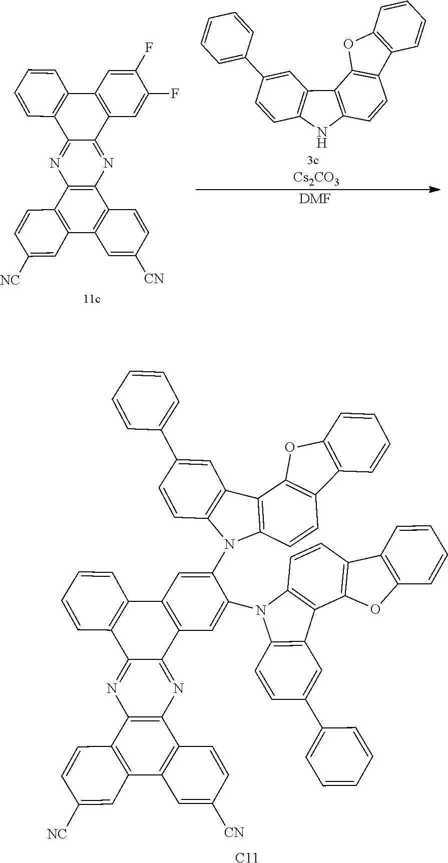

Under a nitrogen stream, a solution of Compound 11c (0.430 g, 0.922 mmol), Compound 3c (0.768 g, 2.30 mmol) and cesium carbonate (1.20 g, 3.69 mmol) dissolved in N,N-dimethylformamide (100 mL) was reacted by stirring under heat at 150° C. for 6 hours. The reaction mixture was restored to room temperature, the reaction was stopped with water, and then the precipitated sediment was filtered out. The residue was washed with methanol, and purified by silica gel column chromatography to give Compound C11 (0.65 g, 0.59 mmol, yield 64%).

MS: m/z: calculated for C₇₈H₄₀N₆O₂[M+H]⁺: 1092.3, found: 1092.3

### (Synthesis Example 12) Synthesis of Compound C12

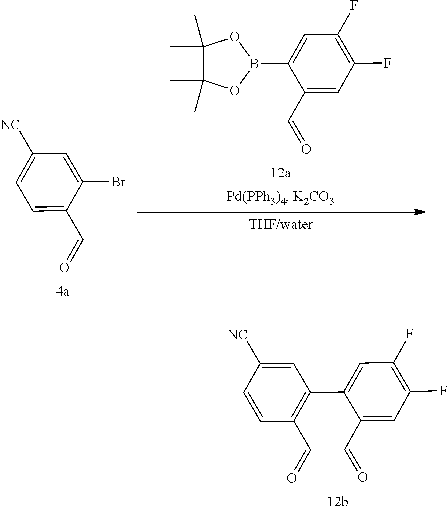 [SMILES: N#Cc1ccc(C=O)c(Br)c1 ; CC1(C)OB(c2cc(F)c(F)cc2C=O)OC1(C)C ; N#Cc1ccc(C=O)c(-c2cc(F)c(F)cc2C=O)c1]

Under a nitrogen stream, a solution of Compound 4a (18.8 g, 89.6 mmol), Compound 12a (21.8 g, 81.4 mmol), tetrakis(triphenylphosphine)palladium (0) (4.71 g, 4.07 mmol) and potassium carbonate (22.5 g, 163 mmol) dissolved in a mixed solvent of tetrahydrofuran/water=300 mL/100 mL was reacted by stirring at 80° C. for 16 hours. The reaction solution was restored to room temperature, and extracted with ethyl acetate. The resultant organic layer was dewatered with anhydrous magnesium sulfate, then filtered, and the solvent was evaporated away from the filtrate. The remaining crude product was purified by silica gel column chromatography to give Compound 12b (15.7 g, 57.9 mmol, yield 71%).

1H NMR (400 MHz, CDCl3) δ 9.88 (s, 1H), 9.69 (d, J=2.3 Hz, 1H), 8.14 (d, J=8.2 Hz, 1H), 7.93 (dd, J=8.0, 1.1 Hz, 1H), 7.88 (dd, J=9.6, 7.8 Hz, 1H), 7.64 (d, J=1.4 Hz, 1H), 7.19 (dd, J=9.8, 7.1 Hz, 1H).

MS: m/z: calculated for C₁₅H₇F₂NO₂[M+H]+: 271.04, found: 272.05.

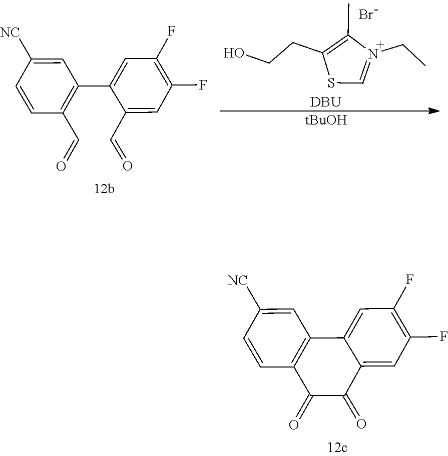

Compound 12b (5.00 g, 18.4 mmol) and 3-ethyl-5-(2-hydroxyethyl)-4-methylthiazolium bromide (0.930 g, 3.69 mmol) were added to tert-butanol (180 mL), and stirred at 60° C. for 10 minutes under a nitrogen stream. 1,8-Diazabicyclo[5.4.0]undec-7-ene (8.42 g, 5.53 mmol) was added to the reaction solution, and reacted by stirring at 60° C. for 4 hours under a nitrogen stream. The reaction solution was restored to room temperature, and extracted with dichloromethane. The organic layer was dewatered with anhydrous magnesium sulfate, then filtered, and the solvent was evaporated away from the filtrate. The resultant solid was reprecipitated in toluene to give Compound 12c (3.80 g, 14.1 mmol, yield 77%).

¹H NMR (400 MHz, CDCl₃): δ 8.32 (d, J=8.0 Hz, 1H), 8.15 (s, 1H), 8.09-8.05 (m, 1H), 7.85-7.79 (m, 2H).

MS: m/z: calculated for C15H5F2NO2 [M+H]+: 269.03, found: 270.07.

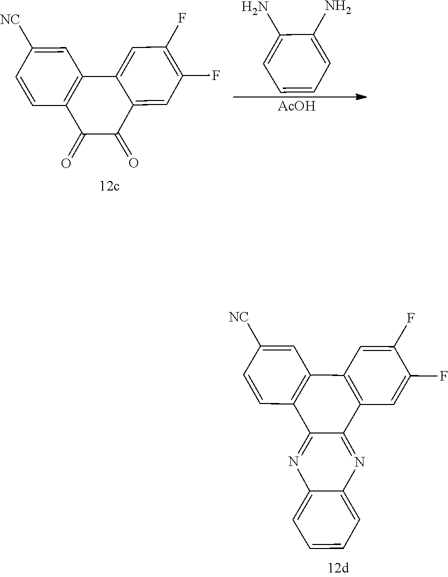 [SMILES: "AcOH" ; N#Cc1ccc2c(c1)-c1cc(F)c(F)cc1C(=O)C2=O ; Nc1ccccc1N ; N#Cc1ccc2c(c1)c1cc(F)c(F)cc1c1nc3ccccc3nc21]

Under a nitrogen stream, Compound 12c (1.50 g, 5.57 mmol) and 1,2-diaminobenzene (0.663 g, 6.13 mmol) were added to acetic acid (250 mL), and reacted by stirring at 130° C. for 16 hours. The reaction solution was restored to room temperature, given methanol added thereto, and the precipitated sediment was filtered out. The resultant solid was reprecipitated in a mixed solvent of chloroform and methanol to give Compound 12d (1.39 g, 4.07 mmol, yield 73%).

MS: m/z: calculated for C21H9F2N3 [M+H]+: 341.08, found: 342.18

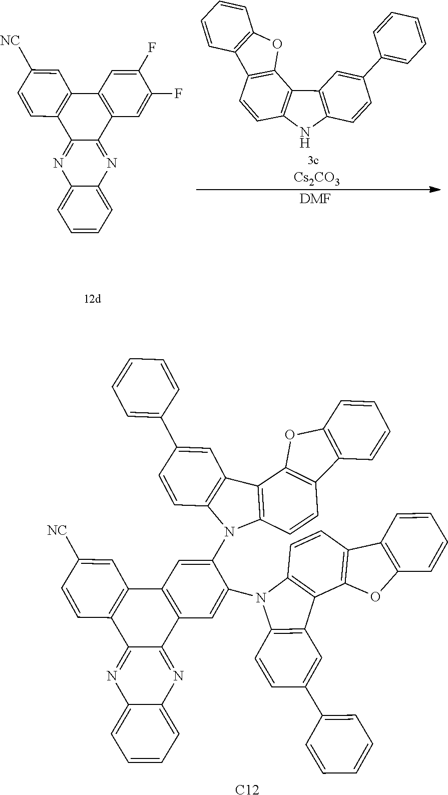

Under a nitrogen stream, a solution of Compound 12d (0.700 g, 2.05 mmol), Compound 3c (1.71 g, 5.13 mmol) and cesium carbonate (2.67 g, 8.20 mmol) dissolved in N,N-dimethylformamide (200 mL) was reacted by stirring under heat at 150° C. for 18 hours. The reaction mixture was restored to room temperature, the reaction was stopped with water, and then the precipitated sediment was filtered out. The residue was washed with methanol, and purified by silica gel column chromatography to give the intended Compound C12 (0.29 g, 0.30 mmol, yield 15%).

¹H NMR (400 MHz, CDCl₃) δ 9.78 (s, 1H), 9.13 (s, 1H), 8.86-8.77 (m, 2H), 8.26-8.13 (m, 4H), 7.75-7.30 (m, 29H)

MS: m/z: calculated for C69H37N5O2 [M+H]⁺: 967.29, found: 968.65.

### (Synthesis Example 13) Synthesis of Compound C13

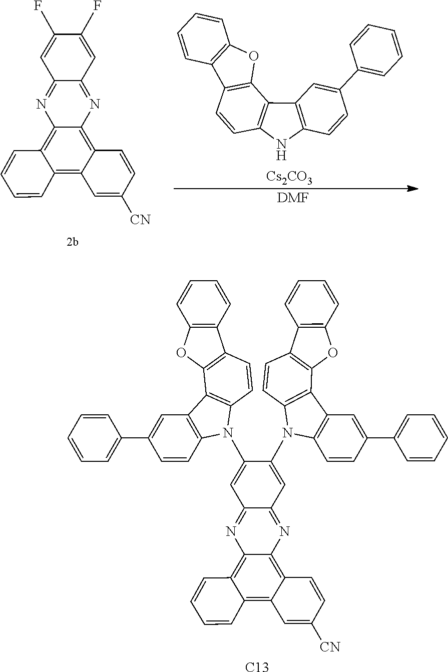

Under a nitrogen stream, a solution of Compound 2b (0.811 g, 2.38 mmol), 2-phenyl-5H-benzofuro[3,2-c]carbazole (1.98 g, 5.94 mmol) and cesium carbonate (2.32 g, 7.13 mmol) dissolved in N,N-dimethylformamide (80 mL) was reacted by stirring under heat at 150° C. for 5 hours. The reaction mixture was restored to room temperature, the reaction was stopped with water, and then the precipitated sediment was filtered out. The residue was washed with methanol, and purified by silica gel column chromatography to give Compound C13 (1.60 g, 1.65 mmol, yield 70%).

¹H NMR (400 MHz, CDCl₃) δ 8.94-8.74 (m, 9H), 8.37 (s, 1H), 8.30 (s, 1H), 8.10-7.95 (m, 3H), 7.94-7.77 (m, 5H), 7.76-7.70 (m, 1H), 7.69-7.62 (m, 2H), 7.62-6.90 (m, 17H). MS: m/z: calculated for C₆₉H₃₇N₅O₂[M+H]⁺: 988.09, found: 968.40.

### (Synthesis Example 14) Synthesis of Compound C14

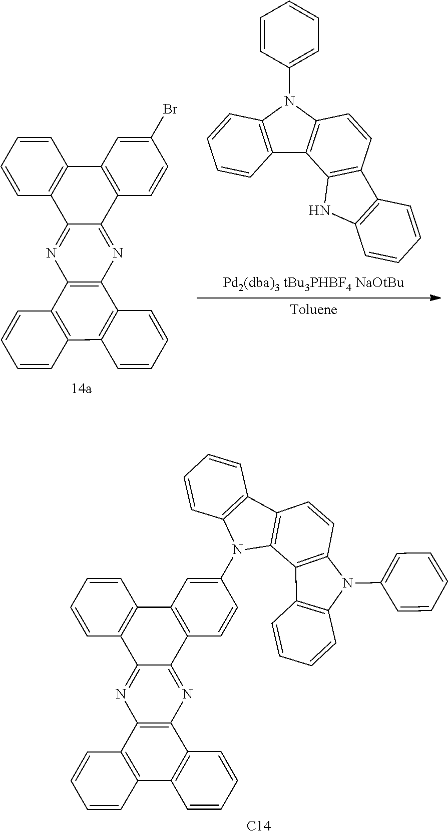 [SMILES: Brc1ccc2c(c1)c1ccccc1c1nc3c4ccccc4c4ccccc4c3nc21 ; c1ccc(-n2c3ccccc3c3c4[nH]c5ccccc5c4ccc32)cc1 ; c1ccc(-n2c3ccccc3c3c2ccc2c4ccccc4n(-c4ccc5c(c4)c4ccccc4c4nc6c7ccccc7c7ccccc7c6nc54)c23)cc1 ; "C14"]

Under a nitrogen atmosphere, Compound 14a (2.00 g, 4.35 mmol), 5-phenyl-5,12-dihydroindolo[3,2-a]carbazole (1.74 g, 5.22 mmol), tris(dibenzylideneacetone) dipalladium (0) (400 mg, 0.435 mmol), tri-tert-butylphosphonium tetrafluoroborate (250 mg, 0.870 mmol) and sodium tert-butoxide (840 mg, 8.71 mmol) were dissolved in toluene (435 ml), and stirred at 130° C. for 14 hours. The reaction solution was cooled down to 80° C., the solid was filtered out, and the resultant solid was purified by silica gel column chromatography (developing solvent: toluene), and washed with acetonitrile to give a yellow solid of Compound C14. (2.60 g, 84%).

¹H NMR (400 MHz, CDCl₃, δ): 9.88 (d, J=8.4 Hz, 1H), 9.68-9.60 (m, 3H), 8.99 (s, 1H), 8.71-8.65 (m, 2H), 8.52 (d, J=7.6 Hz, 1H), 8.27-8.20 (m, 2H), 8.10-8.05 (m, 1H), 7.90-7.71 (m, 6H), 7.69-7.60 (m, 4H), 7.58-7.50 (m, 2H), 7.45-7.35 (m, 3H), 7.26-7.24 (m, 1H), 7.05 (t, J=8.0 Hz, 1H), 6.50 (t, J=8.0 Hz, 1H), 6.22 (d, J=8.4 Hz, 1H).

MS (ASAP): 711.52 (M+H⁺). Calcd for C₅₂H₃₀N₄: 710.25.

### (Synthesis Example 15) Synthesis of Compound C15

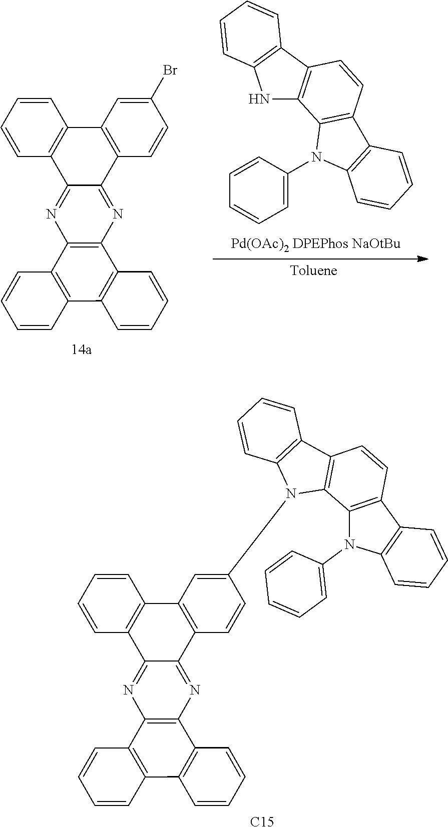 [SMILES: Brc1ccc2c(c1)c1ccccc1c1nc3c4ccccc4c4ccccc4c3nc21 ; c1ccc(-n2c3ccccc3c3ccc4c5ccccc5[nH]c4c32)cc1 ; c1ccc(-n2c3ccccc3c3ccc4c5ccccc5n(-c5ccc6c(c5)c5ccccc5c5nc7c8ccccc8c8ccccc8c7nc65)c4c32)cc1 ; "C15"]

Under a nitrogen atmosphere, Compound 14a (1.00 g, 2.18 mmol), 11,12-dihydro-11-phenylindolo[2,3-a]carbazole (870 mg, 2.61 mmol), palladium acetate (48.9 mg, 0.218 mmol), bis[2-(diphenylphosphino)phenyl] ether (234 mg, 0.435 mmol), and sodium tert-butoxide (420 mg, 4.35 mmol) were dissolved in toluene (220 mL), and stirred at 130° C. for 17 hours. The reaction solution was cooled down to room temperature, the insoluble matter was filtered away, and the solvent was removed from the resultant filtrate. The resultant residue was purified by silica gel column chromatography (developing solvent: toluene/hexane=3/7), and further recrystallized with toluene and acetonitrile to give a yellow solid of Compound C15. (975 mg, 62%).

¹H NMR (400 MHz, 1,1,2,2-Tetrachloroethane-d₂, δ): 9.60-9.52 (m, 3H), 9.29 (d, J=8.8 Hz, 1H), 8.67-8.64 (m, 2H), 8.25-8.15 (m, 5H), 8.00 (s, 1H), 7.85-7.73 (m, 6H), 7.34-7.08 (m, 7H), 6.90-6.42 (m, 4H).

MS (ASAP): 711.63 (M+H⁺). Calcd for C₅₂H₃₀N₄: 710.25.

### (Example 1) Formation of Thin Film of Compound C1

On a quartz substrate, according to a vacuum evaporation method, Compound C1 was deposited under the condition of a vacuum degree of lower than 1×10⁻³ Pa to form a thin film of Compound C1 alone having a thickness of 100 nm, and this is a neat thin film of Example 1. Apart from this, on a quartz substrate, according to a vacuum evaporation method, Compound C1 and mCBP were evaporated from different evaporation sources under the condition of a vacuum degree of lower than 1×10⁻³ Pa to form a thin film having a thickness of 100 nm in which the concentration of compound C1 was 20% by weight, and this is a doped thin film of Example 1.

(Examples 2 to 9) Formation of Thin Films of Compounds C2 to C6, C11 to C13 Neat thin films and doped thin films were formed in the same manner as in Example 1, except that Compounds C2 to C6, and C11 to C13 were used in place of Compound C1.

### (Comparative Example 1) Formation of Thin Film of Comparative Compound 1

A neat thin film and a doped thin film were formed in the same manner as in Example 1, except that Comparative Compound 1 was used in place of Compound C1.

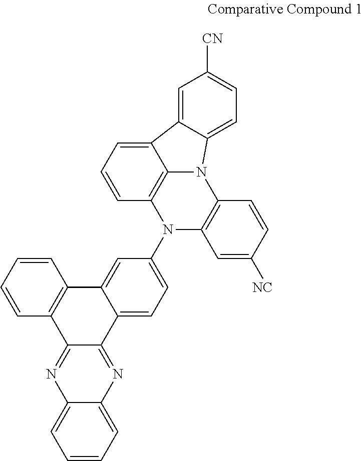 [SMILES: N#Cc1ccc2c(c1)N(c1ccc3c(c1)c1ccccc1c1nc4ccccc4nc31)c1cccc3c4cc(C#N)ccc4n-2c13]

### Evaluation of Thin Films

Absolute values of the HOMO and LUMO energy of the neat thin films formed in Examples 1 to 9 and Comparative Example 1, and the photoluminescence quantum yield (PLQY) and the emission maximum wavelength of the doped thin films formed therein are shown in Table 45. In Table 45, “-” means not measured.

TABLE 45

| | | | | | Emission |
|---|---|---|---|---|---|
| | | | | | Maximum |
| | | PLQY | HOMO | LUMO | Wavelength |
| Example No. | Compound No. | (%) | (eV) | (eV) | (nm) |
| Example 1 | Compound C1 | 72.0 | 5.97 | 3.60 | 562 |
| Example 2 | Compound C2 | 76.0 | 6.02 | 3.79 | 595 |
| Example 3 | Compound C3 | 61.1 | 5.91 | 3.61 | 587 |
| Example 4 | Compound C4 | 64.6 | 5.66 | 3.34 | 583 |
| Example 5 | Compound C5 | 62.0 | 5.72 | 3.09 | 562 |
| Example 6 | Compound C6 | 62.0 | 6.01 | — | 589 |
| Example 7 | Compound C11 | 86.4 | 5.97 | 3.70 | 570 |
| Example 8 | Compound C12 | 76.5 | 5.98 | 3.61 | 560 |
| Example 9 | Compound C13 | 83.8 | 5.98 | 3.82 | 599 |
| Comparative | Comparative | 49.1 | 5.78 | 3.65 | 607 |
| Example 1 | Compound 1 | | | | |

As shown in Table 45, the compound represented by the general formula (1) attained high PLQY in every high-concentration doped thin film. Accordingly, organic light emitting devices using the compound realize high light emission efficiency and good durability.

(Example 10) Formation of Organic Electroluminescent Device using Compound C1

On a glass substrate on which an anode made of indium-tin oxide (ITO) having a film thickness of 50 nm was formed, each thin film was laminated by a vacuum deposition method at a vacuum degree of 5.0×10⁻⁵ Pa. First, HAT-CN was formed at a thickness of 10 nm on the ITO, NPD was formed at a thickness of 35 nm on the HAT-CN, and further PTCz was formed at a thickness of 10 nm on the NPD. Next, H1, Compound C1 and a light emitting material EM1 were co-deposited from different vapor deposition sources in an amount of 69.5% by weight, 30.0% by weight and 0.5% by weight, respectively, thereby forming a light emitting layer having a thickness of 40 nm. Next, after ET1 was formed at a thickness of 10 nm, Liq and SF3-TRZ were co-deposited from different vapor deposition sources to form a layer with a thickness of 20 nm. The contents of Liq and SF3-TRZ in this layer were 30% by mass and 70% by mass, respectively. Furthermore, Liq was formed at a thickness of 2 nm, and aluminum (Al) was vapor-deposited at a thickness of 100 nm to form a cathode, thereby producing an organic electroluminescent device (EL device 1).

(Examples 11 to 15, Comparative Example 2) Formation of Organic Electroluminescent Devices using Compound C2 to C6 or Comparative Compound 1

Organic Electroluminescent devices (EL devices 2 to 6, Comparative EL device 1) were formed in the same manner as in Example 10, except that Compound C2 to C6 or Comparative Compound 1 was used in place of Compound C1.

### Evaluation of EL Device

EL device 1 formed using Compound C1, EL device 2 formed using Compound C2, and Comparative EL device 1 formed using Comparative Compound 1 were continuously driven at 50 mA, and the time taken until the luminance became 95%, LT95 was measured. d. LT95 of each device is expressed as a relative value when LT95 of Comparative EL Device is defined as 1, and LT95 of EL device 1 was 1.19, and EL95 of EL device 2 was 2.46. From the results, it is known that, using the compound represented by the general formula (1) as a material of the light emitting layer, EL devices having good durability can be provided.

(Example 16) Formation of Organic Electroluminescent Device using Compound C14

On a glass substrate on which an anode made of indium-tin oxide (ITO) having a film thickness of 50 nm was formed, each thin film was laminated by a vacuum deposition method at a vacuum degree of 5.0×10⁻⁵ Pa. First, on ITO, HAT-CN was formed at a thickness of 10 nm, and then NPD was formed thereon at a thickness of 30 nm. Further thereon, TrisPCz was formed at a thickness of 10 nm, and thereon EBL1 was formed at a thickness of 5 nm. Next, H2, Compound C14, a delayed fluorescent material TADF1 and a light emitting material EM1 were co-deposited from different vapor deposition sources in an amount of 44.7% by weight, 20.0% by weight, 35.0% by weight and 0.3% by weight, respectively, thereby forming a light emitting layer having a thickness of 40 nm. Next, after SF3-TRZ was formed at a thickness of 10 nm, Liq and SF3-TRZ were co-deposited from different vapor deposition sources to form a layer with a thickness of 30 nm. The contents of Liq and SF3-TRZ in this layer were 30% by mass and 70% by mass, respectively. Furthermore, Liq was formed at a thickness of 2 nm, and aluminum (Al) was vapor-deposited at a thickness of 100 nm to form a cathode, thereby producing an organic electroluminescent device (EL device 7).

(Example 17, Comparative Example 3) Formation of Organic Electroluminescent Devices using Compound C15 or Comparative Compound 2

Organic Electroluminescent devices (EL device 8, Comparative EL device 2) were formed in the same manner as in Example 16, except that Compound C15 or Comparative Compound 2 was used in place of Compound C14.

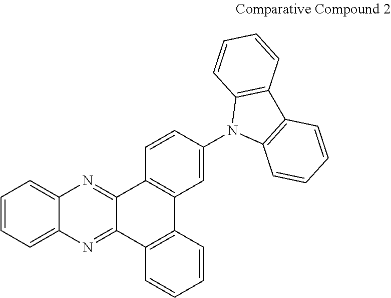 [SMILES: c1ccc2nc3c4ccc(-n5c6ccccc6c6ccccc65)cc4c4ccccc4c3nc2c1]

### Evaluation of EL Device

EL device 7 formed using Compound C14, EL device 8 formed using Compound C15, and Comparative EL device 2 formed using Comparative Compound 2 were continuously driven at 50 mA, and the time taken until the luminance became 95%, LT95 was measured. LT95 of each device is expressed as a relative value, when LT95 of Comparative EL Device 2 is defined as 1. In addition, the drive voltage at 15.4 mA/cm² of each device was measured, and expressed as a relative value based on the drive voltage of Comparative EL device 2. The results are shown in Table 46.

TABLE 46

| | | | | Drive |
|---|---|---|---|---|
| | | | LT95 | Voltage |
| Example | EL Device | Compound | (Relative | (Relative |
| No. | No. | No. | Value) | Value) |
| Example 16 | EL Device 7 | Compound C14 | 4.5 times | −0.23 V |
| Example 17 | EL Device 8 | Compound C15 | 5.0 times | −0.21 V |
| Comparative | Comparative | Comparative | 1 | 0 |
| Example 3 | EL Device 2 | Compound 2 | | |

From the results, it is known that, using the compound represented by the general formula (1) as a host material of the light emitting layer, EL devices having a low drive voltage and good durability can be provided.

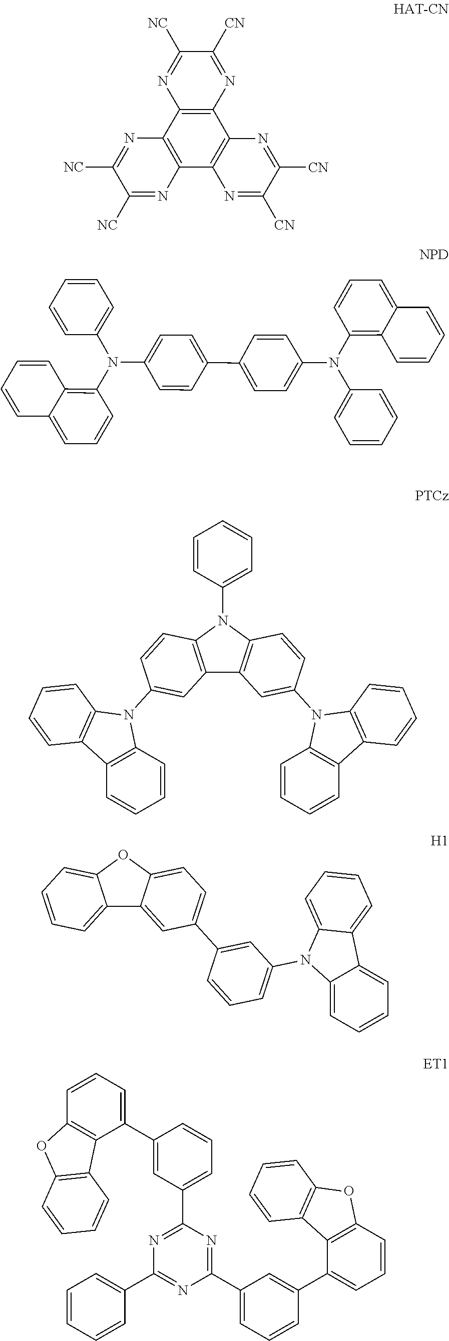

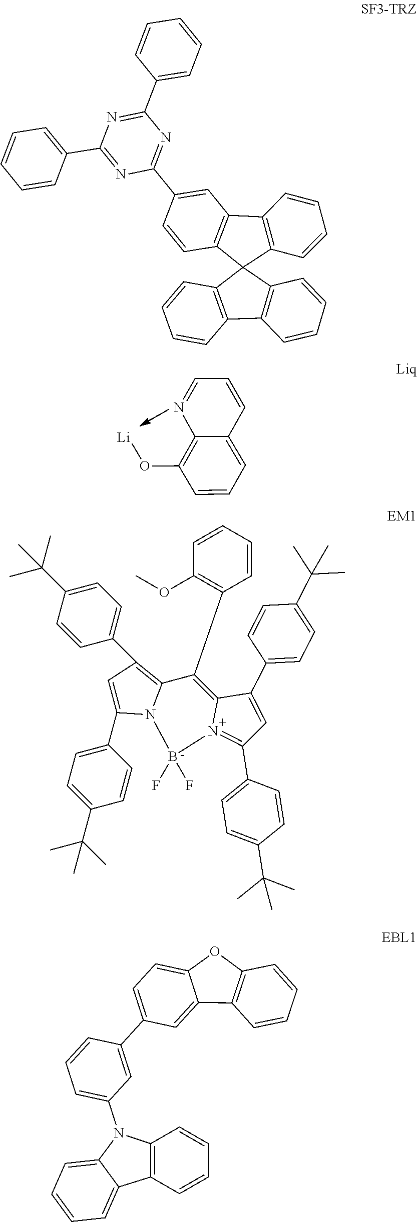

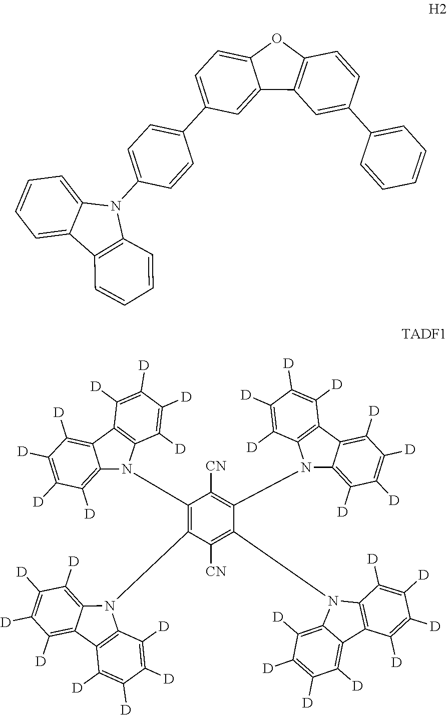

1. A compound represented by the following general formula (1): 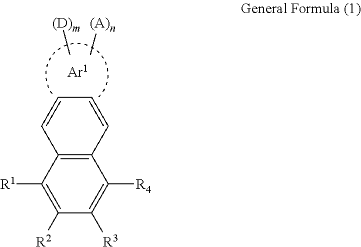
wherein Ar¹ represents a cyclic structure, and represents a benzene ring, a naphthalene ring, an anthracene ring, or a phenanthrene ring;
D represents a donor group, and at least one D is a group represented by the following general formula (2);
A represents one or a combination of two or more groups selected from the group consisting of a cyano group, a phenyl group, a pyrimidyl group, a triazyl group and an alkyl group, but excepting a substituted alkyl group; and
m represents 1, 2 or 3, n represents 0, 1 or 2;
when m is 2 or 3, plural D's can be the same or different;
when n is 2, two A's can be the same or different;
R¹ to R⁴ each independently represent a hydrogen atom, a deuterium atom, or one or a combination of two or more groups selected from the group consisting of an alkyl group, an aryl group, a heteroaryl group and a cyano group;
R¹ and R², and R³ and R⁴ each can bond to each other to form a cyclic structure selected from the group consisting of a benzene ring, a naphthalene ring and a pyridine ring, and the formed cyclic structure can be substituted with one or a combination of two or more groups selected from the group consisting of an alkyl group, an aryl group, a heteroaryl group and a cyano group; 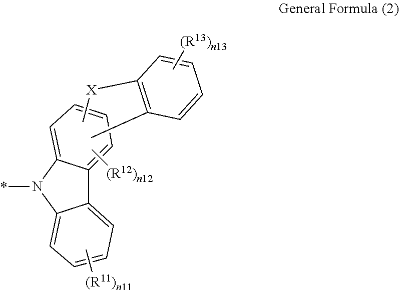
wherein X represents O, S or N—R¹⁴;
R¹¹ to R¹³ each independently represent a deuterium atom, or a substituent;
R¹⁴ represents an aryl group optionally substituted with one or more selected from the group consisting of a deuterium atom, an alkyl group and an aryl group, or an alkyl group optionally substituted with one or more selected from the group consisting of a deuterium atom and an aryl group;
R¹¹ to R¹³ do not bond to any of R¹¹ to R¹⁴ to form a cyclic structure; and
n11 and n13 each independently represent an integer of 0 to 4; n12 represents an integer of 0 to 2.

2. The compound according to claim 1, represented by the following general formula (3): 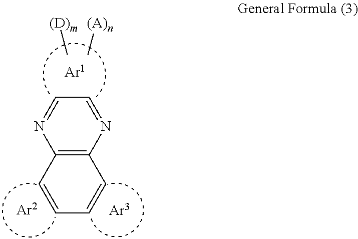
wherein Ar¹ represents a cyclic structure, and represents a benzene ring, a naphthalene ring, an anthracene ring, or a phenanthrene ring;
D represents a donor group, and at least one D is a group represented by the above-mentioned general formula (2);
A represents one or a combination of two or more groups selected from the group consisting of a cyano group, a phenyl group, a pyrimidyl group, a triazyl group and an alkyl group, but excepting a substituted alkyl group;
m represents 1, 2 or 3;
n represents 0, 1 or 2;
when m is 2 or 3, plural D's can be the same or different;
when n is 2, two A's can be the same or different; and
Ar² and Ar³ can each independently form a cyclic structure selected from the group consisting of a benzene ring, a naphthalene ring and a pyridine ring, and the formed cyclic structure can be substituted with one or a combination of two or more groups selected from the group consisting of an alkyl group, an aryl group, a heteroaryl group and a cyano group.

3. The compound according to claim 1, having a skeleton of any of the following: 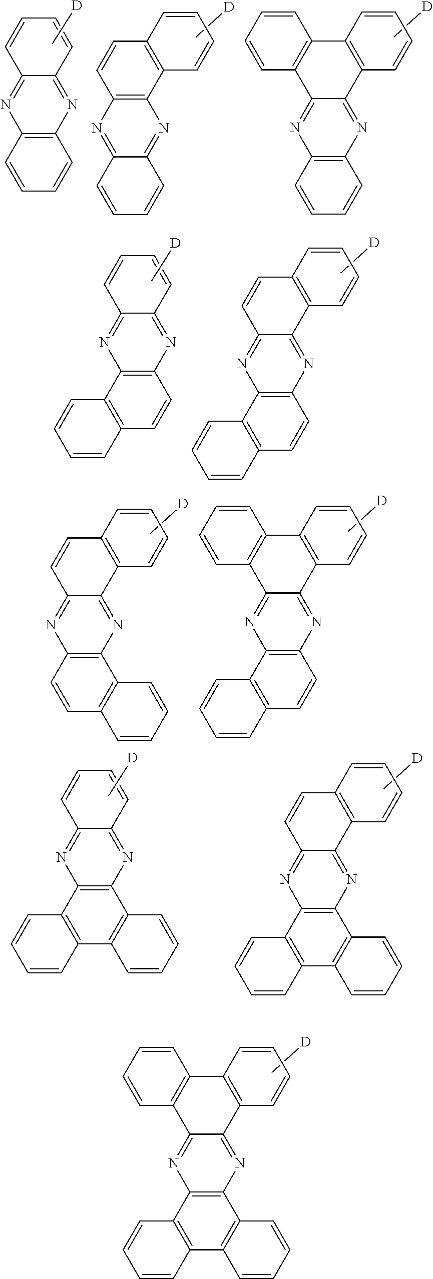
wherein the above skeletons each can have a substituent within the range of the general formula (1), but any further ring is not fused with the skeletons.

4. The compound according to claim 1, represented by any of the following general formulae (4a) to (4g): 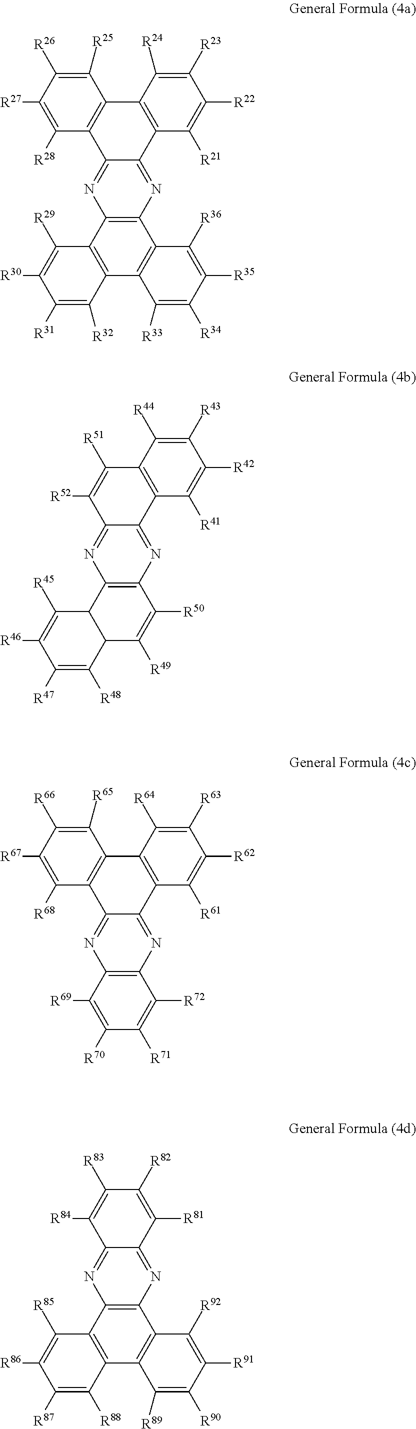 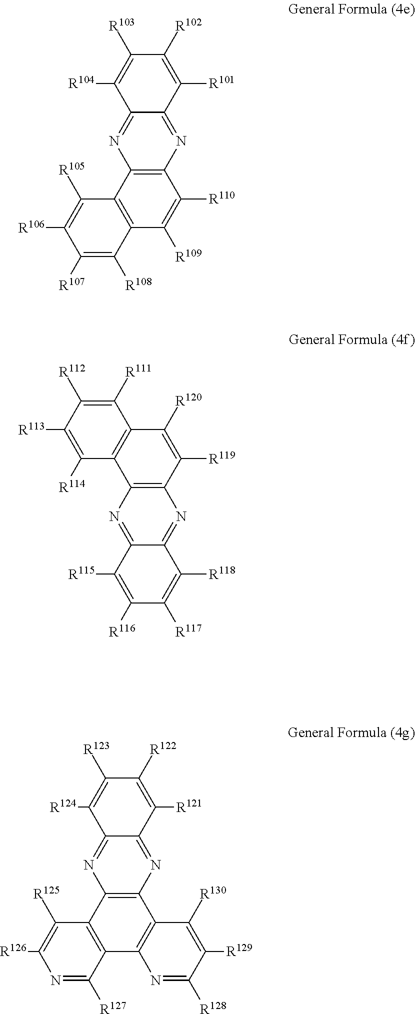
wherein R²¹ to R²⁸, R⁴¹ to R⁴⁴, R⁵¹, R⁵², R⁶¹ to R⁶⁸, R⁸¹ to R⁸⁴, R¹⁰¹ to R¹⁰⁴, R¹¹¹ to R¹¹⁴, R¹¹⁹, R¹²⁰ and R¹²¹ to R¹²⁴ each independently represent a hydrogen atom, a deuterium atom, D or A;
provided that 1 to 3 of R²¹ to R²⁸ are D, and 0 to 2 are A; 1 to 3 of R⁴¹ to R⁴⁴, R⁵¹ and R⁵² are D, and 0 to 2 are A; 1 to 3 of R⁶¹ to R⁶⁸ are D, and 0 to 2 are A; 1 to 3 of R⁸¹ to R⁸⁴ are D, and 0 to 2 are A; 1 to 3 of R¹⁰¹ to R¹⁰⁴ are D and 0 to 2 are A; 1 to 3 of R¹¹¹ to R¹¹⁴, R¹¹⁹ and R¹²⁰ are D, and 0 to 2 are A, 1 to 3 of R¹²¹ to R¹²⁴ are D, and 0 to 2 are A; and
R²⁹ to R³⁶, R⁴⁵ to R⁵⁰, R⁶⁹ to R⁷², R⁸⁵ to R⁹², R¹⁰⁵ to R¹¹⁰, R¹¹⁵ to R¹¹⁸, and R¹²⁵ to R¹³⁰ each independently represent a hydrogen atom, a deuterium atom, or one or a combination of two or more groups selected from the group consisting of an alkyl group, an aryl group and a cyano group.

5. The compound according to claim 1, wherein n is 0.

6-7. (canceled)

8. A film comprising the compound of claim 1.

9. An organic semiconductor device comprising the compound of claim 1.

10. An organic light emitting device comprising the compound of claim 1.

11. The organic light emitting device according to claim 10, wherein the device has a layer containing the compound and the layer also contains a host material.

12. The organic light emitting device according to claim 11, wherein the layer containing the compound also contains a delayed fluorescent material in addition to the host material, and the lowest excited singlet energy of the delayed fluorescent material is lower than that of the host material and higher than that of the compound.

13. The organic light emitting device according to claim 10, wherein the device has a layer containing the compound, and the layer also contains a light emitting material having a structure different from that of the compound.

14. The organic light emitting device according to claim 10, wherein the amount of light emitted from the compound is the largest among the materials contained in the device.

15. The organic light emitting device according to claim 13, wherein the amount of light emitted from the light emitting material is larger than the amount of light emitted from the compound.

16. The organic light emitting device according to claim 10, which emits delayed fluorescence.
